(19) United States
(12) Patent Application Publication
(10) Pub. No.: US 2025/0313641 A1
(43) Pub. Date: Oct. 9, 2025

(54) AGONISTIC ANTI-IL-2RBG HEAVY-CHAIN ANTIBODIES

(71) Applicant: AMGEN INC., Thousand Oaks, CA (US)

(72) Inventors: Jeremy KING, Baldwin Park, CA (US); Kevin GRAHAM, Thousand Oaks, CA (US); Pavel BONDARENKO, Thousand Oaks, CA (US); Shannon HOWELL, Newbury Park, CA (US); Mark KROENKE, Newbury Park, CA (US); Deepali SAWANT, Newark, CA (US)

(73) Assignee: AMGEN INC., Thousand Oaks, CA (US)

(21) Appl. No.: 19/172,869

(22) Filed: Apr. 8, 2025

Related U.S. Application Data
(60) Provisional application No. 63/631,729, filed on Apr. 9, 2024.
(60) Provisional application No. 63/735,080, filed on Dec. 17, 2024.

(51) Int. Cl. C07K 16/28; A61K 9/00; A61K 39/00; A61P 35/00

(52) U.S. Cl. CPC C07K 16/2866; A61K 9/0019; A61P 35/00; C07K 16/2809; A61K 2039/507; A61K 2039/54; C07K 2317/52; C07K 2317/565

(57) ABSTRACT

Disclosed herein are heavy-chain antibodies having activity as agonists of the dimeric interleukin-2 receptor, pharmaceutical compositions comprising the heavy-chain antibodies, and methods of treating certain disorders, such as cancer, including but not limited to, small cell lung cancer. Also disclosed herein are methods of treating DLL3-expressing cancers comprising the administration of a T-cell engaging molecule that binds to DLL3 and an IL-2-based therapy.

### CROSS-REFERENCE TO RELATED APPLICATIONS

This application claims the benefit of priority to U.S. Provisional Patent Application No. 63/631,729, filed Apr. 9, 2024, and U.S. Provisional Patent Application No. 63/735,080, filed Dec. 17, 2024.

### SUBMISSION OF SEQUENCE LISTING

The content of the following Sequence Listing XML is incorporated herein by reference in its entirety: file name: 10880-SL-040725.xml, date created: Apr. 7, 2025; size: 121,999 bytes.

### FIELD

The present disclosure provides heavy-chain antibodies having activity as agonists of the dimeric interleukin-2 (IL-2) receptor. This disclosure also provides pharmaceutical compositions comprising the heavy-chain antibodies, uses, and methods of treating certain disorders, such as cancer, including, but not limited to, DLL3-expressing cancers, such as, for example, small cell lung cancer. Additionally, the present disclosure provides methods of treating DLL3-expressing cancers comprising the administration of a T-cell engaging molecule that binds to DLL3 (e.g., tarlatamab) and an IL-2-based therapy, including, but not limited to, Proleukin® (aldesleukin), an IL-2 mutein, a non-a IL-2 molecule (e.g., bempegaldesleukin), or an agonistic anti-IL2RBG heavy-chain antibody disclosed herein.

### BACKGROUND

The pleiotropic cytokine interleukin-2 (IL-2) is a key regulator of immune cells, inducing both effector T-cell and natural killer (NK) cell proliferation, as well as the proliferation of immunosuppressive regulatory T (T-reg) cells. IL-2 therapy represents a potential strategy for transforming “cold tumors” into treatment-responsive “hot tumors” by expanding and maintaining the population of effector T-cells at a tumor site. The ability to harness the immune system against tumors has been previously established, with IL-2 being one of the first recombinant cytokine proteins to be FDA-approved for the treatment of cancer. Lotze et al., Journal of Immunology 135 (4), 2865-75 (1985); Rosenberg, S. A. J Immunol 192, 5451-58 (2014). Specifically, high-dose recombinant IL-2 (Proleukin®) was developed and approved for the treatment of metastatic melanoma and metastatic renal cell carcinoma, with durable responses observed in 7-12% of patients. McDermott, D. F. et al., J Clin Oncol 23, 133-141 (2004); Payne, R. et al., J Immunother Cancer 2, 13 (2014); Atkins, M. B. et al., J Clin Oncol 17, 2105-2105 (1999); Rosenberg, S. A. et al., Ann Surg 228, 307-319 (1998). However, despite having potent immune-activating activity and the potential to induce durable tumor-regression in cancer patients, the success of wild-type IL-2 cytokine as an immunotherapeutic has been limited by adverse events and poor pharmacokinetic properties. Specifically, Proleukin® is associated with severe dose-limiting toxicities, including vascular leak syndrome, hypotension, and liver toxicities. These adverse effects are believed to be driven by the preferential activation of cells, such as T-reg cells and endothelial cells, that express the high-affinity, trimeric form of the IL-2 receptor (IL-2Raβγ). In contrast to the high-affinity trimeric form, the intermediate affinity dimeric form of the IL-2 receptor is only composed of the IL-2Rβ and IL-2Rγ subunits and is expressed on resting T-cells, CD8+ memory effector T-cells, and NK cells. Choudhry, H. et al, Biomed Res Int 2018, 1-7 (2018). The IL-2Rα subunit, which is only present in the trimeric form of the IL-2 receptor, is not required for downstream JAK-STAT signaling, but its association with IL-2Rβ and IL-2Rγ provides a 100-fold higher affinity to IL-2 compared to the heterodimeric receptor composed only of IL-2Rβ and IL-2Rγ.

### SUMMARY

One aspect of the disclosure provides a heavy-chain antibody comprising an Fc region that comprises an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering.

Another aspect of the disclosure provides a heavy-chain antibody comprising a first heavy chain variable region, a second heavy chain variable region, and an Fc region, wherein the first heavy chain variable region is connected to the Fc region by a first peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids, and the second heavy chain variable region is connected to the Fc region by a second peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids.

Yet another aspect of the disclosure provides a heavy-chain antibody comprising a first heavy chain variable (VH) region that binds to IL2RB, a second heavy chain variable region that binds to IL2RG, and an Fc region that comprises an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering.

Still another aspect of the disclosure provides a heavy-chain antibody comprising a first heavy chain variable (VH) region that binds to IL2RB, a second heavy chain variable region that binds to IL2RG, and an Fc region, wherein the first heavy chain variable region is connected to the Fc region by a first peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids, and the second heavy chain variable region is connected to the Fc region by a second peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids.

A further aspect of the disclosure provides a heavy-chain antibody comprising a first heavy chain that binds to IL2RB comprising the amino acid sequence of SEQ ID NO: 38 and a second heavy chain that binds to IL2RG comprising the amino acid sequence of SEQ ID NO: 39.

Another aspect of the disclosure provides a pharmaceutical composition comprising a heavy-chain antibody disclosed herein and a pharmaceutically acceptable excipient.

Yet another aspect of the disclosure provides a method of treating cancer in a subject in need of such treatment, the method comprising administering to the subject a therapeutically effective amount of a heavy-chain antibody disclosed herein or a pharmaceutical composition comprising a heavy-chain antibody disclosed herein. In some embodiments, the method further comprises administering to the subject a T-cell redirecting therapy (e.g., a bispecific T-cell engaging molecule, such as, e.g., tarlatamab).

Still another aspect of the disclosure provides a heavy-chain antibody disclosed herein for use as a medicament. Another aspect of the disclosure provides a heavy-chain antibody disclosed herein, or a pharmaceutical composition disclosed herein, for use in the treatment of cancer, optionally in combination with a T-cell redirecting therapy (e.g., a bispecific T-cell engaging molecule, such as, e.g., tarlatamab).

Yet another aspect of the disclosure provides a use of a heavy-chain antibody disclosed herein for the manufacture of a medicament for the treatment of cancer.

Another aspect of the disclosure provides a method of treating a DLL3-expressing cancer in a subject in need of such treatment, the method comprising administering to the subject a therapeutically effective amount of an IL-2-based therapy (e.g., an IL-2-based therapy that preferentially binds to the heterodimeric receptor composed only of IL-2Rβ and IL-2Rγ over the trimeric IL-2Raβγ form of the IL-2 receptor) and a T-cell redirecting therapy that binds to DLL3 (e.g., a bispecific T-cell engaging molecule that binds to DLL3, such as, e.g., tarlatamab). Still another aspect of the disclosure provides an IL-2-based therapy (e.g., a heavy-chain antibody disclosed herein), or a pharmaceutical composition comprising the same, for use in the treatment of a DLL3-expressing cancer, in combination with a T-cell redirecting therapy that binds to DLL3 (e.g., a bispecific T-cell engaging molecule that binds to DLL3, such as, e.g., tarlatamab). Yet another aspect of the disclosure provides a use of an IL-2-based therapy (e.g., a heavy-chain antibody disclosed herein) for the manufacture of a medicament for the treatment of a DLL3-expressing cancer, wherein the medicament is adapted for administration in combination with a T-cell redirecting therapy that binds to DLL3 (e.g., a bispecific T-cell engaging molecule that binds to DLL3, such as, e.g., tarlatamab).

Further aspects and advantages will be apparent to those of ordinary skill in the art from a review of the following detailed description. The description hereafter includes specific cases, embodiments, and examples with the understanding that the disclosure is illustrative and is not intended to limit the features of the present disclosure to the specific cases, embodiments, and examples described herein. Illustratively, some example embodiments of the present disclosure include, but are not limited to, the following embodiments E1-E216.

E1. A heavy-chain antibody comprising:
a first heavy chain variable (VH) region that binds to IL2RB comprising:
(1) (a) a VH complementarity determining region one (CDR1) comprising the amino acid sequence:

(SEQ ID NO: 26)
G G S I S S S X1 W,

wherein X1 is D or N;
(b) a VH CDR2 comprising the amino acid sequence:

(SEQ ID NO: 27)
I X2 H S G S T,

wherein X2 is D or S; and
(c) a VH CDR3 comprising the amino acid sequence:

(SEQ ID NO: 28)
X3 R G X4 W E L X5 D A F D I,

wherein X3 is G or A; X4 is S or Q; and X5 is S or T; or
(2) (a) a VH CDR1 comprising the amino acid sequence:

(SEQ ID NO: 29)
G F T F S X1 Y G,

wherein X1 is S or T;
(b) a VH CDR2 comprising the amino acid sequence:

(SEQ ID NO: 30)
I S Y D G S N X2,

wherein X2 is K or R; and
(c) a VH CDR3 comprising the amino acid sequence:

(SEQ ID NO: 31)
A R D L D Y D X3 L T G D P V G G F D I,

wherein X3 is V or I;
a second heavy chain variable region that binds to IL2RG comprising:
(1) (a) a VH CDR1 comprising the amino acid sequence:

(SEQ ID NO: 32)
G F X1 X2 X3 X4 Y Y,

wherein X1 is T or I; X2 is For V; X3 is S, N, or G; and X4 is D or N;
(b) a VH CDR2 comprising the amino acid sequence:

(SEQ ID NO: 33)
I S X5 S G X6 X7 I,

wherein X5 is S or N; X6 is D, S, G, or N; and X7 is T or I; and
(c) a VH CDR3 comprising the amino acid sequence ARGDAVSITGDY (SEQ ID NO: 20); or
(2) a VH CDR1 comprising the amino acid sequence of SEQ ID NO: 15, a VH CDR2 comprising the amino acid sequence of SEQ ID NO: 19, and a VH CDR3 comprising the amino acid sequence of SEQ ID NO: 21; and
an Fc region comprising an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering.

E2. The heavy-chain antibody of E1, wherein the Fc region comprises an amino acid sequence that is at least 95% identical to the amino acid sequence of SEQ ID NO: 34.

E3. The heavy-chain antibody of E1, wherein the Fc region comprises a first polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering.

E4. The heavy-chain antibody of E3, wherein the Fc region further comprises a second polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

E5. The heavy-chain antibody of any one of E1-E4, wherein the Fc region comprises a first polypeptide chain comprising an amino acid sequence that is at least 95% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering.

E6. The heavy-chain antibody of any one of E3-E5, wherein the Fc region further comprises a second polypeptide chain comprising an amino acid sequence that is at least 95% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

E7. The heavy-chain antibody of E1, wherein the Fc region comprises:
a first polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering; and
a second polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

E8. The heavy-chain antibody of claim E7, wherein the first polypeptide chain comprises an amino acid sequence that is at least 95% identical to the amino acid sequence of SEQ ID NO: 34, and the second polypeptide chain comprises an amino acid sequence that is at least 95% identical to the amino acid sequence of SEQ ID NO: 34.

E9. The heavy-chain antibody of any one of E3-E8, wherein the first polypeptide chain comprises the amino acid sequence of SEQ ID NO: 35.

E10. The heavy-chain antibody of any one of E4-E9, wherein the second polypeptide chain comprises the amino acid sequence of SEQ ID NO: 36.

E11. The heavy-chain antibody of any one of E4-E10, wherein the first polypeptide chain comprises the amino acid sequence of SEQ ID NO: 35, and the second polypeptide chain comprises the amino acid sequence of SEQ ID NO: 36.

E12. The heavy-chain antibody of any one of E1-E11, wherein the first heavy chain variable region is connected to the Fc region by a first peptide linker.

E13. The heavy-chain antibody of any one of E1-E12, wherein the second heavy chain variable region is connected to the Fc region by a second peptide linker.

E14. The heavy-chain antibody of any one of E1-E13, wherein the first heavy chain variable region is connected to the Fc region by a first peptide linker, and the second heavy chain variable region is connected to the Fc region by a second peptide linker.

E15. The heavy-chain antibody of any one of E7-E14, wherein:
the first heavy chain variable region is connected to the first polypeptide chain of the Fc region by a first peptide linker; and
the second heavy chain variable region is connected to the second polypeptide chain of the Fc region by a second peptide linker.

E16. The heavy-chain antibody of any one of E7-E14, wherein:
the first heavy chain variable region is connected to the second polypeptide chain of the Fc region by a first peptide linker; and
the second heavy chain variable region is connected to the first polypeptide chain of the Fc region by a second peptide linker.

E17. The heavy-chain antibody of any one of E12-E16, wherein the first peptide linker is a poly-Gly linker.

E18. The heavy-chain antibody of any one of E13-E17, wherein the second peptide linker is a poly-Gly linker.

E19. The heavy-chain antibody of any one of E13-E18, wherein the first peptide linker and the second peptide linker are independently poly-Gly linkers.

E20. The heavy-chain antibody of any one of E12-E19, wherein the first peptide linker comprises the amino acid sequence of GGGG (SEQ ID NO: 37).

E21. The heavy-chain antibody of any one of E13-E20, wherein the second peptide linker comprises the amino acid sequence of GGGG (SEQ ID NO: 37).

E22. The heavy-chain antibody of any one of E13-E21, wherein the first peptide linker and the second peptide linker both comprise the amino acid sequence of GGGG (SEQ ID NO: 37).

E23. A heavy-chain antibody comprising:
a first heavy chain variable (VH) region that binds to IL2RB comprising:
(1) (a) a VH complementarity determining region one (CDR1) comprising the amino acid sequence:

(SEQ ID NO: 26)
G G S I S S S X1 W,

wherein X1 is D or N;
(b) a VH CDR2 comprising the amino acid sequence:

(SEQ ID NO: 27)
I X2 H S G S T,

wherein X2 is D or S; and
(c) a VH CDR3 comprising the amino acid sequence:

(SEQ ID NO: 28)
X3 R G X4 W E L X5 D A F D I,

wherein X3 is G or A; X4 is S or Q; and X5 is S or T; or
(2) (a) a VH CDR1 comprising the amino acid sequence:

(SEQ ID NO: 29)
G F T F S X1 Y G,

wherein X1 is S or T;
(b) a VH CDR2 comprising the amino acid sequence:

(SEQ ID NO: 30)
I S Y D G S N X2,

wherein X2 is K or R; and
(c) a VH CDR3 comprising the amino acid sequence:

(SEQ ID NO: 31)
A R D L D Y D X3 L T G D P V G G F D I,

wherein X3 is V or I;
a second heavy chain variable region that binds to IL2RG comprising:

(1) (a) a VH CDR1 comprising the amino acid sequence:

(SEQ ID NO: 32)
G F X1 X2 X3 X4 Y Y,

wherein X1 is T or I; X2 is For V; X3 is S, N, or G; and X4 is D or N;
(b) a VH CDR2 comprising the amino acid sequence:

(SEQ ID NO: 33)
I S X5 S G X6 X7 I,

wherein X5 is S or N; X6 is D, S, G, or N; and X7 is T or I; and
(c) a VH CDR3 comprising the amino acid sequence ARGDAVSITGDY (SEQ ID NO: 20); or
(2) a VH CDR1 comprising the amino acid sequence of SEQ ID NO: 15, a VH CDR2 comprising the amino acid sequence of SEQ ID NO: 19, and a VH CDR3 comprising the amino acid sequence of SEQ ID NO: 21; and
an Fc region, wherein the first heavy chain variable region is connected to the Fc region by a first peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids, and the second heavy chain variable region is connected to the Fc region by a second peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids.

E24. The heavy-chain antibody of E23, wherein the first peptide linker comprises between 4 and 10 amino acids.

E25. The heavy-chain antibody of E23 or E24, wherein the second peptide linker comprises between 4 and 10 amino acids.

E26. The heavy-chain antibody of any one of E23-E25, wherein the first peptide linker comprises between 4 and 10 amino acids, and the second peptide linker comprises between 4 and 10 amino acids.

E27. The heavy-chain antibody of any one of E23-E26, wherein the first peptide linker comprises 4 amino acids.

E28. The heavy-chain antibody of any one of E23-E27, wherein the second peptide linker comprises 4 amino acids.

E29. The heavy-chain antibody of any one of E23-E28, wherein the first peptide linker comprises 4 amino acids, and the second peptide linker comprises 4 amino acids.

E30. The heavy-chain antibody of any one of E23-E29, wherein the first peptide linker is a flexible linker.

E31. The heavy-chain antibody of any one of E23-E30, wherein the second peptide linker is a flexible linker.

E32. The heavy-chain antibody of any one of E23-E31, wherein the first peptide linker and the second peptide linker are both flexible linkers.

E33. The heavy-chain antibody of any one of E23-E32, wherein the first peptide linker and/or the second peptide linker comprises the amino acid sequence of (Gly-Gly-Gly-Gly-Ser) m, wherein m is a number in the range of 1 to 5 (SEQ ID NO: 40), or the amino acid sequence of (Gly-Gly-Gly-Gly-Gln) n, wherein n is a number in the range of 1 to 5 (SEQ ID NO: 82).

E34. The heavy-chain antibody of any one of E23-E33, wherein the first peptide linker and the second peptide linker independently comprise the amino acid sequence of (Gly-Gly-Gly-Gly-Ser) m, wherein m is a number in the range of 1 to 5 (SEQ ID NO: 40), or the amino acid sequence of (Gly-Gly-Gly-Gly-Gln) n, wherein n is a number in the range of 1 to 5 (SEQ ID NO: 82).

E35. The heavy-chain antibody of any one of E23-E33, wherein the first peptide linker and/or the second peptide linker comprises the amino acid sequence of (Gly) p, wherein p is a number in the range of 4 to 10 (SEQ ID NO: 41).

E36. The heavy-chain antibody of any one of E23-E32, wherein the first peptide linker and the second peptide linker independently comprise the amino acid sequence of (Gly) p, wherein p is a number in the range of 4 to 10 (SEQ ID NO: 41).

E37. The heavy-chain antibody of any one of E23-E32, wherein the first peptide linker comprises the amino acid sequence of GGGG (SEQ ID NO: 37).

E38. The heavy-chain antibody of any one of E23-E32, wherein the second peptide linker comprises the amino acid sequence of GGGG (SEQ ID NO: 37).

E39. The heavy-chain antibody of any one of E23-E32, wherein the first peptide linker and the second peptide linker both comprise the amino acid sequence of GGGG (SEQ ID NO: 37).

E40. The heavy-chain antibody of any one of E23-E32, wherein the first peptide linker only comprises glycine, serine, glutamine, and threonine amino acids.

E41. The heavy-chain antibody of any one of E23-E32, wherein the second peptide linker only comprises glycine, serine, glutamine, and threonine amino acids.

E42. The heavy-chain antibody of any one of E23-E32, wherein the first peptide linker and the second peptide linker both only comprise glycine, serine, glutamine, and threonine amino acids.

E43. The heavy-chain antibody of any one of E23-E42, wherein the Fc region comprises an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering.

E44. The heavy-chain antibody of any one of E23-E43, wherein the Fc region comprises an amino acid sequence that is at least 95% identical to the amino acid sequence of SEQ ID NO: 34.

E45. The heavy-chain antibody of any one of E23-E42, wherein the Fc region comprises:
a first polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering; and
a second polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

E46. The heavy-chain antibody of E45, wherein the first polypeptide chain comprises an amino acid sequence that is at least 95% identical to the amino acid sequence of SEQ ID NO: 34, and the second polypeptide chain comprises an amino acid sequence that is at least 95% identical to the amino acid sequence of SEQ ID NO: 34.

E47. The heavy-chain antibody of E45 or E46, wherein the first polypeptide chain comprises the amino acid sequence of SEQ ID NO: 35, and the second polypeptide chain comprises the amino acid sequence of SEQ ID NO: 36.

E48. The heavy-chain antibody of E23, wherein:
the Fc region comprises a first polypeptide chain comprising the amino acid sequence of SEQ ID NO: 35 and a second polypeptide chain comprising the amino acid sequence of SEQ ID NO: 36; and
the first peptide linker and the second peptide linker are independently poly-Gly linkers.

E49. The heavy-chain antibody of E23 or E48, wherein:
the Fc region comprises a first polypeptide chain comprising the amino acid sequence of SEQ ID NO: 35 and a second polypeptide chain comprising the amino acid sequence of SEQ ID NO: 36; and
the first peptide linker and the second peptide linker both comprise the amino acid sequence of GGGG (SEQ ID NO: 37).

E50. The heavy-chain antibody of any one of E1-E49, wherein the VH CDR1, the VH CDR2, and the VH CDR3 of the first VH region comprise the amino acid sequences of SEQ ID NO: 26, SEQ ID NO: 27, and SEQ ID NO: 28, respectively.

E51. The heavy-chain antibody of any one of E1-E49, wherein the VH CDR1, the VH CDR2, and the VH CDR3 of the first VH region comprise the amino acid sequences of SEQ ID NO: 29, SEQ ID NO: 30, and SEQ ID NO: 31, respectively.

E52. The heavy-chain antibody of any one of E1-E51, wherein the VH CDR1, the VH CDR2, and the VH CDR3 of the second VH region comprise the amino acid sequences of SEQ ID NO: 32, SEQ ID NO: 33, and SEQ ID NO: 20, respectively.

E53. The heavy-chain antibody of any one of E1-E51, wherein the VH CDR1, the VH CDR2, and the VH CDR3 of the second VH region comprise the amino acid sequences of SEQ ID NO: 15, SEQ ID NO: 19, and SEQ ID NO: 21, respectively.

E54. The heavy-chain antibody of any one of E1-E49, wherein:
the VH CDR1, the VH CDR2, and the VH CDR3 of the first VH region comprise the amino acid sequences of SEQ ID NO: 26, SEQ ID NO: 27, and SEQ ID NO: 28, respectively; and
the VH CDR1, the VH CDR2, and the VH CDR3 of the second VH region comprise the amino acid sequences of SEQ ID NO: 32, SEQ ID NO: 33, and SEQ ID NO: 20, respectively.

E55. The heavy-chain antibody of any one of E1-E49, wherein:
the VH CDR1, the VH CDR2, and the VH CDR3 of the first VH region comprise the amino acid sequences of SEQ ID NO: 26, SEQ ID NO: 27, and SEQ ID NO: 28, respectively; and
the VH CDR1, the VH CDR2, and the VH CDR3 of the second VH region comprise the amino acid sequences of SEQ ID NO: 15, SEQ ID NO: 19, and SEQ ID NO: 21, respectively.

E56. The heavy-chain antibody of any one of E1-E49, wherein:
the VH CDR1, the VH CDR2, and the VH CDR3 of the first VH region comprise the amino acid sequences of SEQ ID NO: 29, SEQ ID NO: 30, and SEQ ID NO: 31, respectively; and
the VH CDR1, the VH CDR2, and the VH CDR3 of the second VH region comprise the amino acid sequences of SEQ ID NO: 32, SEQ ID NO: 33, and SEQ ID NO: 20, respectively.

E57. The heavy-chain antibody of any one of E1-E49, wherein:
the VH CDR1, the VH CDR2, and the VH CDR3 of the first VH region comprise the amino acid sequences of SEQ ID NO: 29, SEQ ID NO: 30, and SEQ ID NO: 31, respectively; and
the VH CDR1, the VH CDR2, and the VH CDR3 of the second VH region comprise the amino acid sequences of SEQ ID NO: 15, SEQ ID NO: 19, and SEQ ID NO: 21, respectively.

E58. The heavy-chain antibody of E1-E49, wherein:
(a) the VH CDR1, the VH CDR2, and the VH CDR3 of the first VH region comprise the amino acid sequences of SEQ ID NO: 1 or SEQ ID NO: 2; SEQ ID NO: 4 or SEQ ID NO: 5; and SEQ ID NO: 7, SEQ ID NO: 8, or SEQ ID NO: 9, respectively; or
(b) the VH CDR1, the VH CDR2, and the VH CDR3 of the first VH region comprise the amino acid sequences of SEQ ID NO: 3, SEQ ID NO: 6, and SEQ ID NO: 10, respectively.

E59. The heavy-chain antibody of E58, wherein the VH CDR1, the VH CDR2, and the VH CDR3 of the first VH region comprise the amino acid sequences of SEQ ID NO: 1 or SEQ ID NO: 2; SEQ ID NO: 4 or SEQ ID NO: 5; and SEQ ID NO: 7, SEQ ID NO: 8, or SEQ ID NO: 9, respectively.

E60. The heavy-chain antibody of E58, wherein the VH CDR1, the VH CDR2, and the VH CDR3 of the first VH region comprise the amino acid sequences of SEQ ID NO: 3, SEQ ID NO: 6, and SEQ ID NO: 10, respectively.

E61. The heavy-chain antibody of any one of E1-E49, wherein the VH CDR1, the VH CDR2, and the VH CDR3 of the first VH region comprise the amino acid sequences of:
(a) SEQ ID NO: 1, SEQ ID NO: 4, and SEQ ID NO: 7, respectively; or
(b) SEQ ID NO: 1, SEQ ID NO: 4, and SEQ ID NO: 8, respectively; or
(c) SEQ ID NO: 2, SEQ ID NO: 5, and SEQ ID NO: 9, respectively; or
(d) SEQ ID NO: 3, SEQ ID NO: 6, and SEQ ID NO: 10, respectively.

E62. The heavy-chain antibody of E61, wherein the VH CDR1, the VH CDR2, and the VH CDR3 of the first VH region comprise the amino acid sequences of:
(a) SEQ ID NO: 1, SEQ ID NO: 4, and SEQ ID NO: 7, respectively; or
(b) SEQ ID NO: 1, SEQ ID NO: 4, and SEQ ID NO: 8, respectively; or
(c) SEQ ID NO: 2, SEQ ID NO: 5, and SEQ ID NO: 9, respectively.

E63. The heavy-chain antibody of any one of E1-E49, wherein the first VH region comprises an amino acid sequence that is at least 95% identical to any one of SEQ ID NOs: 11-14.

E64. The heavy-chain antibody of E63, wherein the first VH region comprises an amino acid sequence that is at least 95% identical to any one of SEQ ID NOs: 11-13.

E65. The heavy-chain antibody of any one of E1-E49, wherein the first VH region comprises an amino acid sequence selected from SEQ ID NOs: 11-14.

E66. The heavy-chain antibody of E65, wherein the first VH region comprises an amino acid sequence selected from SEQ ID NOs: 11-13.

E67. The heavy-chain antibody of any one of E1-E66, wherein:
(a) the VH CDR1, the VH CDR2, and the VH CDR3 of the second VH region comprise the amino acid sequences of SEQ ID NO: 15 or SEQ ID NO: 16; SEQ ID NO: 17 or SEQ ID NO: 18; and SEQ ID NO: 20, respectively; or
(b) the VH CDR1, the VH CDR2, and the VH CDR3 of the second VH region comprise the amino acid sequences of SEQ ID NO: 15, SEQ ID NO: 19, and SEQ ID NO: 21, respectively.

E68. The heavy-chain antibody of any one of E1-E67, wherein the VH CDR1, the VH CDR2, and the VH CDR3 of the second VH region comprise the amino acid sequences of SEQ ID NO: 15 or SEQ ID NO: 16; SEQ ID NO: 17 or SEQ ID NO: 18; and SEQ ID NO: 20, respectively.

E69. The heavy-chain antibody of any one of E1-E67, wherein the VH CDR1, the VH CDR2, and the VH CDR3 of the second VH region comprise the amino acid sequences of SEQ ID NO: 15, SEQ ID NO: 19, and SEQ ID NO: 21, respectively.

E70. The heavy-chain antibody of any one of E1-E66, wherein the VH CDR1, the VH CDR2, and the VH CDR3 of the second VH region comprise the amino acid sequences of:
(a) SEQ ID NO: 15, SEQ ID NO: 17, and SEQ ID NO: 20, respectively; or
(b) SEQ ID NO: 15, SEQ ID NO: 18, and SEQ ID NO: 20, respectively; or
(c) SEQ ID NO: 16, SEQ ID NO: 18, and SEQ ID NO: 20, respectively; or
(d) SEQ ID NO: 15, SEQ ID NO: 19, and SEQ ID NO: 21, respectively.

E71. The heavy-chain antibody of E70, wherein the VH CDR1, the VH CDR2, and the VH CDR3 of the second VH region comprise the amino acid sequences of:
(a) SEQ ID NO: 15, SEQ ID NO: 17, and SEQ ID NO: 20, respectively; or
(b) SEQ ID NO: 15, SEQ ID NO: 18, and SEQ ID NO: 20, respectively; or
(c) SEQ ID NO: 16, SEQ ID NO: 18, and SEQ ID NO: 20, respectively.

E72. The heavy-chain antibody of any one of E1-E66, wherein the second VH region comprises an amino acid sequence that is at least 95% identical to any one of SEQ ID NOs: 22-25.

E73. The heavy-chain antibody of E72, wherein the second VH region comprises an amino acid sequence that is at least 95% identical to any one of SEQ ID NOs: 22-24.

E74. The heavy-chain antibody of any one of E1-E66, wherein the second VH region comprises an amino acid sequence selected from SEQ ID NOs: 22-25.

E75. The heavy-chain antibody of E74, wherein the second VH region comprises an amino acid sequence selected from SEQ ID NOs: 22-24.

E76. The heavy-chain antibody of any one of E1-E49, wherein:
the VH CDR1, the VH CDR2, and the VH CDR3 of the first VH region comprise the amino acid sequences of SEQ ID NO: 1, SEQ ID NO: 4, and SEQ ID NO: 7, respectively; and
the VH CDR1, the VH CDR2, and the VH CDR3 of the second VH region comprise the amino acid sequences of SEQ ID NO: 15, SEQ ID NO: 17, and SEQ ID NO: 20, respectively.

E77. The heavy-chain antibody of any one of E1-E49 or E76, wherein the first VH region comprises an amino acid sequence that is at least 95% identical to SEQ ID NO: 11, and the second VH region comprises an amino acid sequence that is at least 95% identical to SEQ ID NO: 22.

E78. The heavy-chain antibody of any one of E1-E49, E76, or E77, wherein the first VH region comprises the amino acid sequence of SEQ ID NO: 11, and the second VH region comprises the amino acid sequence of SEQ ID NO: 22.

E79. The heavy-chain antibody of any one of E1-E49, wherein:
the VH CDR1, the VH CDR2, and the VH CDR3 of the first VH region comprise the amino acid sequences of SEQ ID NO: 1, SEQ ID NO: 4, and SEQ ID NO: 8, respectively; and
the VH CDR1, the VH CDR2, and the VH CDR3 of the second VH region comprise the amino acid sequences of SEQ ID NO: 15, SEQ ID NO: 18, and SEQ ID NO: 20, respectively.

E80. The heavy-chain antibody of any one of E1-E49 or E79, wherein the first VH region comprises an amino acid sequence that is at least 95% identical to SEQ ID NO: 12, and the second VH region comprises an amino acid sequence that is at least 95% identical to SEQ ID NO: 23.

E81. The heavy-chain antibody of any one of E1-E49, E79, or E80, wherein the first VH region comprises the amino acid sequence of SEQ ID NO: 12, and the second VH region comprises the amino acid sequence of SEQ ID NO: 23.

E82. The heavy-chain antibody of any one of E1-E49, wherein:
the VH CDR1, the VH CDR2, and the VH CDR3 of the first VH region comprise the amino acid sequences of SEQ ID NO: 2, SEQ ID NO: 5, and SEQ ID NO: 9, respectively; and
the VH CDR1, the VH CDR2, and the VH CDR3 of the second VH region comprise the amino acid sequences of SEQ ID NO: 15, SEQ ID NO: 18, and SEQ ID NO: 20, respectively.

E83. The heavy-chain antibody of any one of E1-E49 or E82, wherein the first VH region comprises an amino acid sequence that is at least 95% identical to SEQ ID NO: 13, and the second VH region comprises an amino acid sequence that is at least 95% identical to SEQ ID NO: 23.

E84. The heavy-chain antibody of any one of E1-E49, E82, or E83, wherein the first VH region comprises the amino acid sequence of SEQ ID NO: 13, and the second VH region comprises the amino acid sequence of SEQ ID NO: 23.

E85. The heavy-chain antibody of any one of E1-E49, wherein:
the VH CDR1, the VH CDR2, and the VH CDR3 of the first VH region comprise the amino acid sequences of SEQ ID NO: 1, SEQ ID NO: 4, and SEQ ID NO: 8, respectively; and
the VH CDR1, the VH CDR2, and the VH CDR3 of the second VH region comprise the amino acid sequences of SEQ ID NO: 16, SEQ ID NO: 18, and SEQ ID NO: 20, respectively.

E86. The heavy-chain antibody of any one of E1-E49 or E85, wherein the first VH region comprises an amino acid sequence that is at least 95% identical to SEQ ID NO: 12, and the second VH region comprises an amino acid sequence that is at least 95% identical to SEQ ID NO: 24.

E87. The heavy-chain antibody of any one of E1-E49, E85, or E86, wherein the first VH region comprises the amino acid sequence of SEQ ID NO: 12, and the second VH region comprises the amino acid sequence of SEQ ID NO: 24.

E88. The heavy-chain antibody of any one of E1-E49, wherein:
the VH CDR1, the VH CDR2, and the VH CDR3 of the first VH region comprise the amino acid sequences of SEQ ID NO: 1, SEQ ID NO: 4, and SEQ ID NO: 8, respectively; and
the VH CDR1, the VH CDR2, and the VH CDR3 of the second VH region comprise the amino acid sequences of SEQ ID NO: 15, SEQ ID NO: 19, and SEQ ID NO: 21, respectively.

E89. The heavy-chain antibody of any one of E1-E49 or E88, wherein the first VH region comprises an amino acid sequence that is at least 95% identical to SEQ ID NO: 12, and the second VH region comprises an amino acid sequence that is at least 95% identical to SEQ ID NO: 25.

E90. The heavy-chain antibody of any one of E1-E49, E88, or E89, wherein the first VH region comprises the amino acid sequence of SEQ ID NO: 12, and the second VH region comprises the amino acid sequence of SEQ ID NO: 25.

E91. A heavy-chain antibody comprising:
a first heavy chain variable (VH) region that binds to IL2RB in which the full set of VH complementarity determining regions (CDRs) 1, 2, and 3 (combined) is at least 95% identical to the VH CDRs 1, 2, and 3 of any one of SEQ ID NOs: 11-14;
a second VH region that binds to IL2RG in which the full set of VH CDRs 1, 2, and 3 (combined) is at least 95% identical to the VH CDRs 1, 2, and 3 of any one of SEQ ID NOs: 22-25; and
an Fc region comprising an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering.

E92. The heavy-chain antibody of E91, wherein the Fc region comprises an amino acid sequence that is at least 95% identical to the amino acid sequence of SEQ ID NO: 34.

E93. The heavy-chain antibody of E91, wherein the Fc region comprises a first polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering.

E94. The heavy-chain antibody of E93, wherein the Fc region further comprises a second polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

E95. The heavy-chain antibody of any one of E91-E94, wherein the Fc region comprises a first polypeptide chain comprising an amino acid sequence that is at least 95% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering.

E96. The heavy-chain antibody of any one of E93-E95, wherein the Fc region further comprises a second polypeptide chain comprising an amino acid sequence that is at least 95% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

E97. The heavy-chain antibody of E91, wherein the Fc region comprises:
a first polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering; and
a second polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

E98. The heavy-chain antibody of claim E97, wherein the first polypeptide chain comprises an amino acid sequence that is at least 95% identical to the amino acid sequence of SEQ ID NO: 34, and the second polypeptide chain comprises an amino acid sequence that is at least 95% identical to the amino acid sequence of SEQ ID NO: 34.

E99. The heavy-chain antibody of any one of E93-E98, wherein the first polypeptide chain comprises the amino acid sequence of SEQ ID NO: 35.

E100. The heavy-chain antibody of any one of E94-E99, wherein the second polypeptide chain comprises the amino acid sequence of SEQ ID NO: 36.

E101. The heavy-chain antibody of any one of E94-E100, wherein the first polypeptide chain comprises the amino acid sequence of SEQ ID NO: 35, and the second polypeptide chain comprises the amino acid sequence of SEQ ID NO: 36.

E102. The heavy-chain antibody of any one of E91-E101, wherein the first VH region is connected to the Fc region by a first peptide linker.

E103. The heavy-chain antibody of any one of E91-E102, wherein the second VH region is connected to the Fc region by a second peptide linker.

E104. The heavy-chain antibody of any one of E91-E103, wherein the first VH region is connected to the Fc region by a first peptide linker, and the second VH region is connected to the Fc region by a second peptide linker.

E105. The heavy-chain antibody of any one of E97-E104, wherein:
the first VH region is connected to the first polypeptide chain of the Fc region by a first peptide linker; and
the second VH region is connected to the second polypeptide chain of the Fc region by a second peptide linker.

E106. The heavy-chain antibody of any one of E97-E104, wherein:
the first VH region is connected to the second polypeptide chain of the Fc region by a first peptide linker; and
the second VH region is connected to the first polypeptide chain of the Fc region by a second peptide linker.

E107. The heavy-chain antibody of any one of E102-E106, wherein the first peptide linker is a poly-Gly linker.

E108. The heavy-chain antibody of any one of E103-E107, wherein the second peptide linker is a poly-Gly linker.

E109. The heavy-chain antibody of any one of E103-E108, wherein the first peptide linker and the second peptide linker are independently poly-Gly linkers.

E110. The heavy-chain antibody of any one of E102-E109, wherein the first peptide linker comprises the amino acid sequence of GGGG (SEQ ID NO: 37).

E111. The heavy-chain antibody of any one of E103-E110, wherein the second peptide linker comprises the amino acid sequence of GGGG (SEQ ID NO: 37).

E112. The heavy-chain antibody of any one of E103-E111, wherein the first peptide linker and the second peptide linker both comprise the amino acid sequence of GGGG (SEQ ID NO: 37).

E113. A heavy-chain antibody comprising:
a first heavy chain variable (VH) region that binds to IL2RB in which the full set of VH complementarity determining regions (CDRs) 1, 2, and 3 (combined) is at least 95% identical to the VH CDRs 1, 2, and 3 of any one of SEQ ID NOs: 11-14;
a second VH region that binds to IL2RG in which the full set of VH CDRs 1, 2, and 3 (combined) is at least 95% identical to the VH CDRs 1, 2, and 3 of any one of SEQ ID NOs: 22-25; and an Fc region, wherein the first heavy chain variable region is connected to the Fc region by a first peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids, and the second heavy chain variable region is connected to the Fc region by a second peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids.

E114. The heavy-chain antibody of E113, wherein the first peptide linker comprises between 4 and 10 amino acids.

E115. The heavy-chain antibody of E113 or E114, wherein the second peptide linker comprises between 4 and 10 amino acids.

E116. The heavy-chain antibody of any one of E113-E115, wherein the first peptide linker comprises between 4 and 10 amino acids, and the second peptide linker comprises between 4 and 10 amino acids.

E117. The heavy-chain antibody of any one of E113-E116, wherein the first peptide linker comprises 4 amino acids.

E118. The heavy-chain antibody of any one of E113-E117, wherein the second peptide linker comprises 4 amino acids.

E119. The heavy-chain antibody of any one of E113-E118, wherein the first peptide linker comprises 4 amino acids, and the second peptide linker comprises 4 amino acids.

E120. The heavy-chain antibody of any one of E113-E119, wherein the first peptide linker is a flexible linker.

E121. The heavy-chain antibody of any one of E113-E120, wherein the second peptide linker is a flexible linker.

E122. The heavy-chain antibody of any one of E113-E121, wherein the first peptide linker and the second peptide linker are both flexible linkers.

E123. The heavy-chain antibody of any one of E113-E122, wherein the first peptide linker and/or the second peptide linker comprises the amino acid sequence of (Gly-Gly-Gly-Gly-Ser) m, wherein m is a number in the range of 1 to 5 (SEQ ID NO: 40), or the amino acid sequence of (Gly-Gly-Gly-Gly-Gln) n, wherein n is a number in the range of 1 to 5 (SEQ ID NO: 82).

E124. The heavy-chain antibody of any one of E113-E123, wherein the first peptide linker and the second peptide linker independently comprise the amino acid sequence of (Gly-Gly-Gly-Gly-Ser) m, wherein m is a number in the range of 1 to 5 (SEQ ID NO: 40), or the amino acid sequence of (Gly-Gly-Gly-Gly-Gln) n, wherein n is a number in the range of 1 to 5 (SEQ ID NO: 82).

E125. The heavy-chain antibody of any one of E113-E123, wherein the first peptide linker and/or the second peptide linker comprises the amino acid sequence of (Gly) p, wherein p is a number in the range of 4 to 10 (SEQ ID NO: 41).

E126. The heavy-chain antibody of any one of E113-E122, wherein the first peptide linker and the second peptide linker independently comprise the amino acid sequence of (Gly) p, wherein p is a number in the range of 4 to 10 (SEQ ID NO: 41).

E127. The heavy-chain antibody of any one of E113-E122, wherein the first peptide linker comprises the amino acid sequence of GGGG (SEQ ID NO: 37).

E128. The heavy-chain antibody of any one of E113-E122, wherein the second peptide linker comprises the amino acid sequence of GGGG (SEQ ID NO: 37).

E129. The heavy-chain antibody of any one of E113-E122, wherein the first peptide linker and the second peptide linker both comprise the amino acid sequence of GGGG (SEQ ID NO: 37).

E130. The heavy-chain antibody of any one of E113-E122, wherein the first peptide linker only comprises glycine, serine, glutamine, and threonine amino acids.

E131. The heavy-chain antibody of any one of E113-E122, wherein the second peptide linker only comprises glycine, serine, glutamine, and threonine amino acids.

E132. The heavy-chain antibody of any one of E113-E122, wherein the first peptide linker and the second peptide linker both only comprise glycine, serine, glutamine, and threonine amino acids.

E133. The heavy-chain antibody of any one of E113-E132, wherein the Fc region comprises an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering.

E134. The heavy-chain antibody of any one of E113-E133, wherein the Fc region comprises an amino acid sequence that is at least 95% identical to the amino acid sequence of SEQ ID NO: 34.

E135. The heavy-chain antibody of any one of E113-E132, wherein the Fc region comprises:
a first polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering; and
a second polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

E136. The heavy-chain antibody of E135, wherein the first polypeptide chain comprises an amino acid sequence that is at least 95% identical to the amino acid sequence of SEQ ID NO: 34, and the second polypeptide chain comprises an amino acid sequence that is at least 95% identical to the amino acid sequence of SEQ ID NO: 34.

E137. The heavy-chain antibody of E135 or E136, wherein the first polypeptide chain comprises the amino acid sequence of SEQ ID NO: 35, and the second polypeptide chain comprises the amino acid sequence of SEQ ID NO: 36.

E138. The heavy-chain antibody of E113, wherein:
the Fc region comprises a first polypeptide chain comprising the amino acid sequence of SEQ ID NO: 35 and a second polypeptide chain comprising the amino acid sequence of SEQ ID NO: 36; and
the first peptide linker and the second peptide linker are independently poly-Gly linkers.

E139. The heavy-chain antibody of E113 or E138, wherein:
the Fc region comprises a first polypeptide chain comprising the amino acid sequence of SEQ ID NO: 35 and a second polypeptide chain comprising the amino acid sequence of SEQ ID NO: 36; and
the first peptide linker and the second peptide linker both comprise the amino acid sequence of GGGG (SEQ ID NO: 37).

E140. The heavy-chain antibody of any one of E91-E139, wherein the full set of VH CDRs 1, 2, and 3 (combined) in the first VH region is at least 95% identical to the VH CDRs 1, 2, and 3 of any one of SEQ ID NOs: 11-14, wherein the VH CDRs 1, 2, and 3 are defined by any one of Kabat, Chothia, or IMGT.

E141. The heavy-chain antibody of any one of E91-E140, wherein the full set of VH CDRs 1, 2, and 3 (combined) in the first VH region is at least 95% identical to the VH CDRs 1, 2, and 3 of any one of SEQ ID NOs: 11-14, wherein the VH CDRs 1, 2, and 3 are defined by Kabat.

E142. The heavy-chain antibody of any one of E91-E140, wherein the full set of VH CDRs 1, 2, and 3 (combined) in the first VH region is at least 95% identical to the VH CDRs 1, 2, and 3 of any one of SEQ ID NOs: 11-14, wherein the VH CDRs 1, 2, and 3 are defined by Chothia.

E143. The heavy-chain antibody of any one of E91-E140, wherein the full set of VH CDRs 1, 2, and 3 (combined) in the first VH region is at least 95% identical to the VH CDRs 1, 2, and 3 of any one of SEQ ID NOs: 11-14, wherein the VH CDRs 1, 2, and 3 are defined by IMGT.

E144. The heavy-chain antibody of any one of E91-E143, wherein the full set of VH CDRs 1, 2, and 3 (combined) in the second VH region is at least 95% identical to the VH CDRs 1, 2, and 3 of any one of SEQ ID NOs: 22-25, wherein the VH CDRs 1, 2, and 3 are defined by any one of Kabat, Chothia, or IMGT.

E145. The heavy-chain antibody of any one of E91-E144, wherein the full set of VH CDRs 1, 2, and 3 (combined) in the second VH region is at least 95% identical to the VH CDRs 1, 2, and 3 of any one of SEQ ID NOs: 22-25, wherein the VH CDRs 1, 2, and 3 are defined by Kabat.

E146. The heavy-chain antibody of any one of E91-E144, wherein the full set of VH CDRs 1, 2, and 3 (combined) in the second VH region is at least 95% identical to the VH CDRs 1, 2, and 3 of any one of SEQ ID NOs: 22-25, wherein the VH CDRs 1, 2, and 3 are defined by Chothia.

E147. The heavy-chain antibody of any one of E91-E144, wherein the full set of VH CDRs 1, 2, and 3 (combined) in the second VH region is at least 95% identical to the VH CDRs 1, 2, and 3 of any one of SEQ ID NOs: 22-25, wherein the VH CDRs 1, 2, and 3 are defined by IMGT.

E148. The heavy-chain antibody of any one of E91-E139, wherein the full set of VH CDRs 1, 2, and 3 (combined) in the first VH region is identical to the VH CDRs 1, 2, and 3 of any one of SEQ ID NOs: 11-14.

E149. The heavy-chain antibody of any one of E91-E139, wherein the full set of VH CDRs 1, 2, and 3 (combined) in the first VH region is identical to the VH CDRs 1, 2, and 3 of any one of SEQ ID NOs: 11-14, wherein the VH CDRs 1, 2, and 3 are defined by any one of Kabat, Chothia, or IMGT.

E150. The heavy-chain antibody of any one of E91-E139, wherein the full set of VH CDRs 1, 2, and 3 (combined) in the first VH region is identical to the VH CDRs 1, 2, and 3 of any one of SEQ ID NOs: 11-14, wherein the VH CDRs 1, 2, and 3 are defined by Kabat.

E151. The heavy-chain antibody of any one of E91-E139, wherein the full set of VH CDRs 1, 2, and 3 (combined) in the first VH region is identical to the VH CDRs 1, 2, and 3 of any one of SEQ ID NOs: 11-14, wherein the VH CDRs 1, 2, and 3 are defined by Chothia.

E152. The heavy-chain antibody of any one of E91-E139, wherein the full set of VH CDRs 1, 2, and 3 (combined) in the first VH region is identical to the VH CDRs 1, 2, and 3 of any one of SEQ ID NOs: 11-14, wherein the VH CDRs 1, 2, and 3 are defined by IMGT.

E153. The heavy-chain antibody of any one of E91-E139 or E148-E152, wherein the full set of VH CDRs 1, 2, and 3 (combined) in the second VH region is identical to the VH CDRs 1, 2, and 3 of any one of SEQ ID NOs: 22-25.

E154. The heavy-chain antibody of any one of E91-E139 or E148-E152, wherein the full set of VH CDRs 1, 2, and 3 (combined) in the second VH region is identical to the VH CDRs 1, 2, and 3 of any one of SEQ ID NOs: 22-25, wherein the VH CDRs 1, 2, and 3 are defined by any one of Kabat, Chothia, or IMGT.

E155. The heavy-chain antibody of any one of E91-E139 or E148-E152, wherein the full set of VH CDRs 1, 2, and 3 (combined) in the second VH region is identical to the VH CDRs 1, 2, and 3 of any one of SEQ ID NOs: 22-25, wherein the VH CDRs 1, 2, and 3 are defined by Kabat.

E156. The heavy-chain antibody of any one of E91-E139 or E148-E152, wherein the full set of VH CDRs 1, 2, and 3 (combined) in the second VH region is identical to the VH CDRs 1, 2, and 3 of any one of SEQ ID NOs: 22-25, wherein the VH CDRs 1, 2, and 3 are defined by Chothia.

E157. The heavy-chain antibody of any one of E91-E139 or E148-E152, wherein the full set of VH CDRs 1, 2, and 3 (combined) in the second VH region is identical to the VH CDRs 1, 2, and 3 of any one of SEQ ID NOs: 22-25, wherein the VH CDRs 1, 2, and 3 are defined by IMGT.

E158. The heavy-chain antibody of any one of E91-E139, wherein:
the full set of VH CDRs 1, 2, and 3 (combined) in the first VH region is at least 95% identical to the VH CDRs 1, 2, and 3 of SEQ ID NO: 11; and
the full set of VH CDRs 1, 2, and 3 (combined) in the second VH region is at least 95% identical to the VH CDRs 1, 2, and 3 of SEQ ID NO: 22.

E159. The heavy-chain antibody of any one of E91-E139, wherein:
the full set of VH CDRs 1, 2, and 3 (combined) in the first VH region is identical to the VH CDRs 1, 2, and 3 of SEQ ID NO: 11; and
the full set of VH CDRs 1, 2, and 3 (combined) in the second VH region is identical to the VH CDRs 1, 2, and 3 of SEQ ID NO: 22.

E160. The heavy-chain antibody of any one of E91-E139, wherein:
the full set of VH CDRs 1, 2, and 3 (combined) in the first VH region is at least 95% identical to the VH CDRs 1, 2, and 3 of SEQ ID NO: 12; and
the full set of VH CDRs 1, 2, and 3 (combined) in the second VH region is at least 95% identical to the VH CDRs 1, 2, and 3 of SEQ ID NO: 23.

E161. The heavy-chain antibody of any one of E91-E139, wherein:
the full set of VH CDRs 1, 2, and 3 (combined) in the first VH region is identical to the VH CDRs 1, 2, and 3 of SEQ ID NO: 12; and
the full set of VH CDRs 1, 2, and 3 (combined) in the second VH region is identical to the VH CDRs 1, 2, and 3 of SEQ ID NO: 23.

E162. The heavy-chain antibody of any one of E91-E139, wherein:
the full set of VH CDRs 1, 2, and 3 (combined) in the first VH region is at least 95% identical to the VH CDRs 1, 2, and 3 of SEQ ID NO: 13; and
the full set of VH CDRs 1, 2, and 3 (combined) in the second VH region is at least 95% identical to the VH CDRs 1, 2, and 3 of SEQ ID NO: 23.

E163. The heavy-chain antibody of any one of E91-E139, wherein:
the full set of VH CDRs 1, 2, and 3 (combined) in the first VH region is identical to the VH CDRs 1, 2, and 3 of SEQ ID NO: 13; and
the full set of VH CDRs 1, 2, and 3 (combined) in the second VH region is identical to the VH CDRs 1, 2, and 3 of SEQ ID NO: 23.

E164. The heavy-chain antibody of any one of E91-E139, wherein:
the full set of VH CDRs 1, 2, and 3 (combined) in the first VH region is at least 95% identical to the VH CDRs 1, 2, and 3 of SEQ ID NO: 14; and
the full set of VH CDRs 1, 2, and 3 (combined) in the second VH region is at least 95% identical to the VH CDRs 1, 2, and 3 of SEQ ID NO: 22.

E165. The heavy-chain antibody of any one of E91-E139, wherein:
the full set of VH CDRs 1, 2, and 3 (combined) in the first VH region is identical to the VH CDRs 1, 2, and 3 of SEQ ID NO: 14; and
the full set of VH CDRs 1, 2, and 3 (combined) in the second VH region is identical to the VH CDRs 1, 2, and 3 of SEQ ID NO: 22.

E166. The heavy-chain antibody of any one of E91-E139, wherein:
the full set of VH CDRs 1, 2, and 3 (combined) in the first VH region is at least 95% identical to the VH CDRs 1, 2, and 3 of SEQ ID NO: 12; and
the full set of VH CDRs 1, 2, and 3 (combined) in the second VH region is at least 95% identical to the VH CDRs 1, 2, and 3 of SEQ ID NO: 24.

E167. The heavy-chain antibody of any one of E91-E139, wherein:
the full set of VH CDRs 1, 2, and 3 (combined) in the first VH region is identical to the VH CDRs 1, 2, and 3 of SEQ ID NO: 12; and
the full set of VH CDRs 1, 2, and 3 (combined) in the second VH region is identical to the VH CDRs 1, 2, and 3 of SEQ ID NO: 24.

E168. The heavy-chain antibody of any one of E91-E139, wherein:
the full set of VH CDRs 1, 2, and 3 (combined) in the first VH region is at least 95% identical to the VH CDRs 1, 2, and 3 of SEQ ID NO: 12; and
the full set of VH CDRs 1, 2, and 3 (combined) in the second VH region is at least 95% identical to the VH CDRs 1, 2, and 3 of SEQ ID NO: 25.

E169. The heavy-chain antibody of any one of E91-E139, wherein:
the full set of VH CDRs 1, 2, and 3 (combined) in the first VH region is identical to the VH CDRs 1, 2, and 3 of SEQ ID NO: 12; and
the full set of VH CDRs 1, 2, and 3 (combined) in the second VH region is identical to the VH CDRs 1, 2, and 3 of SEQ ID NO: 25.

E170. A heavy-chain antibody comprising:
a first heavy chain that binds to IL2RB comprising the amino acid sequence of SEQ ID NO: 38; and
a second heavy chain that binds to IL2RG comprising the amino acid sequence of SEQ ID NO: 39.

E171. The heavy-chain antibody of E1-E170, wherein:
the heavy-chain antibody has an affinity for IL2R with a Kd in the range of 10⁻¹¹ M to 106 M; and/or
the heavy-chain antibody has an affinity for IL2Rβ with a Kd in the range of 10⁻⁸ M to 2.5×10⁻⁷ M; and/or
the heavy-chain antibody has an affinity for IL2Rγ with a Kd in the range of 10⁻⁹ M to 2.5×10⁻⁸ M.

E172. The heavy-chain antibody of any one of E1-E171, wherein the heavy-chain antibody is an IL-2Rβγ agonist.

E173. A pharmaceutical composition comprising:
a heavy-chain antibody of any one of E1-E172; and
a pharmaceutically acceptable excipient.

E174. The pharmaceutical composition of E173, wherein the pharmaceutical composition is adapted for intravenous or subcutaneous administration.

E175. A method of treating cancer in a subject in need thereof, comprising administering to the subject a heavy-chain antibody of any one of E1-E172 or a pharmaceutical composition of E173 or E174.

E176. A method of treating cancer in a subject in need thereof, comprising administering to the subject a heavy-chain antibody of any one of E1-E172 or a pharmaceutical composition of E173 or E174 in combination with a T-cell redirecting therapy.

E177. A method of enhancing an anti-cancer effect associated with administration of a T-cell redirecting therapy in a subject diagnosed with cancer, comprising administering to the subject a heavy-chain antibody of any one of E1-E172 or a pharmaceutical composition of E173 or E174 in combination with the T-cell redirecting therapy.

E178. The method of E176 or E177, further comprising administering a premedication to the subject prior to the administration of a first dose of the heavy-chain only antibody or a first dose of the T-cell redirecting therapy.

E179. The method of E178, wherein the premedication is selected from antihistamines, glucocorticoids, IL-6 receptor antagonists, and tumor necrosis factor alpha (TNF-α) antagonists.

E180. The method of any one of E176-E179, wherein the T-cell redirecting therapy is a bispecific T-cell engaging molecule.

E181. The method of E180, wherein the bispecific T-cell engaging molecule comprises a first domain that binds to a target cancer cell antigen and a second domain that binds to human CD3.

E182. The method of E181, wherein the target cancer cell antigen is selected from EpCAM, CEA, CD19, CD33, CD70, EGFRVIII, FLT3, GPRC5D, DLL3, BCMA, PSMA, STEAP1, STEAP2, MUC16, MUC17, and CLDN18.2.

E183. The method of E181 or E182, wherein the target cancer cell antigen is DLL3.

E184. The method of any one of E176-E183, wherein the bispecific T-cell engaging molecule further comprises a half-life extension domain.

E185. The method of E184, wherein the half-life extension domain provides a half-life for the bispecific T-cell engaging molecule of at least 24 hours.

E186. The method of E184 or E185, wherein the half-life extension domain is selected from immunoglobulin Fc domains, domains derived from serum albumin (e.g., human serum albumin), albumin-binding domains (e.g., comprising human albumin binding peptides or an antibody fragment that binds to serum albumin), peptides that bind to the neonatal Fc receptor (FcRn), and polyethylene glycol polymers.

E187. The method of any one of E176-E184, wherein the T-cell redirecting therapy is tarlatamab.

E188. The method of any one of E176-E186, wherein the bispecific T-cell engaging molecule is a three-chain antibody-like molecule, a heterodimeric IgG molecule (hetero-IgG), or a half-life extended (HLE) BITE® molecule.

E189. The method of any one of E176-E179, wherein the T-cell redirecting therapy is a chimeric antigen receptor (CAR)-expressing T-cell.

E190. The method of E189, wherein the CAR-expressing T-cell comprises a first domain that binds to a target cancer cell antigen, a transmembrane domain, and an intracellular signaling domain.

E191. The method of E190, wherein the target cancer cell antigen is selected from EpCAM, CEA, CD19, CD33, CD70, EGFRVIII, FLT3, GPRC5D, DLL3, BCMA, PSMA, STEAP1, STEAP2, MUC16, MUC17, and CLDN18.2.

E192. The method of any one of E176-E191, wherein at least one dose of the heavy-chain only antibody is administered to the subject prior to a first dose of the T-cell redirecting therapy.

E193. The method of any one of E176-E192, wherein the method comprises administering the heavy-chain only antibody in combination with the T-cell redirecting therapy in one or more treatment cycles.

E194. The method of E193, wherein each of the one or more treatment cycles comprises a single dose of the heavy-chain only antibody and a single dose of the T-cell redirecting therapy.

E195. The method of E193, wherein each of the one or more treatment cycles comprises multiple doses of the heavy-chain only antibody and a single dose of the T-cell redirecting therapy.

E196. The method of E193, wherein each of the one or more treatment cycles comprises a single dose of the heavy-chain only antibody and multiple doses of the T-cell redirecting therapy.

E197. The method of E193, wherein each of the one or more treatment cycles comprises multiple doses of the heavy-chain only antibody and multiple doses of the T-cell redirecting therapy.

E198. The method of any one of E175-E182, E184-E186, or E188-E197, wherein the cancer is a hematologic cancer.

E199. The method of E198, wherein the cancer is selected from acute myeloid leukemia, acute lymphocytic leukemia, chronic lymphocytic leukemia, chronic myeloid leukemia, multiple myeloma, diffuse large B-cell lymphoma, Burkitt lymphoma, and non-Hodgkin lymphoma.

E200. The method of any one of E175-E197, wherein the cancer is selected from prostate cancer, non-small cell lung cancer, small-cell lung cancer, renal cell carcinoma, hepatocellular carcinoma, bladder cancer, testicular cancer, colorectal cancer, esophageal cancer, glioblastoma, head and neck cancer, pancreatic cancer, breast cancer, gastric cancer, gastroesophageal junction cancer, bone cancer, ovarian cancer, endometrial cancer, and melanoma.

E201. The method of E200, wherein the subject has at least one tumor with low immune infiltration (e.g., low T-cell infiltration) prior to the co-administration.

E202. The method of E200 or E201, wherein the co-administration increases tumor T-cell infiltration.

E203. The method of any one of E200-E202, wherein the co-administration is associated with at least one anti-tumor effect.

E204. The method of E203, wherein the at least one anti-tumor effect is selected from a decrease in tumor volume, a decrease in the number of tumor cells, a decrease in tumor cell proliferation, and a decrease in tumor cell survival.

E205. The method of any one of E176-E204, wherein the co-administration is associated with at least one anti-cancer effect.

E206. The method of E205, wherein the at least one anti-cancer effect is selected from a decrease in the number of cancer cells, a decrease in the number of metastases, an increase in life expectancy, a decrease in cancer cell proliferation, a decrease in cancer cell survival, and an amelioration of various physiological symptoms associated with the cancerous condition.

E207. The method of any one of E175-E206, wherein the heavy-chain only antibody is administered in a pharmaceutical composition adapted for intravenous or subcutaneous delivery.

E208. The method of any one of E176-E207, wherein the T-cell redirecting therapy is administered in a pharmaceutical composition adapted for intravenous or subcutaneous delivery.

E209. The method of E208, wherein the pharmaceutical composition comprises the bispecific T-cell engaging molecule, a buffer, a surfactant, and a stabilizing agent.

E210. The method of E208 or E209, wherein the pharmaceutical composition comprises the bispecific T-cell engaging molecule, a glutamate buffer, polysorbate 20 or polysorbate 80, and sucrose, at a pH in the range of 4.0 to 4.4.

E211. The method of any one of E176-E210, wherein the heavy-chain only antibody and the T-cell redirecting therapy are administered in separate pharmaceutical compositions.

E212. The method of E211, wherein the separate pharmaceutical compositions may be lyophilized and reconstituted prior to administration to the subject.

E213. The method of any one of E176-E212, wherein the heavy-chain only antibody and the T-cell redirecting therapy are administered concurrently.

E214. The method of any one of E176-E212, wherein the heavy-chain only antibody and the T-cell redirecting therapy are administered sequentially.

E215. The method of any one of E175-E214, wherein the subject was previously administered a first line therapy for the cancer.

E216. The method of any one of E175-E125, wherein the subject was previously administered a first line therapy and a second line therapy for the cancer.

### BRIEF DESCRIPTION OF THE DRAWINGS

FIGS. 1A-1D show the binding of a heavy-chain antibody disclosed herein (“HCAb1”) to human CD4⁺ T-cells (FIG. 1A), human CD4⁺CD25⁺CD1271^{b} T-regs (FIG. 1B), human CD8⁺ T-cells (FIG. 1C), and human CD3 CD56+NK cells (FIG. 1D) relative to an IgG1 isotype control antibody (“hulgG1”) as a function of concentration for each test article.

FIGS. 2A-2D show the binding of HCAb1 to cyno CD4⁺ T-cells (FIG. 2A), cyno CD4⁺CD25⁺CD1271^{b} T-regs (FIG. 2B), cyno CD8⁺ T-cells (FIG. 2C), and cyno CD3 CD159a⁺ NK cells (FIG. 2D) relative to an IgG1 isotype control antibody as a function of concentration for each test article.

FIGS. 3A-3D depict STAT5 phosphorylation dose curves in human CD4⁺ Foxp3 T-cells (FIG. 3A), human CD4⁺CD25⁺ Foxp3⁺ regulatory T-cells (FIG. 3B), human CD8⁺ T-cells (FIG. 3C), and human CD3 CD56⁺ NK cells (FIG. 3D) as a function of concentration for HCAb1 and the control molecules (recombinant human IL-2 (“rhIL-2”) and IL-2 variant (“IL-2v”)).

FIGS. 4A-4D show the proliferation (Ki67 dose curves) of human CD4⁺ Foxp3 T-cells (FIG. 4A), human CD4⁺CD25⁺ Foxp3⁺ regulatory T-cells (FIG. 4B), human CD8⁺ T-cells (FIG. 4C), and human CD3 CD56⁺ NK cells (FIG. 4D) as a function of concentration for HCAb1 and the control molecules (rhIL-2 and IL-2v).

FIGS. 5A-5D show the proliferation (Ki67 dose curves) of cyno CD4⁺ Foxp3 T-cells (FIG. 5A), cyno CD4⁺CD25⁺ Foxp3⁺ regulatory T-cells (FIG. 5B), cyno CD8⁺ T-cells (FIG. 5C), and cyno CD3 CD159a⁺ NK cells (FIG. 5D) as a function of concentration for HCAb1 and the control molecules (rhIL-2 and IL-2v).

FIGS. 6A and 6B depict the results from a T-cell dependent cellular cytotoxicity (TDCC) assay (E: T=5:1, 72 hour assay time) using SHP-77 Luc cells to investigate cytotoxicity for tarlatamab (“DLL3-TCE”) alone and in combination with HCAb1, rhIL-2, or IL-2v at high (FIG. 6A) and medium (FIG. 6B) enabler concentrations.

FIGS. 7A-7K show measured cytokine concentrations (IFNγ (FIG. 7A), IL-2 (FIG. 7B), IL-6 (FIG. 7C), IL-10 (FIG. 7D), TNFα (FIG. 7E), Granzyme B (FIG. 7F), GM-CSF (FIG. 7G), IL-1Ra (FIG. 7H), IL-5 (FIG. 7I), MCP-1 (FIG. 7J), and MIP-1B (FIG. 7K)) from the TDCC assay (E: T=5:1, 72 hour assay time) using SHP-77 Luc cells at various tarlatamab (“DLL3-TCE”) concentrations, alone or in combination with high (300 nM, 30 nM, or 10 nM) concentrations of HCAb1, IL-2v, and rhIL-2, respectively.

FIGS. 8A-8K show measured cytokine concentrations (IFNγ (FIG. 8A), IL-2 (FIG. 8B), IL-6 (FIG. 8C), IL-10 (FIG. 8D), TNFα (FIG. 8E), Granzyme B (FIG. 8F), GM-CSF (FIG. 8G), IL-1Ra (FIG. 8H), IL-5 (FIG. 8I), MCP-1 (FIG. 8J), and MIP-1B (FIG. 8K)) from the TDCC assay (E: T=5:1, 72 hour assay time) using SHP-77 Luc cells at various tarlatamab (“DLL3-TCE”) concentrations, alone or in combination with medium (30 nM, 2 nM, or 0.5 nM) concentrations of HCAb1, IL-2v, and rhIL-2, respectively.

FIG. 9 depicts the results from a serial T-cell engaging molecule exposure cytotoxicity assay (E: T=2:1) using NUGC4-CD58 KO cells to investigate exposure-mediated T-cell dysfunction for a T-cell engaging molecule that binds to human EpCAM and CD3 (“EpCAM-TCE”), alone and in combination with HCAb1, IL-2v, or a wild-type human IL-2 molecule conjugated to the same Fc region used in HCAb1 (“wtIL2-Fc”).

FIGS. 10A-10D show measured cytokine concentrations (IL-5 (FIG. 10A), IL-6 (FIG. 10B), IL-10 (FIG. 10C), and TNFα (FIG. 10D)) from the serial T-cell engaging molecule exposure cytotoxicity assay (E: T=2:1) using NUGC4-CD58 KO cells to investigate exposure-mediated T-cell dysfunction for EpCAM-TCE alone and in combination with HCAb1, IL-2v, or wtIL2-Fc.

FIG. 11 shows concentration-dependent viscosity data, including exponential fit curves, for HCAb1 and HCAb2 in a 10 mM acetate, 9% sucrose, 0.01% PS80, pH 5.2 formulation buffer at 5° C. and 25° C.

FIG. 12 depicts the time-dependent percentage of higher-order aggregates present in 5 mg/mL formulations of HCAb1 and HCAb2 over 4 weeks at 40° C.

FIG. 13 is a graph showing tumor volume as a function of time between 11 and 32 days post-tumor implantation for mice in an in vivo combination study assessing HCAb1 and tarlatamab (“DLL3-TCE”).

FIG. 14 is a graph showing relative body weight (%) as a function of time between 11 and 32 days post-tumor implantation for mice in an in vivo combination study assessing HCAb1 and tarlatamab (“DLL3-TCE”).

FIG. 15 is a schematic depicting a non-limiting example structure for a heavy-chain antibody having activity as an agonist of the dimeric interleukin-2 receptor as described herein.

FIG. 16 provides non-human primate (NHP) pharmacokinetic (PK) data in graphical format. NHP (n=3 per group per timepoint) were administered 0.03, 0.1, or 0.3 mg/kg of HCAb1 by IV injection on Day 0 and Day 14. Serum was collected over 4 weeks and assessed by an electrochemiluminescence method using anti-idiotypic monoclonal antibodies.

FIGS. 17A-17D are graphs showing Harvest Titer (FIG. 17A), % SEC Main Peak (FIG. 17B), % HIC Main Peak (FIG. 17C), and % Capillary Electrophoresis non-reduced Main Peak (FIG. 17D) for eight example heavy chain antibodies (HCAb1, HCAb, HCAb3, HCAb4, HCAb5, HCAb6, HCAb7, and HCAb8).

FIG. 18 provides an example size exclusion chromatogram for HCAb2 at a concentration of 10 mg/mL in a 10 mM acetate, 9% sucrose, pH 5.2 formulation buffer after one week at 40° C.

FIG. 19 depicts the time-dependent percentage of higher-order aggregates present in 10 mg/mL formulations of HCAb1, HCAb2, HCAb3, HCAb4, HCAb5, HCAb6, HCAb7, and HCAb8 in 10 mM sodium acetate, 9% sucrose, pH 5.2 formulation buffer over the course of one week at 40° C.

### DETAILED DESCRIPTION

Disclosed herein are heavy-chain antibodies having activity as agonists of the dimeric interleukin-2 receptor, pharmaceutical compositions comprising the heavy-chain antibodies, and uses and methods of treating disorders, such as cancer, with the heavy-chain antibodies and pharmaceutical compositions described herein.

### Definitions

The following definitions are provided to assist in understanding the scope of this disclosure. Unless defined otherwise, all technical and scientific terms used herein have the same meaning as is commonly understood by one of ordinary skill in the art to which this disclosure belongs.

Other than in the Examples, or where otherwise indicated, all numbers expressing quantities (e.g., of components or reaction conditions) used herein should be understood as modified in all instances by the term “about.” As used herein, “about,” when used in connection with a measurable numerical variable, refers to the indicated value of the variable and to all values of the variable that are within the experimental error of the indicated value (e.g., within the 95% confidence interval for the mean) or +10% of the indicated value, whichever is greater.

Where a range of values is provided, it should be understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges also encompassed within the disclosure, subject to any specifically excluded limit in the stated range. Where the stated range includes one or both of the limits, ranges excluding either or both of those included limits are also included in the disclosure.

As used herein, the terms “a” and “an” mean “one or more” unless specifically indicated otherwise. Additionally, “one or more” and “at least one” are used interchangeably herein. Furthermore, unless otherwise required by context, singular terms include pluralities and plural terms include the singular.

As used herein, the term “polypeptide” refers to a polymer of amino acid residues. Polypeptides comprising between two and fifty amino acids may also be referred to as “peptides” herein. “Polypeptide” further encompasses an amino acid polymer in which one or more amino acid residues is an analog or mimetic of a corresponding naturally occurring amino acid, as well as to naturally occurring amino acid polymers. The term can also encompass an amino acid polymer that have been modified, e.g., by the addition of carbohydrate residues to form glycoproteins, or phosphorylated. Polypeptides can be produced by a naturally-occurring and non-recombinant cell, or polypeptides can be produced by a genetically-engineered or recombinant cell, and comprise molecules having the amino acid sequence of the native protein, or molecules having deletions from, additions to, and/or substitutions of one or more amino acids of the native sequence. The terms “polypeptide” and “protein” are used interchangeably herein.

As used herein, a “derivative” of a polypeptide is a polypeptide (e.g., an antigen binding protein such as an antibody) that has been chemically modified in some manner distinct from insertion, deletion, or substitution variants, such as, e.g., via conjugation to another chemical moiety.

As used herein, the term “linker moiety” or “linker” refers to a biologically acceptable peptidyl or non-peptidyl organic group that is covalently bound to a first molecule (e.g., a first polypeptide) and covalently joins or conjugates the molecule to a second molecule (e.g., a second polypeptide). Where the linker moiety consists of a polypeptide or a polypeptide derivative (e.g., a polypeptide that has been chemically modified at one or both of the N-terminus and C-terminus to incorporate a functional group that permits conjugation to the first or second molecule), it may be referred to as a “polypeptide linker” herein. Where the peptidyl linker moiety comprises between two and fifty amino acids, it may also be referred to as a “peptide linker” herein. Additionally, as used herein, a “linker,” such as a “peptide linker,” connected to an Fc region may be connected to a hinge region of the Fc region, wherein the hinge region is a naturally occurring/wild-type hinge region or a hinge region containing one or more modifications relative to a wild-type hinge region. In some embodiments, the linker is connected to a naturally occurring/wild-type hinge region. In alternative embodiments, a “linker,” such as a “peptide linker,” connected to an Fc region may be directly connected to a CH2 domain of the Fc region.

As used herein, the term “antibody” generally refers to a tetrameric immunoglobulin protein comprising two light chain polypeptides (such as, e.g., light chain polypeptides that are about 25 kDa each) and two heavy chain polypeptides (such as, e.g., heavy chain polypeptides that are about 50-70 kDa each). The term “light chain,” as used with respect to an antibody or a fragment thereof, includes a full-length light chain and fragments thereof having sufficient variable region sequence to confer binding specificity. A full-length light chain refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain,” as used with respect to an antibody or a fragment thereof, includes a full-length heavy chain and fragments thereof having sufficient variable region sequence to confer binding specificity. A full-length heavy chain refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (u), delta (A), gamma (γ), alpha (a), and epsilon (¿), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies typically have three constant domains (CH1, CH2, and CH3), whereas the heavy chains in IgM and IgE antibodies typically have four constant domains (CH1, CH2, CH3, and CH4). The heavy chain constant domains can be from any immunoglobulin isotype, including subtypes. The antibody chains are linked together via inter-polypeptide disulfide bonds between the CL domain and the CH1 domain (i.e., between the light and heavy chain) and between the hinge regions of the two antibody heavy chains.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein. From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. The Kabat definition is based on sequence variability, while the Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B-cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlanet al. “Identification of specificity-determining residues in antibodies.” Faseb J. 1995; 9:133-139., each of which is herein specifically incorporated by reference. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309 (3): 657-670; 2001). Unless otherwise indicated, specific CDRs identified herein are defined by IMGT.

“Framework Region” or “FR” residues are those variable domain residues other than the hypervariable region/CDR residues as defined herein.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies, single domain antibodies, antibody fragments, and the like mean residue numbering by the EU numbering system.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. In contrast to polyclonal antibody preparations which typically include different antibodies directed against different determinants (epitopes), a monoclonal antibody is generally directed against a single determinant on the antigen. As non-limiting examples, monoclonal antibodies in accordance with the present disclosure can be made by the hybridoma method first described by Kohler et al. (1975) Nature 256:495, and can also be made via recombinant protein production methods (see, e.g., U.S. Pat. No. 4,816,567).

The term “human antibody,” as used herein, is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences that are derived from the germline of another mammalian species, such as, e.g., a mouse, have been grafted onto human framework sequences.

As used herein, the term “chimeric antibody” refers to an antibody comprising amino acid sequences from at least two different Ig loci, e.g., a transgenic antibody comprising a portion encoded by a human Ig locus and a portion encoded by a rat Ig locus. Chimeric antibodies include transgenic antibodies with non-human Fc-regions or artificial Fc-regions, and human idiotypes. Such immunoglobulins can be isolated from animals of the disclosure that have been engineered to produce such chimeric antibodies.

As used herein, the term “antibody construct” refers to a molecule in which the structure and/or function is/are based on the structure and/or function of an antibody, e.g., of a full-length immunoglobulin molecule. An antibody construct binds to its target or antigen, and/or it comprises the heavy chain variable region (VH) and/or the light chain variable region (VL) of an antibody, or comprises domains derived therefrom.

As used herein, the term “heavy-chain antibody” or “heavy chain-only antibody” refers to an immunoglobulin protein consisting of two heavy chain polypeptides (such as, e.g., heavy chain polypeptides that are about 50-70 kDa each). A “heavy-chain antibody” is an antibody fragment that lacks the two light chain polypeptides found in a conventional antibody. Heavy-chain antibodies constitute about one-fourth of the IgG antibodies produced by the camelids, e.g., camels and llamas (Hamers-Casterman C., et al. Nature. 363, 446-448 (1993)). These antibodies are formed by two heavy chains but are devoid of light chains. As a consequence, the variable antigen-binding part is referred to as the VHH domain, and it represents the smallest naturally occurring, intact, antigen-binding site, being only around 120 amino acids in length (Desmyter, A., et al. J. Biol. Chem. 276, 26285-26290 (2001)). Heavy-chain antibodies with a high specificity and affinity can be generated against a variety of antigens through immunization (van der Linden, R. H., et al. Biochim. Biophys. Acta. 1431, 37-46 (1999)), and the VHH portion can be readily cloned and expressed in yeast (Frenken, L. G. J., et al. J. Biotechnol. 78, 11-21 (2000)). Their levels of expression, solubility and stability are significantly higher than those of classical F (ab) or Fv fragments (Ghahroudi, M. A. et al. FEBS Lett. 414, 521-526 (1997)). Sharks have also been shown to have a single VH-like domain in their antibodies, termed VNAR. (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003).)

In some embodiments, a “heavy-chain antibody” is a homodimeric antibody comprising a VH antigen-binding domain and the CH2 and CH3 constant domains, in the absence of the CH1 domain. In some embodiments, a heavy-chain antibody is composed of a variable region antigen-binding domain composed of framework 1, CDR1, framework 2, CDR2, framework 3, CDR3, and framework 4. In some embodiments, a heavy-chain antibody is composed of an antigen-binding domain, at least part of a hinge region, and CH2 and CH3 domains (e.g., in the absence of a CH1 domain). In some embodiments, a heavy-chain antibody is composed of an antigen-binding domain, at least part of a hinge region, and a CH2 domain. In some embodiments, a heavy-chain antibody is composed of an antigen-binding domain, at least part of a hinge region, and a CH3 domain. Heavy-chain antibodies in which the CH2 and/or CH3 domain is truncated are also included herein. The heavy-chain antibodies described herein may belong to the IgG subclass, but heavy-chain antibodies belonging to other subclasses, such as IgM, IgA, IgD, and IgE subclass, are also included herein. In some embodiments, a heavy-chain antibody may belong to the IgG1, IgG2, IgG3, or IgG4 subtype, e.g., the IgG1 or IgG4 subtype. In some embodiments, a heavy-chain antibody is of the IgG1 or IgG4 subtype, wherein one or more of the CH domains is modified to alter an effector function of the heavy-chain antibody. In some embodiments, a heavy-chain antibody is of the IgG4 subtype, wherein one or more of the CH domains is modified to alter an effector function of the heavy-chain antibody. In some embodiments, a heavy-chain antibody is of the IgG1 subtype, wherein one or more of the CH domains is modified to alter an effector function of the heavy-chain antibody. Modifications of CH domains that alter effector function are further described herein. Non-limiting examples of heavy-chain antibodies are described, for example, in WO2018/039180, the disclosure of which is incorporated herein by reference herein in its entirety.

As used herein, an “antibody fragment” generally refers to a fragment of a full-length antibody or heavy-chain antibody, such as, e.g., VH, VHH, VL, (s) dAb, Fv, light chain (VL-CL), Fd (VH-CH1), heavy chain, Fab, Fab′, F(ab′) 2 or “r IgG” (“half antibody” consisting of a heavy chain and a light chain) or a modified fragment of a full-length antibody, such as, e.g., three-chain antibody-like molecule, heavy-chain only antibody, single-chain variable fragment (scFv), di-scFv or bi(s)-scFv, scFv-Fc, scFv-zipper, single-chain Fab (scFab), Fab2, Fab3, diabodies, single-chain diabodies, tandem diabodies (Tandabs), tandem di-scFv, tandem tri-scFv, “minibodies” exemplified by a structure which is as follows: (VH-VL-CH3) 2, (scFv-CH3) 2, ((scFv) 2-CH3⁺CH3), ((scFv) 2-CH3) or (scFv-CH3-scFv) 2, multibodies, such as triabodies or tetrabodies, and single domain antibodies, such as nanobodies or single variable domain antibodies comprising merely one variable region, which might be VHH, VH or VL, that binds to an antigen or target independently of other variable regions or domains.

As used herein, a “single domain antibody” refers to a single polypeptide chain that contains all or part of the heavy chain variable domain or all or part of the light chain variable domain of an antibody. In some embodiments, the single domain antibody is a human single domain antibody.

As used herein, the term “three-chain antibody like molecule,” “TCA” or “three-chain antibody fragment” refers to antibody-like molecules or antibody fragments comprising, consisting essentially of, or consisting of three polypeptide subunits, two of which comprise, consist essentially of, or consist of one heavy and one light chain of a monoclonal antibody, or antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy chain polypeptide comprising an Fc portion comprising CH2 and/or CH3 and/or CH4 domains, in the absence of a CH1 domain, and one or more antigen binding domains (such as, e.g., two antigen binding domains) that binds an epitope of a second antigen or a different epitope of the first antigen, wherein such binding domain is derived from or has sequence identity with the variable region of an antibody heavy chain.

As used herein, an “antigen-binding fragment” is a portion of an antibody or a heavy-chain antibody that lacks at least some of the amino acids present in a heavy chain (in the case of an antibody or heavy-chain antibody) and/or light chain (in the case of an antibody), but which is still capable of specifically binding to an antigen. An antigen-binding fragment includes, but is not limited to, a single-chain variable fragment (scFv), a nanobody (e.g., VH domain of camelid heavy-chain antibodies; VHH fragment, see Cortez-Retamozo et al., Cancer Research, Vol. 64:2853-57, 2004), a Fab fragment, a Fab′ fragment, a F(ab′) 2 fragment, a Fv fragment, a Fd fragment, and a CDR fragment, and can be derived from any mammalian source, such as human, mouse, rat, rabbit, or camelid.

Papain digestion of antibodies produces two identical antigen-binding fragments, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment.

As used herein, an “Fc region” may be a native-sequence Fc region or a variant Fc region. An “Fc region,” as used herein, may comprise a hinge region. The “Fc region” of an immunoglobulin generally comprises two constant domains, a CH2 domain and a CH3 domain, and optionally comprises a CH4 domain. In some embodiments, the Fc region may be an Fc region from an IgG1, IgG2, IgG3, or IgG4 immunoglobulin. In some embodiments, the Fc region comprises CH2 and CH3 domains from a human IgG1 or human IgG2 immunoglobulin. In some embodiments, the Fc region comprises a hinge region, a CH2 domain, and a CH3 domain. The Fc region may retain effector function, such as Clq binding, complement dependent cytotoxicity (CDC), Fc receptor binding, antibody-dependent cell-mediated cytotoxicity (ADCC), and phagocytosis. In other embodiments, the Fc region may be modified to reduce or eliminate effector function.

A “functional Fc region” possesses an “effector function” of a native-sequence Fc region. Non-limiting examples of effector functions include Clq binding, CDC; Fc-receptor binding, ADCC, ADCP, down-regulation of cell-surface receptors (e.g., B-cell receptor), etc. Such effector functions generally require the Fc region to interact with a receptor, such as, e.g., the FcγRI; FcγRIIA; FcγRIIB1; FcγRIIB2; FcγRIIIA; FcγRIIIB receptors, and the low affinity FcRn receptor; and can be assessed using various assays known in the art.

A “dead” or “silenced” Fc is one that has been mutated to retain activity with respect to, for example, prolonging serum half-life, but which does not activate a high affinity Fc receptor, or which has a reduced affinity to an Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, for example, one or more (e.g., two or more, three or more, four or more) amino acid substitution(s). Illustratively, in some embodiments, the variant Fc region has at least one amino acid substitution compared to a native-sequence Fc region or to the Fc region of a parent polypeptide, e.g., from one to ten amino acid substitutions, e.g., from one to five amino acid substitutions in a native-sequence Fc region or in the Fc region of the parent polypeptide. In some embodiments, the variant Fc region herein will possess at least 80% homology with a native-sequence Fc region and/or with an Fc region of a parent polypeptide, e.g., at least 85% homology therewith, e.g., at least 90% homology therewith, e.g., at least 95% homology therewith, e.g., at least 99% homology therewith.

As used herein, “heterodimerizing alterations” refer to alterations in the A and B chains of an Fc region (i.e., the two chains comprising the Fc region, wherein one chain is referred to herein as the “A” chain and the other is referred to herein as the “B” chain) that facilitate the formation of heterodimeric Fc regions, that is, Fc regions in which the A chain and the B chain of the Fc region do not have identical amino acid sequences. In some embodiments, heterodimerizing alterations can be asymmetric, that is, an A chain having a certain alteration can pair with a B chain having a different alteration. These alterations facilitate heterodimerization and disfavor homodimerization. Whether hetero- or homo-dimers have formed can be assessed, for example, by size differences as determined by polyacrylamide gel electrophoresis in situations where one polypeptide chain is a dummy Fc and the other is an scFv-Fc. One non-limiting example of such paired heterodimerizing alterations are the so-called “knobs and holes” substitutions. See, e.g., U.S. Pat. No. 7,695,936 and U.S. Patent Application Publication No. 2003/0078385. As used herein, an Fc region that comprises one pair of knobs and holes substitutions, comprises one substitution in the A chain and another in the B chain. For example, the following knobs and holes substitutions in the A and B chains of an IgG1 Fc region have been found to increase heterodimer formation as compared with that found with unmodified A and B chains and may be employed in some non-limiting embodiments of this disclosure: 1) Y407T in one chain and T366Y in the other; 2) Y407A in one chain and T366W in the other; 3) F405A in one chain and T394W in the other; 4) F405W in one chain and T394S in the other; 5) Y407T in one chain and T366Y in the other; 6) T366Y and F405A in one chain and T394W and Y407T in the other; 7) T366W and F405W in one chain and T394S and Y407A in the other; 8) F405W and Y407A in one chain and T366W and T394S in the other; and 9) T366W in one polypeptide of the Fc and T366S, L368A, and Y407V in the other. Alternatively or in addition to such alterations, substitutions creating new disulfide bridges can facilitate heterodimer formation. See, e.g., U.S. Patent Application Publication No. 2003/0078385. Such alterations in an IgG1 Fc region include, but are not limited to, the following substitutions: Y349C in one Fc polypeptide chain and S354C in the other; Y349C in one Fc polypeptide chain and E356C in the other; Y349C in one Fc polypeptide chain and E357C in the other; L351C in one Fc polypeptide chain and S354C in the other; T394C in one Fc polypeptide chain and E397C in the other; or D399C in one Fc polypeptide chain and K392C in the other. Additionally or alternatively, substitutions changing the charge of a one or more residue(s), for example, in the CH3-CH3 interface, can enhance heterodimer formation, as described, for example, in WO 2009/089004, which is incorporated by reference herein. Such substitutions are referred to herein as “charge pair substitutions,” and an Fc region comprising one pair of charge pair substitutions comprises one substitution in the A chain and a different substitution in the B chain. Non-limiting examples of charge pair substitutions include the following: 1) K409D or K409E in one chain plus D399K or D399R in the other; 2) K392D or K392E in one chain plus D399K or D399R in the other; 3) K439D or K439E in one chain plus E356K or E356R in the other; and 4) K370D or K370E in one chain plus E357K or E357R in the other. In addition, the substitutions R355D, R355E, K360D, or K360R in both chains can stabilize heterodimers when used with other heterodimerizing alterations. Specific charge pair substitutions can be used either alone or with other charge pair substitutions. Specific examples of single pairs of charge pair substitutions and combinations thereof include, but are not limited to, the following: 1) K409E in one chain plus D399K in the other; 2) K409E in one chain plus D399R in the other; 3) K409D in one chain plus D399K in the other; 4) K409D in one chain plus D399R in the other; 5) K392E in one chain plus D399R in the other; 6) K392E in one chain plus D399K in the other; 7) K392D in one chain plus D399R in the other; 8) K392D in one chain plus D399K in the other; 9) K409D and K360D in one chain plus D399K and E356K in the other; 10) K409D and K370D in one chain plus D399K and E357K in the other; 11) K409D and K392D in one chain plus D399K, E356K, and E357K in the other; 12) K409D and K392D on one chain and D399K on the other; 13) K409D and K392D on one chain plus D399K and E356K on the other; 14) K409D and K392D on one chain plus D399K and D357K on the other; 15) K409D and K370D on one chain plus D399K and D357K on the other; 16) D399K on one chain plus K409D and K360D on the other; and 17) K409D and K439D on one chain plus D399K and E356K on the other. In some embodiments, any of these heterodimerizing alterations can be used in polypeptides comprising variant Fc regions as described herein.

In some non-limiting embodiments, variant Fc sequences may include three amino acid substitutions in the CH2 region to reduce FcγRI binding at EU index positions 234, 235, and 237 (see Duncan et al., (1988) Nature 332:563). Two amino acid substitutions in the complement Clq binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 or IgG2 residues at positions 233-236 and IgG4 residues at positions 327, 330, and 331 can reduce ADCC and CDC (see, for example, Armour K L. et al., 1999 Eur J Immunol. 29 (8): 2613-24; and Shields R. L. et al., 2001. J Biol Chem. 276 (9): 6591-604). Silenced IgG1 is described, for example, in Boesch, A. W., et al., “Highly parallel characterization of IgG Fc binding interactions.” MAbs, 2014. 6 (4): p. 915-27, the disclosure of which is incorporated herein by reference in its entirety.

Further, an Fc variant can be constructed to remove or substantially reduce effector functions by substituting (mutating), deleting, or adding amino acid residues to effect complement binding or Fc receptor binding. For example, and not by way of limitation, a deletion may occur in a complement-binding site, such as a Clq-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

Antibodies and antibody fragments with reduced effector function include, but are not limited to, those with substitution of one or more of Fc region residues 238, 265, 269, 270, 297, 327, and 329, according to EU numbering (see, e.g., U.S. Pat. No. 6,737,056). In some embodiments, variant Fc regions with reduced effector function comprise substitutions at two or more of amino acid positions 265, 269, 270, 297, and 327, according to EU numbering, including the so-called “DANA” Fc mutant with substitution of residues 265 and 297 to alanine according to EU numbering (i.e., D265A and N297A according to EU numbering) (see, e.g., U.S. Pat. No. 7,332,581). In some embodiments, a variant Fc region with reduced effector function comprises the following two amino acid substitutions: D265A and N297A.

In some embodiments, effector function is reduced through a mutation in a constant region that eliminates glycosylation, e.g., an “effector-less mutation.” In some embodiments, the effector-less mutation is an N297A or a DANA mutation (D265A+N297A) in the CH2 region. Shields et al., J. Biol. Chem. 276 (9): 6591-6604 (2001). In some embodiments, the effector-less mutation is an N297G or a DANG mutation (D265A+N297G) in the CH2 region. In some embodiments, the variant Fc region lacks glycosylation at N297, e.g., the variant Fc region is a variant Fc region lacking glycosylation at N297 as described in International Patent Publication No. WO 2014/153063, which is incorporated by reference herein. Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli) or in host cells which result in an altered glycosylation pattern that is ineffective or less effective at promoting effector function (e.g., Shinkawa et al., J. Biol. Chem. 278 (5): 3466-3473 (2003)).

In some embodiments, the proline at position 329 (EU numbering) (P329) of a wild-type human Fc region is substituted with glycine or arginine or an amino acid residue large enough to destroy the proline sandwich within the Fc/Fcγ receptor interface, that is formed between the P329 of the Fc and tryptophan residues W87 and W110 of FcγRIII (Sondermann et al., Nature 406, 267-273 (20 Jul. 2000)). In some further embodiments, at least one further amino acid substitution in the Fc variant region is S228P, E233P, L234A, L235A, L235E, N297A, N297D, or P331S. In some embodiments, the at least one further amino acid substitution is L234A and L235A of the human IgG1 Fc region or S228P and L235E of the human IgG4 Fc region, all according to EU numbering (see, e.g., U.S. Pat. No. 8,969,526, which is incorporated by reference in its entirety).

In some embodiments, the variant Fc region has P329 of the human IgG Fc region substituted with glycine, wherein the variant Fc region comprises at least two further amino acid substitutions at L234A and L235A of the human IgG1 Fc region or S228P and L235E of the human IgG4 Fc region, and wherein the residues are numbered according to the EU numbering (see, e.g., U.S. Pat. No. 8,969,526). In some embodiments, the variant Fc region comprising the P329G, L234A and L235A (EU numbering) substitutions exhibits a reduced affinity to the human FcγRIIIA and FcγRIIA.

In some embodiments, the variant Fc region comprises a triple mutation: an amino acid substitution at position P329, a L234A, and a L235A mutation according to EU numbering (P329/LALA) (see, e.g., U.S. Pat. No. 8,969,526). In some embodiments, the variant Fc region comprises the following amino acid substitutions: P329G, L234A, and L235A according to EU numbering.

In some embodiments, an antibody, heavy-chain antibody, or antibody fragment comprises a variant human IgG4 CH3 domain sequence comprising a T366W mutation, which can optionally be referred to herein as an IgG4 CH3 knob sequence. In some embodiments, an antibody, heavy-chain antibody, or antibody fragment comprises a variant human IgG4 CH3 domain sequence comprising a T366S mutation, an L368A mutation, and a Y407V mutation, which can optionally be referred to herein as an IgG4 CH3 hole sequence. The IgG4 CH3 mutations described herein can be utilized in any suitable manner so as to place a “knob” on a first heavy chain constant region of a first monomer in an antibody dimer, and a “hole” on a second heavy chain constant region of a second monomer in an antibody dimer, thereby facilitating proper pairing (heterodimerization) of the desired pair of heavy chain polypeptide subunits in the antibody.

In some embodiments, an antibody, heavy-chain antibody, or antibody fragment comprises a heavy chain polypeptide subunit comprising a variant human IgG4 Fc region comprising an S228P mutation, an F234A mutation, an L235A mutation, and a T366W mutation (knob). In some embodiments, an antibody, heavy-chain antibody, or antibody fragment comprises a heavy chain polypeptide subunit comprising a variant human IgG4 Fc region comprising an S228P mutation, an F234A mutation, an L235A mutation, a T366S mutation, an L368A mutation, and a Y407V mutation (hole).

As used herein, a “Fab′ fragment” is a Fab fragment having at the C-terminus of the CH1 domain one or more cysteine residues from the antibody hinge region.

As used herein, a “F(ab′) 2 fragment” is a bivalent fragment including two Fab′ fragments linked by a disulfide bridge between the heavy chains at the hinge region.

As used herein, a “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

As used herein, a “single-chain variable fragment” or “scFv fragment” comprises the VH and VL regions of an antibody, wherein these regions are present in a single polypeptide chain, and optionally comprises a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

A “nanobody” is the heavy chain variable region of a heavy-chain antibody. Such variable domains are the smallest fully functional antigen-binding fragment of such heavy-chain antibodies with a molecular mass of only about 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks, and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only having the structure H2L2 (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold and may provide a framework for a long penetrating loop structure.

Antibodies, heavy-chain antibodies, and antibody fragments of the present disclosure may be multispecific, meaning they possess more than one binding specificity. As used herein, the term “multi-specific” includes “bispecific” (i.e., two binding specificities) and “trispecific” (i.e., three binding specificities), as well as higher-order independent specific binding affinities, such as higher-order polyepitopic specificity.

As used herein, an “isolated” molecule (such as, e.g., an antibody, heavy-chain antibody, antibody fragment, single domain antibody) is a molecule which has been identified and separated and/or recovered from a component of its natural environment. Contaminant components of its natural environment are materials which may interfere with diagnostic or therapeutic uses for the molecule, such as, e.g., enzymes, hormones, and other proteinaceous or non-proteinaceous solutes. In some embodiments, the isolated molecule will be purified (1) to greater than 95% by weight of the molecule as determined by the Lowry method, such as, e.g., more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, e.g., silver stain. In some embodiments, an isolated molecule will be prepared by a process comprising at least one purification step.

Aspects of the present disclosure include antibodies, heavy-chain antibodies, and antibody fragments comprising a heavy chain-only variable region in a monovalent or bivalent configuration. As used herein, the term “monovalent configuration,” as used in reference to a heavy chain-only variable region domain, means that only one heavy chain-only variable region domain is present, having a single binding site. In contrast, the term “bivalent configuration” as used in reference to a heavy chain-only variable region domain means that two heavy chain-only variable region domains are present (each having a single binding site), and are connected by a linker sequence. Non-limiting examples of linker sequences are discussed further herein, and include, without limitation, GS linker sequences of various lengths. When a heavy chain-only variable region is in a bivalent configuration, each of the two heavy chain-only variable region domains can bind to the same antigen, or to different antigens (e.g., to different epitopes on the same protein; to two different proteins, etc.). However, unless specifically noted otherwise, a heavy chain-only variable region denoted as being in a “bivalent configuration” is understood to contain two identical heavy chain-only variable region domains, connected by a linker sequence, wherein each of the two identical heavy chain-only variable region domains binds to the same target antigen.

Aspects of the present disclosure also include antibodies, heavy-chain antibodies, and antibody fragments having multi-specific configurations, which include, without limitation, bispecific, trispecific, etc. configurations. A large variety of methods and protein configurations are known and used in bispecific monoclonal antibodies (BsMAB), tri-specific antibodies, etc.

Various methods for the production of multivalent artificial antibodies have been developed by recombinantly fusing variable domains of two or more antibodies. In some embodiments, a first and a second antigen-binding domain on a polypeptide are connected by a polypeptide linker. One non-limiting example of such a polypeptide linker is a GS linker, having an amino acid sequence of four glycine residues, followed by one serine residue, and wherein the sequence is repeated n times, where n is an integer ranging from 1 to 10 (SEQ ID NO: 42), such as 2, 3, 4, 5, 6, 7, 8, or 9. Non-limiting examples of such linkers include GGGGS (SEQ ID NO: 43) (n=1) and GGGGSGGGGS (SEQ ID NO: 44) (n=2). Other suitable linkers can also be used, and are described, for example, in Chen et al., Adv Drug Deliv Rev. 2013 Oct. 15; 65 (10): 1357-69, the disclosure of which is incorporated herein by reference in its entirety.

As used herein, the term “amino acid” or “amino acid residue” refers to an amino acid having its art recognized definition, such as, e.g., an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

As used herein, “amino acid modifications” include, but are not limited to, deletions from, and/or insertions into, and/or substitutions of, residues within an amino acid sequence. Any combination of deletion, insertion, and substitution may be made to arrive at a final construct, provided that the final construct possesses the desired characteristics. The amino acid changes also may alter post-translational processes of the antibody constructs, such as changing the number or position of glycosylation sites. Non-limiting example substitutions (or replacements) are conservative substitutions. However, any substitution (including non-conservative substitutions) is envisaged as long as the final construct retains its capability to bind to the target antigen.

One of skill in the art will realize that conservative variants of the antibodies, heavy-chain antibodies, antibody fragments, and antigen-binding fragments thereof described herein can be produced. Such conservative variants employed in antibody fragments, such as dsFv fragments or in scFv fragments, will retain critical amino acid residues necessary for correct folding and stabilizing between the VH and the VL regions, and will retain the charge characteristics of the residues in order to preserve the low pl and low toxicity of the molecules. In some embodiments, amino acid substitutions (such as, e.g., at most one, at most two, at most three, at most four, or at most five amino acid substitutions) can be made in the VH and/or the VL regions to increase yield. Conservative amino acid substitution tables providing functionally similar amino acids are well-known to one of ordinary skill in the art, such as, e.g., those described in TABLE 1.

TABLE 1
Example Conservative Substitutions

| | | Specific Example |
|---|---|---|
| Original | Example Substitutions | Substitutions |
| Ala (A) | Val, Leu, Ile | Val |
| Arg (R) | Lys, Gln, Asn | Lys |
| Asn (N) | Gln, His, Asp, Lys, Arg | Gln |
| Asp (D) | Glu, Asn | Glu |
| Cys (C) | Ser, Ala | Ser |
| Gln (Q) | Asn, Glu | Asn |
| Glu (E) | Asp, Gln | Asp |
| Gly (G) | Ala | Ala |
| His (H) | Asn, Gln, Lys, Arg | Arg |
| Ile (I) | Leu, Val, Met, Ala, Phe | Leu |
| Leu (L) | Norleucine, Ile, Val, Met, Ala | Ile |
| Lys (K) | Arg, Gln, Asn | Arg |
| Met (M) | Leu, Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala | Leu |

As used herein, “% identical,” “percent (%) amino acid sequence identity,” or “percent (%) sequence identity,” with respect to a reference polypeptide sequence, is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

As used herein, the term “cancer” refers to various conditions caused by the abnormal, uncontrolled growth of cells and includes neoplasms, primary tumors, secondary tumors, and other metastatic lesions. Cancer can be detected in a number of ways including, but not limited to, the presence of a tumor in a tissue as detected by clinical or radiological means, detection of cancerous or abnormal cells in a biological sample (e.g., tissue biopsy), detection of a biomarker indicative of a cancer or a pre-cancerous condition, or detection of a genotype indicative of cancer or the risk of developing cancer. The term “cancer” encompasses various cancerous conditions regardless of stage, grade, invasiveness, aggressiveness, or tissue type. Cancers that may be treated according to the methods of the present disclosure include, but are not limited to, leukemia (e.g., myeloid leukemia, chronic lymphocytic leukemia, chronic myelogenous leukemia, acute lymphoblastic leukemia), lymphoma (e.g., diffuse large B-cell lymphoma, Burkitt lymphoma, Non-Hodgkin lymphoma, follicular lymphoma), multiple myeloma, lung cancer (e.g., small-cell lung cancer (SCLC), non-small cell lung cancer (NSCLC)), glioma, glioblastoma, melanoma, prostate cancer (e.g., castration-resistant prostate cancer, neuroendocrine prostate cancer), pancreatic cancer, breast cancer, bone cancer, cervical cancer, colon cancer, colorectal cancer, endometrial cancer, head and neck cancer, liver cancer, ovarian cancer, gastric cancer, gastroesophageal junction cancer, testicular cancer, thyroid cancer, adrenal cancer, renal cancer, bladder cancer, uterine cancer, esophageal cancer, urothelial cancer, carcinoma, and sarcoma, and metastatic cancer derived from any of the foregoing.

As used herein, the term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by prevention of the occurrence of cancer in the first place. As used herein, the term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The terms “IL2” and “IL-2,” as used interchangeably herein, refer to interleukin-2, which is a 15.5 to 16 kDa cytokine signaling protein molecule that regulates the activity of certain immune cells by binding to IL2 receptor complexes expressed by lymphocytes. The term “IL2” includes an IL2 protein of any human and non-human animal species, and specifically includes human IL2 as well as IL2 of non-human mammals. The human IL-2 sequence (UniProtKB No. P60568) is provided herein as SEQ ID NO: 48. The term “human IL2” as used herein includes any variants, isoforms, and species homologs of human IL2, regardless of its source or mode of preparation. Thus, “human IL2” includes human IL2 naturally expressed by cells and IL2 expressed on cells transfected with the human IL2 gene.

The terms “IL2R,” “IL-2R,” “IL2 receptor,” and “IL-2 receptor,” as used interchangeably herein, refer generally to the IL2 receptor complex, which is composed of three polypeptide subunits, or chains, referred to as the alpha, A, or a chain, the beta, B, or β chain, and the gamma, G, or γ chain. IL-2R is a heterodimeric protein expressed on the surface of various immune cells, which serves as a cognate ligand for interleukin 2 (IL-2). The term “IL2R” includes any IL2R protein or any subunit of the IL2 receptor complex, of any human and non-human animal species, and specifically includes human IL2R as well as IL2R of non-human mammals. The term “human IL2R” as used herein includes any variants, isoforms, and species homologs of human IL2R, regardless of its source or mode of preparation. Thus, “human IL2R” includes human IL2R naturally expressed by cells and IL2R expressed on cells transfected with the human IL2R gene.

The term “IL2RA” or “IL2Rα” is also referred to as CD25, and the human IL2RA sequence (UniProtKB No. P01589) is provided herein as SEQ ID NO: 45.

The term “IL2RB” or “IL2Rβ” is also referred to as CD122, and the human IL2Rβ sequence (UniProtKB No. P14784) is provided herein as SEQ ID NO: 46.

The term “IL2RG” or “IL2Rγ” is also referred to as CD132, and the human IL2RG sequence (UniProtKB No. P31785) is provided herein as SEQ ID NO: 47.

The terms “anti-IL2R heavy chain-only antibody,” “IL2R heavy chain-only antibody,” “anti-IL2R heavy chain antibody,” and “IL2R heavy chain antibody” are used herein interchangeably to refer to a heavy-chain antibody as hereinabove defined, that binds to IL2R, including human IL2R, as hereinabove defined. The definition includes, without limitation, human heavy-chain antibodies produced by transgenic animals, such as transgenic rats or transgenic mice expressing human immunoglobulin, including UniRats™ producing human anti-IL2R UniAb™ antibodies. Similarly, as used herein, the term “anti-IL2Rβγ heavy chain-only antibody” or “anti-IL2Rβγ heavy-chain antibody” refers to a heavy-chain antibody that binds to IL2Rβ and IL2Rγ.

As used herein, the term “agonist” refers to a molecule that causes an increase in a function or activity as compared to the same function or activity in the absence of the molecule. An “agonist” of a signaling pathway is therefore a molecule whose presence causes an increase in a function or activity of the signaling pathway. The term “agonize,” as used herein, refers to causing an increase in a function or activity. In some embodiments, the agonist function of an antibody, antibody fragment, or antigen-binding fragment thereof may be determined using an assay described herein.

As used herein, an “epitope” is the site on the surface of an antigen molecule to which a single antigen-binding molecule binds. Generally, an antigen has several or many different epitopes and reacts with many different antibodies. The term specifically includes linear epitopes and conformational epitopes.

The term “valent,” as used herein, refers to a specified number of binding sites in a molecule.

A “monovalent” antibody has one binding site. Thus, a monovalent antibody is also monospecific.

A “multi-valent” antibody has two or more binding sites. Thus, the terms “bivalent,” “trivalent,” and “tetravalent” refer to the presence of two binding sites, three binding sites, and four binding sites, respectively. Thus, a bispecific antibody according to the disclosure is at least bivalent and may be trivalent, tetravalent, or otherwise multi-valent. A bivalent antibody in accordance with embodiments of the disclosure may have two binding sites to the same epitope (i.e., bivalent, monoparatopic), or to two different epitopes (i.e., bivalent, biparatopic).

A large variety of methods and protein configurations are known and used for the preparation of bispecific monoclonal antibodies (BsMAB), tri-specific antibodies, and the like.

As used herein, the term “effector cell” refers to an immune cell which is involved in the effector phase of an immune response, as opposed to the cognitive and activation phases of an immune response. Some effector cells express specific Fc receptors and carry out specific immune functions. In some embodiments, an effector cell such as a natural killer cell is capable of inducing antibody-dependent cellular cytotoxicity (ADCC). For example, monocytes and macrophages, which express FcR, are involved in specific killing of target cells and presenting antigens to other components of the immune system, or binding to cells that present antigens. In some embodiments, an effector cell may phagocytose a target antigen or target cell.

“Human effector cells” are leukocytes which express receptors such as T-cell receptors or FcRs and perform effector functions. For example, in some embodiments, the cells express at least FcγRIII and perform ADCC effector function. Non-limiting examples of human leukocytes which mediate ADCC include natural killer (NK) cells, monocytes, cytotoxic T-cells, and neutrophils. The effector cells may be isolated from a native source thereof, e.g., from blood or PBMCs as described herein.

The term “immune cell” is used herein in the broadest sense, including, without limitation, cells of myeloid or lymphoid origin, for instance, lymphocytes (such as B-cells and T-cells including cytolytic T-cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include, but are not limited to, Clq binding; complement dependent cytotoxicity (CDC); Fc receptor binding; antibody-dependent cell-mediated cytotoxicity (ADCC); phagocytosis; down regulation of cell surface receptors (e.g., B-cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (Clq) to a molecule (e.g., an antibody) complexed with a cognate antigen. To assess complement activation, a CDC assay, e.g., as described in Gazzano-Santoro et al., J. Immunol. Methods 202:163 (1996), may be performed.

“Binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art. Low-affinity antibodies generally bind antigen slowly and tend to dissociate readily, whereas high-affinity antibodies generally bind antigen faster and tend to remain bound.

The term “K_{D}” (M), “Kd,” or “Kd value,” as used herein, refers to the equilibrium dissociation constant of a particular antigen binding interaction as determined by BioLayer Interferometry, using an Octet QK384 instrument (Fortebio Inc., Menlo Park, CA) in kinetics mode. For example, anti-mouse Fc sensors are loaded with mouse-Fc fused antigen and then dipped into antibody-containing wells to measure concentration dependent association rates (kₒₙ). Antibody dissociation rates (k_{off}) are measured in the final step, where the sensors are dipped into wells containing buffer only. The K_{D} is the ratio of k_{off}/kₒₙ. (For further details see, Concepcion, J, et al., Comb Chem High Throughput Screen, 12 (8), 791-800, 2009).

In some embodiments, a molecule described herein as binding to a target antigen specifically binds to the target antigen. As used herein, a molecule (such as, e.g., an antibody, antibody fragment, or antigen-binding fragment) “specifically binds” to a target antigen when it has a significantly higher binding affinity for, and consequently is capable of distinguishing, that antigen compared to its affinity for other unrelated proteins, under similar binding assay conditions. For example, molecules that specifically bind an antigen may bind to that antigen with an equilibrium dissociation constant (K_{D})≤1×10⁻⁶ M. Molecules specifically bind antigen with “high affinity” when the K_{D} is ≤1×10⁻⁸ M. In some embodiments, molecules described herein bind to a target antigen with a K_{D} of ≤5×10⁻⁷ M. In some embodiments, molecules described herein bind to a target antigen with a K_{D} of ≤1×10⁻⁷ M. In some embodiments, molecules described herein bind to a target antigen with a K_{D} of ≤5×10⁻⁸ M. In some embodiments, molecules described herein bind to a target antigen with a K_{D} of ≤2×10⁻⁸ M. In some embodiments, molecules described herein bind to a target antigen with a K_{D} of ≤1×10⁻⁸ M. In some embodiments, molecules described herein bind to a target antigen with a K_{D} of ≤1×10⁻⁹ M.

Affinity may be determined using a variety of techniques, a non-limiting example of which is an affinity ELISA assay. In some embodiments, affinity is determined by a surface plasmon resonance assay (e.g., BIAcore®-based assay). Using this methodology, the association rate constant (kₐ in M⁻¹s⁻¹) and the dissociation rate constant (kₐ in s⁻¹) can be measured. The equilibrium dissociation constant (K_{D} in M) can then be calculated from the ratio of the kinetic rate constants (k_{d}/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹) can be measured. The dissociation rate constant (kₐ in s⁻¹) can be calculated from these values (K_{D}×kₐ). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (kₐ and kₐ) and affinity (K_{D}) constants can be calculated in real-time using the bio-layer interferometry method.

As used herein, the term “administer” and its cognates (e.g., “administering”) includes both self-administration and administration to the subject by another person (e.g., a medical professional or caretaker).

As used herein, the term “in combination with,” in the context of administration, as well as “co-administer,” “combined administration,” and their cognates (e.g., “co-administering”), means administration of two or more therapeutic agents in a coordinated fashion to a single subject and includes, but is not limited to, concurrent administration. Specifically, “co-administration” encompasses administration of a co-formulation or simultaneous administration of separate therapeutic compositions, as well as serial or sequential administration, provided that administration of one therapeutic agent is conditioned in some way on administration of another therapeutic agent. The therapeutic agents are not necessarily administered at the same time and/or by the same route of administration. Illustratively, one therapeutic agent may be administered only after a different therapeutic agent has been administered and allowed to act for a prescribed period of time. Additionally, in some embodiments, co-administered therapeutic agents are present in the subject (PK), or otherwise induce an effect (PD), at similar, identical, or partially overlapping periods of time.

It is envisaged that “prior to”, in the context of a first therapeutic agent being administered prior to a second therapeutic agent, means within 72 hours, 48 hours, 36 hours, 24 hours, 18 hours, 16 hours, 12 hours, 6 hours, 5 hours, 4 hours, or 3 hours, e.g., within 120 minutes, 90 minutes, 60 minutes, or 30 minutes before the start of administration of the second therapeutic agent.

In some embodiments, the combination partners may be administered entirely separately or be entirely separate pharmaceutical dosage forms. Additionally, in some embodiments, the combination partners may be pharmaceutical compositions that are also sold independently of each other, where instructions for their combined use are provided in the package equipment, e.g., leaflet or the like, or in other information, e.g., provided to physicians and medical staff (e.g., oral communications, communications in writing, or the like).

In some embodiments, the combination partners may be brought together into a combination therapy: (i) prior to release of the combination product to physicians (e.g., in the case of a kit comprising the combination partners); (ii) by the physician themselves (or under the guidance of a physician) shortly before administration; or (iii) in the patient themselves, e.g., during sequential administration of the combination partners.

As used herein, a “combination product” refers to a pharmaceutical product that results from the mixing or combining of more than one active ingredient and includes both fixed and non-fixed combinations of the active ingredients (which may also be combined). A combination product includes a kit of components for combined administration.

As used herein, the term “non-fixed combination” refers to therapeutic agents that are administered to a patient as separate entities either simultaneously, concurrently, or sequentially with no specific time limits, wherein such administration provides therapeutically effective levels of both therapeutic agents. The latter also applies to cocktail therapy, e.g., the administration of three or more active ingredients. In a non-fixed combination, the combination partners may be dosed independently of each other or by use of different fixed combinations with distinguished amounts of the combination partners.

As used herein, the term “fixed combination” refers to at least two therapeutic agents that are both administered to a patient simultaneously in the form of a single entity or dosage (i.e., the therapeutic agents are present in one dosage form).

As used herein, the term “treatment” and its cognates (e.g., “treating”) encompass any improvement of a disease in the subject, including the slowing or stopping of the progression of a disease in the subject, a decrease in the number or severity of the symptoms of the disease, or an increase in frequency or duration of periods where the patient is free from the symptoms of the disease.

As used herein, a “therapeutically effective amount” refers to an amount of active agent that imparts a therapeutic benefit to a subject. For example, a “therapeutically effective amount” is an amount which induces, ameliorates, or otherwise causes an improvement in the pathological symptoms, disease progression, or physiological conditions associated with a disease or which improves resistance to a disorder.

The terms “subject,” “individual,” and “patient” are used interchangeably herein to refer to a mammal being assessed for treatment and/or being treated. Subjects may be human, but also include other mammals, particularly those mammals useful as laboratory models for human disease, e.g., mouse, rat, etc. In some embodiments, the mammal is a human.

The term “pharmaceutical composition” refers to a preparation which is in such form as to permit the biological activity of the active ingredient to be effective, and which contains no additional components which are unacceptably toxic to a subject to which the formulation would be administered. Such compositions are sterile. “Pharmaceutically acceptable” excipients (e.g., vehicles, additives) are those which can reasonably be administered to a subject to provide an effective dose of the active ingredient employed.

As used herein, a “sterile” composition is aseptic or free or essentially free from all living microorganisms and their spores. As used herein, a “frozen” composition is one at a temperature below 0° C.

As used herein, a “stable” composition is one in which the protein therein essentially retains its physical stability and/or chemical stability and/or biological activity upon storage. In some embodiments, the composition essentially retains its physical and chemical stability, as well as its biological activity upon storage. The storage period is generally selected based on the intended shelf-life of the composition. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10:29-90) (1993), for example. Stability can be measured at a selected temperature for a selected time period. Stability can be evaluated qualitatively and/or quantitatively in a variety of different ways, including evaluation of aggregate formation (e.g., using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomerization), clipping/hydrolysis/fragmentation (e.g., hinge region fragmentation), succinimide formation, unpaired cysteine(s), N-terminal extension, C-terminal processing, glycosylation differences, etc.

As used herein, the term “vector” refers to a nucleic acid molecule capable of transporting another nucleic acid to which it has been linked. One type of vector is a “plasmid,” which refers to a circular double-stranded DNA loop into which additional DNA segments may be ligated. Another type of vector is a viral vector, wherein additional DNA segments may be ligated into the viral genome. Certain vectors are capable of autonomous replication in a host cell into which they are introduced (such as, e.g., bacterial vectors having a bacterial origin of replication and episomal mammalian vectors). Other vectors (such as, e.g., non-episomal mammalian vectors) may be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors.” In some embodiments, expression vectors for use in recombinant DNA techniques are in the form of plasmids.

As used herein, the term “host cell” refers to a cell into which an expression vector has been introduced. It should be understood that “host cell” is intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Example recombinant host cells include, but are not limited to, transfectomas, such as CHO cells, HEK293 cells, NS/0 cells, and lymphocytic cells.

As used herein, the term “autologous” refers to any material derived from an individual to whom the material is intended to be re-introduced.

As used herein, the term “allogeneic” refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

As used herein, the term “Chimeric Antigen Receptor,” or alternatively a “CAR,” refers to a recombinant polypeptide construct comprising at least an extracellular antigen binding domain, a transmembrane domain, and a cytoplasmic signaling domain (also referred to herein as “an intracellular signaling domain”) comprising a functional signaling domain derived from a stimulatory molecule. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains.

In some embodiments, the stimulatory molecule is the zeta chain associated with the T-cell receptor complex. In some embodiments, the cytoplasmic signaling domain comprises a primary signaling domain (e.g., a primary signaling domain of CD3-zeta). In some embodiments, the cytoplasmic signaling domain further comprises one or more functional signaling domains derived from at least one costimulatory molecule. In some embodiments, the costimulatory molecule is selected from 4-1BB (i.e., CD137), CD27, ICOS, and CD28. In some embodiments, the CAR comprises a chimeric fusion protein comprising an extracellular antigen binding domain, a transmembrane domain, and an intracellular signaling domain comprising a functional signaling domain derived from a stimulatory molecule. In some embodiments, the CAR comprises a chimeric fusion protein comprising an extracellular antigen binding domain, a transmembrane domain, and an intracellular signaling domain comprising a functional signaling domain derived from a co-stimulatory molecule and a functional signaling domain derived from a stimulatory molecule. In some embodiments, the CAR comprises a chimeric fusion protein comprising an extracellular antigen binding domain, a transmembrane domain, and an intracellular signaling domain comprising two functional signaling domains derived from one or more co-stimulatory molecule(s) and a functional signaling domain derived from a stimulatory molecule. In some embodiments, the CAR comprises a chimeric fusion protein comprising an extracellular antigen binding domain, a transmembrane domain, and an intracellular signaling domain comprising at least two functional signaling domains derived from one or more co-stimulatory molecule(s) and a functional signaling domain derived from a stimulatory molecule. In some embodiments, the CAR comprises an optional leader sequence at the amino-terminus (N-term) of the CAR fusion protein. In some embodiments, the CAR further comprises a leader sequence at the N-terminus of the extracellular antigen binding domain, wherein the leader sequence is optionally cleaved from the antigen recognition domain (e.g., a scFv) during cellular processing and localization of the CAR to the cellular membrane.

As used herein, the term “signaling domain” refers to the functional portion of a protein which acts by transmitting information within the cell to regulate cellular activity via defined signaling pathways by generating second messengers or functioning as effectors by responding to such messengers. In some embodiments, the signaling domain of a CAR described herein is derived from a stimulatory molecule or co-stimulatory molecule, or is a synthesized or engineered signaling domain.

As used herein, an “intracellular signaling domain” refers to an intracellular portion of a molecule. The intracellular signaling domain generates a signal that promotes an immune effector function of the CAR-expressing cell, e.g., a CAR-T cell or CAR-expressing NK cell. Non-limiting examples of immune effector function, e.g., in a CAR-T cell or CAR-expressing NK cell, include cytolytic activity and helper activity, including the secretion of cytokines. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some embodiments, the intracellular signaling domain may comprise a primary intracellular signaling domain. Example primary intracellular signaling domains include, but are not limited to, those derived from the molecules responsible for primary stimulation, or antigen dependent stimulation. In some embodiments, the intracellular signaling domain comprises a costimulatory intracellular domain. Example costimulatory intracellular signaling domains include, but are not limited to, those derived from molecules responsible for costimulatory signals, or antigen independent stimulation. In some embodiments, the intracellular signaling domain is synthesized or engineered. For example, in the case of a CAR-expressing immune effector cell, e.g., CAR-T cell or CAR-expressing NK cell, a primary intracellular signaling domain may comprise a cytoplasmic sequence of a T cell receptor, a primary intracellular signaling domain may comprise a cytoplasmic sequence of a T cell receptor, and a costimulatory intracellular signaling domain may comprise cytoplasmic sequence from co-receptor or costimulatory molecule.

In some embodiments, a primary intracellular signaling domain comprises a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta, CD3 gamma, CD3 delta, CD3 epsilon, CDS, CD22, CD79a, CD79^{b}, CD278 (“ICOS”), FcERI CD66d, DAP10, and DAP12.

As used herein, the term “costimulatory molecule” refers to the cognate binding partner on a T cell that binds with a costimulatory ligand, thereby mediating a costimulatory response by the T cell, including, but not limited to, proliferation. Costimulatory molecules are cell surface molecules other than antigen receptors or their ligands that are required for an efficient immune response. Costimulatory molecules include, but are not limited to, an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11^{b}, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that binds with CD83.

In some embodiments, a costimulatory intracellular signaling domain can be the intracellular portion of a costimulatory molecule. The intracellular signaling domain can comprise the entire intracellular portion, or the entire native intracellular signaling domain, of the molecule from which it is derived, or a functional fragment thereof.

As used herein, the term “zeta,” or alternatively “zeta chain” or “CD3-zeta,” is defined as the protein provided as GenBank Acc. No. BAG36664.1, or the equivalent residues from a non-human species, e.g., mouse, rodent, monkey, ape, and the like, and a “zeta stimulatory domain,” or alternatively a “CD3-zeta stimulatory domain,” is defined as the amino acid residues from the cytoplasmic domain of the zeta chain that are sufficient to functionally transmit an initial signal necessary for T cell activation. In some embodiments, the cytoplasmic domain of zeta comprises residues 52 through 164 of GenBank Acc. No. BAG36664.1 or the equivalent residues from a non-human species, e.g., mouse, rodent, monkey, ape and the like, which are functional orthologs thereof.

As used herein, the term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like.

As used herein, the term “T-cell redirecting therapy” refers to a therapeutic agent, such as a T-cell engaging molecule or a CAR T-cell, capable of recruiting T-cells to a target cell or tissue.

As used herein, the term “T-cell engaging molecule” refers to a molecule that comprises at least one domain in which the structure is derived from or comprises the minimum structural features of an antibody, e.g., of a full-length immunoglobulin molecule, that allow for specific binding to an antigen on the surface of a T cell, such as CD3. Thus, a T-cell engaging molecule according to the present disclosure generally comprises one or more binding domains, each of which will typically comprise the minimum structural requirements of an antibody that allow for specific target binding. This minimum requirement may, for example, be defined by the presence of at least three light chain “complementarity determining regions” or CDRs (i.e., CDRL1, CDRL2 and CDRL3 of a VL region) and/or three heavy chain CDRs (i.e., CDRH1, CDRH2 and CDRH3 of a VH region), such as, e.g., all six CDRs from both the light and heavy chain variable regions. The T-cell engaging molecules according to the present disclosure may comprise domains or regions (e.g., CDRs or variable regions) from monoclonal, chimeric, humanized and human antibodies. In some embodiments, the T-cell engaging molecules used in the methods of the present disclosure are proteins and comprise one or more polypeptide chains. In some embodiments, the T-cell engaging molecules administered according to the methods of the present disclosure are single-chain polypeptides. In other embodiments, the T-cell engaging molecules administered according to the methods of the present disclosure comprise two or more polypeptide chains—e.g., are polypeptide dimers or multimers. In certain embodiments, the T-cell engaging molecules administered according to the methods of the present disclosure comprise four polypeptide chains, and may, e.g., have the format of an antibody or an immunoglobulin protein.

As used herein, the term “bispecific T-cell engaging molecule” refers to a molecule capable of specifically binding to two different antigens. In the context of the present disclosure, bispecific T-cell engaging molecules specifically bind to a cancer cell antigen (e.g., human cancer cell antigen) on the cell surface of target cells and CD3 (e.g., human CD3) on the cell surface of T cells. In some embodiments, the T-cell engaging molecules may bind to more than one cancer cell antigen (e.g., human cancer cell antigen) on the cell surface of target cells as well as to CD3 (e.g., human CD3) on the cell surface of T cells. Thus, in such embodiments, the T-cell engaging molecules are “multitargeting” in that they are capable of specifically binding to two or more different cancer cell antigens and redirecting T cells to more than one type of cancer cell or cancer cells expressing the two or more antigens.

In some embodiments, the T-cell engaging molecules or binding domains thereof used in the methods of the disclosure bind to that antigen with an equilibrium dissociation constant (K_{D})≤1×10⁻⁶ M. In one embodiment, the T-cell engaging molecules or binding domains thereof used in the methods of the present disclosure bind to a human cancer cell antigen and/or human CD3 with a K_{D} of ≤5×10⁻⁷ M. In another embodiment, the T-cell engaging molecules or binding domains thereof used in the methods of the present disclosure bind to a human cancer cell antigen and/or human CD3 with a K_{D} of ≤1×10⁻⁷ M. In yet another embodiment, the T-cell engaging molecules or binding domains thereof used in the methods of the present disclosure bind to a human cancer cell antigen and/or human CD3 with a K_{D} of ≤5×10⁻⁸ M. In another embodiment, the T-cell engaging molecules or binding domains thereof used in the methods of the present disclosure bind to a human cancer cell antigen and/or human CD3 with a K_{D} of ≤2×10⁻⁸ M. In certain embodiments, the T-cell engaging molecules or binding domains thereof used in the methods of the present disclosure bind to a human cancer cell antigen and/or human CD3 with a K_{D} of ≤1×10⁻⁸ M. In other embodiments, the T-cell engaging molecules or binding domains thereof used in the methods of the present disclosure bind to a human cancer cell antigen and/or human CD3 with a K_{D} of ≤1×10⁻⁹ M.

In some embodiments, the T-cell engaging molecules or binding domains thereof described herein exhibit desirable characteristics such as binding avidity as measured by kₐ (dissociation rate constant) for a human cancer cell antigen and/or human CD3 of 10⁻², 10⁻³, 10⁻⁴, 10⁻⁵, 10⁻⁶, 10⁻⁷, 10⁻⁸, 10⁻⁹, 10⁻¹⁰ s⁻¹ or lower (lower values indicating higher binding avidity), and/or binding affinity as measured by K_{D} (equilibrium dissociation constant) for a human cancer cell antigen and/or human CD3 of 10⁻⁷, 10⁻⁸, 10⁻⁹, 10⁻¹⁰, 10⁻¹¹ M or lower (lower values indicating higher binding affinity).

In some embodiments, bispecific T-cell engaging molecules used in the methods of the present disclosure may be antibodies and have the general structure of a full-length immunoglobulin. For example, the bispecific T-cell engaging molecules may comprise two full-length antibody heavy chains and two full-length antibody light chains. In particular embodiments, the bispecific T-cell engaging molecules are heterodimeric antibodies (used interchangeably herein with “hetero immunoglobulins” or “hetero Igs”), which refer to antibodies comprising two different light chains and two different heavy chains. For instance, in some embodiments, the heterodimeric antibody comprises a light chain and heavy chain from an antibody that binds to a cancer cell antigen, such as the cancer cell antigens described further herein, and a light chain and heavy chain from an antibody that binds to CD3.

The bispecific T-cell engaging molecules employed in the methods of the present disclosure may also comprise fragments of full-length antibodies, such as VH, VHH, VL, (s) dAb, Fv, light chain (VL-CL), Fd (VH-CH1), heavy chain, Fab, Fab′, F(ab′) 2 or “r IgG” (“half antibody” consisting of a heavy chain and a light chain). Bispecific T-cell engaging molecules according to the present disclosure may also comprise modified fragments of antibodies. Examples of such modified fragments include, but are not limited to, single-chain variable fragment (scFv), di-scFv or bi(s)-scFv, scFv-Fc, scFv-zipper, single-chain Fab (scFab), Fab2, Fab3, diabodies, single-chain diabodies, tandem diabodies (Tandabs), tandem di-scFv, tandem tri-scFv, “minibodies” exemplified by a structure which is as follows: (VH-VL-CH3) 2, (scFv-CH3) 2, ((scFv) 2-CH3⁺CH3), ((scFv) 2-CH3) or (scFv-CH3-scFv) 2, multibodies, such as triabodies or tetrabodies, and single domain antibodies, such as nanobodies or single variable domain antibodies comprising merely one variable region, which might be VHH, VH or VL, that binds to an antigen or target independently of other variable regions or domains.

In some embodiments, the bispecific T-cell engaging molecule is a three-chain antibody-like molecule. In some embodiments, the bispecific T-cell engaging molecule is a heterodimeric IgG molecule (hetero-IgG). In some embodiments, the bispecific T-cell engaging molecule is a half-life extended (HLE) BiTE® molecule.

In certain embodiments, the bispecific T-cell engaging molecules used in the methods of the present disclosure are multivalent. The valency of the T-cell engaging molecule denotes the number of individual antigen-binding domains within the T-cell engaging molecule. For example, the terms “monovalent,” “bivalent,” and “tetravalent,” with reference to the T-cell engaging molecules in the context of the present disclosure, refer to T-cell engaging molecules with one, two, and four antigen-binding domains, respectively. Thus, a multivalent T-cell engaging molecule comprises two or more antigen-binding domains. A T-cell engaging molecule can have more antigen-binding domains (e.g., a higher valency) than specificities. For example, a T-cell engaging molecule having two antigen-binding domains for a first target (e.g., cancer cell antigen) and one antigen-binding domain for a second target (CD3)—or vice versa—is considered to be trivalent (three antigen-binding domains) and bispecific (binds to two antigens). In certain embodiments, the bispecific T-cell engaging molecules used in the methods of the present disclosure are bivalent. Thus, such bispecific, bivalent T-cell engaging molecules contain two antigen binding domains: one antigen-binding domain for a cancer cell antigen (e.g., a human cancer cell antigen) and one antigen-binding domain for CD3 (e.g., human CD3). In other embodiments, the T-cell engaging molecules used in the methods of the present disclosure are trivalent, trispecific T-cell engaging molecules and comprise three antigen binding domains: one antigen binding domain for a first cancer cell antigen, another antigen binding domain for a second cancer cell antigen, and a third binding domain for CD3. In still other embodiments, the T-cell engaging molecules used in the methods of the present disclosure are tetravalent, trispecific T-cell engaging molecules and comprise four antigen binding domains: one antigen binding domain for a first cancer cell antigen, another antigen binding domain for a second cancer cell antigen, and two antigen binding domains for CD3.

In some embodiments, the bispecific T-cell engaging molecules employed in the methods of the present disclosure comprise a first binding domain that binds to a target cancer cell antigen (e.g., a human target cancer cell antigen) and a second binding domain that binds to CD3 (e.g., human CD3). As used herein, the term “antigen-binding domain,” which is used interchangeably with “binding domain,” refers to the region of the T-cell engaging molecule that contains the amino acid residues that interact with the antigen and confer on the T-cell engaging molecule its specificity and affinity for the antigen. In certain embodiments, one or more binding domains of the T-cell engaging molecules may be derived from an antibody or antigen-binding fragment thereof. For instance, the binding domains of the bispecific T-cell engaging molecules used in the methods of the present disclosure may comprise one or more CDRs from the light and heavy chain variable regions of antibodies that specifically bind to a human target cancer cell antigen and/or human CD3. In some embodiments, the anti-cancer cell antigen binding domain of the bispecific T-cell engaging molecules comprises all six CDRs of the heavy and light chain variable regions of an antibody that binds to that human target cancer cell antigen and the anti-CD3 binding domain of the bispecific T-cell engaging molecules comprises all six CDRs of the heavy and light chain variable regions of an anti-CD3 antibody. In some embodiments, the binding domains (the anti-cancer cell antigen binding domain, the anti-CD3 binding domain or both) of the bispecific T-cell engaging molecules used in the methods of the present disclosure comprise a Fab, a Fab′, a F(ab′) 2, a Fv, a single-chain variable fragment (scFv), or a nanobody. In one embodiment, both binding domains of the bispecific T-cell engaging molecule are Fab fragments. In another embodiment, one binding domain of the bispecific T-cell engaging molecule is a Fab fragment and the other binding domain is a scFv. In yet another embodiment, both binding domains of the bispecific T-cell engaging molecule are scFvs.

### Anti-Il-2Rbg Heavy-Chain Antibodies

Provided herein is a heavy-chain antibody (e.g., an anti-IL2RBG heavy-chain antibody) comprising an Fc region that comprises an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering. For example, provided herein is a heavy-chain antibody comprising a first heavy chain variable (VH) region that binds to IL2Rβ, a second heavy chain variable region that binds to IL2RG, and an Fc region that comprises an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering.

Also provided herein is a heavy-chain antibody (e.g., an anti-IL2RBG heavy-chain antibody) comprising a first heavy chain variable region, a second heavy chain variable region, and an Fc region, wherein the first heavy chain variable region is connected to the Fc region by a first peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids, and the second heavy chain variable region is connected to the Fc region by a second peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids. For example, provided herein is a heavy-chain antibody comprising a first heavy chain variable (VH) region that binds to IL2Rβ, a second heavy chain variable region that binds to IL2RG, and an Fc region, wherein the first heavy chain variable region is connected to the Fc region by a first peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids, and the second heavy chain variable region is connected to the Fc region by a second peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids.

In some embodiments, the VH region that binds to IL2Rβ or IL2RG was described in WO 2022/212848, which is incorporated by reference herein. For example, in some embodiments, the first VH region that binds to IL2Rβ comprises CDR sequences having the following amino acid sequences as defined by IMGT. Heavy-chain antibodies comprising these CDR sequences can be referred to herein as IL2Rβ_F09 antibodies. An “X” indicates a variable amino acid, which may, in some embodiments, be a specific amino acid listed below:
CDR1 (IL2Rβ_F09)

(SEQ ID NO: 26)
G G S I S S S X₁ W,

wherein X₁ is D or N;

CDR2 (IL2RB_F09)
(SEQ ID NO: 27)
I X₂ H S G S T

wherein X₂ is D or S; and

CDR3 (IL2RB_F09)
(SEQ ID NO: 28)
X₃ R G X₄ W E L X₅ D A F D I

wherein X₃ is G or A; X₄ is S or Q; and X₅ is S or T.

In other embodiments, the first VH region that binds to IL2Rβ comprises CDR sequences having the following amino acid sequences as defined by IMGT. Heavy-chain antibodies comprising these CDR sequences can be referred to herein as IL2Rβ_F18 antibodies. An “X” indicates a variable amino acid, which may, in some embodiments, be a specific amino acid listed below:

CDR1 (IL2RB_F18)
(SEQ ID NO: 29)
G F T F S X₁ Y G

wherein X₁ is S or T;

CDR2 (IL2RB_F18)
(SEQ ID NO: 30)
I S Y D G S N X₂

wherein X₂ is K or R; and

CDR3 (IL2RB_F18)
(SEQ ID NO: 31)
A R D L D Y D X₃ L T G D P V G G F D I

wherein X₃ is V or I.

In some embodiments, the first VH region that binds to IL2Rβ comprises the VH CDR1, CDR2, and CDR3 sequences set forth in TABLE 2. The specific CDRs identified in TABLE 2 are defined by IMGT. In some embodiments, the first VH region that binds to IL2Rβ comprises a heavy chain variable region (VH) sequence set forth in TABLE 3.

TABLE 2
Anti-IL2RB Heavy-Chain Antibody Unique CDR Amino Acid Sequences (IMGT)

| Clone ID | Family | | | |
|---|---|---|---|---|
| No. | ID No. | CDR1 | CDR2 | CDR3 |
| 387205 | IL2RB_F09C | GGSISSSDW | IDHSGST | GRGSWELSDAFDI |
| | | (SEQ ID NO: 1) | (SEQ ID NO: 4) | (SEQ ID NO: 7) |
| 387172 | IL2RB_F09G | GGSISSSDW | IDHSGST | ARGSWELTDAFDI |
| | | (SEQ ID NO: 1) | (SEQ ID NO: 4) | (SEQ ID NO: 8) |
| 387111 | IL2RB_F09K | GGSISSSNW | ISHSGST | GRGSWELTDAFDI |
| | | (SEQ ID NO: 2) | (SEQ ID NO: 5) | (SEQ ID NO: 9) |
| 388252 | IL2RB_F18E | GFTFSSYG | ISYDGSNK | ARDLDYDVLTGDPVGG |
| | | (SEQ ID NO: 3) | (SEQ ID NO: 6) | FDI |
| | | | | (SEQ ID NO: 10) |

TABLE 3
Anti-IL2RB Heavy-Chain Antibody Variable Region Amino Acid Sequences

| Clone ID | Family | |
|---|---|---|
| No. | ID No. | VH Sequence |
| 387205 | IL2RB_F09C | QVQLQESGPGLVKPSGTLSLTCAVSGGSISSSDWWSWVRQ |
| | | PPGKGLEWIGEIDHSGSTNYNPSLMSRVTISVDKSKNQFSL |
| | | KLSSVTAADTAVYFCGRGSWELSDAFDIRGQGTLVTVSS |
| | | (SEQ ID NO: 11) |
| 387172 | IL2RB_F09G | QVQLQESGPGLVKSSETLSLTCTVSGGSISSSDWWSWVRQP |
| | | PGKGLEWIGEIDHSGSTNYNPSLMSRVTISVDKSKNQFSLK |
| | | LSSVTAADTAVYFCARGSWELTDAFDIRGQGTLVTVSS |
| | | (SEQ ID NO: 12) |
| 387111 | IL2RB_F09K | QVQLQESSPGLVKPSETLSLTCTVSGGSISSSNWWSWVRQP |
| | | PGKGLEWIGEISHSGSTNYNPSLKSRVTISVDKSKNQFSLRL |
| | | SSVTAADTAVYFCGRGSWELTDAFDIRGQGTLVTVSS (SEQ |
| | | ID NO: 13) |
| 388252 | IL2RB_F18E | QVQLVESGGGVVQPGRSLRLSCAASGFTFSSYGMHWVRQ |
| | | APGKEREWVAVISYDGSNKYYTDSVKGRFTISRDNSKNTL |
| | | YLEMNSLRAEDTAVYYCARDLDYDVLTGDPVGGFDIWGQ |
| | | GTLVTVSS (SEQ ID NO: 14) |

In some embodiments, the first VH region comprises a VH CDR1 comprising an amino acid sequence having at most two amino acid modifications relative to any one of SEQ ID NOs: 1-3; a VH CDR2 comprising an amino acid sequence having at most two amino acid modifications relative to any one of SEQ ID NOs: 4-6; and a VH CDR3 comprising an amino acid sequence having at most two amino acid modifications relative to any one of SEQ ID NOs: 7-10. In some embodiments, the first VH region comprises a VH CDR1 comprising an amino acid sequence having at most two amino acid modifications relative to SEQ ID NO: 1 or SEQ ID NO: 2; a VH CDR2 comprising an amino acid sequence having at most two amino acid modifications relative to SEQ ID NO: 4 or SEQ ID NO: 5; and a VH CDR3 comprising an amino acid sequence having at most two amino acid modifications relative to SEQ ID NO: 7, SEQ ID NO: 8, or SEQ ID NO: 9.

In some embodiments, the first VH region comprises a VH CDR1 comprising an amino acid sequence having at most two amino acid modifications relative to SEQ ID NO: 1; a VH CDR2 comprising an amino acid sequence having at most two amino acid modifications relative to SEQ ID NO: 4; and a VH CDR3 comprising an amino acid sequence having at most two amino acid modifications relative to SEQ ID NO: 7.

In some embodiments, the first VH region comprises a VH CDR1 comprising an amino acid sequence having at most two amino acid modifications relative to SEQ ID NO: 1; a VH CDR2 comprising an amino acid sequence having at most two amino acid modifications relative to SEQ ID NO: 4; and a VH CDR3 comprising an amino acid sequence having at most two amino acid modifications relative to SEQ ID NO: 8.

In some embodiments, the first VH region comprises a VH CDR1 comprising an amino acid sequence having at most two amino acid modifications relative to SEQ ID NO: 2; a VH CDR2 comprising an amino acid sequence having at most two amino acid modifications relative to SEQ ID NO: 5; and a VH CDR3 comprising an amino acid sequence having at most two amino acid modifications relative to SEQ ID NO: 9.

In some embodiments, the first VH region comprises a VH CDR1 comprising an amino acid sequence having at most two amino acid modifications relative to SEQ ID NO: 3; a VH CDR2 comprising an amino acid sequence having at most two amino acid modifications relative to SEQ ID NO: 6; and a VH CDR3 comprising an amino acid sequence having at most two amino acid modifications relative to SEQ ID NO: 10.

In some embodiments, each amino acid modification, if any, is an amino acid substitution, an amino acid addition, or an amino acid deletion. In some embodiments, each amino acid modification, if any, is an amino acid substitution. In some embodiments, each amino acid modification, if any, is a conservative amino acid substitution.

In some embodiments, the first VH region comprises a VH CDR1 comprising an amino acid sequence having at most one amino acid modification relative to any one of SEQ ID NOs: 1-3; a VH CDR2 comprising an amino acid sequence having at most one amino acid modification relative to any one of SEQ ID NOs: 4-6; and a VH CDR3 comprising an amino acid sequence having at most one amino acid modification relative to any one of SEQ ID NOs: 7-10. In some embodiments, the first VH region comprises a VH CDR1 comprising an amino acid sequence having at most one amino acid modification relative to SEQ ID NO: 1 or SEQ ID NO: 2; a VH CDR2 comprising an amino acid sequence having at most one amino acid modification relative to SEQ ID NO: 4 or SEQ ID NO: 5; and a VH CDR3 comprising an amino acid sequence having at most one amino acid modification relative to SEQ ID NO: 7, SEQ ID NO: 8, or SEQ ID NO: 9.

In some embodiments, the first VH region comprises a VH CDR1 comprising an amino acid sequence having at most one amino acid modification relative to SEQ ID NO: 1; a VH CDR2 comprising an amino acid sequence having at most one amino acid modification relative to SEQ ID NO: 4; and a VH CDR3 comprising an amino acid sequence having at most one amino acid modification relative to SEQ ID NO: 7.

In some embodiments, the first VH region comprises a VH CDR1 comprising an amino acid sequence having at most one amino acid modification relative to SEQ ID NO: 1; a VH CDR2 comprising an amino acid sequence having at most one amino acid modification relative to SEQ ID NO: 4; and a VH CDR3 comprising an amino acid sequence having at most one amino acid modification relative to SEQ ID NO: 8.

In some embodiments, the first VH region comprises a VH CDR1 comprising an amino acid sequence having at most one amino acid modification relative to SEQ ID NO: 2; a VH CDR2 comprising an amino acid sequence having at most one amino acid modification relative to SEQ ID NO: 5; and a VH CDR3 comprising an amino acid sequence having at most one amino acid modification relative to SEQ ID NO: 9.

In some embodiments, the first VH region comprises a VH CDR1 comprising an amino acid sequence having at most one amino acid modification relative to SEQ ID NO: 3; a VH CDR2 comprising an amino acid sequence having at most one amino acid modification relative to SEQ ID NO: 6; and a VH CDR3 comprising an amino acid sequence having at most one amino acid modification relative to SEQ ID NO: 10.

In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition. In some embodiments, each amino acid modification, if any, is an amino acid substitution. In some embodiments, each amino acid modification, if any, is a conservative amino acid substitution.

In some embodiments, the VH CDR1, the VH CDR2, and the VH CDR3 of the first VH region comprise the amino acid sequences of SEQ ID NO: 1 or SEQ ID NO: 2; SEQ ID NO: 4 or SEQ ID NO: 5; and SEQ ID NO: 7, SEQ ID NO: 8, or SEQ ID NO: 9, respectively. In other embodiments, the VH CDR1, the VH CDR2, and the VH CDR3 of the first VH region comprise the amino acid sequences of SEQ ID NO: 3, SEQ ID NO: 6, and SEQ ID NO: 10, respectively.

In some embodiments, the VH CDR1, the VH CDR2, and the VH CDR3 of the first VH region comprise the amino acid sequences of:
(a) SEQ ID NO: 1, SEQ ID NO: 4, and SEQ ID NO: 7, respectively; or
(b) SEQ ID NO: 1, SEQ ID NO: 4, and SEQ ID NO: 8, respectively; or
(c) SEQ ID NO: 2, SEQ ID NO: 5, and SEQ ID NO: 9, respectively; or
(d) SEQ ID NO: 3, SEQ ID NO: 6, and SEQ ID NO: 10, respectively.

In some embodiments, the VH CDR1, the VH CDR2, and the VH CDR3 of the first VH region comprise the amino acid sequences of:
(a) SEQ ID NO: 1, SEQ ID NO: 4, and SEQ ID NO: 7, respectively; or
(b) SEQ ID NO: 1, SEQ ID NO: 4, and SEQ ID NO: 8, respectively; or
(c) SEQ ID NO: 2, SEQ ID NO: 5, and SEQ ID NO: 9, respectively.

In some embodiments, the VH CDR1, the VH CDR2, and the VH CDR3 of the first VH region comprise the amino acid sequences of SEQ ID NO: 1, SEQ ID NO: 4, and SEQ ID NO: 7, respectively.

In some embodiments, the VH CDR1, the VH CDR2, and the VH CDR3 of the first VH region comprise the amino acid sequences of SEQ ID NO: 1, SEQ ID NO: 4, and SEQ ID NO: 8, respectively.

In some embodiments, the VH CDR1, the VH CDR2, and the VH CDR3 of the first VH region comprise the amino acid sequences of SEQ ID NO: 2, SEQ ID NO: 5, and SEQ ID NO: 9, respectively.

In some embodiments, the VH CDR1, the VH CDR2, and the VH CDR3 of the first VH region comprise the amino acid sequences of SEQ ID NO: 3, SEQ ID NO: 6, and SEQ ID NO: 10, respectively.

In some embodiments, the first VH region comprises an amino acid sequence that is at least 80% (e.g., at least 85%, at least 90%, at least 95%; 80%, 85%, 90%, 95%) identical to any one of SEQ ID NOs: 11-14. In some embodiments, the first VH region comprises an amino acid sequence that is at least 80% (e.g., at least 85%, at least 90%, at least 95%; 80%, 85%, 90%, 95%) identical to any one of SEQ ID NOs: 11-13.

In some embodiments, the first VH region comprises an amino acid sequence that is at least 80% (e.g., at least 85%, at least 90%, at least 95%; 80%, 85%, 90%, 95%) identical to the amino acid sequence of SEQ ID NO: 11. In some embodiments, the first VH region comprises an amino acid sequence that is at least 80% (e.g., at least 85%, at least 90%, at least 95%; 80%, 85%, 90%, 95%) identical to the amino acid sequence of SEQ ID NO: 12. In some embodiments, the first VH region comprises an amino acid sequence that is at least 80% (e.g., at least 85%, at least 90%, at least 95%; 80%, 85%, 90%, 95%) identical to the amino acid sequence of SEQ ID NO: 13. In some embodiments, the first VH region comprises an amino acid sequence that is at least 80% (e.g., at least 85%, at least 90%, at least 95%; 80%, 85%, 90%, 95%) identical to the amino acid sequence of SEQ ID NO: 14.

In some embodiments, the first VH region comprises an amino acid sequence selected from SEQ ID NOs: 11-14. In some embodiments, the first VH region comprises an amino acid sequence selected from SEQ ID NOs: 11-13.

In some embodiments, the first VH region comprises the amino acid sequence of SEQ ID NO: 11. In some embodiments, the first VH region comprises the amino acid sequence of SEQ ID NO: 12. In some embodiments, the first VH region comprises the amino acid sequence of SEQ ID NO: 13. In some embodiments, the first VH region comprises the amino acid sequence of SEQ ID NO: 14.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of any one of SEQ ID NOs: 11-14. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of any one of SEQ ID NOs: 11-13.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 11. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 14.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of any one of SEQ ID NOs: 11-14, wherein the VH CDRs 1, 2, and 3 are defined by any one of Kabat, Chothia, or IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of any one of SEQ ID NOs: 11-13, wherein the VH CDRs 1, 2, and 3 are defined by any one of Kabat, Chothia, or IMGT.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 11, wherein the VH CDRs 1, 2, and 3 are defined by any one of Kabat, Chothia, or IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12, wherein the VH CDRs 1, 2, and 3 are defined by any one of Kabat, Chothia, or IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13, wherein the VH CDRs 1, 2, and 3 are defined by any one of Kabat, Chothia, or IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 14, wherein the VH CDRs 1, 2, and 3 are defined by any one of Kabat, Chothia, or IMGT.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of any one of SEQ ID NOs: 11-14, wherein the VH CDRs 1, 2, and 3 are defined by Kabat. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of any one of SEQ ID NOs: 11-13, wherein the VH CDRs 1, 2, and 3 are defined by Kabat.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 11, wherein the VH CDRs 1, 2, and 3 are defined by Kabat. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12, wherein the VH CDRs 1, 2, and 3 are defined by Kabat. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13, wherein the VH CDRs 1, 2, and 3 are defined by Kabat. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 14, wherein the VH CDRs 1, 2, and 3 are defined by Kabat.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of any one of SEQ ID NOs: 11-14, wherein the VH CDRs 1, 2, and 3 are defined by Chothia. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of any one of SEQ ID NOs: 11-13, wherein the VH CDRs 1, 2, and 3 are defined by Chothia.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 11, wherein the VH CDRs 1, 2, and 3 are defined by Chothia. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12, wherein the VH CDRs 1, 2, and 3 are defined by Chothia. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13, wherein the VH CDRs 1, 2, and 3 are defined by Chothia. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 14, wherein the VH CDRs 1, 2, and 3 are defined by Chothia.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of any one of SEQ ID NOs: 11-14, wherein the VH CDRs 1, 2, and 3 are defined by IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of any one of SEQ ID NOs: 11-13, wherein the VH CDRs 1, 2, and 3 are defined by IMGT.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 11, wherein the VH CDRs 1, 2, and 3 are defined by IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12, wherein the VH CDRs 1, 2, and 3 are defined by IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13, wherein the VH CDRs 1, 2, and 3 are defined by IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 14, wherein the VH CDRs 1, 2, and 3 are defined by IMGT.

In some embodiments, the full set of VH CDRs 1, 2, and 3 (combined) in the first VH region is identical to the VH CDRs 1, 2, and 3 of any one of SEQ ID NOs: 11-14. In some embodiments, the full set of VH CDRs 1, 2, and 3 (combined) in the first VH region is identical to the VH CDRs 1, 2, and 3 of any one of SEQ ID NOs: 11-13.

In some embodiments, the full set of VH CDRs 1, 2, and 3 (combined) in the first VH region is identical to the VH CDRs 1, 2, and 3 of SEQ ID NO: 11. In some embodiments, the full set of VH CDRs 1, 2, and 3 (combined) in the first VH region is identical to the VH CDRs 1, 2, and 3 of SEQ ID NO: 12. In some embodiments, the full set of VH CDRs 1, 2, and 3 (combined) in the first VH region is identical to the VH CDRs 1, 2, and 3 of SEQ ID NO: 13. In some embodiments, the full set of VH CDRs 1, 2, and 3 (combined) in the first VH region is identical to the VH CDRs 1, 2, and 3 of SEQ ID NO: 14.

In some embodiments, the full set of VH CDRs 1, 2, and 3 (combined) in the first VH region is identical to the VH CDRs 1, 2, and 3 of any one of SEQ ID NOs: 11-14, wherein the VH CDRs 1, 2, and 3 are defined by any one of Kabat, Chothia, or IMGT. In some embodiments, the full set of VH CDRs 1, 2, and 3 (combined) in the first VH region is identical to the VH CDRs 1, 2, and 3 of any one of SEQ ID NOs: 11-13, wherein the VH CDRs 1, 2, and 3 are defined by any one of Kabat, Chothia, or IMGT.

In some embodiments, the full set of VH CDRs 1, 2, and 3 (combined) in the first VH region is identical to the VH CDRs 1, 2, and 3 of SEQ ID NO: 11, wherein the VH CDRs 1, 2, and 3 are defined by any one of Kabat, Chothia, or IMGT. In some embodiments, the full set of VH CDRs 1, 2, and 3 (combined) in the first VH region is identical to the VH CDRs 1, 2, and 3 of SEQ ID NO: 12, wherein the VH CDRs 1, 2, and 3 are defined by any one of Kabat, Chothia, or IMGT. In some embodiments, the full set of VH CDRs 1, 2, and 3 (combined) in the first VH region is identical to the VH CDRs 1, 2, and 3 of SEQ ID NO: 13, wherein the VH CDRs 1, 2, and 3 are defined by any one of Kabat, Chothia, or IMGT. In some embodiments, the full set of VH CDRs 1, 2, and 3 (combined) in the first VH region is identical to the VH CDRs 1, 2, and 3 of SEQ ID NO: 14, wherein the VH CDRs 1, 2, and 3 are defined by any one of Kabat, Chothia, or IMGT.

In some embodiments, the full set of VH CDRs 1, 2, and 3 (combined) in the first VH region is identical to the VH CDRs 1, 2, and 3 of any one of SEQ ID NOs: 11-14, wherein the VH CDRs 1, 2, and 3 are defined by Kabat. In some embodiments, the full set of VH CDRs 1, 2, and 3 (combined) in the first VH region is identical to the VH CDRs 1, 2, and 3 of any one of SEQ ID NOs: 11-13, wherein the VH CDRs 1, 2, and 3 are defined by Kabat.

In some embodiments, the full set of VH CDRs 1, 2, and 3 (combined) in the first VH region is identical to the VH CDRs 1, 2, and 3 of SEQ ID NO: 11, wherein the VH CDRs 1, 2, and 3 are defined by Kabat. In some embodiments, the full set of VH CDRs 1, 2, and 3 (combined) in the first VH region is identical to the VH CDRs 1, 2, and 3 of SEQ ID NO: 12, wherein the VH CDRs 1, 2, and 3 are defined by Kabat. In some embodiments, the full set of VH CDRs 1, 2, and 3 (combined) in the first VH region is identical to the VH CDRs 1, 2, and 3 of SEQ ID NO: 13, wherein the VH CDRs 1, 2, and 3 are defined by Kabat. In some embodiments, the full set of VH CDRs 1, 2, and 3 (combined) in the first VH region is identical to the VH CDRs 1, 2, and 3 of SEQ ID NO: 14, wherein the VH CDRs 1, 2, and 3 are defined by Kabat.

In some embodiments, the full set of VH CDRs 1, 2, and 3 (combined) in the first VH region is identical to the VH CDRs 1, 2, and 3 of any one of SEQ ID NOs: 11-14, wherein the VH CDRs 1, 2, and 3 are defined by Chothia. In some embodiments, the full set of VH CDRs 1, 2, and 3 (combined) in the first VH region is identical to the VH CDRs 1, 2, and 3 of any one of SEQ ID NOs: 11-13, wherein the VH CDRs 1, 2, and 3 are defined by Chothia.

In some embodiments, the full set of VH CDRs 1, 2, and 3 (combined) in the first VH region is identical to the VH CDRs 1, 2, and 3 of SEQ ID NO: 11, wherein the VH CDRs 1, 2, and 3 are defined by Chothia. In some embodiments, the full set of VH CDRs 1, 2, and 3 (combined) in the first VH region is identical to the VH CDRs 1, 2, and 3 of SEQ ID NO: 12, wherein the VH CDRs 1, 2, and 3 are defined by Chothia. In some embodiments, the full set of VH CDRs 1, 2, and 3 (combined) in the first VH region is identical to the VH CDRs 1, 2, and 3 of SEQ ID NO: 13, wherein the VH CDRs 1, 2, and 3 are defined by Chothia. In some embodiments, the full set of VH CDRs 1, 2, and 3 (combined) in the first VH region is identical to the VH CDRs 1, 2, and 3 of SEQ ID NO: 14, wherein the VH CDRs 1, 2, and 3 are defined by Chothia.

In some embodiments, the full set of VH CDRs 1, 2, and 3 (combined) in the first VH region is identical to the VH CDRs 1, 2, and 3 of any one of SEQ ID NOs: 11-14, wherein the VH CDRs 1, 2, and 3 are defined by IMGT. In some embodiments, the full set of VH CDRs 1, 2, and 3 (combined) in the first VH region is identical to the VH CDRs 1, 2, and 3 of any one of SEQ ID NOs: 11-13, wherein the VH CDRs 1, 2, and 3 are defined by IMGT.

In some embodiments, the full set of VH CDRs 1, 2, and 3 (combined) in the first VH region is identical to the VH CDRs 1, 2, and 3 of SEQ ID NO: 11, wherein the VH CDRs 1, 2, and 3 are defined by IMGT. In some embodiments, the full set of VH CDRs 1, 2, and 3 (combined) in the first VH region is identical to the VH CDRs 1, 2, and 3 of SEQ ID NO: 12, wherein the VH CDRs 1, 2, and 3 are defined by IMGT. In some embodiments, the full set of VH CDRs 1, 2, and 3 (combined) in the first VH region is identical to the VH CDRs 1, 2, and 3 of SEQ ID NO: 13, wherein the VH CDRs 1, 2, and 3 are defined by IMGT. In some embodiments, the full set of VH CDRs 1, 2, and 3 (combined) in the first VH region is identical to the VH CDRs 1, 2, and 3 of SEQ ID NO: 14, wherein the VH CDRs 1, 2, and 3 are defined by IMGT.

In some embodiments, the second VH region that binds to IL2RG comprises CDR sequences having the following amino acid sequences as defined by IMGT. Heavy-chain antibodies comprising these CDR sequences can be referred to herein as IL2RG_F16 antibodies. An “X” indicates a variable amino acid, which may, in some embodiments, be a specific amino acid listed below:

CDR1 (IL2RG_F16)
(SEQ ID NO: 32)
G F X₁ X₂ X₃ X₄ Y Y

wherein X₁ is T or I; X₂ is For V; X₃ is S, N, or G; and X₄ is D or N;

CDR2 (IL2RG_F16)
(SEQ ID NO: 33)
I S X₅ S G X₆ X₇ I

wherein X₅ is S or N; X₆ is D, S, G, or N; and X₇ is T or I; and

CDR3 (IL2RG_F16)
(SEQ ID NO: 20)
ARGDAVSITGDY.

In other embodiments, the second VH region that binds to IL2RG comprises a VH CDR1 sequence comprising GFTFSDYY (SEQ ID NO: 15), a VH CDR2 (IL2RG_F18) sequence comprising ISSSGTTT (SEQ ID NO: 19), and a VH CDR3 (IL2RG_F18) sequence comprising ARGAAVAPGFDS (SEQ ID NO: 21). Heavy-chain antibodies of comprising these CDR sequences can be referred to herein as IL2RG_F18 antibodies.

In some embodiments, the second VH region that binds to IL2RG comprises the VH CDR1, CDR2, and CDR3 sequences set forth in TABLE 4. The specific CDRs identified in TABLE 4 are defined by IMGT. In some embodiments, the second VH region that binds to IL2RG comprises a heavy chain variable region (VH) sequence set forth in TABLE 5.

TABLE 4
Anti-IL2RG Heavy-Chain Antibody CDR1, CDR2 and CDR3 Amino Acid Sequences

| Clone ID | Family | | | |
|---|---|---|---|---|
| No. | ID No. | CDR1 | CDR2 | CDR3 |
| 363256 | IL2RG_F16A | GFTFSDYY | ISSSGDTI | ARGDAVSITGDY |
| | | (SEQ ID NO: 15) | (SEQ ID NO: 17) | (SEQ ID NO: 20) |
| 363544 | IL2RG_F16B | GFTFSDYY | ISSSGSTI | ARGDAVSITGDY |
| | | (SEQ ID NO: 15) | (SEQ ID NO: 18) | (SEQ ID NO: 20) |
| 388582 | IL2RG_F16C | GFTFNDYY | ISSSGSTI | ARGDAVSITGDY |
| | | (SEQ ID NO: 16) | (SEQ ID NO: 18) | (SEQ ID NO: 20) |
| 363435 | IL2RG_F18A | GFTFSDYY | ISSSGTTT | ARGAAVAPGFDS |
| | | (SEQ ID NO: 15) | (SEQ ID NO: 19) | (SEQ ID NO: 21) |

TABLE 5
Anti-IL2RG Heavy-Chain Antibody Variable Region Amino Acid Sequences

| Clone ID | Family | |
|---|---|---|
| No. | ID No. | VH Sequence |
| 363256 | IL2RG_F16A | QVQLVESGGGLVKPGGSLRLSCAASGFTFSDYYMSWIRQAP |
| | | GKGLEWVSSISSSGDTIYYADSVQGRFTLSRDNAENSLFLQ |
| | | MNSLRAEDTAVYYCARGDAVSITGDYRGQGTLVTVSS |
| | | (SEQ ID NO: 22) |
| 363544 | IL2RG_F16B | QVQLVESGGGLVKPGGSLRLSCAASGFTFSDYYMSWIRQAP |
| | | GKGLEWVSYISSSGSTIYYADSVKGRFTISRDNAKNSLYLQ |
| | | MNSLRAEDTAVYYCARGDAVSITGDYRGQGTLVTVSS |
| | | (SEQ ID NO: 23) |
| 388582 | IL2RG_F16C | QVQLVESGGGLVKPGGSLRLSCAASGFTFNDYYMSWIRQA |
| | | PGKGLEWVSHISSSGSTIYYADSVKGRFTVSRDNANNSLYL |
| | | QMHSLRAEDTAVYYCARGDAVSITGDYRGQGTLVTVSS |
| | | (SEQ ID NO: 24) |
| 363435 | IL2RG_F18A | QVQLVESGGDLVKPGGSLRLSCAASGFTFSDYYMSWLRQA |
| | | PGKELEWVSHISSSGTTTYYADSVEGRFTITRDNAKNSLYLQ |
| | | MNSLRAEDTAVYYCARGAAVAPGFDSRGQGTLVTVSS |
| | | (SEQ ID NO: 25) |

In some embodiments, the second VH region comprises a VH CDR1 comprising an amino acid sequence having at most two amino acid modifications relative to SEQ ID NO: 15 or SEQ ID NO: 16; a VH CDR2 comprising an amino acid sequence having at most two amino acid modifications relative to any one of SEQ ID NOs: 17-19; and a VH CDR3 comprising an amino acid sequence having at most two amino acid modifications relative to SEQ ID NO: 20 or SEQ ID NO: 21.

In some embodiments, the second VH region comprises a VH CDR1 comprising an amino acid sequence having at most two amino acid modifications relative to SEQ ID NO: 15 or SEQ ID NO: 16; a VH CDR2 comprising an amino acid sequence having at most two amino acid modifications relative to SEQ ID NO: 17 or SEQ ID NO: 18; and a VH CDR3 comprising an amino acid sequence having at most two amino acid modifications relative to SEQ ID NO: 20.

In some embodiments, the second VH region comprises a VH CDR1 comprising an amino acid sequence having at most two amino acid modifications relative to SEQ ID NO: 15; a VH CDR2 comprising an amino acid sequence having at most two amino acid modifications relative to SEQ ID NO: 17; and a VH CDR3 comprising an amino acid sequence having at most two amino acid modifications relative to SEQ ID NO: 20.

In some embodiments, the second VH region comprises a VH CDR1 comprising an amino acid sequence having at most two amino acid modifications relative to SEQ ID NO: 15; a VH CDR2 comprising an amino acid sequence having at most two amino acid modifications relative to SEQ ID NO: 18; and a VH CDR3 comprising an amino acid sequence having at most two amino acid modifications relative to SEQ ID NO: 20.

In some embodiments, the second VH region comprises a VH CDR1 comprising an amino acid sequence having at most two amino acid modifications relative to SEQ ID NO: 16; a VH CDR2 comprising an amino acid sequence having at most two amino acid modifications relative to SEQ ID NO: 18; and a VH CDR3 comprising an amino acid sequence having at most two amino acid modifications relative to SEQ ID NO: 20.

In some embodiments, the second VH region comprises a VH CDR1 comprising an amino acid sequence having at most two amino acid modifications relative to SEQ ID NO: 15; a VH CDR2 comprising an amino acid sequence having at most two amino acid modifications relative to SEQ ID NO: 19; and a VH CDR3 comprising an amino acid sequence having at most two amino acid modifications relative to SEQ ID NO: 21.

In some embodiments, each amino acid modification, if any, is an amino acid substitution, an amino acid addition, or an amino acid deletion. In some embodiments, each amino acid modification, if any, is an amino acid substitution. In some embodiments, each amino acid modification, if any, is a conservative amino acid substitution.

In some embodiments, the second VH region comprises a VH CDR1 comprising an amino acid sequence having at most one amino acid modification relative to SEQ ID NO: 15 or SEQ ID NO: 16; a VH CDR2 comprising an amino acid sequence having at most one amino acid modification relative to any one of SEQ ID NOs: 17-19; and a VH CDR3 comprising an amino acid sequence having at most one amino acid modification relative to SEQ ID NO: 20 or SEQ ID NO: 21.

In some embodiments, the second VH region comprises a VH CDR1 comprising an amino acid sequence having at most one amino acid modification relative to SEQ ID NO: 15 or SEQ ID NO: 16; a VH CDR2 comprising an amino acid sequence having at most one amino acid modification relative to SEQ ID NO: 17 or SEQ ID NO: 18; and a VH CDR3 comprising an amino acid sequence having at most one amino acid modification relative to SEQ ID NO: 20.

In some embodiments, the second VH region comprises a VH CDR1 comprising an amino acid sequence having at most one amino acid modification relative to SEQ ID NO: 15; a VH CDR2 comprising an amino acid sequence having at most one amino acid modification relative to SEQ ID NO: 17; and a VH CDR3 comprising an amino acid sequence having at most one amino acid modification relative to SEQ ID NO: 20.

In some embodiments, the second VH region comprises a VH CDR1 comprising an amino acid sequence having at most one amino acid modification relative to SEQ ID NO: 15; a VH CDR2 comprising an amino acid sequence having at most one amino acid modification relative to SEQ ID NO: 18; and a VH CDR3 comprising an amino acid sequence having at most one amino acid modification relative to SEQ ID NO: 20.

In some embodiments, the second VH region comprises a VH CDR1 comprising an amino acid sequence having at most one amino acid modification relative to SEQ ID NO: 16; a VH CDR2 comprising an amino acid sequence having at most one amino acid modification relative to SEQ ID NO: 18; and a VH CDR3 comprising an amino acid sequence having at most one amino acid modification relative to SEQ ID NO: 20.

In some embodiments, the second VH region comprises a VH CDR1 comprising an amino acid sequence having at most one amino acid modification relative to SEQ ID NO: 15; a VH CDR2 comprising an amino acid sequence having at most one amino acid modification relative to SEQ ID NO: 19; and a VH CDR3 comprising an amino acid sequence having at most one amino acid modification relative to SEQ ID NO: 21.

In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition. In some embodiments, each amino acid modification, if any, is an amino acid substitution. In some embodiments, each amino acid modification, if any, is a conservative amino acid substitution.

In some embodiments, the VH CDR1, the VH CDR2, and the VH CDR3 of the second VH region comprise the amino acid sequences of SEQ ID NO: 15 or SEQ ID NO: 16; SEQ ID NO: 17 or SEQ ID NO: 18; and SEQ ID NO: 20, respectively. In other embodiments, the VH CDR1, the VH CDR2, and the VH CDR3 of the second VH region comprise the amino acid sequences of SEQ ID NO: 15, SEQ ID NO: 19, and SEQ ID NO: 21, respectively.

In some embodiments, the VH CDR1, the VH CDR2, and the VH CDR3 of the second VH region comprise the amino acid sequences of:
(a) SEQ ID NO: 15, SEQ ID NO: 17, and SEQ ID NO: 20, respectively; or
(b) SEQ ID NO: 15, SEQ ID NO: 18, and SEQ ID NO: 20, respectively; or
(c) SEQ ID NO: 16, SEQ ID NO: 18, and SEQ ID NO: 20, respectively; or
(d) SEQ ID NO: 15, SEQ ID NO: 19, and SEQ ID NO: 21, respectively.

In some embodiments, the VH CDR1, the VH CDR2, and the VH CDR3 of the second VH region comprise the amino acid sequences of:
(a) SEQ ID NO: 15, SEQ ID NO: 17, and SEQ ID NO: 20, respectively; or
(b) SEQ ID NO: 15, SEQ ID NO: 18, and SEQ ID NO: 20, respectively; or
(c) SEQ ID NO: 16, SEQ ID NO: 18, and SEQ ID NO: 20, respectively.

In some embodiments, the VH CDR1, the VH CDR2, and the VH CDR3 of the second VH region comprise the amino acid sequences of SEQ ID NO: 15, SEQ ID NO: 17, and SEQ ID NO: 20, respectively.

In some embodiments, the VH CDR1, the VH CDR2, and the VH CDR3 of the second VH region comprise the amino acid sequences of SEQ ID NO: 15, SEQ ID NO: 18, and SEQ ID NO: 20, respectively.

In some embodiments, the VH CDR1, the VH CDR2, and the VH CDR3 of the second VH region comprise the amino acid sequences of SEQ ID NO: 16, SEQ ID NO: 18, and SEQ ID NO: 20, respectively.

In some embodiments, the VH CDR1, the VH CDR2, and the VH CDR3 of the second VH region comprise the amino acid sequences of SEQ ID NO: 15, SEQ ID NO: 19, and SEQ ID NO: 21, respectively.

In some embodiments, the second VH region comprises an amino acid sequence that is at least 80% (e.g., at least 85%, at least 90%, at least 95%; 80%, 85%, 90%, 95%) identical to any one of SEQ ID NOs: 22-25. In some embodiments, the second VH region comprises an amino acid sequence that is at least 80% (e.g., at least 85%, at least 90%, at least 95%; 80%, 85%, 90%, 95%) identical to any one of SEQ ID NOs: 22-24.

In some embodiments, the second VH region comprises an amino acid sequence that is at least 80% (e.g., at least 85%, at least 90%, at least 95%; 80%, 85%, 90%, 95%) identical to SEQ ID NO: 22. In some embodiments, the second VH region comprises an amino acid sequence that is at least 80% (e.g., at least 85%, at least 90%, at least 95%; 80%, 85%, 90%, 95%) identical to SEQ ID NO: 23. In some embodiments, the second VH region comprises an amino acid sequence that is at least 80% (e.g., at least 85%, at least 90%, at least 95%; 80%, 85%, 90%, 95%) identical to SEQ ID NO: 24. In some embodiments, the second VH region comprises an amino acid sequence that is at least 80% (e.g., at least 85%, at least 90%, at least 95%; 80%, 85%, 90%, 95%) identical to SEQ ID NO: 25.

In some embodiments, the second VH region comprises an amino acid sequence selected from SEQ ID NOs: 22-25. In some embodiments, the second VH region comprises an amino acid sequence selected from SEQ ID NOs: 22-24.

In some embodiments, the second VH region comprises the amino acid sequence of SEQ ID NO: 22. In some embodiments, the second VH region comprises the amino acid sequence of SEQ ID NO: 23. In some embodiments, the second VH region comprises the amino acid sequence of SEQ ID NO: 24. In some embodiments, the second VH region comprises the amino acid sequence of SEQ ID NO: 25.

In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of any one of SEQ ID NOs: 22-25. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of any one of SEQ ID NOs: 22-24.

In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 22. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 24. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 25.

In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of any one of SEQ ID NOs: 22-25, wherein the VH CDRs 1, 2, and 3 are defined by any one of Kabat, Chothia, or IMGT. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of any one of SEQ ID NOs: 22-24, wherein the VH CDRs 1, 2, and 3 are defined by any one of Kabat, Chothia, or IMGT.

In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 22, wherein the VH CDRs 1, 2, and 3 are defined by any one of Kabat, Chothia, or IMGT. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23, wherein the VH CDRs 1, 2, and 3 are defined by any one of Kabat, Chothia, or IMGT. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 24, wherein the VH CDRs 1, 2, and 3 are defined by any one of Kabat, Chothia, or IMGT. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 25, wherein the VH CDRs 1, 2, and 3 are defined by any one of Kabat, Chothia, or IMGT.

In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of any one of SEQ ID NOs: 22-25, wherein the VH CDRs 1, 2, and 3 are defined by Kabat. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of any one of SEQ ID NOs: 22-24, wherein the VH CDRs 1, 2, and 3 are defined by Kabat.

In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 22, wherein the VH CDRs 1, 2, and 3 are defined by Kabat. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23, wherein the VH CDRs 1, 2, and 3 are defined by Kabat. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 24, wherein the VH CDRs 1, 2, and 3 are defined by Kabat. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 25, wherein the VH CDRs 1, 2, and 3 are defined by Kabat.

In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of any one of SEQ ID NOs: 22-25, wherein the VH CDRs 1, 2, and 3 are defined by Chothia. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of any one of SEQ ID NOs: 22-24, wherein the VH CDRs 1, 2, and 3 are defined by Chothia.

In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 22, wherein the VH CDRs 1, 2, and 3 are defined by Chothia. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23, wherein the VH CDRs 1, 2, and 3 are defined by Chothia. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 24, wherein the VH CDRs 1, 2, and 3 are defined by Chothia. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 25, wherein the VH CDRs 1, 2, and 3 are defined by Chothia.

In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of any one of SEQ ID NOs: 22-25, wherein the VH CDRs 1, 2, and 3 are defined by IMGT. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of any one of SEQ ID NOs: 22-24, wherein the VH CDRs 1, 2, and 3 are defined by IMGT.

In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 22, wherein the VH CDRs 1, 2, and 3 are defined by IMGT. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23, wherein the VH CDRs 1, 2, and 3 are defined by IMGT. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 24, wherein the VH CDRs 1, 2, and 3 are defined by IMGT. In some embodiments, the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 25, wherein the VH CDRs 1, 2, and 3 are defined by IMGT.

In some embodiments, the full set of VH CDRs 1, 2, and 3 (combined) in the second VH region is identical to the VH CDRs 1, 2, and 3 of any one of SEQ ID NOs: 22-25. In some embodiments, the full set of VH CDRs 1, 2, and 3 (combined) in the second VH region is identical to the VH CDRs 1, 2, and 3 of any one of SEQ ID NOs: 22-24.

In some embodiments, the full set of VH CDRs 1, 2, and 3 (combined) in the second VH region is identical to the VH CDRs 1, 2, and 3 of SEQ ID NO: 22. In some embodiments, the full set of VH CDRs 1, 2, and 3 (combined) in the second VH region is identical to the VH CDRs 1, 2, and 3 of SEQ ID NO: 23. In some embodiments, the full set of VH CDRs 1, 2, and 3 (combined) in the second VH region is identical to the VH CDRs 1, 2, and 3 of SEQ ID NO: 24. In some embodiments, the full set of VH CDRs 1, 2, and 3 (combined) in the second VH region is identical to the VH CDRs 1, 2, and 3 of SEQ ID NO: 25.

In some embodiments, the full set of VH CDRs 1, 2, and 3 (combined) in the second VH region is identical to the VH CDRs 1, 2, and 3 of any one of SEQ ID NOs: 22-25, wherein the VH CDRs 1, 2, and 3 are defined by any one of Kabat, Chothia, or IMGT. In some embodiments, the full set of VH CDRs 1, 2, and 3 (combined) in the second VH region is identical to the VH CDRs 1, 2, and 3 of any one of SEQ ID NOs: 22-24, wherein the VH CDRs 1, 2, and 3 are defined by any one of Kabat, Chothia, or IMGT.

In some embodiments, the full set of VH CDRs 1, 2, and 3 (combined) in the second VH region is identical to the VH CDRs 1, 2, and 3 of SEQ ID NO: 22, wherein the VH CDRs 1, 2, and 3 are defined by any one of Kabat, Chothia, or IMGT. In some embodiments, the full set of VH CDRs 1, 2, and 3 (combined) in the second VH region is identical to the VH CDRs 1, 2, and 3 of SEQ ID NO: 23, wherein the VH CDRs 1, 2, and 3 are defined by any one of Kabat, Chothia, or IMGT. In some embodiments, the full set of VH CDRs 1, 2, and 3 (combined) in the second VH region is identical to the VH CDRs 1, 2, and 3 of SEQ ID NO: 24, wherein the VH CDRs 1, 2, and 3 are defined by any one of Kabat, Chothia, or IMGT. In some embodiments, the full set of VH CDRs 1, 2, and 3 (combined) in the second VH region is identical to the VH CDRs 1, 2, and 3 of SEQ ID NO: 25, wherein the VH CDRs 1, 2, and 3 are defined by any one of Kabat, Chothia, or IMGT.

In some embodiments, the full set of VH CDRs 1, 2, and 3 (combined) in the second VH region is identical to the VH CDRs 1, 2, and 3 of any one of SEQ ID NOs: 22-25, wherein the VH CDRs 1, 2, and 3 are defined by Kabat. In some embodiments, the full set of VH CDRs 1, 2, and 3 (combined) in the second VH region is identical to the VH CDRs 1, 2, and 3 of any one of SEQ ID NOs: 22-24, wherein the VH CDRs 1, 2, and 3 are defined by Kabat.

In some embodiments, the full set of VH CDRs 1, 2, and 3 (combined) in the second VH region is identical to the VH CDRs 1, 2, and 3 of SEQ ID NO: 22, wherein the VH CDRs 1, 2, and 3 are defined by Kabat. In some embodiments, the full set of VH CDRs 1, 2, and 3 (combined) in the second VH region is identical to the VH CDRs 1, 2, and 3 of SEQ ID NO: 23, wherein the VH CDRs 1, 2, and 3 are defined by Kabat. In some embodiments, the full set of VH CDRs 1, 2, and 3 (combined) in the second VH region is identical to the VH CDRs 1, 2, and 3 of SEQ ID NO: 24, wherein the VH CDRs 1, 2, and 3 are defined by Kabat. In some embodiments, the full set of VH CDRs 1, 2, and 3 (combined) in the second VH region is identical to the VH CDRs 1, 2, and 3 of SEQ ID NO: 25, wherein the VH CDRs 1, 2, and 3 are defined by Kabat.

In some embodiments, the full set of VH CDRs 1, 2, and 3 (combined) in the second VH region is identical to the VH CDRs 1, 2, and 3 of any one of SEQ ID NOs: 22-25, wherein the VH CDRs 1, 2, and 3 are defined by Chothia. In some embodiments, the full set of VH CDRs 1, 2, and 3 (combined) in the second VH region is identical to the VH CDRs 1, 2, and 3 of any one of SEQ ID NOs: 22-24, wherein the VH CDRs 1, 2, and 3 are defined by Chothia.

In some embodiments, the full set of VH CDRs 1, 2, and 3 (combined) in the second VH region is identical to the VH CDRs 1, 2, and 3 of SEQ ID NO: 22, wherein the VH CDRs 1, 2, and 3 are defined by Chothia. In some embodiments, the full set of VH CDRs 1, 2, and 3 (combined) in the second VH region is identical to the VH CDRs 1, 2, and 3 of SEQ ID NO: 23, wherein the VH CDRs 1, 2, and 3 are defined by Chothia. In some embodiments, the full set of VH CDRs 1, 2, and 3 (combined) in the second VH region is identical to the VH CDRs 1, 2, and 3 of SEQ ID NO: 24, wherein the VH CDRs 1, 2, and 3 are defined by Chothia. In some embodiments, the full set of VH CDRs 1, 2, and 3 (combined) in the second VH region is identical to the VH CDRs 1, 2, and 3 of SEQ ID NO: 25, wherein the VH CDRs 1, 2, and 3 are defined by Chothia.

In some embodiments, the full set of VH CDRs 1, 2, and 3 (combined) in the second VH region is identical to the VH CDRs 1, 2, and 3 of any one of SEQ ID NOs: 22-25, wherein the VH CDRs 1, 2, and 3 are defined by IMGT. In some embodiments, the full set of VH CDRs 1, 2, and 3 (combined) in the second VH region is identical to the VH CDRs 1, 2, and 3 of any one of SEQ ID NOs: 22-24, wherein the VH CDRs 1, 2, and 3 are defined by IMGT.

In some embodiments, the full set of VH CDRs 1, 2, and 3 (combined) in the second VH region is identical to the VH CDRs 1, 2, and 3 of SEQ ID NO: 22, wherein the VH CDRs 1, 2, and 3 are defined by IMGT. In some embodiments, the full set of VH CDRs 1, 2, and 3 (combined) in the second VH region is identical to the VH CDRs 1, 2, and 3 of SEQ ID NO: 23, wherein the VH CDRs 1, 2, and 3 are defined by IMGT. In some embodiments, the full set of VH CDRs 1, 2, and 3 (combined) in the second VH region is identical to the VH CDRs 1, 2, and 3 of SEQ ID NO: 24, wherein the VH CDRs 1, 2, and 3 are defined by IMGT. In some embodiments, the full set of VH CDRs 1, 2, and 3 (combined) in the second VH region is identical to the VH CDRs 1, 2, and 3 of SEQ ID NO: 25, wherein the VH CDRs 1, 2, and 3 are defined by IMGT.

IL2Rβ_F09, IL2Rβ_F18, IL2RG_F16, and IL2RG_F18 antibodies are cross-reactive with the IL2R protein of Cynomolgus macaque, which facilitates the use of Cynomolgus macaque as an animal model for validating, e.g., mechanism of action, pharmacokinetics, toxicology, and other attributes of the heavy chain-only antibodies and antigen-binding fragments described herein.

In some embodiments, the VH CDR sequences of the first VH region or the second VH region may be situated, as an example, in the region of around amino acid residues 26-33; 51-58; and 97-116 for VH CDR1, VH CDR2, and VH CDR3, respectively, of the provided example variable region sequences set forth in SEQ ID NOs: 11-14 and 22-25. It will be understood by one of ordinary skill in the art that the CDR sequences may be in different positions if a different framework sequence is selected, although generally the order of the sequences will remain the same.

In some embodiments, the VH CDR1, the VH CDR2, and the VH CDR3 of the first VH region comprise the amino acid sequences of SEQ ID NO: 26, SEQ ID NO: 27, and SEQ ID NO: 28, respectively; and the VH CDR1, the VH CDR2, and the VH CDR3 of the second VH region comprise the amino acid sequences of SEQ ID NO: 32, SEQ ID NO: 33, and SEQ ID NO: 20, respectively.

In some embodiments, the VH CDR1, the VH CDR2, and the VH CDR3 of the first VH region comprise the amino acid sequences of SEQ ID NO: 26, SEQ ID NO: 27, and SEQ ID NO: 28, respectively; and the VH CDR1, the VH CDR2, and the VH CDR3 of the second VH region comprise the amino acid sequences of SEQ ID NO: 15, SEQ ID NO: 19, and SEQ ID NO: 21, respectively.

In some embodiments, the VH CDR1, the VH CDR2, and the VH CDR3 of the first VH region comprise the amino acid sequences of SEQ ID NO: 29, SEQ ID NO: 30, and SEQ ID NO: 31, respectively; and the VH CDR1, the VH CDR2, and the VH CDR3 of the second VH region comprise the amino acid sequences of SEQ ID NO: 32, SEQ ID NO: 33, and SEQ ID NO: 20, respectively.

In some embodiments, the VH CDR1, the VH CDR2, and the VH CDR3 of the first VH region comprise the amino acid sequences of SEQ ID NO: 29, SEQ ID NO: 30, and SEQ ID NO: 31, respectively; and the VH CDR1, the VH CDR2, and the VH CDR3 of the second VH region comprise the amino acid sequences of SEQ ID NO: 15, SEQ ID NO: 19, and SEQ ID NO: 21, respectively.

In some embodiments, the first VH region comprises a VH CDR1 comprising an amino acid sequence having at most two amino acid modifications relative to any one of SEQ ID NOs: 1-3; a VH CDR2 comprising an amino acid sequence having at most two amino acid modifications relative to any one of SEQ ID NOs: 4-6; and a VH CDR3 comprising an amino acid sequence having at most two amino acid modifications relative to any one of SEQ ID NOs: 7-10; and the second VH region comprises a VH CDR1 comprising an amino acid sequence having at most two amino acid modifications relative to SEQ ID NO: 15 or SEQ ID NO: 16; a VH CDR2 comprising an amino acid sequence having at most two amino acid modifications relative to any one of SEQ ID NOs: 17-19; and a VH CDR3 comprising an amino acid sequence having at most two amino acid modifications relative to SEQ ID NO: 20 or SEQ ID NO: 21.

In some embodiments, each amino acid modification, if any, is an amino acid substitution, an amino acid addition, or an amino acid deletion. In some embodiments, each amino acid modification, if any, is an amino acid substitution. In some embodiments, each amino acid modification, if any, is a conservative amino acid substitution.

In some embodiments, the first VH region comprises a VH CDR1 comprising an amino acid sequence having at most one amino acid modification relative to any one of SEQ ID NOs: 1-3; a VH CDR2 comprising an amino acid sequence having at most one amino acid modification relative to any one of SEQ ID NOs: 4-6; and a VH CDR3 comprising an amino acid sequence having at most one amino acid modification relative to any one of SEQ ID NOs: 7-10; and the second VH region comprises a VH CDR1 comprising an amino acid sequence having at most one amino acid modification relative to SEQ ID NO: 15 or SEQ ID NO: 16; a VH CDR2 comprising an amino acid sequence having at most one amino acid modification relative to any one of SEQ ID NOs: 17-19; and a VH CDR3 comprising an amino acid sequence having at most one amino acid modification relative to SEQ ID NO: 20 or SEQ ID NO: 21.

In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition. In some embodiments, each amino acid modification, if any, is an amino acid substitution. In some embodiments, each amino acid modification, if any, is a conservative amino acid substitution.

In some embodiments, the first VH region comprises a VH CDR1 comprising an amino acid sequence having at most two amino acid modifications relative to SEQ ID NO: 1; a VH CDR2 comprising an amino acid sequence having at most two amino acid modifications relative to SEQ ID NO: 4; and a VH CDR3 comprising an amino acid sequence having at most two amino acid modifications relative to SEQ ID NO: 8; and the second VH region comprises a VH CDR1 comprising an amino acid sequence having at most two amino acid modifications relative to SEQ ID NO: 15; a VH CDR2 comprising an amino acid sequence having at most two amino acid modifications relative to SEQ ID NO: 17; and a VH CDR3 comprising an amino acid sequence having at most two amino acid modifications relative to SEQ ID NO: 20.

In some embodiments, the first VH region comprises a VH CDR1 comprising an amino acid sequence having at most one amino acid modification relative to SEQ ID NO: 1; a VH CDR2 comprising an amino acid sequence having at most one amino acid modification relative to SEQ ID NO: 4; and a VH CDR3 comprising an amino acid sequence having at most one amino acid modification relative to SEQ ID NO: 7; and the second VH region comprises a VH CDR1 comprising an amino acid sequence having at most one amino acid modification relative to SEQ ID NO: 15; a VH CDR2 comprising an amino acid sequence having at most one amino acid modification relative to SEQ ID NO: 17; and a VH CDR3 comprising an amino acid sequence having at most one amino acid modification relative to SEQ ID NO: 20.

In some embodiments, the VH CDR1, the VH CDR2, and the VH CDR3 of the first VH region comprise the amino acid sequences of SEQ ID NO: 1, SEQ ID NO: 4, and SEQ ID NO: 7, respectively; and the VH CDR1, the VH CDR2, and the VH CDR3 of the second VH region comprise the amino acid sequences of SEQ ID NO: 15, SEQ ID NO: 17, and SEQ ID NO: 20, respectively.

In some embodiments, the first VH region comprises an amino acid sequence that is at least 80% (e.g., at least 85%, at least 90%, at least 95%; 80%, 85%, 90%, 95%) identical to SEQ ID NO: 11, and the second VH region comprises an amino acid sequence that is at least 80% (e.g., at least 85%, at least 90%, at least 95%; 80%, 85%, 90%, 95%) identical to SEQ ID NO: 22. In some embodiments, the first VH region comprises the amino acid sequence of SEQ ID NO: 11, and the second VH region comprises the amino acid sequence of SEQ ID NO: 22.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 11; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 22. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 11; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 22, wherein the VH CDRs 1, 2, and 3 are defined by any one of Kabat, Chothia, or IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 11; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 22, wherein the VH CDRs 1, 2, and 3 are defined by Kabat. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 11; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 22, wherein the VH CDRs 1, 2, and 3 are defined by Chothia. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 11; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 22, wherein the VH CDRs 1, 2, and 3 are defined by IMGT.

In some embodiments, the full set of VH CDRs 1, 2, and 3 (combined) in the first VH region is identical to the VH CDRs 1, 2, and 3 of SEQ ID NO: 11; and the full set of VH CDRs 1, 2, and 3 (combined) in the second VH region is identical to the VH CDRs 1, 2, and 3 of SEQ ID NO: 22. In some embodiments, the full set of VH CDRs 1, 2, and 3 (combined) in the first VH region is identical to the VH CDRs 1, 2, and 3 of SEQ ID NO: 11; and the full set of VH CDRs 1, 2, and 3 (combined) in the second VH region is identical to the VH CDRs 1, 2, and 3 of SEQ ID NO: 22, wherein the VH CDRs 1, 2, and 3 are defined by any one of Kabat, Chothia, or IMGT. In some embodiments, the full set of VH CDRs 1, 2, and 3 (combined) in the first VH region is identical to the VH CDRs 1, 2, and 3 of SEQ ID NO: 11; and the full set of VH CDRs 1, 2, and 3 (combined) in the second VH region is identical to the VH CDRs 1, 2, and 3 of SEQ ID NO: 22, wherein the VH CDRs 1, 2, and 3 are defined by Kabat. In some embodiments, the full set of VH CDRs 1, 2, and 3 (combined) in the first VH region is identical to the VH CDRs 1, 2, and 3 of SEQ ID NO: 11; and the full set of VH CDRs 1, 2, and 3 (combined) in the second VH region is identical to the VH CDRs 1, 2, and 3 of SEQ ID NO: 22, wherein the VH CDRs 1, 2, and 3 are defined by Chothia. In some embodiments, the full set of VH CDRs 1, 2, and 3 (combined) in the first VH region is identical to the VH CDRs 1, 2, and 3 of SEQ ID NO: 11; and the full set of VH CDRs 1, 2, and 3 (combined) in the second VH region is identical to the VH CDRs 1, 2, and 3 of SEQ ID NO: 22, wherein the VH CDRs 1, 2, and 3 are defined by IMGT.

In some embodiments, the first VH region comprises a VH CDR1 comprising an amino acid sequence having at most two amino acid modifications relative to SEQ ID NO: 1; a VH CDR2 comprising an amino acid sequence having at most two amino acid modifications relative to SEQ ID NO: 4; and a VH CDR3 comprising an amino acid sequence having at most two amino acid modifications relative to SEQ ID NO: 8; and the second VH region comprises a VH CDR1 comprising an amino acid sequence having at most two amino acid modifications relative to SEQ ID NO: 15; a VH CDR2 comprising an amino acid sequence having at most two amino acid modifications relative to SEQ ID NO: 18; and a VH CDR3 comprising an amino acid sequence having at most two amino acid modifications relative to SEQ ID NO: 20.

In some embodiments, the first VH region comprises a VH CDR1 comprising an amino acid sequence having at most one amino acid modification relative to SEQ ID NO: 1; a VH CDR2 comprising an amino acid sequence having at most one amino acid modification relative to SEQ ID NO: 4; and a VH CDR3 comprising an amino acid sequence having at most one amino acid modification relative to SEQ ID NO: 8; and the second VH region comprises a VH CDR1 comprising an amino acid sequence having at most one amino acid modification relative to SEQ ID NO: 15; a VH CDR2 comprising an amino acid sequence having at most one amino acid modification relative to SEQ ID NO: 18; and a VH CDR3 comprising an amino acid sequence having at most one amino acid modification relative to SEQ ID NO: 20.

In some embodiments, the VH CDR1, the VH CDR2, and the VH CDR3 of the first VH region comprise the amino acid sequences of SEQ ID NO: 1; SEQ ID NO: 4, and SEQ ID NO: 8, respectively; and the VH CDR1, the VH CDR2, and the VH CDR3 of the second VH region comprise the amino acid sequences of SEQ ID NO: 15, SEQ ID NO: 18, and SEQ ID NO: 20, respectively.

In some embodiments, the first VH region comprises an amino acid sequence that is at least 80% (e.g., at least 85%, at least 90%, at least 95%; 80%, 85%, 90%, 95%) identical to SEQ ID NO: 12, and the second VH region comprises an amino acid sequence that is at least 80% (e.g., at least 85%, at least 90%, at least 95%; 80%, 85%, 90%, 95%) identical to SEQ ID NO: 23. In some embodiments, the first VH region comprises the amino acid sequence of SEQ ID NO: 12, and the second VH region comprises the amino acid sequence of SEQ ID NO: 23.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23, wherein the VH CDRs 1, 2, and 3 are defined by any one of Kabat, Chothia, or IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23, wherein the VH CDRs 1, 2, and 3 are defined by Kabat. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23, wherein the VH CDRs 1, 2, and 3 are defined by Chothia. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23, wherein the VH CDRs 1, 2, and 3 are defined by IMGT.

In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 23. In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 23, wherein the VH CDRs 1, 2, and 3 are defined by any one of Kabat, Chothia, or IMGT. In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 23, wherein the VH CDRs 1, 2, and 3 are defined by Kabat. In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 23, wherein the VH CDRs 1, 2, and 3 are defined by Chothia. In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 23, wherein the VH CDRs 1, 2, and 3 are defined by IMGT.

In some embodiments, the first VH region comprises a VH CDR1 comprising an amino acid sequence having at most two amino acid modifications relative to SEQ ID NO: 2; a VH CDR2 comprising an amino acid sequence having at most two amino acid modifications relative to SEQ ID NO: 5; and a VH CDR3 comprising an amino acid sequence having at most two amino acid modifications relative to SEQ ID NO: 9; and the second VH region comprises a VH CDR1 comprising an amino acid sequence having at most two amino acid modifications relative to SEQ ID NO: 15; a VH CDR2 comprising an amino acid sequence having at most two amino acid modifications relative to SEQ ID NO: 18; and a VH CDR3 comprising an amino acid sequence having at most two amino acid modifications relative to SEQ ID NO: 20.

In some embodiments, the first VH region comprises a VH CDR1 comprising an amino acid sequence having at most one amino acid modification relative to SEQ ID NO: 2; a VH CDR2 comprising an amino acid sequence having at most one amino acid modification relative to SEQ ID NO: 5; and a VH CDR3 comprising an amino acid sequence having at most one amino acid modification relative to SEQ ID NO: 9; and the second VH region comprises a VH CDR1 comprising an amino acid sequence having at most one amino acid modification relative to SEQ ID NO: 15; a VH CDR2 comprising an amino acid sequence having at most one amino acid modification relative to SEQ ID NO: 18; and a VH CDR3 comprising an amino acid sequence having at most one amino acid modification relative to SEQ ID NO: 20.

In some embodiments, the VH CDR1, the VH CDR2, and the VH CDR3 of the first VH region comprise the amino acid sequences of SEQ ID NO: 2, SEQ ID NO: 5, and SEQ ID NO: 9, respectively; and the VH CDR1, the VH CDR2, and the VH CDR3 of the second VH region comprise the amino acid sequences of SEQ ID NO: 15, SEQ ID NO: 18, and SEQ ID NO: 20, respectively.

In some embodiments, the first VH region comprises an amino acid sequence that is at least 80% (e.g., at least 85%, at least 90%, at least 95%; 80%, 85%, 90%, 95%) identical to SEQ ID NO: 13, and the second VH region comprises an amino acid sequence that is at least 80% (e.g., at least 85%, at least 90%, at least 95%; 80%, 85%, 90%, 95%) identical to SEQ ID NO: 23. In some embodiments, the first VH region comprises the amino acid sequence of SEQ ID NO: 13, and the second VH region comprises the amino acid sequence of SEQ ID NO: 23.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23, wherein the VH CDRs 1, 2, and 3 are defined by any one of Kabat, Chothia, or IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23, wherein the VH CDRs 1, 2, and 3 are defined by Kabat. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23, wherein the VH CDRs 1, 2, and 3 are defined by Chothia. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23, wherein the VH CDRs 1, 2, and 3 are defined by IMGT.

In some embodiments, the full set of VH CDRs 1, 2, and 3 (combined) in the first VH region is identical to the VH CDRs 1, 2, and 3 of SEQ ID NO: 13; and the full set of VH CDRs 1, 2, and 3 (combined) in the second VH region is identical to the VH CDRs 1, 2, and 3 of SEQ ID NO: 23. In some embodiments, the full set of VH CDRs 1, 2, and 3 (combined) in the first VH region is identical to the VH CDRs 1, 2, and 3 of SEQ ID NO: 13; and the full set of VH CDRs 1, 2, and 3 (combined) in the second VH region is identical to the VH CDRs 1, 2, and 3 of SEQ ID NO: 23, wherein the VH CDRs 1, 2, and 3 are defined by any one of Kabat, Chothia, or IMGT. In some embodiments, the full set of VH CDRs 1, 2, and 3 (combined) in the first VH region is identical to the VH CDRs 1, 2, and 3 of SEQ ID NO: 13; and the full set of VH CDRs 1, 2, and 3 (combined) in the second VH region is identical to the VH CDRs 1, 2, and 3 of SEQ ID NO: 23, wherein the VH CDRs 1, 2, and 3 are defined by Kabat. In some embodiments, the full set of VH CDRs 1, 2, and 3 (combined) in the first VH region is identical to the VH CDRs 1, 2, and 3 of SEQ ID NO: 13; and the full set of VH CDRs 1, 2, and 3 (combined) in the second VH region is identical to the VH CDRs 1, 2, and 3 of SEQ ID NO: 23, wherein the VH CDRs 1, 2, and 3 are defined by Chothia. In some embodiments, the full set of VH CDRs 1, 2, and 3 (combined) in the first VH region is identical to the VH CDRs 1, 2, and 3 of SEQ ID NO: 13; and the full set of VH CDRs 1, 2, and 3 (combined) in the second VH region is identical to the VH CDRs 1, 2, and 3 of SEQ ID NO: 23, wherein the VH CDRs 1, 2, and 3 are defined by IMGT.

In some embodiments, the first VH region comprises a VH CDR1 comprising an amino acid sequence having at most two amino acid modifications relative to SEQ ID NO: 1; a VH CDR2 comprising an amino acid sequence having at most two amino acid modifications relative to SEQ ID NO: 4; and a VH CDR3 comprising an amino acid sequence having at most two amino acid modifications relative to SEQ ID NO: 8; and the second VH region comprises a VH CDR1 comprising an amino acid sequence having at most two amino acid modifications relative to SEQ ID NO: 16; a VH CDR2 comprising an amino acid sequence having at most two amino acid modifications relative to SEQ ID NO: 18; and a VH CDR3 comprising an amino acid sequence having at most two amino acid modifications relative to SEQ ID NO: 20.

In some embodiments, the first VH region comprises a VH CDR1 comprising an amino acid sequence having at most one amino acid modification relative to SEQ ID NO: 1; a VH CDR2 comprising an amino acid sequence having at most one amino acid modification relative to SEQ ID NO: 4; and a VH CDR3 comprising an amino acid sequence having at most one amino acid modification relative to SEQ ID NO: 8; and the second VH region comprises a VH CDR1 comprising an amino acid sequence having at most one amino acid modification relative to SEQ ID NO: 16; a VH CDR2 comprising an amino acid sequence having at most one amino acid modification relative to SEQ ID NO: 18; and a VH CDR3 comprising an amino acid sequence having at most one amino acid modification relative to SEQ ID NO: 20.

In some embodiments, the VH CDR1, the VH CDR2, and the VH CDR3 of the first VH region comprise the amino acid sequences of SEQ ID NO: 1, SEQ ID NO: 4, and SEQ ID NO: 8, respectively; and the VH CDR1, the VH CDR2, and the VH CDR3 of the second VH region comprise the amino acid sequences of SEQ ID NO: 16, SEQ ID NO: 18, and SEQ ID NO: 20, respectively.

In some embodiments, the first VH region comprises an amino acid sequence that is at least 80% (e.g., at least 85%, at least 90%, at least 95%; 80%, 85%, 90%, 95%) identical to SEQ ID NO: 12, and the second VH region comprises an amino acid sequence that is at least 80% (e.g., at least 85%, at least 90%, at least 95%; 80%, 85%, 90%, 95%) identical to SEQ ID NO: 24. In some embodiments, the first VH region comprises the amino acid sequence of SEQ ID NO: 12, and the second VH region comprises the amino acid sequence of SEQ ID NO: 24.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 24. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 24, wherein the VH CDRs 1, 2, and 3 are defined by any one of Kabat, Chothia, or IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 24, wherein the VH CDRs 1, 2, and 3 are defined by Kabat. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 24, wherein the VH CDRs 1, 2, and 3 are defined by Chothia. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 24, wherein the VH CDRs 1, 2, and 3 are defined by IMGT.

In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 24. In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 24, wherein the VH CDRs 1, 2, and 3 are defined by any one of Kabat, Chothia, or IMGT. In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 24, wherein the VH CDRs 1, 2, and 3 are defined by Kabat. In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 24, wherein the VH CDRs 1, 2, and 3 are defined by Chothia. In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 24, wherein the VH CDRs 1, 2, and 3 are defined by IMGT.

In some embodiments, the first VH region comprises a VH CDR1 comprising an amino acid sequence having at most two amino acid modifications relative to SEQ ID NO: 1; a VH CDR2 comprising an amino acid sequence having at most two amino acid modifications relative to SEQ ID NO: 4; and a VH CDR3 comprising an amino acid sequence having at most two amino acid modifications relative to SEQ ID NO: 8; and the second VH region comprises a VH CDR1 comprising an amino acid sequence having at most two amino acid modifications relative to SEQ ID NO: 15; a VH CDR2 comprising an amino acid sequence having at most two amino acid modifications relative to SEQ ID NO: 19; and a VH CDR3 comprising an amino acid sequence having at most two amino acid modifications relative to SEQ ID NO: 21.

In some embodiments, the first VH region comprises a VH CDR1 comprising an amino acid sequence having at most one amino acid modification relative to SEQ ID NO: 1; a VH CDR2 comprising an amino acid sequence having at most one amino acid modification relative to SEQ ID NO: 4; and a VH CDR3 comprising an amino acid sequence having at most one amino acid modification relative to SEQ ID NO: 8; and the second VH region comprises a VH CDR1 comprising an amino acid sequence having at most one amino acid modification relative to SEQ ID NO: 15; a VH CDR2 comprising an amino acid sequence having at most one amino acid modification relative to SEQ ID NO: 19; and a VH CDR3 comprising an amino acid sequence having at most one amino acid modification relative to SEQ ID NO: 21.

In some embodiments, the VH CDR1, the VH CDR2, and the VH CDR3 of the first VH region comprise the amino acid sequences of SEQ ID NO: 1, SEQ ID NO: 4, and SEQ ID NO: 8, respectively; and the VH CDR1, the VH CDR2, and the VH CDR3 of the second VH region comprise the amino acid sequences of SEQ ID NO: 15, SEQ ID NO: 19, and SEQ ID NO: 21, respectively.

In some embodiments, the first VH region comprises an amino acid sequence that is at least 80% (e.g., at least 85%, at least 90%, at least 95%; 80%, 85%, 90%, 95%) identical to SEQ ID NO: 12, and the second VH region comprises an amino acid sequence that is at least 80% (e.g., at least 85%, at least 90%, at least 95%; 80%, 85%, 90%, 95%) identical to SEQ ID NO: 25. In some embodiments, the first VH region comprises the amino acid sequence of SEQ ID NO: 12, and the second VH region comprises the amino acid sequence of SEQ ID NO: 25.

In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 25. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 25, wherein the VH CDRs 1, 2, and 3 are defined by any one of Kabat, Chothia, or IMGT. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 25, wherein the VH CDRs 1, 2, and 3 are defined by Kabat. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 25, wherein the VH CDRs 1, 2, and 3 are defined by Chothia. In some embodiments, the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 12; and the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 25, wherein the VH CDRs 1, 2, and 3 are defined by IMGT.

In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 25. In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 25, wherein the VH CDRs 1, 2, and 3 are defined by any one of Kabat, Chothia, or IMGT. In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 25, wherein the VH CDRs 1, 2, and 3 are defined by Kabat. In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 25, wherein the VH CDRs 1, 2, and 3 are defined by Chothia. In some embodiments, the full set of VH CDRs 1, 2, and 3 (combined) in the first VH region is identical to the VH CDRs 1, 2, and 3 of SEQ ID NO: 12; and the full set of VH CDRs 1, 2, and 3 (combined) in the second VH region is identical to the VH CDRs 1, 2, and 3 of SEQ ID NO: 25, wherein the VH CDRs 1, 2, and 3 are defined by IMGT.

Some embodiments of the present disclosure relate to heavy chain antibodies (e.g., anti-IL2RBG heavy-chain antibodies) comprising an Fc region that comprises an amino acid sequence that is at least 90% (e.g., at least 91%, at least 92%, at least 93%, at least 94%, at least 95%, at least 96%, at least 97%, at least 98%; 91%, 92%, 93%, 94%, 95%, 96%, 97%, 98%) identical to the amino acid sequence of SEQ ID NO: 34, wherein the amino acid sequence comprises a glycine at position 297, a cysteine at position 292, and a cysteine at position 302, all according to EU numbering.

In some embodiments, the Fc region comprises an amino acid sequence that is at least 91% identical to the amino acid sequence of SEQ ID NO: 34. In some embodiments, the Fc region comprises an amino acid sequence that is at least 92% identical to the amino acid sequence of SEQ ID NO: 34. In some embodiments, the Fc region comprises an amino acid sequence that is at least 93% identical to the amino acid sequence of SEQ ID NO: 34. In some embodiments, the Fc region comprises an amino acid sequence that is at least 94% identical to the amino acid sequence of SEQ ID NO: 34. In some embodiments, the Fc region comprises an amino acid sequence that is at least 95% identical to the amino acid sequence of SEQ ID NO: 34. In some embodiments, the Fc region comprises an amino acid sequence that is at least 96% identical to the amino acid sequence of SEQ ID NO: 34. In some embodiments, the Fc region comprises an amino acid sequence that is at least 97% identical to the amino acid sequence of SEQ ID NO: 34.

In some embodiments, the Fc region comprises an amino acid sequence that is 97.4% identical to the amino acid sequence of SEQ ID NO: 34. In some embodiments, the Fc region comprises an amino acid sequence that is 97.8% identical to the amino acid sequence of SEQ ID NO: 34. In some embodiments, the Fc region comprises an amino acid sequence that is 98.2% identical to the amino acid sequence of SEQ ID NO: 34. In some embodiments, the Fc region comprises an amino acid sequence that is 98.6% identical to the amino acid sequence of SEQ ID NO: 34.

In some embodiments, the Fc region comprises a first polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering. In some embodiments, the Fc region comprises a first polypeptide chain comprising an amino acid sequence that is at least 95% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering. In some embodiments, the Fc region comprises a first polypeptide chain comprising an amino acid sequence that is between 90% and 97.8% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering.

In some embodiments, the first polypeptide chain comprises the amino acid sequence of SEQ ID NO: 35.

In some embodiments, the Fc region further comprises a second polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering. In some embodiments, the Fc region further comprises a second polypeptide chain comprising an amino acid sequence that is at least 95% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering. In some embodiments, the Fc region further comprises a second polypeptide chain comprising an amino acid sequence that is between 90% and 97.4% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

In some embodiments, the second polypeptide chain comprises the amino acid sequence of SEQ ID NO: 36.

In some embodiments, the Fc region comprises a first polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering; and a second polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

In some embodiments, the Fc region comprises a first polypeptide chain comprising an amino acid sequence that is at least 95% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering; and a second polypeptide chain comprising an amino acid sequence that is at least 95% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

In some embodiments, the Fc region comprises a first polypeptide chain comprising an amino acid sequence that is between 90% and 97.8% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering; and a second polypeptide chain comprising an amino acid sequence that is between 90% and 97.4% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

In some embodiments, the first polypeptide chain comprises the amino acid sequence of SEQ ID NO: 35, and the second polypeptide chain comprises the amino acid sequence of SEQ ID NO: 36.

In some embodiments, the first heavy chain variable region is connected to the Fc region by a first peptide linker.

In some embodiments, the second heavy chain variable region is connected to the Fc region by a second peptide linker.

In some embodiments, the first heavy chain variable region is connected to the Fc region by a first peptide linker, and the second heavy chain variable region is connected to the Fc region by a second peptide linker.

In some embodiments, the first heavy chain variable region is connected to the first polypeptide chain of the Fc region by a first peptide linker, and the second heavy chain variable region is connected to the second polypeptide chain of the Fc region by a second peptide linker.

In some embodiments, the first heavy chain variable region is connected to the second polypeptide chain of the Fc region by a first peptide linker, and the second heavy chain variable region is connected to the first polypeptide chain of the Fc region by a second peptide linker.

In some embodiments, the first peptide linker and the second peptide linker have the same amino acid sequence. In other embodiments, the first peptide linker and the second peptide linker have different amino acid sequences.

In some embodiments, the first peptide linker comprises at least four amino acids (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10). In some embodiments, the second peptide linker comprises at least four amino acids (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10). In some embodiments, the first peptide linker comprises at least four amino acids, and the second peptide linker comprises at least four amino acids.

In some embodiments, the first peptide linker comprises between 4 and 10 amino acids. In some embodiments, the second peptide linker comprises between 4 and 10 amino acids. In some embodiments, the first peptide linker comprises between 4 and 10 amino acids, and the second peptide linker comprises between 4 and 10 amino acids.

In some embodiments, the first peptide linker comprises four amino acids. In some embodiments, the second peptide linker comprises four amino acids. In some embodiments, the first peptide linker comprises four amino acids, and the second peptide linker comprises four amino acids.

In some embodiments, the first peptide linker is a flexible linker. In some embodiments, the second peptide linker is a flexible linker. In some embodiments, the first peptide linker and the second peptide linker are both flexible linkers.

In some embodiments, the first peptide linker comprises glycine, serine, glutamine, and threonine amino acids. In some embodiments, the second peptide linker comprises glycine, serine, glutamine, and threonine amino acids. In some embodiments, the first peptide linker and the second peptide linker both comprise glycine, serine, glutamine, and threonine amino acids.

In some embodiments, the first peptide linker comprises glycine, serine, and threonine amino acids. In some embodiments, the second peptide linker comprises glycine, serine, and threonine amino acids. In some embodiments, the first peptide linker and the second peptide linker both comprise glycine, serine, and threonine amino acids.

In some embodiments, the first peptide linker comprises glycine, serine, glutamine, and threonine amino acids only. In some embodiments, the second peptide linker comprises glycine, serine, glutamine, and threonine amino acids only. In some embodiments, the first peptide linker and the second peptide linker both comprise glycine, serine, glutamine, and threonine amino acids only.

In some embodiments, the first peptide linker comprises glycine, serine, and threonine amino acids only. In some embodiments, the second peptide linker comprises glycine, serine, and threonine amino acids only. In some embodiments, the first peptide linker and the second peptide linker both comprise glycine, serine, and threonine amino acids only.

In some embodiments, the first peptide linker comprises the amino acid sequence of (Gly-Gly-Gly-Gly-Ser) m, wherein m is a number in the range of 1 to 5 (SEQ ID NO: 40). In some embodiments, the second peptide linker comprises the amino acid sequence of (Gly-Gly-Gly-Gly-Ser) m, wherein m is a number in the range of 1 to 5 (SEQ ID NO: 40). In some embodiments, the first peptide linker and the second peptide linker both independently comprise amino acid sequences of (Gly-Gly-Gly-Gly-Ser) m, wherein m is a number in the range of 1 to 5 (SEQ ID NO: 40).

In some embodiments, the first peptide linker comprises the amino acid sequence of (Gly-Gly-Gly-Gly-Gln) n, wherein n is a number in the range of 1 to 5 (SEQ ID NO: 82). In some embodiments, the second peptide linker comprises the amino acid sequence of (Gly-Gly-Gly-Gly-Gln) n, wherein n is a number in the range of 1 to 5 (SEQ ID NO: 82). In some embodiments, the first peptide linker and the second peptide linker both independently comprise amino acid sequences of (Gly-Gly-Gly-Gly-Gln) n, wherein n is a number in the range of 1 to 5 (SEQ ID NO: 82).

In some embodiments, the first peptide linker is a poly-Gly linker. In some embodiments, the second peptide linker is a poly-Gly linker. In some embodiments, the first peptide linker and the second peptide linker are independently poly-Gly linkers.

In some embodiments, the first peptide linker comprises the amino acid sequence of (Gly) p, wherein p is a number in the range of 4 to 10 (SEQ ID NO: 41). In some embodiments, the second peptide linker comprises the amino acid sequence of (Gly) p, wherein p is a number in the range of 4 to 10 (SEQ ID NO: 41). In some embodiments, the first peptide linker and the second peptide linker both independently comprise amino acid sequences of (Gly) p, wherein p is a number in the range of 4 to 10 (SEQ ID NO: 41).

In some embodiments, the first peptide linker comprises the amino acid sequence of GGGG (SEQ ID NO: 37). In some embodiments, the second peptide linker comprises the amino acid sequence of GGGG (SEQ ID NO: 37). In some embodiments, the first peptide linker and the second peptide linker both comprise the amino acid sequence of GGGG (SEQ ID NO: 37).

Some embodiments of the present disclosure relate to heavy-chain antibodies (e.g., anti-IL2RBG heavy-chain antibodies) comprising a first heavy chain variable region, a second heavy chain variable region, and an Fc region, wherein the first heavy chain variable region is connected to the Fc region by a first peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids, and the second heavy chain variable region is connected to the Fc region by a second peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids.

In some embodiments, the first peptide linker and the second peptide linker have the same amino acid sequence. In other embodiments, the first peptide linker and the second peptide linker have different amino acid sequences.

In some embodiments, the first peptide linker comprises between 4 and 10 amino acids. In some embodiments, the second peptide linker comprises between 4 and 10 amino acids. In some embodiments, the first peptide linker comprises between 4 and 10 amino acids, and the second peptide linker comprises between 4 and 10 amino acids.

In some embodiments, the first peptide linker comprises four amino acids. In some embodiments, the second peptide linker comprises four amino acids. In some embodiments, the first peptide linker comprises four amino acids, and the second peptide linker comprises four amino acids.

In some embodiments, the first peptide linker is a flexible linker. In some embodiments, the second peptide linker is a flexible linker. In some embodiments, the first peptide linker and the second peptide linker are both flexible linkers.

In some embodiments, the first peptide linker comprises glycine, serine, glutamine, and threonine amino acids. In some embodiments, the second peptide linker comprises glycine, serine, glutamine, and threonine amino acids. In some embodiments, the first peptide linker and the second peptide linker both comprise glycine, serine, glutamine, and threonine amino acids.

In some embodiments, the first peptide linker comprises glycine, serine, and threonine amino acids. In some embodiments, the second peptide linker comprises glycine, serine, and threonine amino acids. In some embodiments, the first peptide linker and the second peptide linker both comprise glycine, serine, and threonine amino acids.

In some embodiments, the first peptide linker comprises glycine, serine, glutamine, and threonine amino acids only. In some embodiments, the second peptide linker comprises glycine, serine, glutamine, and threonine amino acids only. In some embodiments, the first peptide linker and the second peptide linker both comprise glycine, serine, glutamine, and threonine amino acids only.

In some embodiments, the first peptide linker comprises glycine, serine, and threonine amino acids only. In some embodiments, the second peptide linker comprises glycine, serine, and threonine amino acids only. In some embodiments, the first peptide linker and the second peptide linker both comprise glycine, serine, and threonine amino acids only.

In some embodiments, the first peptide linker comprises the amino acid sequence of (Gly-Gly-Gly-Gly-Ser) m, wherein m is a number in the range of 1 to 5 (SEQ ID NO: 40). In some embodiments, the second peptide linker comprises the amino acid sequence of (Gly-Gly-Gly-Gly-Ser) m, wherein m is a number in the range of 1 to 5 (SEQ ID NO: 40). In some embodiments, the first peptide linker and the second peptide linker both independently comprise amino acid sequences of (Gly-Gly-Gly-Gly-Ser) m, wherein m is a number in the range of 1 to 5 (SEQ ID NO: 40).

In some embodiments, the first peptide linker comprises the amino acid sequence of (Gly-Gly-Gly-Gly-Gln) n, wherein n is a number in the range of 1 to 5 (SEQ ID NO: 82). In some embodiments, the second peptide linker comprises the amino acid sequence of (Gly-Gly-Gly-Gly-Gln) n, wherein n is a number in the range of 1 to 5 (SEQ ID NO: 82). In some embodiments, the first peptide linker and the second peptide linker both independently comprise amino acid sequences of (Gly-Gly-Gly-Gly-Gln) n, wherein n is a number in the range of 1 to 5 (SEQ ID NO: 82).

In some embodiments, the first peptide linker is a poly-Gly linker. In some embodiments, the second peptide linker is a poly-Gly linker. In some embodiments, the first peptide linker and the second peptide linker are independently poly-Gly linkers.

In some embodiments, the first peptide linker comprises the amino acid sequence of (Gly) p, wherein p is a number in the range of 4 to 10 (SEQ ID NO: 41). In some embodiments, the second peptide linker comprises the amino acid sequence of (Gly) p, wherein p is a number in the range of 4 to 10 (SEQ ID NO: 41). In some embodiments, the first peptide linker and the second peptide linker both independently comprise amino acid sequences of (Gly) p, wherein p is a number in the range of 4 to 10 (SEQ ID NO: 41).

In some embodiments, the first peptide linker comprises the amino acid sequence of GGGG (SEQ ID NO: 37). In some embodiments, the second peptide linker comprises the amino acid sequence of GGGG (SEQ ID NO: 37). In some embodiments, the first peptide linker and the second peptide linker both comprise the amino acid sequence of GGGG (SEQ ID NO: 37).

In some embodiments, the Fc region is a variant Fc region. In some embodiments, the variant Fc region comprises heterodimerizing alterations. In some embodiments, the Fc region is a silenced Fc region.

In some embodiments, the Fc region comprises an amino acid sequence that is at least 90% (e.g., at least 91%, at least 92%, at least 93%, at least 94%, at least 95%, at least 96%, at least 97%, at least 98%; 91%, 92%, 93%, 94%, 95%, 96%, 97%, 98%) identical to the amino acid sequence of SEQ ID NO: 34, wherein the amino acid sequence comprises a glycine at position 297, a cysteine at position 292, and a cysteine at position 302, all according to EU numbering.

In some embodiments, the Fc region comprises an amino acid sequence that is at least 91% identical to the amino acid sequence of SEQ ID NO: 34. In some embodiments, the Fc region comprises an amino acid sequence that is at least 92% identical to the amino acid sequence of SEQ ID NO: 34. In some embodiments, the Fc region comprises an amino acid sequence that is at least 93% identical to the amino acid sequence of SEQ ID NO: 34. In some embodiments, the Fc region comprises an amino acid sequence that is at least 94% identical to the amino acid sequence of SEQ ID NO: 34. In some embodiments, the Fc region comprises an amino acid sequence that is at least 95% identical to the amino acid sequence of SEQ ID NO: 34. In some embodiments, the Fc region comprises an amino acid sequence that is at least 96% identical to the amino acid sequence of SEQ ID NO: 34. In some embodiments, the Fc region comprises an amino acid sequence that is at least 97% identical to the amino acid sequence of SEQ ID NO: 34.

In some embodiments, the Fc region comprises an amino acid sequence that is 97.4% identical to the amino acid sequence of SEQ ID NO: 34. In some embodiments, the Fc region comprises an amino acid sequence that is 97.8% identical to the amino acid sequence of SEQ ID NO: 34. In some embodiments, the Fc region comprises an amino acid sequence that is 98.2% identical to the amino acid sequence of SEQ ID NO: 34. In some embodiments, the Fc region comprises an amino acid sequence that is 98.6% identical to the amino acid sequence of SEQ ID NO: 34.

In some embodiments, the Fc region comprises a first polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering. In some embodiments, the Fc region comprises a first polypeptide chain comprising an amino acid sequence that is at least 95% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering. In some embodiments, the Fc region comprises a first polypeptide chain comprising an amino acid sequence that is between 90% and 97.8% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering.

In some embodiments, the first polypeptide chain comprises the amino acid sequence of SEQ ID NO: 35.

In some embodiments, the Fc region further comprises a second polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering. In some embodiments, the Fc region further comprises a second polypeptide chain comprising an amino acid sequence that is at least 95% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering. In some embodiments, the Fc region further comprises a second polypeptide chain comprising an amino acid sequence that is between 90% and 97.4% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

In some embodiments, the second polypeptide chain comprises the amino acid sequence of SEQ ID NO: 36.

In some embodiments, the Fc region comprises a first polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering; and a second polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

In some embodiments, the Fc region comprises a first polypeptide chain comprising an amino acid sequence that is at least 95% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering; and a second polypeptide chain comprising an amino acid sequence that is at least 95% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

In some embodiments, the Fc region comprises a first polypeptide chain comprising an amino acid sequence that is between 90% and 97.8% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering; and a second polypeptide chain comprising an amino acid sequence that is between 90% and 97.4% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

In some embodiments, the first polypeptide chain comprises the amino acid sequence of SEQ ID NO: 35, and the second polypeptide chain comprises the amino acid sequence of SEQ ID NO: 36.

In some embodiments, the first heavy chain variable region is connected to the first polypeptide chain of the Fc region by the first peptide linker, and the second heavy chain variable region is connected to the second polypeptide chain of the Fc region by the second peptide linker.

In some embodiments, the first heavy chain variable region is connected to the second polypeptide chain of the Fc region by the first peptide linker, and the second heavy chain variable region is connected to the first polypeptide chain of the Fc region by the second peptide linker.

In some embodiments, the Fc region comprises a first polypeptide chain comprising the amino acid sequence of SEQ ID NO: 35 and a second polypeptide chain comprising the amino acid sequence of SEQ ID NO: 36; and the first peptide linker and the second peptide linker independently comprise the amino acid sequence of (Gly-Gly-Gly-Gly-Ser) m, wherein m is a number in the range of 1 to 5 (SEQ ID NO: 40).

In some embodiments, the Fc region comprises a first polypeptide chain comprising the amino acid sequence of SEQ ID NO: 35 and a second polypeptide chain comprising the amino acid sequence of SEQ ID NO: 36; and the first peptide linker and the second peptide linker independently comprise the amino acid sequence of (Gly-Gly-Gly-Gly-Gln) n, wherein n is a number in the range of 1 to 5 (SEQ ID NO: 82).

In some embodiments, the Fc region comprises a first polypeptide chain comprising the amino acid sequence of SEQ ID NO: 35 and a second polypeptide chain comprising the amino acid sequence of SEQ ID NO: 36; and the first peptide linker and the second peptide linker are independently poly-Gly linkers.

In some embodiments, the Fc region comprises a first polypeptide chain comprising the amino acid sequence of SEQ ID NO: 35 and a second polypeptide chain comprising the amino acid sequence of SEQ ID NO: 36; and the first peptide linker and the second peptide linker independently comprise the amino acid sequence of (Gly) p, wherein p is a number in the range of 4 to 10 (SEQ ID NO: 41).

In some embodiments, the Fc region comprises a first polypeptide chain comprising the amino acid sequence of SEQ ID NO: 35 and a second polypeptide chain comprising the amino acid sequence of SEQ ID NO: 36; and the first peptide linker and the second peptide linker both comprise the amino acid sequence of GGGG (SEQ ID NO: 37).

Also provided herein is a heavy-chain antibody comprising:
a first VH region that binds to IL2Rβ, wherein the first VH region comprises a VH CDR1, a VH CDR2, and a VH CDR3 comprising the amino acid sequences of SEQ ID NO: 2, SEQ ID NO: 5, and SEQ ID NO: 9, respectively;
a second VH region that binds to IL2RG, wherein the second VH region comprises a VH CDR1, a VH CDR2, and a VH CDR3 comprising the amino acid sequences of SEQ ID NO: 15, SEQ ID NO: 18, and SEQ ID NO: 20, respectively; and
an Fc region that comprises an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering.

Also provided herein is a heavy-chain antibody comprising:
a first VH region that binds to IL2Rβ, wherein the first VH region comprises an amino acid sequence that is at least 80% (e.g., at least 85%, at least 90%, at least 95%; 80%, 85%, 90%, 95%) identical to SEQ ID NO: 13;
a second VH region that binds to IL2RG, wherein the second VH region comprises an amino acid sequence that is at least 80% (e.g., at least 85%, at least 90%, at least 95%; 80%, 85%, 90%, 95%) identical to SEQ ID NO: 23; and
an Fc region that comprises an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering.

In some embodiments, the first VH region comprises the amino acid sequence of SEQ ID NO: 13. In some embodiments, the second VH region comprises the amino acid sequence of SEQ ID NO: 23. In some embodiments, the first VH region comprises the amino acid sequence of SEQ ID NO: 13, and the second VH region comprises the amino acid sequence of SEQ ID NO: 23.

Also provided herein is a heavy-chain antibody comprising:
a first VH region that binds to IL2Rβ, wherein the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13;
a second VH region that binds to IL2RG, wherein the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23; and
an Fc region that comprises an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering.

Also provided herein is a heavy-chain antibody comprising:
a first VH region that binds to IL2Rβ, wherein the first VH region comprises a VH CDR1, a VH CDR2, and a VH CDR3 comprising the amino acid sequences of SEQ ID NO: 2, SEQ ID NO: 5, and SEQ ID NO: 9, respectively;
a second VH region that binds to IL2RG, wherein the second VH region comprises a VH CDR1, a VH CDR2, and a VH CDR3 comprising the amino acid sequences of SEQ ID NO: 15, SEQ ID NO: 18, and SEQ ID NO: 20, respectively; and
an Fc region, wherein the first heavy chain variable region is connected to the Fc region by a first peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids, and the second heavy chain variable region is connected to the Fc region by a second peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids.

Also provided herein is a heavy-chain antibody comprising:
a first VH region that binds to IL2Rβ, wherein the first VH region comprises an amino acid sequence that is at least 80% (e.g., at least 85%, at least 90%, at least 95%; 80%, 85%, 90%, 95%) identical to SEQ ID NO: 13;
a second VH region that binds to IL2RG, wherein the second VH region comprises an amino acid sequence that is at least 80% (e.g., at least 85%, at least 90%, at least 95%; 80%, 85%, 90%, 95%) identical to SEQ ID NO: 23; and
an Fc region, wherein the first heavy chain variable region is connected to the Fc region by a first peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids, and the second heavy chain variable region is connected to the Fc region by a second peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids.

In some embodiments, the first VH region comprises the amino acid sequence of SEQ ID NO: 13. In some embodiments, the second VH region comprises the amino acid sequence of SEQ ID NO: 23. In some embodiments, the first VH region comprises the amino acid sequence of SEQ ID NO: 13, and the second VH region comprises the amino acid sequence of SEQ ID NO: 23.

Also provided herein is a heavy-chain antibody comprising:
a first VH region that binds to IL2Rβ, wherein the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13;
a second VH region that binds to IL2RG, wherein the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23; and
an Fc region, wherein the first heavy chain variable region is connected to the Fc region by a first peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids, and the second heavy chain variable region is connected to the Fc region by a second peptide linker comprising at least four (e.g., at least 5, at least 6, at least 7, at least 8, at least 9, at least 10; between 4 and 10, between 4 and 9, between 4 and 8, between 4 and 7, between 4 and 6; 4, 5, 6, 7, 8, 9, 10) amino acids.

Also provided herein is a heavy-chain antibody comprising:
a first VH region that binds to IL2Rβ, wherein the first VH region comprises a VH CDR1, a VH CDR2, and a VH CDR3 comprising the amino acid sequences of SEQ ID NO: 2, SEQ ID NO: 5, and SEQ ID NO: 9, respectively;
a second VH region that binds to IL2RG, wherein the second VH region comprises a VH CDR1, a VH CDR2, and a VH CDR3 comprising the amino acid sequences of SEQ ID NO: 15, SEQ ID NO: 18, and SEQ ID NO: 20, respectively; and
an Fc region comprising a first polypeptide chain and a second polypeptide chain, wherein the first polypeptide chain comprises the amino acid sequence of SEQ ID NO: 35, and the second polypeptide chain comprises the amino acid sequence of SEQ ID NO: 36.

In some embodiments, the first VH region is connected to the Fc region by a first peptide linker. In some embodiments, the second VH region is connected to the Fc region by a second peptide linker. In some embodiments, the first heavy chain variable region is connected to the first polypeptide chain of the Fc region by a first peptide linker, and the second heavy chain variable region is connected to the second polypeptide chain of the Fc region by a second peptide linker.

In some embodiments, the first peptide linker and the second peptide linker independently comprise the amino acid sequence of (Gly-Gly-Gly-Gly-Ser) m, wherein m is a number in the range of 1 to 5 (SEQ ID NO: 40).

In some embodiments, the first peptide linker and the second peptide linker independently comprise the amino acid sequence of (Gly-Gly-Gly-Gly-Gln) n, wherein n is a number in the range of 1 to 5 (SEQ ID NO: 82).

In some embodiments, the first peptide linker and the second peptide linker independently comprise the amino acid sequence of (Gly) p, wherein p is a number in the range of 4 to 10 (SEQ ID NO: 41).

In some embodiments, the first peptide linker and the second peptide linker both comprise the amino acid sequence of GGGG (SEQ ID NO: 37).

Also provided herein is a heavy-chain antibody comprising:
a first VH region that binds to IL2Rβ, wherein the first VH region comprises an amino acid sequence that is at least 80% (e.g., at least 85%, at least 90%, at least 95%; 80%, 85%, 90%, 95%) identical to SEQ ID NO: 13;
a second VH region that binds to IL2RG, wherein the second VH region comprises an amino acid sequence that is at least 80% (e.g., at least 85%, at least 90%, at least 95%; 80%, 85%, 90%, 95%) identical to SEQ ID NO: 23; and
an Fc region comprising a first polypeptide chain and a second polypeptide chain, wherein the first polypeptide chain comprises the amino acid sequence of SEQ ID NO: 35, and the second polypeptide chain comprises the amino acid sequence of SEQ ID NO: 36.

In some embodiments, the first VH region comprises the amino acid sequence of SEQ ID NO: 13. In some embodiments, the second VH region comprises the amino acid sequence of SEQ ID NO: 23. In some embodiments, the first VH region comprises the amino acid sequence of SEQ ID NO: 13, and the second VH region comprises the amino acid sequence of SEQ ID NO: 23.

In some embodiments, the first VH region is connected to the Fc region by a first peptide linker. In some embodiments, the second VH region is connected to the Fc region by a second peptide linker. In some embodiments, the first heavy chain variable region is connected to the first polypeptide chain of the Fc region by a first peptide linker, and the second heavy chain variable region is connected to the second polypeptide chain of the Fc region by a second peptide linker.

In some embodiments, the first peptide linker and the second peptide linker independently comprise the amino acid sequence of (Gly-Gly-Gly-Gly-Ser) m, wherein m is a number in the range of 1 to 5 (SEQ ID NO: 40).

In some embodiments, the first peptide linker and the second peptide linker independently comprise the amino acid sequence of (Gly-Gly-Gly-Gly-Gln) n, wherein n is a number in the range of 1 to 5 (SEQ ID NO: 82).

In some embodiments, the first peptide linker and the second peptide linker independently comprise the amino acid sequence of (Gly) p, wherein p is a number in the range of 4 to 10 (SEQ ID NO: 41).

In some embodiments, the first peptide linker and the second peptide linker both comprise the amino acid sequence of GGGG (SEQ ID NO: 37).

Also provided herein is a heavy-chain antibody comprising:
a first VH region that binds to IL2Rβ, wherein the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13;
a second VH region that binds to IL2RG, wherein the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23; and
an Fc region comprising a first polypeptide chain and a second polypeptide chain, wherein the first polypeptide chain comprises the amino acid sequence of SEQ ID NO: 35, and the second polypeptide chain comprises the amino acid sequence of SEQ ID NO: 36.

In some embodiments, the first VH region is connected to the Fc region by a first peptide linker. In some embodiments, the second VH region is connected to the Fc region by a second peptide linker. In some embodiments, the first heavy chain variable region is connected to the first polypeptide chain of the Fc region by a first peptide linker, and the second heavy chain variable region is connected to the second polypeptide chain of the Fc region by a second peptide linker.

In some embodiments, the first peptide linker and the second peptide linker independently comprise the amino acid sequence of (Gly-Gly-Gly-Gly-Ser) m, wherein m is a number in the range of 1 to 5 (SEQ ID NO: 40).

In some embodiments, the first peptide linker and the second peptide linker independently comprise the amino acid sequence of (Gly-Gly-Gly-Gly-Gln) n, wherein n is a number in the range of 1 to 5 (SEQ ID NO: 82).

In some embodiments, the first peptide linker and the second peptide linker independently comprise the amino acid sequence of (Gly) p, wherein p is a number in the range of 4 to 10 (SEQ ID NO: 41).

In some embodiments, the first peptide linker and the second peptide linker both comprise the amino acid sequence of GGGG (SEQ ID NO: 37).

Also provided herein is a heavy-chain antibody comprising:
a first VH region that binds to IL2Rβ, wherein the first VH region comprises a VH CDR1, a VH CDR2, and a VH CDR3 comprising the amino acid sequences of SEQ ID NO: 2, SEQ ID NO: 5, and SEQ ID NO: 9, respectively;
a second VH region that binds to IL2RG, wherein the second VH region comprises a VH CDR1, a VH CDR2, and a VH CDR3 comprising the amino acid sequences of SEQ ID NO: 15, SEQ ID NO: 18, and SEQ ID NO: 20, respectively; and
an Fc region, wherein the first heavy chain variable region is connected to the Fc region by a first peptide linker and the second heavy chain variable region is connected to the Fc region by a second peptide linker, wherein the first peptide linker and the second peptide linker independently comprise the amino acid sequence of (Gly-Gly-Gly-Gly-Ser) m, wherein m is a number in the range of 1 to 5 (SEQ ID NO: 40), (Gly-Gly-Gly-Gly-Gln) n, wherein n is a number in the range of 1 to 5 (SEQ ID NO: 82), or the amino acid sequence of (Gly) p, wherein p is a number in the range of 4 to 10 (SEQ ID NO: 41).

In some embodiments, the first peptide linker and the second peptide linker independently comprise the amino acid sequence of (Gly-Gly-Gly-Gly-Ser) m, wherein m is a number in the range of 1 to 5 (SEQ ID NO: 40).

In some embodiments, the first peptide linker and the second peptide linker independently comprise the amino acid sequence of (Gly-Gly-Gly-Gly-Gln) n, wherein n is a number in the range of 1 to 5 (SEQ ID NO: 82).

In some embodiments, the first peptide linker and the second peptide linker independently comprise the amino acid sequence of (Gly) p, wherein p is a number in the range of 4 to 10 (SEQ ID NO: 41).

In some embodiments, the first peptide linker and the second peptide linker both comprise the amino acid sequence of GGGG (SEQ ID NO: 37).

In some embodiments, the Fc region is a variant Fc region. In some embodiments, the variant Fc region comprises heterodimerizing alterations. In some embodiments, the Fc region is a silenced Fc region.

Also provided herein is a heavy-chain antibody comprising:
a first VH region that binds to IL2Rβ, wherein the first VH region comprises an amino acid sequence that is at least 80% (e.g., at least 85%, at least 90%, at least 95%; 80%, 85%, 90%, 95%) identical to SEQ ID NO: 13;
a second VH region that binds to IL2RG, wherein the second VH region comprises an amino acid sequence that is at least 80% (e.g., at least 85%, at least 90%, at least 95%; 80%, 85%, 90%, 95%) identical to SEQ ID NO: 23; and
an Fc region, wherein the first heavy chain variable region is connected to the Fc region by a first peptide linker and the second heavy chain variable region is connected to the Fc region by a second peptide linker, wherein the first peptide linker and the second peptide linker independently comprise the amino acid sequence of (Gly-Gly-Gly-Gly-Ser) m, wherein m is a number in the range of 1 to 5 (SEQ ID NO: 40), the amino acid sequence of (Gly-Gly-Gly-Gly-Gln) n, wherein n is a number in the range of 1 to 5 (SEQ ID NO: 82), or the amino acid sequence of (Gly) p, wherein p is a number in the range of 4 to 10 (SEQ ID NO: 41).

In some embodiments, the first peptide linker and the second peptide linker independently comprise the amino acid sequence of (Gly-Gly-Gly-Gly-Ser) m, wherein m is a number in the range of 1 to 5 (SEQ ID NO: 40).

In some embodiments, the first peptide linker and the second peptide linker independently comprise the amino acid sequence of (Gly-Gly-Gly-Gly-Gln) n, wherein n is a number in the range of 1 to 5 (SEQ ID NO: 82).

In some embodiments, the first peptide linker and the second peptide linker independently comprise the amino acid sequence of (Gly) p, wherein p is a number in the range of 4 to 10 (SEQ ID NO: 41).

In some embodiments, the first peptide linker and the second peptide linker both comprise the amino acid sequence of GGGG (SEQ ID NO: 37).

In some embodiments, the Fc region is a variant Fc region. In some embodiments, the variant Fc region comprises heterodimerizing alterations. In some embodiments, the Fc region is a silenced Fc region.

In some embodiments, the first VH region comprises the amino acid sequence of SEQ ID NO: 13. In some embodiments, the second VH region comprises the amino acid sequence of SEQ ID NO: 23. In some embodiments, the first VH region comprises the amino acid sequence of SEQ ID NO: 13, and the second VH region comprises the amino acid sequence of SEQ ID NO: 23.

Also provided herein is a heavy-chain antibody comprising:
a first VH region that binds to IL2Rβ, wherein the full set of VH CDRs 1, 2, and 3 (combined) in the first VH region is at least 80% (e.g., at least 85%, at least 90%, at least 95%; 80%, 85%, 90%, 95%) identical to the VH CDRs 1, 2, and 3 of SEQ ID NO: 13;
a second VH region that binds to IL2RG, wherein the full set of VH CDRs 1, 2, and 3 (combined) in the second VH region is at least 80% (e.g., at least 85%, at least 90%, at least 95%; 80%, 85%, 90%, 95%) identical to the VH CDRs 1, 2, and 3 of SEQ ID NO: 23; and
an Fc region, wherein the first heavy chain variable region is connected to the Fc region by a first peptide linker and the second heavy chain variable region is connected to the Fc region by a second peptide linker, wherein the first peptide linker and the second peptide linker independently comprise the amino acid sequence of (Gly-Gly-Gly-Gly-Ser) m, wherein m is a number in the range of 1 to 5 (SEQ ID NO: 40), the amino acid sequence of (Gly-Gly-Gly-Gly-Gln) n, wherein n is a number in the range of 1 to 5 (SEQ ID NO: 82), or the amino acid sequence of (Gly) p, wherein p is a number in the range of 4 to 10 (SEQ ID NO: 41).

In some embodiments, the first peptide linker and the second peptide linker independently comprise the amino acid sequence of (Gly-Gly-Gly-Gly-Ser) m, wherein m is a number in the range of 1 to 5 (SEQ ID NO: 40).

In some embodiments, the first peptide linker and the second peptide linker independently comprise the amino acid sequence of (Gly-Gly-Gly-Gly-Gln) n, wherein n is a number in the range of 1 to 5 (SEQ ID NO: 82).

In some embodiments, the first peptide linker and the second peptide linker independently comprise the amino acid sequence of (Gly) p, wherein p is a number in the range of 4 to 10 (SEQ ID NO: 41).

In some embodiments, the first peptide linker and the second peptide linker both comprise the amino acid sequence of GGGG (SEQ ID NO: 37).

In some embodiments, the Fc region is a variant Fc region. In some embodiments, the variant Fc region comprises heterodimerizing alterations. In some embodiments, the Fc region is a silenced Fc region.

Also provided herein is a heavy-chain antibody comprising a first heavy chain that binds to IL2Rβ comprising the amino acid sequence of SEQ ID NO: 38; and a second heavy chain that binds to IL2RG comprising the amino acid sequence of SEQ ID NO: 39.

TABLE 6 provides the amino acid sequences of human IgG1 and IgG4 Fc regions, as well as versions of these sequences that incorporate additional mutations (variants) that impart specific properties. TABLE 7 provides sequence information for two example heavy-chain antibodies tested in the Examples of this application.

TABLE 6
Human IgG1 and IgG4 Fc Region Sequences and Variants Thereof

| Description | Amino Acid Sequence |
|---|---|
| Human IgG1 | ASTKGPSVFPLAPSSKSTSGGTAALGCLVKDYFPEPVTVSWNSGA |
| (UniProt No. P01857) | LTSGVHTFPAVLQSSGLYSLSSVVTVPSSSLGTQTYICNVNHKPS |
| | NTKVDKKVEPKSCDKTHTCPPCPAPELLGGPSVFLFPPKPKDTLM |
| | ISRTPEVTCVVVDVSHEDPEVKFNWYVDGVEVHNAKTKPREEQ |
| | YNSTYRVVSVLTVLHQDWLNGKEYKCKVSNKALPAPIEKTISKA |
| | KGQPREPQVYTLPPSRDELTKNQVSLTCLVKGFYPSDIAVEWESN |
| | GQPENNYKTTPPVLDSDGSFFLYSKLTVDKSRWQQGNVFSCSVM |
| | HEALHNHYTQKSLSLSPGK (SEQ ID NO: 49) |
| Human IgG4 | ASTKGPSVFPLAPCSRSTSESTAALGCLVKDYFPEPVTVSWNSGA |
| (UniProt No. P01861) | LTSGVHTFPAVLQSSGLYSLSSVVTVPSSSLGTKTYTCNVDHKPS |
| | NTKVDKRVESKYGPPCPSCPAPEFLGGPSVFLFPPKPKDTLMISRT |
| | PEVTCVVVDVSQEDPEVQFNWYVDGVEVHNAKTKPREEQFNST |
| | YRVVSVLTVLHQDWLNGKEYKCKVSNKGLPSSIEKTISKAKGQP |
| | REPQVYTLPPSQEEMTKNQVSLTCLVKGFYPSDIAVEWESNGQP |
| | ENNYKTTPPVLDSDGSFFLYSRLTVDKSRWQEGNVFSCSVMHEA |
| | LHNHYTQKSLSLSLGK (SEQ ID NO: 50) |
| Human IgG1 Variant | DKTHTCPPCPAPELLGGPSVFLFPPKPKDTLMISRTPEVTCVVVD |
| Fc Region | VSHEDPEVKFNWYVDGVEVHNAKTKPREEQYNSTYRVVSVLTV |
| | LHQDWLNGKEYKCKVSNKALPAPIEKTISKAKGQPREPQVYTLP |
| | PSREEMTKNQVSLTCLVKGFYPSDIAVEWESNGQPENNYKTTPP |
| | VLDSDGSFFLYSKLTVDKSRWQQGNVFSCSVMHEALHNHYTQK |
| | SLSLSPGK (SEQ ID NO: 34) |
| Human IgG1 with | ASTKGPSVFPLAPSSKSTSGGTAALGCLVKDYFPEPVTVSWNSGA |
| silencing mutations (Fc | LTSGVHTFPAVLQSSGLYSLSSVVTVPSSSLGTQTYICNVNHKPS |
| region) | NTKVDKKVEPKSCDKTHTCPPCPAPEAAGGPSVFLFPPKPKDTL |
| | MISRTPEVTCVVVDVSHEDPEVKFNWYVDGVEVHNAKTKPREE |
| | QYNSTYRVVSVLTVLHQDWLNGKEYKCKVSNKALPAPIEKTISK |
| | AKGQPREPQVYTLPPSREEMTKNQVSLTCLVKGFYPSDIAVEWE |
| | SNGQPENNYKTTPPVLDSDGSFFLYSKLTVDKSRWQQGNVFSCS |
| | VMHEALHNHYTQKSLSLSPGK (SEQ ID NO: 51) |
| Human IgG4 with | ASTKGPSVFPLAPCSRSTSESTAALGCLVKDYFPEPVTVSWNSGA |
| silencing mutations (Fc | LTSGVHTFPAVLQSSGLYSLSSVVTVPSSSLGTKTYTCNVDHKPS |
| region) | NTKVDKRVESKYGPPCPPCPAPEAAGGPSVFLFPPKPKDTLMISR |
| | TPEVTCVVVDVSQEDPEVQFNWYVDGVEVHNAKTKPREEQFNS |
| | TYRVVSVLTVLHQDWLNGKEYKCKVSNKGLPSSIEKTISKAKGQ |
| | PREPQVYTLPPSQEEMTKNQVSLTCLVKGFYPSDIAVEWESNGQ |
| | PENNYKTTPPVLDSDGSFFLYSRLTVDKSRWQEGNVFSCSVMHE |
| | ALHNHYTQKSLSLSLGK (SEQ ID NO: 52) |
| Human IgG4 hinge | ESKYGPPCPSCPA (SEQ ID NO: 53) |
| region (wild type) | |
| Human IgG4 hinge | ESKYGPPCPPCPA (SEQ ID NO: 54) |
| region (S228P) | |
| Human IgG4 CH2 | APEFLGGPSVFLFPPKPKDTLMISRTPEVTCVVVDVSQEDPEVQF |
| domain sequence (wild | NWYVDGVEVHNAKTKPREEQFNSTYRVVSVLTVLHQDWLNGK |
| type) | EYKCKVSNKGLPSSIEKTISKAK (SEQ ID NO: 55) |
| Human IgG4 CH2 | APEAAGGPSVFLFPPKPKDTLMISRTPEVTCVVVDVSQEDPEVQF |
| domain sequence | NWYVDGVEVHNAKTKPREEQFNSTYRVVSVLTVLHQDWLNGK |
| (F234A, L235A) | EYKCKVSNKGLPSSIEKTISKAK (SEQ ID NO: 56) |
| Human IgG4 CH3 | GQPREPQVYTLPPSQEEMTKNQVSLTCLVKGFYPSDIAVEWESN |
| domain sequence (wild | GQPENNYKTTPPVLDSDGSFFLYSRLTVDKSRWQEGNVFSCSVM |
| type) | HEALHNHYTQKSLSLSLGK (SEQ ID NO: 57) |
| Human IgG4 CH3 | GQPREPQVYTLPPSQEEMTKNQVSLWCLVKGFYPSDIAVEWESN |
| domain sequence | GQPENNYKTTPPVLDSDGSFFLYSRLTVDKSRWQEGNVFSCSVM |
| (knob, T366W) | HEALHNHYTQKSLSLSLGK (SEQ ID NO: 58) |
| Human IgG4 CH3 | GQPREPQVYTLPPSQEEMTKNQVSLSCAVKGFYPSDIAVEWESN |
| domain sequence (hole, | GQPENNYKTTPPVLDSDGSFFLVSRLTVDKSRWQEGNVFSCSVM |
| T366S, L368A, | HEALHNHYTQKSLSLSLGK (SEQ ID NO: 59) |
| Y407V) | |

TABLE 7
Amino Acid Sequences

| Description | Amino Acid Sequence |
|---|---|
| IgG1 SEFL2.2 KK Fc | DKTHTCPPCPAPELLGGPSVFLFPPKPKDTLMISRTPEVTCVVVDVS |
| region | HEDPEVKFNWYVDGVEVHNAKTKPCEEQYGSTYRCVSVLTVLHQ |
| | DWLNGKEYKCKVSNKALPAPIEKTISKAKGQPREPQVYTLPPSRKE |
| | MTKNQVSLTCLVKGFYPSDIAVEWESNGQPENNYKTTPPVLKSDG |
| | SFFLYSKLTVDKSRWQQGNVFSCSVMHEALHNHYTQKSLSLSPGK |
| | (SEQ ID NO: 35) |
| IgG1 SEFL2.2 DDD Fc | DKTHTCPPCPAPELLGGPSVFLFPPKPKDTLMISRTPEVTCVVVDVS |
| region | HEDPEVKFNWYVDGVEVHNAKTKPCEEQYGSTYRCVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYDTTPPVLDSDG |
| | SFFLYSDLTVDKSRWQQGNVFSCSVMHEALHNHYTQDSLSLSPGK |
| | (SEQ ID NO: 36) |
| Peptide linker | GGGG (SEQ ID NO: 37) |
| IL2RB_F09K IgG1 HC | QVQLQESSPGLVKPSETLSLTCTVSGGSISSSNWWSWVRQPPGKGL |
| (SEFL2.2 DDD side) | EWIGEISHSGSTNYNPSLKSRVTISVDKSKNQFSLRLSSVTAADTAV |
| | YFCGRGSWELTDAFDIRGQGTLVTVSSGGGGDKTHTCPPCPAPELL |
| | GGPSVFLFPPKPKDTLMISRTPEVTCVVVDVSHEDPEVKFNWYVDG |
| | VEVHNAKTKPCEEQYGSTYRCVSVLTVLHQDWLNGKEYKCKVSN |
| | KALPAPIEKTISKAKGQPREPQVYTLPPSREEMTKNQVSLTCLVKGF |
| | YPSDIAVEWESNGQPENNYDTTPPVLDSDGSFFLYSDLTVDKSRWQ |
| | QGNVFSCSVMHEALHNHYTQDSLSLSPGK (SEQ ID NO: 38) |
| IL2RB_F16B IgG1 HC | QVQLVESGGGLVKPGGSLRLSCAASGFTFSDYYMSWIRQAPGKGL |
| (SEFL2.2 KK side) | EWVSYISSSGSTIYYADSVKGRFTISRDNAKNSLYLQMNSLRAEDT |
| | AVYYCARGDAVSITGDYRGQGTLVTVSSGGGGDKTHTCPPCPAPE |
| | LLGGPSVFLFPPKPKDTLMISRTPEVTCVVVDVSHEDPEVKFNWYV |
| | DGVEVHNAKTKPCEEQYGSTYRCVSVLTVLHQDWLNGKEYKCKV |
| | SNKALPAPIEKTISKAKGQPREPQVYTLPPSRKEMTKNQVSLTCLV |
| | KGFYPSDIAVEWESNGQPENNYKTTPPVLKSDGSFFLYSKLTVDKS |
| | RWQQGNVFSCSVMHEALHNHYTQKSLSLSPGK (SEQ ID NO: 39) |
| IL2RB_F09K IgG4 | QVQLQESSPGLVKPSETLSLTCTVSGGSISSSNWWSWVRQPPGKGL |
| Hole Fc Region | EWIGEISHSGSTNYNPSLKSRVTISVDKSKNQFSLRLSSVTAADTAV |
| | YFCGRGSWELTDAFDIRGQGTLVTVSSESKYGPPCPPCPAPEAAGG |
| | PSVFLFPPKPKDTLMISRTPEVTCVVVDVSQEDPEVQFNWYVDGVE |
| | VHNAKTKPREEQFNSTYRVVSVLTVLHQDWLNGKEYKCKVSNKG |
| | LPSSIEKTISKAKGQPREPQVYTLPPSQEEMTKNQVSLSCAVKGFYP |
| | SDIAVEWESNGQPENNYKTTPPVLDSDGSFFLVSRLTVDKSRWQEG |
| | NVFSCSVMHEALHNHYTQKSLSLSLGK (SEQ ID NO: 60) |
| IL2RG_F16B IgG4 | QVQLVESGGGLVKPGGSLRLSCAASGFTFSDYYMSWIRQAPGKGL |
| Knob Fc Region | EWVSYISSSGSTIYYADSVKGRFTISRDNAKNSLYLQMNSLRAEDT |
| | AVYYCARGDAVSITGDYRGQGTLVTVSSESKYGPPCPPCPAPEAAG |
| | GPSVFLFPPKPKDTLMISRTPEVTCVVVDVSQEDPEVQFNWYVDGV |
| | EVHNAKTKPREEQFNSTYRVVSVLTVLHQDWLNGKEYKCKVSNK |
| | GLPSSIEKTISKAKGQPREPQVYTLPPSQEEMTKNQVSLWCLVKGF |
| | YPSDIAVEWESNGQPENNYKTTPPVLDSDGSFFLYSRLTVDKSRWQ |
| | EGNVFSCSVMHEALHNHYTQKSLSLSLGK (SEQ ID NO: 61) |

### Biological Activity

In some embodiments, the heavy-chain antibodies disclosed herein are IL-2R agonists. In some embodiments, the heavy-chain antibodies disclosed herein have an affinity for IL2R with a Kd in the range of 10⁻¹¹ M to 10⁻⁶ M (e.g., in the range of 10⁻¹⁰ M to 10⁻⁶ M; in the range of 10⁻⁹ M to 10⁻⁶ M; in the range of 10⁻⁸ M to 10⁻⁶ M; in the range of 10⁻¹¹ M to 10⁻⁸ M; in the range of 10⁻¹⁰ M to 10⁻⁸ M; in the range of 10⁻⁹ M to 10⁻⁸ M; in the range of 10⁻¹¹ M to 10⁻⁹ M; in the range of 10⁻¹⁰ M to 10⁻⁹ M). In some embodiments, the heavy-chain antibodies disclosed herein have an affinity for IL2R with a Kd in the range of 10⁻¹¹ M to 10⁻⁶ M (e.g., in the range of 10⁻¹⁰ M to 10⁻⁶ M; in the range of 10⁻⁹ M to 10⁻⁶ M; in the range of 10⁻⁸ M to 10⁻⁶ M; in the range of 10⁻¹¹ M to 10⁻⁸ M; in the range of 10⁻¹⁰ M to 10⁻⁸ M; in the range of 10⁻⁹ M to 10⁻⁸ M; in the range of 10⁻¹¹ M to 10⁻⁹ M; in the range of 10⁻¹⁰ M to 10⁻⁹ M) as measured by BioLayer Interferometry, using an Octet QK384 instrument (Fortebio Inc., Menlo Park, CA) in kinetics mode. In some embodiments, the Kd value is measured using a ForteBio Octet QK384 instrument comprising an anti-human Fc capture (AHC, 18-5005) sensor in kinetics mode.

In some embodiments, the heavy-chain antibodies disclosed herein are IL-2Rβγ agonists.

In some embodiments, the heavy-chain antibodies disclosed herein have an affinity for IL2Rβ with a Kd in the range of 10⁻⁸ M to 2.5×10⁻⁷ M. In some embodiments, the heavy-chain antibodies disclosed herein have an affinity for IL2Rβ with a Kd in the range of 10⁻⁸ M to 2.5×10⁻⁷ M as measured by BioLayer Interferometry, using an Octet QK384 instrument (Fortebio Inc., Menlo Park, CA) in kinetics mode. In some embodiments, the Kd value is measured using a ForteBio Octet QK384 instrument comprising an anti-human Fc capture (AHC, 18-5005) sensor in kinetics mode.

In some embodiments, the heavy-chain antibodies disclosed herein have an affinity for IL2Rγ with a Kd in the range of 10⁻⁹ M to 2.5×10⁻⁸ M. In some embodiments, the heavy-chain antibodies disclosed herein have an affinity for IL2Rγ with a Kd in the range of 10⁻⁹ M to 2.5×10⁻⁸ M as measured by BioLayer Interferometry, using an Octet QK384 instrument (Fortebio Inc., Menlo Park, CA) in kinetics mode. In some embodiments, the Kd value is measured using a ForteBio Octet QK384 instrument comprising an anti-human Fc capture (AHC, 18-5005) sensor in kinetics mode.

In some embodiments, the heavy-chain antibodies disclosed herein have an affinity for IL2Rβ with a Kd in the range of 10⁻⁸ M to 2.5×10⁻⁷ M and an affinity for IL2Rγ with a Kd in the range of 109 M to 2.5×10⁻⁸ M. In some embodiments, the heavy-chain antibodies disclosed herein have an affinity for IL2Rβ with a Kd in the range of 10⁻⁸ M to 2.5×10⁻⁷ M and an affinity for IL2Rγ with a Kd in the range of 10⁻⁹ M to 2.5×10⁻⁸ M as measured by BioLayer Interferometry, using an Octet QK384 instrument (Fortebio Inc., Menlo Park, CA) in kinetics mode. In some embodiments, the Kd value is measured using a ForteBio Octet QK384 instrument comprising an anti-human Fc capture (AHC, 18-5005) sensor in kinetics mode.

TABLE 8 provides example amino acid sequences related to human IL-2R.

TABLE 8
Human IL-2R-Related Amino Acid Sequences

| | UniProt | |
|---|---|---|
| Protein Name | KB No. | Amino Acid Sequence |
| Human IL2RA | P01589 | MDSYLLMWGLLTFIMVPGCQAELCDDDPPEIPHATF |
| | | KAMAYKEGTMLNCECKRGFRRIKSGSLYMLCTGNSS |
| | | HSSWDNQCQCTSSATRNTTKQVTPQPEEQKERKTTE |
| | | MQSPMQPVDQASLPGHCREPPPWENEATERIYHFVV |
| | | GQMVYYQCVQGYRALHRGPAESVCKMTHGKTRWT |
| | | QPQLICTGEMETSQFPGEEKPQASPEGRPESETSCLVT |
| | | TTDFQIQTEMAATMETSIFTTEYQVAVAGCVFLLISVL |
| | | LLSGLTWQRRQRKSRRTI (SEQ ID NO: 45) |
| Human IL2RB | P14784 | MAAPALSWRLPLLILLLPLATSWASAAVNGTSQFTCF |
| | | YNSRANISCVWSQDGALQDTSCQVHAWPDRRRWNQ |
| | | TCELLPVSQASWACNLILGAPDSQKLTTVDIVTLRVL |
| | | CREGVRWRVMAIQDFKPFENLRLMAPISLQVVHVET |
| | | HRCNISWEISQASHYFERHLEFEARTLSPGHTWEEAPL |
| | | LTLKQKQEWICLETLTPDTQYEFQVRVKPLQGEFTTW |
| | | SPWSQPLAFRTKPAALGKDTIPWLGHLLVGLSGAFGF |
| | | IILVYLLINCRNTGPWLKKVLKCNTPDPSKFFSQLSSE |
| | | HGGDVQKWLSSPFPSSSFSPGGLAPEISPLEVLERDKV |
| | | TQLLLQQDKVPEPASLSSNHSLTSCFTNQGYFFFHLPD |
| | | ALEIEACQVYFTYDPYSEEDPDEGVAGAPTGSSPQPL |
| | | QPLSGEDDAYCTFPSRDDLLLFSPSLLGGPSPPSTAPG |
| | | GSGAGEERMPPSLQERVPRDWDPQPLGPPTPGVPDLV |
| | | DFQPPPELVLREAGEEVPDAGPREGVSFPWSRPPGQG |
| | | EFRALNARLPLNTDAYLSLQELQGQDPTHLV (SEQ ID |
| | | NO: 46) |
| Human IL2RG | P31785 | MLKPSLPFTSLLFLQLPLLGVGLNTTILTPNGNEDTTA |
| | | DFFLTTMPTDSLSVSTLPLPEVQCFVFNVEYMNCTWN |
| | | SSSEPQPTNLTLHYWYKNSDNDKVQKCSHYLFSEEIT |
| | | SGCQLQKKEIHLYQTFVVQLQDPREPRRQATQMLKL |
| | | QNLVIPWAPENLTLHKLSESQLELNWNNRFLNHCLE |
| | | HLVQYRTDWDHSWTEQSVDYRHKFSLPSVDGQKRY |
| | | TFRVRSRFNPLCGSAQHWSEWSHPIHWGSNTSKENPF |
| | | LFALEAVVISVGSMGLIISLLCVYFWLERTMPRIPTLK |
| | | NLEDLVTEYHGNFSAWSGVSKGLAESLQPDYSERLC |
| | | LVSEIPPKGGALGEGPGASPCNQHSPYWAPPCYTLKP |
| | | ET (SEQ ID NO: 47) |
| Human IL2 | P60568 | MYRMQLLSCIALSLALVTNSAPTSSSTKKTQLQLEHL |
| | | LLDLQMILNGINNYKNPKLTRMLTFKFYMPKKATEL |
| | | KHLQCLEEELKPLEEVLNLAQSKNFHLRPRDLISNINV |
| | | IVLELKGSETTFMCEYADETATIVEFLNRWITFCQSIIS |
| | | TLT (SEQ ID NO: 48) |

In some embodiments, heavy-chain antibodies disclosed herein, once bound to a target (e.g., IL2R), internalize into cells, wherein internalization is at least 10%, at least 20%, at least 30%, at least 40%, at least 50%, at least 60%, at least 70%, at least 80%, or at least 90%, at least 100%, at least 110%, at least 120%, at least 130%, at least 140%, at least 150%, at least 160%, at least 170%, at least 180%, at least 190%, or at least 200% or more, in comparison to one or more control antibodies that do not internalize.

### Preparation of Anti-Il2R Heavy-Chain Antibodies

The anti-IL2Rβ/γ heavy-chain antibodies disclosed herein can be prepared by methods known in the art. In some embodiments, the anti-IL2Rβ/γ heavy-chain antibodies can be produced by transgenic animals, including transgenic mice and rats, e.g., transgenic rats, in which the endogenous immunoglobulin genes are knocked out or disabled. In some embodiments, the anti-IL-2Rβγ heavy-chain antibodies described herein are produced in a UniRat™. UniRat™ have their endogenous immunoglobulin genes silenced and use a human immunoglobulin heavy-chain translocus to express a diverse, naturally optimized repertoire of fully human heavy-chain antibodies (HCAbs). While endogenous immunoglobulin loci in rats can be knocked out or silenced using a variety of technologies, in UniRat™, the zinc-finger (endo) nuclease (ZNF) technology was used to inactivate the endogenous rat heavy chain J-locus, light chain Ck locus and light chain CA locus. ZNF constructs for microinjection into oocytes can produce IgH and IgL knock out (KO) lines. For details, see, e.g., Geurts et al., 2009, Science 325:433. Characterization of Ig heavy chain knockout rats has been reported by Menoret et al., 2010, Eur. J. Immunol. 40:2932-2941. Advantages of the ZNF technology include that non-homologous end joining to silence a gene or locus via deletions up to several kb can also provide a target site for homologous integration (Cui et al., 2011, Nat Biotechnol 29:64-67). Human heavy-chain antibodies produced in UniRat™ are called UniAbs™.

In addition to UniAbs™, disclosed herein are heavy-chain antibodies lacking the camelid VHH framework and mutations, and their functional VH regions. Such heavy-chain antibodies can, for example, be produced in transgenic rats or mice which comprise fully human heavy chain-only gene loci as described, e.g., in WO2006/008548, but other transgenic mammals, such as rabbit, guinea pig, and rat can also be used. Heavy-chain antibodies, including their VHH or VH functional fragments, can also be produced by recombinant DNA technology, by expression of the encoding nucleic acid in a suitable eukaryotic or prokaryotic host, including, for example, mammalian cells (e.g., CHO cells), E. coli, or yeast.

In some embodiments, the present disclosure provides a polynucleotide encoding a heavy-chain antibody disclosed herein. In some embodiments, the present disclosure provides a vector comprising a polynucleotide that encodes a heavy-chain antibody disclosed herein. In some embodiments, the present disclosure provides a host cell (e.g., a CHO cell) comprising a vector which comprises a polynucleotide that encodes a heavy-chain antibody disclosed herein.

In some embodiments, a heavy-chain antibody disclosed herein includes a substitution of a native amino acid residue at the first position of the FR4 region (amino acid position 101 according to the Kabat numbering system) by another amino acid residue, wherein the substitution is capable of disrupting a surface-exposed hydrophobic patch comprising or associated with the native amino acid residue at that position. Such hydrophobic patches are normally buried in the interface with the antibody light chain constant region but become surface exposed in heavy-chain antibodies. In some embodiments, the substituted amino acid residue is charged. In some embodiments, the substituted amino acid residue is positively charged, such as, e.g., lysine (Lys, K), arginine (Arg, R) or histidine (His, H), e.g., arginine (R). In some embodiments, the heavy-chain antibodies derived from the transgenic animals contain a Trp to Arg mutation at position 101.

Heavy-chain antibodies that specifically bind to non-overlapping epitopes on an IL2R protein can be identified by competition binding assays, such as enzyme-linked immunoassays (ELISA assays) or flow cytometric competitive binding assays. For example, one can use competition between known antibodies binding to the target antigen and the heavy-chain antibody to identify heavy-chain antibodies that compete with the reference antibodies, as well as those that do not. The non-competing heavy-chain antibodies are identified as binding to a distinct epitope that does not overlap with the epitope bound by the reference antibody. Often, one antibody is immobilized, the antigen is bound, and a second, labeled (e.g., biotinylated) antibody is tested in an ELISA assay for ability to bind the captured antigen. This can also be performed by using surface plasmon resonance (SPR) platforms, including, for example, ProteOn XPR36 (BioRad, Inc), Biacore 2000 and Biacore T200 (GE Healthcare Life Sciences), and MX96 SPR Imager (Ibis Technologies B.V.), as well as on biolayer interferometry platforms, such as Octet Red384 and Octet HTX (ForteBio, Pall Inc).

In some cases, a heavy-chain antibody “competes” with a reference antibody if it causes a 15% to 100% reduction in the binding of the reference antibody to the target antigen, as determined by standard techniques, such as by the competition binding assays described above. In some embodiments, the relative inhibition is at least 15%, at least 20%, at least 25%, at least 30%, at least 35%, at least 40%, at least 45%, at least 50% at least 55%, at least 60%, at least 65%, at least 70%, at least 75%, at least 80%, at least 85%, at least 90%, or at least 95% or higher. In some embodiments, competitive binding is measured using an enzyme-linked immunoassay (ELISA assay). In some embodiments, competitive binding is measured using a flow cytometric competitive binding assay.

### Formulation and Route of Administration

While it may be possible to administer a heavy-chain antibody disclosed herein alone in the uses described, the heavy-chain antibody will normally be administered as an active ingredient in a pharmaceutical composition. Thus, further provided herein is a pharmaceutical composition comprising a heavy-chain antibody disclosed herein and a pharmaceutically acceptable excipient. Non-limiting examples of pharmaceutically acceptable excipients include calcium carbonate, calcium phosphate, sugars (e.g., lactose, glucose, or sucrose), starches, cellulose derivatives, gelatin, vegetable oils, polyethylene glycols, and physiologically compatible solvents. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018.

In some cases, a pharmaceutical composition described herein comprises a therapeutically effective amount of a heavy-chain antibody disclosed herein. In some embodiments, the pharmaceutical composition is made in the form of a dosage unit containing a particular amount of the active ingredient.

The heavy-chain antibodies disclosed herein may be administered by any suitable route in the form of a pharmaceutical composition adapted to such a route and in a dose effective for the treatment intended. The heavy-chain antibodies and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients. For example, in some embodiments, a pharmaceutical composition disclosed herein may be provided for peripheral administration, such as parenteral (e.g., subcutaneous, intravenous, intramuscular), continuous infusion (e.g., intravenous drip, intravenous bolus, intravenous infusion), topical, nasal, or oral administration. In some embodiments, a pharmaceutical composition disclosed herein may be provided for parenteral (e.g., subcutaneous, intravenous, intramuscular) or continuous infusion (e.g., intravenous drip, intravenous bolus, intravenous infusion). In some embodiments, a pharmaceutical composition disclosed herein may be provided for intravenous or subcutaneous administration.

For injection administration, heavy-chain antibodies disclosed herein may be formulated in aqueous solutions, e.g., in physiologically-compatible buffers, to reduce potential discomfort at the site of injection. Such a solution may contain carriers, excipients, or stabilizers. Alternatively, heavy-chain antibodies disclosed herein may be in a lyophilized form for reconstitution with a suitable vehicle, e.g., sterile pyrogen-free water, before use. Reconstitution volumes will depend on the protein content following lyophilization and the desired concentration of active ingredient in the reconstituted solution, but, in some cases, may be in the range of 0.5 mL to 5 mL. The solution following reconstitution can be further diluted with a diluent (e.g., saline and/or intravenous solution stabilizer (IVSS)) prior to administration to the subject as appropriate in order to administer a particular amount of the active ingredient.

Pharmaceutical compositions disclosed herein may be prepared for storage by mixing active ingredients having the desired degree of purity with optional pharmaceutically acceptable carriers, excipients, or stabilizers (see, e.g., Remington's Pharmaceutical Sciences 16th edition, Osol, A. Ed. (1980)), such as in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants such as ascorbic acid and methionine; preservatives (such as, e.g., octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG). Non-limiting example formulations are disclosed in U.S. Pat. No. 9,034,324, US20160355591, and US20160166689.

Pharmaceutical compositions disclosed herein may be sterilized by conventional sterilization techniques or may be sterile-filtered. In some embodiments, such compositions comprise sterile water. In some embodiments, such compositions can contain pharmaceutically acceptable auxiliary substances as required to approximate physiological conditions, such as, e.g., pH buffering agents.

In some embodiments, the pharmaceutical composition is a parenteral composition. In some embodiments, the pharmaceutical composition is a parenteral composition for injection. In some embodiments, the pharmaceutical composition is a parenteral composition for infusion.

In some embodiments, a pharmaceutical composition disclosed herein is provided for parenteral administration, is sterile and substantially isotonic, and is manufactured under Good Manufacturing Practice (GMP) conditions.

In some embodiments, a form of repository or “depot” slow release preparation may be used so that therapeutically effective amounts of the preparation are delivered into the bloodstream over many hours or days following subcutaneous injection or other delivery method. The desired isotonicity may be accomplished using sodium chloride or other pharmaceutically acceptable excipients, such as, e.g., dextrose, boric acid, sodium tartrate, propylene glycol, polyols (such as, e.g., mannitol and sorbitol), or other inorganic or organic solutes.

### Methods of Use

The heavy-chain antibodies described herein can specifically bind to and agonize the intermediate affinity IL-2 receptor. Without intending to be bound by any particular theory, the heavy-chain antibodies of the disclosure can, in some cases, induce T-cell and NK cell proliferation, leading to immune system activation that may be useful in the treatment of diseases that are mediated by activation of IL-2R signaling in immune cells, including but not limited to, infectious diseases, autoimmune disorders, cancer, inflammatory diseases, and diseases associated with deficient IL-2-mediated signaling, deficient T-cell proliferation, or T-cell dysfunction. In some cases, the heavy-chain antibodies of the disclosure can increase the number of CD3⁺ T cells, increase the number of CD4⁺ T cells, increase the number of CD8⁺ T cells, increase the number of CD8⁺ effector T cells (e.g., CTLs), increase the number of NK cells, increase the ratio of CD8⁺ T cells to CD4⁺ T cells, decrease the proportion of T-regs, induce activation of immune effector cells without preferentially activating T-regs, or a combination of any of the foregoing.

Besides being useful for human treatment, the heavy-chain antibodies provided herein may be useful for veterinary treatment of companion animals, exotic animals, and farm animals, including mammals, rodents, and the like. For example, animals including horses, dogs, and cats may be treated with heavy-chain antibodies provided herein.

In some embodiments, as disclosed elsewhere herein, a method of treating a patient is provided. In some embodiments, the method comprises administering a therapeutic amount of a heavy-chain antibody disclosed herein to a patient. In some embodiments, the patient is suffering from an infectious disease, an autoimmune disorder (e.g., Crohn's disease, multiple sclerosis), a cancer, an inflammatory disease (e.g., arthritis), or a disease or disorder associated with deficient IL-2-mediated signaling, deficient T cell proliferation, or T cell dysfunction.

In some embodiments, the infectious disease can be a chronic, persistent, latent, or slow infection. In some embodiments, the infectious disease can be a bacterial, viral, fungal, or parasitic infection. In some embodiments, the infectious disease can be an infection with Helicobacter pylori, Epstein-Barr virus (EBV), human immunodeficiency virus (HIV), hepatitis B, or hepatitis C.

Another aspect of the disclosure provides methods of using the heavy-chain antibodies disclosed herein or the pharmaceutical compositions of the present disclosure to treat an infectious disease, an autoimmune disorder (e.g., Crohn's disease, multiple sclerosis), a cancer, an inflammatory disease (e.g., arthritis), or a disease or disorder associated with deficient IL-2-mediated signaling, deficient T cell proliferation, or T cell dysfunction.

Another aspect of the disclosure provides methods of using the heavy-chain antibodies disclosed herein or the pharmaceutical compositions of the present disclosure to treat cancer, including but not limited, to hematologic cancers (e.g., acute myeloid leukemia, acute lymphocytic leukemia, chronic lymphocytic leukemia, chronic myeloid leukemia, multiple myeloma, diffuse large B-cell lymphoma, Burkitt lymphoma, and non-Hodgkin lymphoma) and solid tumors (e.g., prostate cancer, non-small cell lung cancer, small-cell lung cancer, renal cell carcinoma, hepatocellular carcinoma, bladder cancer, testicular cancer, colorectal cancer, esophageal cancer, glioblastoma, head and neck cancer, pancreatic cancer, breast cancer, gastric cancer, gastroesophageal junction cancer, bone cancer, ovarian cancer, endometrial cancer, and melanoma).

Still another aspect of the disclosure provides a heavy-chain antibody or a pharmaceutical composition disclosed herein for use in treating an infectious disease, an autoimmune disorder (e.g., Crohn's disease, multiple sclerosis), a cancer, an inflammatory disease (e.g., arthritis), or a disease or disorder associated with deficient IL-2-mediated signaling, deficient T cell proliferation, or T cell dysfunction.

Yet another aspect of the disclosure provides a heavy-chain antibody or a pharmaceutical composition disclosed herein for use in treating cancer.

In some embodiments, the cancer is a hematologic cancer. In some embodiments, the hematologic cancer is selected from acute myeloid leukemia, acute lymphocytic leukemia, chronic lymphocytic leukemia, chronic myeloid leukemia, multiple myeloma, diffuse large B-cell lymphoma, Burkitt lymphoma, and non-Hodgkin lymphoma.

In some embodiments, the cancer is a solid tumor. In some embodiments, the cancer is selected from prostate cancer, non-small cell lung cancer, small-cell lung cancer, renal cell carcinoma, hepatocellular carcinoma, bladder cancer, testicular cancer, colorectal cancer, esophageal cancer, glioblastoma, head and neck cancer, pancreatic cancer, breast cancer, gastric cancer, gastroesophageal junction cancer, bone cancer, ovarian cancer, endometrial cancer, and melanoma. In some embodiments, the cancer is small-cell lung cancer.

In some embodiments, the subject to be treated has at least one tumor with low immune infiltration (e.g., low T-cell infiltration) prior to the administration. In some embodiments, the administration increases tumor T-cell infiltration. In some embodiments, the administration is associated with at least one anti-tumor effect. In some embodiments, the at least one anti-tumor effect is selected from a decrease in the number of cancer cells, a decrease in the number of metastases, an increase in life expectancy, a decrease in cancer cell proliferation, a decrease in cancer cell survival, and an amelioration of various physiological symptoms associated with the cancerous condition.

In some embodiments, the at least one anti-tumor effect is a decrease in the number of cancer cells. In some embodiments, the at least one anti-tumor effect is a decrease in the number of metastases. In some embodiments, the at least one anti-tumor effect is an increase in life expectancy. In some embodiments, the at least one anti-tumor effect is a decrease in cancer cell proliferation. In some embodiments, the at least one anti-tumor effect is a decrease in cancer cell survival. In some embodiments, the at least one anti-tumor effect is an amelioration of various physiological symptoms associated with the cancerous condition.

In some embodiments, the subject to be treated was previously administered a first line therapy for the cancer. In some embodiments, the subject to be treated was previously administered a first line therapy and a second line therapy for the cancer.

Still another aspect of the disclosure provides a use of a heavy-chain antibody or a pharmaceutical composition disclosed herein in the preparation of a medicament for treating an infectious disease, an autoimmune disorder (e.g., Crohn's disease, multiple sclerosis), a cancer, an inflammatory disease (e.g., arthritis), or a disease or disorder associated with deficient IL-2-mediated signaling, deficient T cell proliferation, or T cell dysfunction.

Yet another aspect of the disclosure provides a use of a heavy-chain antibody or a pharmaceutical composition disclosed herein in the preparation of a medicament for treating cancer.

In some embodiments, the cancer is a hematologic cancer. In some embodiments, the hematologic cancer is selected from acute myeloid leukemia, acute lymphocytic leukemia, chronic lymphocytic leukemia, chronic myeloid leukemia, multiple myeloma, diffuse large B-cell lymphoma, Burkitt lymphoma, and non-Hodgkin lymphoma.

In some embodiments, the cancer is a solid tumor. In some embodiments, the cancer is selected from prostate cancer, non-small cell lung cancer, small-cell lung cancer, renal cell carcinoma, hepatocellular carcinoma, bladder cancer, testicular cancer, colorectal cancer, esophageal cancer, glioblastoma, head and neck cancer, pancreatic cancer, breast cancer, gastric cancer, gastroesophageal junction cancer, bone cancer, ovarian cancer, endometrial cancer, and melanoma. In some embodiments, the cancer is small-cell lung cancer.

A further aspect provided by the disclosure is a method of treating an infectious disease, an autoimmune disorder (e.g., Crohn's disease, multiple sclerosis), a cancer, an inflammatory disease (e.g., arthritis), or a disease or disorder associated with deficient IL-2-mediated signaling, deficient T cell proliferation, or T cell dysfunction in a subject in need thereof, the method comprising administering to the subject a therapeutically effective amount of a heavy-chain antibody disclosed herein or a pharmaceutical composition disclosed herein.

A further aspect provided by the disclosure is a method of treating cancer in a subject in need thereof, the method comprising administering to the subject a therapeutically effective amount of a heavy-chain antibody disclosed herein or a pharmaceutical composition disclosed herein.

In some embodiments, the cancer is a hematologic cancer. In some embodiments, the hematologic cancer is selected from acute myeloid leukemia, acute lymphocytic leukemia, chronic lymphocytic leukemia, chronic myeloid leukemia, multiple myeloma, diffuse large B-cell lymphoma, Burkitt lymphoma, and non-Hodgkin lymphoma.

In some embodiments, the cancer is a solid tumor. In some embodiments, the cancer is selected from prostate cancer, non-small cell lung cancer, small-cell lung cancer, renal cell carcinoma, hepatocellular carcinoma, bladder cancer, testicular cancer, colorectal cancer, esophageal cancer, glioblastoma, head and neck cancer, pancreatic cancer, breast cancer, gastric cancer, gastroesophageal junction cancer, bone cancer, ovarian cancer, endometrial cancer, and melanoma. In some embodiments, the cancer is small-cell lung cancer.

In some embodiments, the subject has at least one tumor with low immune infiltration (e.g., low T-cell infiltration) prior to the administration. In some embodiments, the administration increases tumor T-cell infiltration. In some embodiments, the administration is associated with at least one anti-tumor effect. In some embodiments, the at least one anti-tumor effect is selected from a decrease in the number of cancer cells, a decrease in the number of metastases, an increase in life expectancy, a decrease in cancer cell proliferation, a decrease in cancer cell survival, and an amelioration of various physiological symptoms associated with the cancerous condition.

In some embodiments, the at least one anti-tumor effect is a decrease in the number of cancer cells. In some embodiments, the at least one anti-tumor effect is a decrease in the number of metastases. In some embodiments, the at least one anti-tumor effect is an increase in life expectancy. In some embodiments, the at least one anti-tumor effect is a decrease in cancer cell proliferation. In some embodiments, the at least one anti-tumor effect is a decrease in cancer cell survival. In some embodiments, the at least one anti-tumor effect is an amelioration of various physiological symptoms associated with the cancerous condition.

In some embodiments, the subject was previously administered a first line therapy for the cancer. In some embodiments, the subject was previously administered a first line therapy and a second line therapy for the cancer.

### Combination Therapy

The present disclosure also provides methods for combination therapies in which a therapeutic agent useful in the treatment of a disease that is mediated by activation of IL-2R signaling in immune cells, including but not limited to, infectious diseases, autoimmune disorders, cancer, inflammatory diseases, and diseases associated with deficient IL-2-mediated signaling, deficient T-cell proliferation, or T-cell dysfunction, is used in combination with a heavy-chain antibody disclosed herein. In one aspect, such therapy includes, but is not limited to, the combination of one or more heavy-chain antibodies of the disclosure with a chemotherapeutic agent, an immune checkpoint inhibitor, a T-cell redirecting therapy (e.g., a CAR T-cell; a bispecific T-cell engaging molecule (e.g., tarlatamab)), or a combination of any of the foregoing to provide a synergistic or additive therapeutic effect for the treatment of cancer.

The heavy-chain antibodies of the disclosure can be administered simultaneously, separately, or sequentially with an effective amount of a second therapeutic agent in any of the methods described herein. In some cases, the second therapeutic agent is a chemotherapeutic agent, an immune checkpoint inhibitor, or a T-cell redirecting therapy (e.g., a CAR T-cell; a bispecific T-cell engaging molecule (e.g., tarlatamab)). In some cases, the second therapeutic agent is administered as a pharmaceutical composition comprising the second therapeutic agent and a pharmaceutically acceptable excipient.

CHEMOTHERAPEUTIC AGENTS. In some cases, the heavy-chain antibodies of the disclosure can be administered simultaneously, separately, or sequentially with an effective amount of a chemotherapeutic agent in any of the methods described herein. A chemotherapeutic agent, which also may be referred to as an antineoplastic agent, is a therapeutic agent that directly or indirectly stops the growth of rapidly dividing cells in the body, both cancerous and noncancerous. Example chemotherapeutic agents for use in the methods provided herein include, but are not limited to, alkylating agents, plant alkaloids, antitumor antibiotics, antimetabolites, and topoisomerase inhibitors.

IMMUNE CHECKPOINT INHIBITORS. In some cases, the heavy-chain antibodies of the disclosure can be administered simultaneously, separately, or sequentially with an effective amount of an immune checkpoint inhibitor in any of the methods described herein. An immune checkpoint inhibitor is a therapeutic agent that blocks one or more proteins known as checkpoints from binding with their partner proteins (e.g., PD-L1 and PD-1), thus blocking proteins that stop the immune system from attacking cancer cells. Example immune checkpoint inhibitors for use in the methods provided herein include, but are not limited to, CTLA-4 inhibitors (e.g., ipilimumab, tremelimumab, quavonlimab), PD-1 inhibitors (e.g., nivolumab, cemiplimab, pembrolizumab), PD-L1 inhibitors (e.g., atezolizumab, avelumab, durvalumab), LAG3 inhibitors (e.g., relatlimab), and TIGIT inhibitors (e.g., tiragolumab).

T-CELL REDIRECTING THERAPIES. In some cases, the heavy-chain antibodies of the disclosure can be administered simultaneously, separately, or sequentially with an effective amount of a T-cell redirecting therapy in any of the methods described herein. A T-cell redirecting therapy is a therapeutic agent capable of recruiting T-cells to a target cell or tissue. Example T-cell redirecting therapies for use in the methods provided herein include, but are not limited to, bispecific T-cell engaging molecules and CAR T-cells.

Illustratively, provided herein is a method of treating cancer in a subject in need thereof, comprising administering to the subject a heavy-chain antibody disclosed herein, or a pharmaceutical composition disclosed herein, in combination with a T-cell redirecting therapy. Further provided herein is a method of enhancing an anti-cancer effect associated with administration of a T-cell redirecting therapy in a subject diagnosed with cancer, comprising administering to the subject a heavy-chain antibody disclosed herein, or a pharmaceutical composition described herein, in combination with the T-cell redirecting therapy.

T-cell redirecting therapies, such as bispecific T-cell engaging molecules and CAR-expressing T-cells, represent a promising class of immunotherapies under development for the treatment of various solid and liquid cancers. While any therapeutic agent capable of recruiting T-cells to a target cell or tissue is envisioned for use in the methods described herein, specific embodiments related to bispecific T-cell engaging molecules and CAR-expressing T-cells are provided for the sake of illustration.

In some embodiments, the T-cell redirecting therapy used in a method described herein is a bispecific T-cell engaging molecule. The bispecific T-cell engaging molecules employed in these methods generally comprise a first domain that binds to a target cancer cell antigen (e.g., CEA, CD19, CD33, CD70, EGFRVIII, EpCAM, FLT3, GPRC5D, DLL3, BCMA, PSMA, STEAP1, STEAP2, MUC16, MUC17, or CLDN18.2), a second domain that binds to human CD3, and a half-life extension domain that provides a half-life for the molecule of greater than 24 hours. The half-life extension domain can be an immunoglobulin Fc domain, a domain derived from serum albumin (e.g., human serum albumin), an albumin-binding domain (e.g., comprising human albumin binding peptides or an antibody fragment that binds to serum albumin), peptides that bind to the neonatal Fc receptor (FcRn), and polyethylene glycol polymers. In certain embodiments, the bispecific T-cell engaging molecules used in these methods comprise an immunoglobulin Fc domain. In some such embodiments, the bispecific T-cell engaging molecule can be a bispecific antibody and have the general structure of a full-length immunoglobulin. For instance, in some embodiments, the bispecific T-cell engaging molecule can be a heterodimeric antibody comprising a light chain and heavy chain from an antibody that binds to a target cancer cell antigen, and a light chain and heavy chain from an antibody that binds to human CD3. In other embodiments, the bispecific T-cell engaging molecule can be an antibody fragment (e.g., a TCA) comprising a heavy chain from a heavy-chain antibody that binds to a target cancer cell antigen, and a light chain and a heavy chain from an antibody that binds to human CD3. In still other embodiments, the bispecific T-cell engaging molecule employed in the methods of the present disclosure comprises, in an amino to carboxyl order: (i) a first domain that binds to a target cancer cell antigen; (ii) a second domain that binds to human CD3; and (iii) an Fc domain comprising two Fc monomers, each monomer comprising an immunoglobulin hinge region, a CH2 domain, and a CH3 domain, wherein said two monomers are fused to each other via a peptide linker. In such embodiments, the bispecific T-cell engaging molecule can be a single chain polypeptide where all three domains are linked together, optionally via peptide linkers, to form a single polypeptide chain.

In certain embodiments, the binding domains of the bispecific T-cell engaging molecules comprise an immunoglobulin heavy chain variable region (VH) and an immunoglobulin light chain variable region (VL) of an antibody or antibody fragment which binds to the desired antigen. For instance, the anti-cancer cell antigen binding domain of the bispecific T-cell engaging molecules of the present disclosure comprises a VH region and a VL region from an antibody that binds to a target cancer cell antigen and the anti-CD3 binding domain comprises a VH region and a VL region from an antibody that binds to CD3. The binding domains that specifically bind to a human cancer cell antigen or human CD3 can be derived from known antibodies to these antigens or from new antibodies or antibody fragments obtained by de novo immunization methods using the antigen proteins or fragments thereof, by phage display, or other methods known in the art. The antibodies from which the binding domains for the bispecific T-cell engaging molecules are derived can be monoclonal antibodies, recombinant antibodies, chimeric antibodies, human antibodies, or humanized antibodies. In certain embodiments, the antibodies from which the binding domains are derived are monoclonal antibodies. In these and other embodiments, the antibodies may be human antibodies or humanized antibodies and can be of the IgG1-, IgG2-, IgG3-, or IgG4-type.

In some cases, the first binding domain of the bispecific T-cell engaging molecule binds to a target cancer cell antigen, such as, e.g., a human target cancer cell antigen. This binding domain is referred to herein as an anti-cancer cell antigen binding domain. The term “target cancer cell antigen” refers to an antigen expressed on the surface of a malignant cell, tumor cell, or other type of cancerous cell. A target cancer cell antigen may be expressed exclusively in cancer cells or may be overexpressed in cancer cells relative to normal cells. A target cancer cell antigen may also include a mutated or aberrant form of a protein expressed in cancer cells but not normal cells. Examples of a target cancer cell antigen include, but are not limited to, 5T4, AFP, BCMA, beta-catenin, BRCA1, CD19, CD20, CD22, CD33, CD70, CD123, CDH3, CDH19, CDK4, CEA, CLDN18.2, DLL3, DLL4, EGFR, EGFRvIII, EpCAM, EphA2, FLT3, FOLR1, gpA33, GPRC5D, HER2, IGFR, MAGE-1, MAGE-2, MAGE-3, MAGE-4, MAGE-6, MAGE-12, MSLN, MUC1, MUC2, MUC3, MUC4, MUC5, MUC16, MUC17, PSCA, PSMA, RAGE proteins, STEAP1, STEAP2, TRP1, and TRP2. In certain embodiments, the first domain of the bispecific T-cell engaging molecule binds to a target cancer cell antigen selected from MUC17, CLDN18.2, CD19, CD33, FLT3, DLL3, BCMA and PSMA.

In certain embodiments, the first domain of the bispecific T-cell engaging molecule binds to DLL3. Bispecific T-cell engaging molecules that specifically bind to DLL3 are disclosed, for example, in International Patent Publication Nos. WO2017/021349 and WO2023/215829, which are incorporated by reference herein in their entireties.

For example, a bispecific T-cell engaging molecule used in the methods described herein can include one or more amino acid sequences set forth in TABLE 9, TABLE 10, or TABLE 11A. The specific CDRs identified in TABLE 9 and TABLE 10 are defined by Kabat.

TABLE 9
DLL3-Related Amino Acid Sequences

| Description | Amino Acid Sequence | SEQ ID NO: |
|---|---|---|
| VH CDR1 | SYYWS | 62 |
| VH CDR2 | YVYYSGTTNYNPSLKS | 63 |
| VH CDR3 | IAVTGFYFDY | 64 |
| VL CDR1 | RASQRVNNNYLA | 65 |
| VL CDR2 | GASSRAT | 66 |
| VL CDR3 | QQYDRSPLT | 67 |
| VH | QVQLQESGPGLVKPSETLSLTCTVSGGSISSYYWSWIRQPPGK | 68 |
| | CLEWIGYVYYSGTTNYNPSLKSRVTISVDTSKNQFSLKLSSVT | |
| | AADTAVYYCASIAVTGFYFDYWGQGTLVTVSS | |
| VL | EIVLTQSPGTLSLSPGERVTLSCRASQRVNNNYLAWYQQRPG | 69 |
| | QAPRLLIYGASSRATGIPDRFSGSGSGTDFTLTISRLEPEDFAV | |
| | YYCQQYDRSPLTFGCGTKLEIK | |
| scFv | QVQLQESGPGLVKPSETLSLTCTVSGGSISSYYWSWIRQPPGK | 70 |
| | CLEWIGYVYYSGTTNYNPSLKSRVTISVDTSKNQFSLKLSSVT | |
| | AADTAVYYCASIAVTGFYFDYWGQGTLVTVSSGGGGSGGG | |
| | GSGGGGSEIVLTQSPGTLSLSPGERVTLSCRASQRVNNNYLA | |
| | WYQQRPGQAPRLLIYGASSRATGIPDRFSGSGSGTDFTLTISR | |
| | LEPEDFAVYYCQQYDRSPLTFGCGTKLEIK | |

TABLE 10
CD3-Related Amino Acid Sequences

| Description | Amino Acid Sequence | SEQ ID NO: |
|---|---|---|
| VH CDR1 | KYAMN | 71 |
| VH CDR2 | RIRSKYNNYATYYADSVKD | 72 |
| VH CDR3 | HGNFGNSYISYWAY | 73 |
| VL CDR1 | GSSTGAVTSGNYPN | 74 |
| VL CDR2 | GTKFLAP | 75 |
| VL CDR3 | VLWYSNRWV | 76 |
| VH | EVQLVESGGGLVQPGGSLKLSCAASGFTFNKYAMNWVRQA | 77 |
| | PGKGLEWVARIRSKYNNYATYYADSVKDRFTISRDDSKNTA | |
| | YLQMNNLKTEDTAVYYCVRHGNFGNSYISYWAYWGQGTL | |
| | VTVSS | |
| VL | QTVVTQEPSLTVSPGGTVTLTCGSSTGAVTSGNYPNWVQQK | 78 |
| | PGQAPRGLIGGTKFLAPGTPARFSGSLLGGKAALTLSGVQPE | |
| | DEAEYYCVLWYSNRWVFGGGTKLTVL | |
| scFv | EVQLVESGGGLVQPGGSLKLSCAASGFTFNKYAMNWVRQA | 79 |
| | PGKGLEWVARIRSKYNNYATYYADSVKDRFTISRDDSKNTA | |
| | YLQMNNLKTEDTAVYYCVRHGNFGNSYISYWAYWGQGTL | |
| | VTVSSGGGGSGGGGSGGGGSQTVVTQEPSLTVSPGGTVTLT | |
| | CGSSTGAVTSGNYPNWVQQKPGQAPRGLIGGTKFLAPGTPA | |
| | RFSGSLLGGKAALTLSGVQPEDEAEYYCVLWYSNRWVFGG | |
| | GTKLTVL | |

TABLE 11A
DLL3xCD3 Bispecific T-cell Engaging Molecule Related Amino Acid Sequences

| Description | Amino Acid Sequence | SEQ ID NO: |
|---|---|---|
| Bispecific | QVQLQESGPGLVKPSETLSLTCTVSGGSISSYYWSWIRQPPGK | 80 |
| Molecule | CLEWIGYVYYSGTTNYNPSLKSRVTISVDTSKNQFSLKLSSVT | |
| | AADTAVYYCASIAVTGFYFDYWGQGTLVTVSSGGGGSGGG | |
| | GSGGGGSEIVLTQSPGTLSLSPGERVTLSCRASQRVNNNYLA | |
| | WYQQRPGQAPRLLIYGASSRATGIPDRFSGSGSGTDFTLTISR | |
| | LEPEDFAVYYCQQYDRSPLTFGCGTKLEIKSGGGGSEVQLVE | |
| | SGGGLVQPGGSLKLSCAASGFTFNKYAMNWVRQAPGKGLE | |
| | WVARIRSKYNNYATYYADSVKDRFTISRDDSKNTAYLQMN | |
| | NLKTEDTAVYYCVRHGNFGNSYISYWAYWGQGTLVTVSSG | |
| | GGGSGGGGSGGGGSQTVVTQEPSLTVSPGGTVTLTCGSSTG | |
| | AVTSGNYPNWVQQKPGQAPRGLIGGTKFLAPGTPARFSGSLL | |
| | GGKAALTLSGVQPEDEAEYYCVLWYSNRWVFGGGTKLTVL | |
| Bispecific | QVQLQESGPGLVKPSETLSLTCTVSGGSISSYYWSWIRQPPGK | 81 |
| HLE | CLEWIGYVYYSGTTNYNPSLKSRVTISVDTSKNQFSLKLSSVT | |
| Molecule | AADTAVYYCASIAVTGFYFDYWGQGTLVTVSSGGGGSGGG | |
| | GSGGGGSEIVLTQSPGTLSLSPGERVTLSCRASQRVNNNYLA | |
| | WYQQRPGQAPRLLIYGASSRATGIPDRFSGSGSGTDFTLTISR | |
| | LEPEDFAVYYCQQYDRSPLTFGCGTKLEIKSGGGGSEVQLVE | |
| | SGGGLVQPGGSLKLSCAASGFTFNKYAMNWVRQAPGKGLE | |
| | WVARIRSKYNNYATYYADSVKDRFTISRDDSKNTAYLQMN | |
| | NLKTEDTAVYYCVRHGNFGNSYISYWAYWGQGTLVTVSSG | |
| | GGGSGGGGSGGGGSQTVVTQEPSLTVSPGGTVTLTCGSSTG | |
| | AVTSGNYPNWVQQKPGQAPRGLIGGTKFLAPGTPARFSGSLL | |
| | GGKAALTLSGVQPEDEAEYYCVLWYSNRWVFGGGTKLTVL | |
| | GGGGDKTHTCPPCPAPELLGGPSVFLFPPKPKDTLMISRTPEV | |
| | TCVVVDVSHEDPEVKFNWYVDGVEVHNAKTKPCEEQYGST | |
| | YRCVSVLTVLHQDWLNGKEYKCKVSNKALPAPIEKTISKAK | |
| | GQPREPQVYTLPPSREEMTKNQVSLTCLVKGFYPSDIA VEWE | |
| | SNGQPENNYKTTPPVLDSDGSFFLYSKLTVDKSRWQQGNVF | |
| | SCSVMHEALHNHYTQKSLSLSPGGGGSGGGGSGGGGSGGG | |
| | GSGGGGSGGGGSDKTHTCPPCPAPELLGGPSVFLFPPKPKDT | |
| | LMISRTPEVTCVVVDVSHEDPEVKFNWYVDGVEVHNAKTKP | |
| | CEEQYGSTYRCVSVLTVLHQDWLNGKEYKCKVSNKALPAPI | |
| | EKTISKAKGQPREPQVYTLPPSREEMTKNQVSLTCLVKGFYP | |
| | SDIA VEWESNGQPENNYKTTPPVLDSDGSFFLYSKLTVDKSR | |
| | WQQGNVFSCSVMHEALHNHYTQKSLSLSPGK | |

In some embodiments, the bispecific T-cell engaging molecule used in the methods described herein binds to DLL3 and comprises:
a heavy chain variable (VH) region comprising a VH CDR1, a VH CDR2, and a VH CDR3, wherein the VH CDR1, VH CDR2, and VH CDR3 comprise the amino acid sequences of SEQ ID NOs: 62, 63, and 64, respectively; and
a light chain variable (VL) region comprising a VL CDR1, a VL CDR2, and a VL CDR3, wherein the VL CDR1, VL CDR2, and VL CDR3 comprise the amino acid sequences of SEQ ID NOs: 65, 66, and 67, respectively.

In some embodiments, the bispecific T-cell engaging molecule used in the methods described herein binds to DLL3 and comprises a VH region comprising the amino acid sequence of SEQ ID NO: 68 and a VL region comprising the amino acid sequence of SEQ ID NO: 69.

In some embodiments, the bispecific T-cell engaging molecule used in the methods described herein binds to DLL3 and comprises the amino acid sequence of SEQ ID NO: 70.

In some embodiments, the bispecific T-cell engaging molecule used in the methods described comprises a first domain that binds to DLL3 and a second domain that binds to CD3, wherein:
the first domain comprises a first heavy chain variable (VH) region comprising a VH CDR1, a VH CDR2, and a VH CDR3, wherein the VH CDR1, VH CDR2, and VH CDR3 comprise the amino acid sequences of SEQ ID NOs: 62, 63, and 64, respectively; and a first light chain variable (VL) region comprising a VL CDR1, a VL CDR2, and a VL CDR3, wherein the VL CDR1, VL CDR2, and VL CDR3 comprise the amino acid sequences of SEQ ID NOs: 65, 66, and 67, respectively; and
the second domain comprises a second VH region comprising a VH CDR1, a VH CDR2, and a VH CDR3, wherein the VH CDR1, VH CDR2, and VH CDR3 comprise the amino acid sequences of SEQ ID NOs: 71, 72, and 73, respectively; and a second VL region comprising a VL CDR1, a VL CDR2, and a VL CDR3, wherein the VL CDR1, VL CDR2, and VL CDR3 comprise the amino acid sequences of SEQ ID NOs: 74, 75, and 76, respectively.

In some embodiments, the bispecific T-cell engaging molecule used in the methods described comprises a first domain that binds to DLL3 and a second domain that binds to CD3, wherein:
the first domain comprises a first heavy chain variable (VH) region comprising the amino acid sequence of SEQ ID NO: 68 and a first light chain variable (VL) region comprising the amino acid sequence of SEQ ID NO: 69; and
the second domain comprises a second VH region comprising the amino acid sequence of SEQ ID NO: 77 and a second VL region comprising the amino acid sequence of SEQ ID NO: 78.

In some embodiments, the bispecific T-cell engaging molecule used in the methods described comprises a first domain that binds to DLL3 and a second domain that binds to CD3, wherein the first domain comprises the amino acid sequence of SEQ ID NO: 70 and the second domain comprises the amino acid sequence of SEQ ID NO: 79.

In some embodiments, the bispecific T-cell engaging molecule used in the methods described herein binds to DLL3 and CD3 and comprises the amino acid sequence of SEQ ID NO: 80 or SEQ ID NO: 81. In some embodiments, the bispecific T-cell engaging molecule comprises the amino acid sequence of SEQ ID NO: 80. In some embodiments, the bispecific T-cell engaging molecule comprises the amino acid sequence of SEQ ID NO: 81.

In some embodiments, the bispecific T-cell engaging molecule comprises a variant of the amino acid sequence of SEQ ID NO: 81 in which the C-terminal lysine of SEQ ID NO: 81 is clipped.

In some embodiments, the bispecific T-cell engaging molecule used in the methods described herein binds to DLL3 and CD3 and consists of the amino acid sequence of SEQ ID NO: 80 or SEQ ID NO: 81. In some embodiments, the bispecific T-cell engaging molecule consists of the amino acid sequence of SEQ ID NO: 80. In some embodiments, the bispecific T-cell engaging molecule consists of the amino acid sequence of SEQ ID NO: 81.

In some embodiments, the bispecific T-cell engaging molecule consists of a variant of the amino acid sequence of SEQ ID NO: 81 in which the C-terminal lysine of SEQ ID NO: 81 is clipped.

In some embodiments, the bispecific T-cell engaging molecule used in the methods described herein is tarlatamab. Tarlatamab is a DLL3-targeted bispecific T-cell engaging molecule under investigation for the treatment of small cell lung cancer and neuroendocrine prostate cancer. An intention-to-treat analysis from the global Phase 2 DeLLphi-301 study that included 100 patients with advanced stage small cell lung cancer (SCLC) who had failed two or more prior lines of treatment at a 10 mg dose for tarlatamab demonstrated an objective response rate (ORR; primary endpoint) of 40% (97.5% Confidence Interval (CI): 29, 52), with a median follow-up of 10.6 months. For key secondary endpoints, median progression-free survival (mPFS) was 4.9 months (95% CI: 2.9, 6.7) and median overall survival (mOS) was 14.3 months (95% CI: 10.8, NE). Median response duration was not reached. Of the patients who responded to treatment with tarlatamab at 10 mg dose, 58% experienced at least six months of response and 55% of responses were ongoing at data cutoff.

In other embodiments, a T-cell engaging molecule used in the methods described herein binds to DLL3 and is a single chain polypeptide comprising one or more scFvs fused to one or more single domain monoclonal antibodies (sdmAb). Optionally, the scFv-sdmAb format may comprise a domain that serves to extend the half-life of the protein. For example, the anti-DLL3 T-cell engaging molecule may comprise, from N to C terminus, an scFv that binds CD3, a linker peptide, a sdmAb that binds serum albumin, a peptide linker, and a sdmAb that binds DLL3, such as, for example, a polypeptide comprising the amino acid sequence of SEQ ID NO: 83. An alternate format for the anti-DLL3 T-cell engaging molecule comprises, from N- to C-terminus, a sdmAb that binds DLL3, a peptide linker, a sdmAb that binds serum albumin, a peptide linker, and an scFv that binds CD3. A non-limiting example of such an anti-DLL3 T-cell engaging molecule comprises the amino acid sequence of SEQ ID NO: 84. Anti-DLL3 T-cell engaging molecules comprising these formats are further described in, e.g., International Patent Publication No. WO 2020/069028, which is incorporated by reference with respect to its disclosure relating to anti-DLL3 T-cell engaging molecules and their associated amino acid sequences. For example, an anti-DLL3 T-cell engaging molecule used in the methods described herein can include one or more amino acid sequences set forth in TABLE 11B.

TABLE 11B
Anti-DLL3 T-cell Engaging Molecule Related Amino Acid Sequences

| SEQ ID | | | |
|---|---|---|---|
| NO: | Target | Description | Amino Acid Sequence |
| 83 | DLL3x | scFv-single | EVQLVESGGGLVQPGGSLKLSCAASGFTFNKYAI |
| | CD3 | domain mAb | NWVRQAPGKGLEWVARIRSKYNNYATYYADQV |
| | | (sdmAb) format; | KDRFTISRDDSKNTAYLQMNNLKTEDTAVYYCV |
| | | anti-CD3 scFv- | RHANFGNSYISYWAYWGQGTL |
| | | anti-DLL3 | VTVSSGGGGSGGGGSGGGGSQTVVTQEPSLTVSP |
| | | sdmAb | GGTVTLTCASSTGAVTSGNYPNWVQQKPGQAPR |
| | | | GLIGGTKFLVPGTPARFSGSLLGGKAALTLSGVQP |
| | | | EDEAEYYCTLWYSNRWVFGGGTKLTVLGGGGSG |
| | | | GGSEVQLVESGGGLVQPGNSLRLSCAASGFTFSKF |
| | | | GMSWVRQAPGKGLEWVSSISGSGRDTLYADSVK |
| | | | GRFTISRDNAKTTLYLQMNSLRPEDTAVYYCTIG |
| | | | GSL |
| | | | SVSSQGTLVTVSSGGGGSGGGSEVQLVESGGGLV |
| | | | QPGGSLTLSCAASSSSVSLLSLAWYRQAPGKKRE |
| | | | LVAGISDDGSIVYMDSVKGRFTISRDNAKNSVYL |
| | | | QMNSLRAEDTAVYYCYAY |
| | | | SWITRSPYWGQGTLVTVSSHHHHHH |
| 84 | DLL3x | scFv-single | EVQLVESGGGLVQPGGSLTLSCAASSSSVSLLSLA |
| | CD3 | domain mAb | WYRQAPGKKRELVAGISDDGSIVYMDSVKGRFTI |
| | | (sdmAb) format; | SRDNAKNSVYLQMNSLRAEDTAVYYCYAYSWIT |
| | | anti-DLL3 | RSPYWGQGTLVTVSSGGG |
| | | sdmAb-anti- | GSGGGSEVQLVESGGGLVQPGNSLRLSCAASGFT |
| | | CD3 scFv | FSKFGMSWVRQAPGKGLEWVSSISGSGRDTLYAD |
| | | | SVKGRFTISRDNAKTTLYLQMNSLRPEDTAVYYC |
| | | | TIGGSLSVSSQGTLVTVS |
| | | | SGGGGSGGGSEVQLVESGGGLVQPGGSLKLSCAA |
| | | | SGFTFNKYAINWVRQAPGKGLEWVARIRSKYNN |
| | | | YATYYADQVKDRFTISRDDSKNTAYLQMNNLKT |
| | | | EDTAVYYCVRHANFGNSYIS |
| | | | YWAYWGQGTLVTVSSGGGGSGGGGSGGGGSQT |
| | | | VVTQEPSLTVSPGGTVTLTCASSTGAVTSGNYPN |
| | | | WVQQKPGQAPRGLIGGTKFLVPGTPARFSGSLLG |
| | | | GKAALTLSGVQPEDEAEYYCTLWYSNRWVFGGG |
| | | | TKLTVLHHHHHH |

In other embodiments, a T-cell engaging molecule used in the methods described herein binds to DLL3 and takes the form of a traditional bispecific IgG-like antibody, wherein one arm binds DLL3 and the second arm binds CD3. In some embodiments, such an antibody construct comprises two arms comprising, from N- to C-terminus, a VL domain, a CL domain, a peptide linker, a VH domain, a CHIdomain, a peptide linker, and an Fc domain (scFab-Fc). In some embodiments, the anti-DLL3 T-cell engaging molecule comprises a first domain binds to DLL3 (e.g., human DLL3) and comprises (a) a heavy chain variable region (VH) that comprises (i) a VH complementarity determining region one (CDR-H1) comprising the amino acid sequence of SEQ ID NO: 85, (ii) a CDR-H2 comprising the amino acid sequence of SEQ ID NO: 86, and (iii) a CDR-H3 comprising the amino acid sequence of SEQ ID NO: 87; and (b) a light chain variable region (VL) that comprises (i) a VL complementarity determining region one (CDR-L1) comprising the amino acid sequence of SEQ ID NO: 88, (ii) a CDR-L2 comprising the amino acid sequence of SEQ ID NO: 89, and (iii) a CDR-L3 comprising the amino acid sequence of SEQ ID NO: 90. Optionally, the first domain binds DLL3 and comprises a VH region comprising the amino acid sequence of SEQ ID NO: 91 and a VL region comprising the amino acid sequence of SEQ ID NO: 92. In some embodiments, the DLL3 binding region comprises SEQ ID NO: 93. The second domain binds to CD3 (e.g., human CD3) and comprises (a) a VH that comprises (i) a CDR-H1 comprising the amino acid sequence of SEQ ID NO: 95, (ii) a CDR-H2 comprising the amino acid sequence of SEQ ID NO: 96, and (iii) a CDR-H3 comprising the amino acid sequence of SEQ ID NO: 97; and (b) a VL that comprises (i) a CDR-L1 comprising the amino acid sequence of SEQ ID NO: 98, (ii) a CDR-L2 comprising the amino acid sequence of SEQ ID NO: 99, and (iii) a CDR-L3 comprising the amino acid sequence of SEQ ID NO: 100. Optionally, the second domain binds CD3 and comprises a VH region comprising the amino acid sequence of SEQ ID NO: 101 and a VL region comprising the amino acid sequence of SEQ ID NO: 102. In some embodiments, the CD3 binding region of the anti-DLL3 T-cell engaging molecule comprises SEQ ID NO: 103. In some embodiments, the anti-DLL3 T-cell engaging molecule optionally comprises the amino acid sequence of SEQ ID NO: 94 and the amino acid sequence of SEQ ID NO: 104. Anti-DLL3 T-cell engaging molecules are further described in, e.g., International Patent Publication No. WO 2019/234220, which is incorporated by reference with respect to its disclosure relating to anti-DLL3 T-cell engaging molecules and their associated amino acid sequences. Illustratively, an anti-DLL3 T-cell engaging molecule used in the methods described herein can include one or more amino acid sequences set forth in TABLE 11C.

TABLE 11C
Anti-DLL3 T-cell Engaging Molecule Related Amino Acid Sequences

| SEQ ID | | | |
|---|---|---|---|
| NO: | Target | Description | Amino Acid Sequence |
| 85 | DLL3 | VH CDR1 | GYTFTSYYVH |
| 86 | DLL3 | VH CDR2 | IINPGGGTTSYAQKFLG |
| 87 | DLL3 | VH CDR3 | GEAVTGNYFYYGMDV |
| 88 | DLL3 | VL CDR1 | RASQGISNYLV |
| 89 | DLL3 | VL CDR2 | AVSSLYS |
| 90 | DLL3 | VL CDR3 | LQHDSYPYT |
| 91 | DLL3 | VH | QVQLVQSGAEVKKPGASVKVSCKASGYTFTSYY |
| | | | VHWVRQAPGQGLEWMVIINPGGGTTSYAQKFLG |
| | | | RVTMTRDTSTNTVYMELKSLRSEDTAVYYCARG |
| | | | EAVTGNYFYYGMDVWGQGTTVTVSS |
| 92 | DLL3 | VL | DIQMTQSPSAMSASVGDRVTITCRASQGISNYLV |
| | | | WFQQKPGKAPKRLIYAVSSLYSGVPSRFSGSGSGT |
| | | | EFTLTISSLQPEDFATYYCLQHDSYPYTFGQGTKL |
| | | | EIK |
| 93 | DLL3 | scFab | DIQMTQSPSAMSASVGDRVTITCRASQGISNYLV |
| | | | WFQQKPGKAPKRLIYAVSSLYSGVPSRFSGSGSGT |
| | | | EFTLTISSLQPEDFATYYCLQHDSYPYTFGQGTKL |
| | | | EIKRTVAAPSVFIFPP |
| | | | SDEQLKSGTASVVCLLNNFYPREAKVQWKVDNA |
| | | | LQSGNSQESVTEQDSKDSTYSLSSTLTLSKADYEK |
| | | | HKVYACEVTHQGLSSPVTKSFNRGECGGGGSEGK |
| | | | SSGSGSESKSTEGKSSGS |
| | | | GSESKSTGGGGSQVQLVQSGAEVKKPGASVKVSC |
| | | | KASGYTFTSYYVHWVRQAPGQGLEWMVIINPGG |
| | | | GTTSYAQKFLGRVTMTRDTSTNTVYMELKSLRSE |
| | | | DTAVYYCARGEAVTGNYFYYGMDVWGQGTTVT |
| | | | VSSASTKGPSVFPLAPSSKSTSGGTAALGCLVKDY |
| | | | FPEPVTVSWNSGALTSGVHTFPAVLQSSGLYSLSS |
| | | | VVTVPSSSLGTQTYICNVNHKPSNTKVDKRVEPK |
| | | | SC |
| 94 | DLL3 | scFab-Fc | DIQMTQSPSAMSASVGDRVTITCRASQGISNYLV |
| | | | WFQQKPGKAPKRLIYAVSSLYSGVPSRFSGSGSGT |
| | | | EFTLTISSLQPEDFATYYCLQHDSYPYTFGQGTKL |
| | | | EIKRTVAAPSVFIFPPSDEQLKSGTASVVCLLNNFY |
| | | | PREAKVQWKVDNALQSGNSQESVTEQDSKDSTY |
| | | | SLSSTLTLSKADYEKHKVYACEVTHQGLSSPVTK |
| | | | SFNRGECGGGGSEGKSSGSGSESKSTEGKSSGSGS |
| | | | ESKSTGGGGSQVQLVQSGAEVKKPGASVKVSCK |
| | | | ASGYTFTSYYVHWVRQAPGQGLEWMVIINPGGG |
| | | | TTSYAQKFLGRVTMTRDTSTNTVYMELKSLRSED |
| | | | TAVYYCARGEAVTGNYFYYGMDVWGQGTTVTV |
| | | | SSASTKGPSVFPLAPSSKSTSGGTAALGCLVKDYF |
| | | | PEPVTVSWNSGALTSGVHTFPAVLQSSGLYSLSSV |
| | | | VTVPSSSLGTQTYICNVNHKPSNTKVDKRVEPKSC |
| | | | DKTHTCPPCPAPEAAGGPSVFLFPPKPKDTLMISR |
| | | | TPEVTCVVVDVSHEDPEVKFNWYVDGVEVHNAK |
| | | | TKPREEQYNSTYRVVSVLTVLHQDWLNGKEYKC |
| | | | KVSNKALPAPIEKTISKAKGQPREPQVYTLPPSREE |
| | | | MTKNQVSLWCLVKGFYPSDIAVEWESNGQPENN |
| | | | YKTTPPVLDSDGSFFLYSKLTVDKSRWQQGNVFS |
| | | | CSVMHEALHNHYTQKSLSLSPG |
| 95 | CD3 | VH CDR1 | GFTFNTYAMN |
| 96 | CD3 | VH CDR2 | RIRSKYNNYATYYADSVKD |
| 97 | CD3 | VH CDR3 | HGNFGNSYVSWFAY |
| 98 | CD3 | VL CDR1 | RSSTGAVTTSNYAN |
| 99 | CD3 | VL CDR2 | GTNKRAP |
| 100 | CD3 | VL CDR3 | ALWYSNLWV |
| 101 | CD3 | VH | EVQLVESGGGLVQPGGSLKLSCAASGFTFNTYAM |
| | | | NWVRQAPGKGLEWVARIRSKYNNYATYYADSV |
| | | | KDRFTISRDDSKNTAYLQMNNLKTEDTAVYYCV |
| | | | RHGNFGNSYVSWFAYWGQGTLVTVSA |
| 102 | CD3 | VL | EAVVTQEPSLTVSPGGTVTLTCRSSTGAVTTSNYA |
| | | | NWVQEKPGQLPRGLIGGTNKRAPWVPARFSGSLL |
| | | | GGKAALTLSGAQPEDEAEYFCALWYSNLWVFGG |
| | | | GTKLTVL |
| 103 | CD3 | scFab | EAVVTQEPSLTVSPGGTVTLTCRSSTGAVTTSNYA |
| | | | NWVQEKPGQLPRGLIGGTNKRAPWVPARFSGSLL |
| | | | GGKAALTLSGAQPEDEAEYFCALWYSNLWVFGG |
| | | | GTKLTVLGQPKAAPSVTL |
| | | | FPPSSEELQANKATLVCLISDFYPGAVKVAWKAD |
| | | | GSPVNTGVETTTPSKQSNNKYAASSYLSLTPEQW |
| | | | KSHRSYSCQVTHEGSTVEKTVAPAECSGGGGSEG |
| | | | KSSGSGSESKSTEGKSSG |
| | | | SGSESKSTGGGGSEVQLVESGGGLVQPGGSLKLS |
| | | | CAASGFTFNTYAMNWVRQAPGKGLEWVARIRSK |
| | | | YNNYATYYADSVKDRFTISRDDSKNTAYLQMNN |
| | | | LKTEDTAVYYCVRHGNFGNS |
| | | | YVSWFAYWGQGTLVTVSAASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNV |
| | | | NHKPSNTKVDKRVEPKS |
| 104 | CD3 | scFab-Fc | EAVVTQEPSLTVSPGGTVTLTCRSSTGAVTTSNYA |
| | | | NWVQEKPGQLPRGLIGGTNKRAPWVPARFSGSLL |
| | | | GGKAALTLSGAQPEDEAEYFCALWYSNLWVFGG |
| | | | GTKLTVLGQPKAAPSVTL |
| | | | FPPSSEELQANKATLVCLISDFYPGAVKVAWKAD |
| | | | GSPVNTGVETTTPSKQSNNKYAASSYLSLTPEQW |
| | | | KSHRSYSCQVTHEGSTVEKTVAPAECSGGGGSEG |
| | | | KSSGSGSESKSTEGKSSG |
| | | | SGSESKSTGGGGSEVQLVESGGGLVQPGGSLKLS |
| | | | CAASGFTFNTYAMNWVRQAPGKGLEWVARIRSK |
| | | | YNNYATYYADSVKDRFTISRDDSKNTAYLQMNN |
| | | | LKTEDTAVYYCVRHGNFGNS |
| | | | YVSWFAYWGQGTLVTVSAASTKGPSVFPLAPSSK |
| | | | STSGGTAALGCLVKDYFPEPVTVSWNSGALTSGV |
| | | | HTFPAVLQSSGLYSLSSVVTVPSSSLGTQTYICNV |
| | | | NHKPSNTKVDKRVEPKSCDKTHTCPPCPAPEAAG |
| | | | GPSVFLFPPKPKDTLMISRTPEVTCVVVDVSHEDP |
| | | | EVKFNWYVDGVEVHNAKTKPREEQYNSTYRVVS |
| | | | VLTVLHQDWLNGKEYKCKVSNKALPAPIEKTISK |
| | | | AKGQPREPQVYTLPPSREEMTKNQVSLSCAVKGF |
| | | | YPSDIA VEWESNGQPENNYKTTPPVLDSDGSFFLV |
| | | | SKLTVDKSRWQQGNVFSCSVMHEALHNRFTQKS |
| | | | LSLSPG |

Combination therapies comprising an anti-DLL3 T-cell engaging molecule and a heavy-chain antibody described herein may be useful for the treatment of a DLL3-expressing cancer.

In some embodiments, the DLL3-expressing cancer is a neuroendocrine cancer (i.e., neuroendocrine neoplasm). In this regard, the DLL3-expressing cancer may be a pulmonary neuroendocrine cancer or an extrapulmonary neuroendocrine cancer. In some embodiments, a subject to be treated with the combination therapy is suffering from a pulmonary neuroendocrine cancer, such as small cell lung cancer (SCLC), examples of which include relapsed/refractory SCLC (RR SCLC), extensive stage SCLC (ES SCLC) (which may be relapsed or refractory), and limited-stage SCLC (LS-SCLC) (which may be relapsed or refractory). LS-SCLC often refers to SCLC cancers found only on one side of the chest (e.g., only in one lung), enabling treatment with a single radiation field. In some embodiments, LS-SCLC has not progressed in the subject following concurrent chemoradiation therapy. ES SCLC includes cancers that have spread more widely throughout the lung and/or to the other lung, and often to other locations within the body. Relapsed SCLC include cancers which, e.g., reemerge within three months of a first line of treatment (e.g., chemotherapy and/or radiation).

In certain embodiments, the DLL3-expressing cancer is an extrapulmonary neuroendocrine cancer, such as glioma, glioblastoma, melanoma, prostate cancer (e.g., neuroendocrine prostate cancer), neuroendocrine pancreatic cancer, hepatoblastoma, large cell pulmonary neuroendocrine cancer, pancreatic neuroendocrine cancer, bladder neuroendocrine cancer, gastric neuroendocrine cancer, adrenal exocrine tumors, Merkel cell carcinoma, neuroblastoma, head and neck carcinoid or neuroendocrine cancer, head and neck paraganglioma, or cervical small cell neuroendocrine cancer. In some embodiments, the extrapulmonary neuroendocrine cancer is neuroendocrine pancreatic cancer (NEPC).

In some embodiments, the subject to be treated with the combination therapy suffers from a DLL3-expressing cancer (e.g., SCLC) and has not received any prior line of cancer treatment. By “prior line of treatment” is meant a previous treatment with another anticancer therapeutic, e.g., a first- or second-line cancer therapy or standard of care therapy, before administration of the combination therapy. In other embodiments, the subject suffers from a DLL3-expressing cancer (e.g., SCLC) which has not progressed or relapsed after one or more prior lines of treatment. For example, in some embodiments, the subject has received a first-line cancer therapy, after which the subject's disease has not progressed (defined as ongoing response or stable disease per Response Evaluation Criteria in Solid Tumors Version 1.1 (RECIST 1.1)). In other embodiments, the subject suffers from a DLL3-expressing cancer (e.g., SCLC) which has recurred after one or more prior lines of treatment in the subject, such as a SCLC recurrence after two or more prior lines of treatment in the subject. Illustratively, in some embodiments, the subject has received at least one, two, three, four, five, or six prior lines of treatment for the DLL3-expressing cancer (e.g., SCLC). In some embodiments, at least one of the prior lines of treatment is a platinum-based chemotherapeutic. Alternatively or in addition, at least one of the prior lines of treatment is anti-PD1 antibody or anti-PD-L1 antibody therapy. For instance, the subject may have undergone a combination of therapies including platinum-based chemotherapy and anti-PD1 antibody or anti-PD-L1 antibody therapy. Where the subject has undergone prior lines of treatment, administration of the combination therapy may occur at any timepoint following completion of the prior lines of treatment. For instance, the combination therapy may be administered at least 28 days (e.g., at least 60 days or at least 90 days) after the completion of any prior lines of treatment for the DLL3-expressing cancer (e.g., SCLC).

The second binding domain of the bispecific T-cell engaging molecules used in the methods of the present disclosure can specifically bind to CD3, such as, e.g., human CD3. This binding domain is referred to herein as an anti-CD3 binding domain. “CD3” (cluster of differentiation 3) is a T-cell co-receptor composed of four chains. In mammals, the CD3 protein complex contains a CD3Y (gamma) chain, a CD3δ (delta) chain, and two CD3ε (epsilon) chains. These four chains associate with the T cell receptor (TCR) and the so-called ζ (zeta) chain to form the “T cell receptor complex” and to generate an activation signal in T lymphocytes. The CD3γ (gamma), CD3δ (delta), and CD3ε (epsilon) chains are highly related cell-surface proteins of the immunoglobulin superfamily and each contain a single extracellular immunoglobulin domain. The intracellular tails of the CD3 molecules contain a single conserved motif known as an immunoreceptor tyrosine-based activation motif (ITAM), which is essential for the signaling capacity of the TCR. The CD3 epsilon molecule is a polypeptide, which in humans is encoded by the CD3E gene which resides on chromosome 11.

The redirected lysis of target cells via the recruitment of T-cells by a T-cell engaging molecule which binds to CD3 on the T cell and to a target protein (e.g., cancer cell antigen) on the target cell (e.g., tumor cell) generally involves cytolytic synapse formation and delivery of perforin and granzymes. The engaged T-cells are capable of serial target cell lysis and are not affected by immune escape mechanisms interfering with peptide antigen processing and presentation, or clonal T cell differentiation; see, for example, WO 2007/042261.

In certain embodiments, the second binding domain of the bispecific T-cell engaging molecules used in the methods of the present disclosure binds to CD3 on the surface of a T cell, such as, e.g., to human CD3 on the surface of a T cell. In some embodiments, the second binding domain of the bispecific T-cell engaging molecules binds to CD3 epsilon, such as, e.g., human CD3 epsilon, e.g., human CD3 epsilon on the surface of a T-cell. In some embodiments, the second binding domain of the bispecific T-cell engaging molecules binds to CD3 delta/epsilon, such as, e.g., human CD3 delta/epsilon, e.g., human CD3 epsilon on the surface of a T-cell. Non-limiting examples of anti-CD3 antibodies or anti-CD3 binding domains from which the second binding domain of the bispecific T-cell engaging molecules used in the methods of the present disclosure can be constructed or derived are described in WO 2007/042261, WO 2008/119567, WO 2017/053856, WO 2017/201493, WO 2017/223111, WO 2018/052503, and WO 2019/224717, all of which are hereby incorporated by reference in their entireties. In certain embodiments, the second domain of the bispecific T-cell engaging molecules used in the methods of the present disclosure binds to an epitope in the extracellular domain of human CD3 epsilon.

In some embodiments, the bispecific T-cell engaging molecule used in the methods described herein binds to CD3 and comprises:
a heavy chain variable (VH) region comprising a VH CDR1, a VH CDR2, and a VH CDR3, wherein the VH CDR1, VH CDR2, and VH CDR3 comprise the amino acid sequences of SEQ ID NOs: 71, 72, and 73, respectively; and
a light chain variable (VL) region comprising a VL CDR1, a VL CDR2, and a VL CDR3, wherein the VL CDR1, VL CDR2, and VL CDR3 comprise the amino acid sequences of SEQ ID NOs: 74, 75, and 76, respectively.

In some embodiments, the bispecific T-cell engaging molecule used in the methods described herein binds to CD3 and comprises a VH region comprising the amino acid sequence of SEQ ID NO: 77 and a VL region comprising the amino acid sequence of SEQ ID NO: 78.

In some embodiments, the bispecific T-cell engaging molecule used in the methods described herein binds to CD3 and comprises the amino acid sequence of SEQ ID NO: 79.

Bispecific T-cell engaging molecules suitable for use in the methods of the present disclosure may comprise additional domains, which, e.g., can modulate the pharmacokinetic profile of the molecule. For instance, a bispecific T-cell engaging molecule may further comprise a domain or moiety that increases the elimination half-life of the molecule. The elimination half-life refers to the time it takes for the concentration of a drug in the plasma or the total amount in the body to be reduced by 50%. Thus, after one half-life, the concentration of the drug in the body will be half of the starting dose. For example, the bispecific T-cell engaging molecules may comprise a half-life extension moiety that provides a half-life for the molecule of greater than 24 hours, greater than 48 hours, greater than 72 hours, greater than 5 days, greater than 7 days, greater than 10 days, greater than 14 days, or greater than 21 days. Accordingly, bispecific T-cell engaging molecules suitable for use in the methods of the present disclosure may have a half-life of 2 days to 21 days, 3 days to 14 days, 5 days to 15 days, 3 days to 7 days, or 2 days to 5 days. Examples of half-life extension moieties that can be incorporated into the bispecific T-cell engaging molecules used in the methods of the present disclosure can include, but are not limited to, an immunoglobulin Fc domain, a domain derived from serum albumin (e.g., human serum albumin), or an albumin-binding domain (e.g., comprising human albumin binding peptides), peptides that bind to the neonatal Fc receptor (FcRn), and polyethylene glycol polymers. Non-limiting examples of domains derived from human serum albumin or variants thereof that can be incorporated into the bispecific T-cell engaging molecules are described, for example, in WO 2011/051489, WO 2012/059486, WO 2013/075066, WO 2013/135896, and WO 2014/072481, all of which are hereby incorporated by reference in their entireties. In some embodiments, the half-life extension moiety incorporated into the bispecific T-cell engaging molecules used in the methods of the present disclosure is an albumin-binding domain, such as a domain comprising an albumin-binding peptide or an antibody fragment (e.g., single domain antibodies or scFv domains) that binds to serum albumin. Non-limiting examples of albumin-binding domains that may be incorporated into the bispecific T-cell engaging molecules suitable for use in the methods of the present disclosure are described in, for example, WO 2013/128027, WO 2014/140358, and WO 2017/201488, all of which are hereby incorporated by reference in their entireties.

In certain embodiments, the bispecific T-cell engaging molecules used in the methods of the present disclosure comprise an immunoglobulin Fc domain. The immunoglobulin Fc domain may comprise one or more Fc monomers. Each “Fc monomer” typically comprises at least a CH2 domain and a CH3 domain from an immunoglobulin molecule. The Fc monomer may comprise the CH2 and CH3 domains from an IgG1, IgG2, IgG3, or IgG4 immunoglobulin. As a non-limiting example, the CH2 domain comprises amino acids 231 to 340 of an IgG1 immunoglobulin and the CH3 domain comprises amino acids 341 to 446 of an IgG1 immunoglobulin, where the amino acid numbering is according to the EU numbering system described in Edelman et al., Proc. Natl. Acad. USA, Vol. 63:78-85 (1969) and Kabat et al., Sequences of Proteins of Immunological Interest, 5th Ed. Public Health Service, National Institutes of Health Publication No. 91-3242, Bethesda, MD (1991). The boundaries of the CH2 and CH3 domains may vary slightly from one IgG isoform to another, but the CH2 and CH3 domains in IgG2, IgG3, and IgG4 can be ascertained by alignment with the CH2 and CH3 domains in IgG1.

In some embodiments, the Fc monomer may comprise an immunoglobulin hinge region or portion thereof. The immunoglobulin hinge region is typically the region defined by amino acids 216 to 231 (according to the EU numbering system) of IgG immunoglobulins. In certain embodiments, the Fc monomer comprises a hinge region from an IgG1 immunoglobulin or a portion thereof. In certain embodiments, the Fc monomer comprises, in amino to carboxyl order, an immunoglobulin hinge region, an immunoglobulin CH2 domain, and an immunoglobulin CH3 domain.

In certain embodiments, the bispecific T-cell engaging molecules comprise an Fc domain having one Fc monomer. In alternative embodiments, the bispecific T-cell engaging molecules comprise an Fc domain having two or more Fc monomers. For instance, in some embodiments, the bispecific T-cell engaging molecules used in the methods of the present disclosure comprise an Fc domain having two Fc monomers. The two Fc monomers can be present on separate polypeptide chains and associate to form a dimer, e.g., via non-covalent interactions and/or disulfide bonds (e.g., between cysteine residues in the hinge regions of Fc monomers). In other embodiments, the two Fc monomers are fused to each other via a peptide linker, such as, e.g., a linker sufficient in length to allow the Fc monomers to associate and form an intra-chain dimer. The fusion of two Fc monomers to form a single polypeptide chain is referred to herein as a single-chain Fc domain (scFc domain) and is described in more detail below.

The peptide linker, by which the Fc monomers are fused to each other to form a single-chain Fc domain, may comprise at least 25 amino acid residues (e.g., 25, 26, 27, 28, 29, 30 or more). For instance, in some embodiments, this peptide linker comprises at least 30 amino acid residues (e.g., 30, 31, 32, 33, 34, 35 or more). In some embodiments, the linker comprises up to 40 amino acid residues, such as, e.g., up to 35 amino acid residues, e.g., exactly 30 amino acid residues.

The Fc monomer may contain one or more amino acid substitutions relative to the native CH2 or CH3 immunoglobulin amino acid sequences, e.g., to modulate effector function, alter glycosylation, or enhance stability. For instance, in some embodiments, the glycosylation site in the CH2 domain at amino acid position 297 according to EU numbering is removed by substituting a different amino acid for the asparagine residue at this position. A N297G substitution is employed in some embodiments. Stability-enhancing mutations include the substitution of one or more amino acids in the CH2 and/or CH3 domains with cysteine residues to promote disulfide bond formation. In some embodiments, specific pairs of residues are substituted with cysteine such that they preferentially form a disulfide bond with each other, thus limiting or preventing disulfide bond scrambling. Example pairs include, but are not limited to, A287C and L306C, V259C and L306C, R292C and V302C, and V323C and 1332C, with the amino acid positions numbered according to the EU numbering system. In some embodiments, the Fc monomer(s) incorporated into the Fc domain of the bispecific T-cell engaging molecules comprises N297G, R292C, and V302C substitutions, with the amino acid positions numbered according to the EU numbering system.

In certain embodiments, the bispecific T-cell engaging molecules used in the methods of the present disclosure comprise an Fc domain, which is a single-chain Fc domain. Accordingly, in certain such embodiments, the Fc domain comprises two Fc monomers, each monomer comprising an immunoglobulin hinge region, an immunoglobulin CH2 domain, and an immunoglobulin CH3 domain, wherein the two Fc monomers are fused to each other via a peptide linker as described herein.

In certain embodiments, the bispecific T-cell engaging molecules used in the methods of the present disclosure comprise, in an amino to carboxyl order:
a first domain that binds to a target cancer cell antigen (e.g., a human cancer cell antigen) comprising a first immunoglobulin heavy chain variable region (VH1) and a first immunoglobulin light chain variable region (VL1);
a second domain that binds to CD3 (e.g., human CD3) comprising a second immunoglobulin heavy chain variable region (VH2) and a second immunoglobulin light chain variable region (VL2); and
an Fc domain comprising two Fc monomers.

In certain embodiments, the bispecific T-cell engaging molecules used in the methods of the present disclosure are single chain polypeptides or single chain fusion proteins. As used herein, a “single chain polypeptide” or “single chain fusion protein” refers to a molecule consisting of only one polypeptide chain, i.e., all of the domains in the bispecific T-cell engaging molecule are linked together, optionally via peptide linkers, to form a single polypeptide chain. One non-limiting example of such a single chain polypeptide or single chain fusion protein in the context of the present disclosure is a single chain polypeptide comprising, in an amino to carboxyl order, an anti-cancer cell antigen scFv domain, a first peptide linker, an anti-CD3 scFv domain, a second peptide linker, and an scFc domain.

In some embodiments, the subject to be treated according to the methods of the present disclosure is diagnosed with or has leukemia or lymphoma, such as diffuse large B-cell lymphoma, Burkitt lymphoma, follicular lymphoma, Non-Hodgkin lymphoma, or acute lymphoblastic leukemia, and the anti-cancer cell antigen binding domain of the bispecific T-cell engaging molecule binds to CD19.

In other embodiments, the subject to be treated according to the methods of the present disclosure is diagnosed with myeloid leukemia, such as, e.g., acute myeloid leukemia, and the anti-cancer cell antigen binding domain of the bispecific T-cell engaging molecule binds to CD33 or FLT3.

In other embodiments, the subject to be treated according to the methods of the present disclosure is diagnosed with or has a DLL3-expressing cancer, such as small-cell lung cancer, neuroendocrine prostate cancer, melanoma, or glioblastoma, and the anti-cancer cell antigen binding domain of the bispecific T-cell engaging molecule binds to DLL3.

In certain embodiments, the subject to be treated according to the methods of the present disclosure is diagnosed with or has a BCMA-positive cancer, and the anti-cancer cell antigen binding domain of the bispecific T-cell engaging molecule binds to BCMA. In some embodiments, the BCMA-positive cancer is multiple myeloma. The multiple myeloma may be refractory and/or relapsed multiple myeloma.

In certain other embodiments, the subject to be treated according to the methods of the present disclosure is diagnosed with or has a PSMA-expressing cancer, such as prostate cancer, non-small cell lung cancer, small cell lung cancer, renal cell carcinoma, hepatocellular carcinoma, bladder cancer, testicular cancer, colon cancer, glioblastoma, breast cancer, ovarian cancer, endometrial cancer, or melanoma, and the anti-cancer cell antigen binding domain of the bispecific T-cell engaging molecule binds to PSMA. In some embodiments, the PSMA-expressing cancer is prostate cancer. The prostate cancer may be castration-resistant prostate cancer (i.e., prostate cancer that is resistant to androgen deprivation therapy). In these and other embodiments, the prostate cancer can be metastatic prostate cancer, e.g., metastatic castration-resistant prostate cancer.

In some embodiments, the subject to be treated according to the methods of the present disclosure is diagnosed with a CLDN18.2-expressing cancer, such as colorectal cancer, pancreatic cancer, ovarian cancer, lung cancer, and gastrointestinal cancer, such as, e.g., gastric cancer, esophageal cancer, and gastroesophageal junction cancer, and the anti-cancer cell antigen binding domain of the bispecific T-cell engaging molecule binds to CLDN18.2.

In other embodiments, the subject to be treated according to the methods of the present disclosure is diagnosed with a MUC17-expressing cancer, such as colorectal cancer, pancreatic cancer, and gastrointestinal cancer, such as, e.g., gastric cancer and gastroesophageal junction cancer, and the anti-cancer cell antigen binding domain of the bispecific T-cell engaging molecule binds to MUC17.

Bispecific T-cell engaging molecules for use in the methods of the present disclosure may be prepared by any of a number of conventional techniques. For example, the bispecific T-cell engaging molecules described herein may be produced by recombinant expression systems, using any technique known in the art. See, e.g., Monoclonal Antibodies, Hybridomas: A New Dimension in Biological Analyses, Kennet et al. (eds.) Plenum Press, New York (1980); and Antibodies: A Laboratory Manual, Harlow and Lane (eds.), Cold Spring Harbor Laboratory Press, Cold Spring Harbor, N.Y. (1988).

Bispecific T-cell engaging molecules or components thereof (e.g., Fv fragments, Fc monomers) can be expressed in hybridoma cell lines or in cell lines other than hybridomas. Expression vectors or constructs encoding the bispecific T-cell engaging molecules can be used to transform a mammalian, insect, or microbial host cell. Examples of vectors include, but are not limited to, plasmids, viral vectors, non-episomal mammalian vectors, and expression vectors, for example, recombinant expression vectors. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site, and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired.

Recombinant expression vectors or constructs will typically comprise a nucleic acid molecule encoding a polypeptide comprising one or more of the following: one or more CDRs provided herein; a light chain constant region; a light chain variable region; a heavy chain constant region (e.g., CH1, CH2 and/or CH3); a heavy chain variable region; hinge region, Fc domain, and/or another scaffold portion of an antibody specifically binding to a cancer cell antigen or anti-CD3 antibody. These nucleic acid sequences are inserted into an appropriate expression vector using standard ligation techniques. In embodiments in which the bispecific T-cell engaging molecule is a single chain polypeptide or single chain fusion protein, the nucleic acid comprised in the recombinant expression vector will typically encode the full-length single chain polypeptide (e.g., full-length single chain fusion protein). The vector is typically selected to be functional in the particular host cell employed (i.e., the vector is compatible with the host cell machinery, permitting amplification and/or expression of the gene to occur). In some embodiments, vectors are used that employ protein-fragment complementation assays using protein reporters, such as dihydrofolate reductase (see, for example, U.S. Pat. No. 6,270,964, which is hereby incorporated by reference). Suitable expression vectors can be purchased, for example, from Invitrogen Life Technologies or BD Biosciences (formerly “Clontech”). Other useful vectors for cloning and expressing the antibody constructs and fragments include those described in Bianchi and McGrew, 2003, Biotech. Biotechnol. Bioeng. 84:439-44, which is hereby incorporated by reference. Additional suitable expression vectors are discussed, for example, in Methods Enzymol., vol. 185 (D. V. Goeddel, ed.), 1990, New York: Academic Press.

Typically, expression vectors used in any of the host cells to produce a bispecific T-cell engaging molecule will contain sequences for cloning and expression of exogenous nucleotide sequences encoding the bispecific T-cell engaging molecule or components thereof. Such sequences, collectively referred to as “flanking sequences,” in certain embodiments will include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Optionally, the vector may contain a “tag”-encoding sequence, i.e., an oligonucleotide molecule located at the 5′ or 3′ end of the bispecific T-cell engaging molecule coding sequence; the oligonucleotide sequence encodes polyHis (such as hexaHis), or another “tag” such as FLAG® tag, HA (hemagglutinin influenza virus), or myc, for which commercially available antibodies exist. This tag is typically fused to the polypeptide upon expression of the polypeptide and can serve as a means for affinity purification or detection of the bispecific T-cell engaging molecule from the host cell. Affinity purification can be accomplished, for example, by column chromatography using antibodies against the tag as an affinity matrix. Optionally, the tag can subsequently be removed from the purified T-cell engaging molecule by various means such as using certain peptidases for cleavage.

Expression and cloning vectors will typically contain a promoter that is recognized by the host cell and operably linked to the nucleic acid molecule encoding a bispecific T-cell engaging molecule. The term “operably linked” as used herein refers to the linkage of two or more nucleic acid sequences in such a manner that a nucleic acid molecule capable of directing the transcription of a given gene and/or the synthesis of a desired protein molecule is produced. For example, a control sequence in a vector that is “operably linked” to a protein coding sequence is ligated thereto so that expression of the protein coding sequence is achieved under conditions compatible with the transcriptional activity of the control sequences. More specifically, a promoter and/or enhancer sequence, including any combination of cis-acting transcriptional control elements, is operably linked to a coding sequence if it stimulates or modulates the transcription of the coding sequence in an appropriate host cell or other expression system. A large number of promoters, recognized by a variety of potential host cells, are well known to those of skill in the art. For example, suitable promoters for use with mammalian host cells include those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus, and Simian Virus 40 (SV40). A suitable promoter is operably linked to the polynucleotide encoding, e.g., a bispecific T-cell engaging molecule or component thereof, by removing the promoter from the source nucleic acid by restriction enzyme digestion and inserting the desired promoter sequence into the vector.

The expression vectors for recombinant production of the bispecific T-cell engaging molecules described herein may be constructed from a starting vector such as a commercially available vector. Such vectors may or may not contain all of the desired flanking sequences. Where one or more of the desired flanking sequences are not already present in the vector, they may be individually obtained and ligated into the vector. Methods used for obtaining each of the flanking sequences are well-known to one skilled in the art. The expression vectors can be introduced into host cells to thereby produce the bispecific T-cell engaging molecules encoded by the nucleic acids present in the vectors.

After the vector has been constructed and one or more nucleic acid molecules encoding the bispecific T-cell engaging molecule or component thereof has been inserted into the proper site(s) of the vector or vectors, the completed vector(s) may be inserted into a suitable host cell for amplification and/or polypeptide expression. The term includes the progeny of the parent cell, whether or not the progeny is identical in morphology or in genetic make-up to the original parent cell, so long as the gene of interest is present. A host cell that comprises an isolated polynucleotide or nucleic acid encoding a bispecific T-cell engaging molecule, which may be operably linked to at least one expression control sequence (e.g., promoter or enhancer), is a “recombinant host cell.”

The transformation of an expression vector for a polypeptide into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used.

A host cell, when cultured under appropriate conditions, synthesizes a bispecific T-cell engaging molecule that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule. Suitable host cells include, but are not limited to, prokaryotic cells (e.g., E. coli, B. subtilis), yeast cells (Saccharomyces cerevisiae, Pichia pastoris), and mammalian cells (e.g., Chinese hamster ovary (CHO), human embryonic kidney (HEK)). In some embodiments, CHO cells are employed as host cells for expressing the bispecific T-cell engaging molecules.

Host cells are transformed or transfected with the above-described expression vectors for production of the T-cell engaging molecules and are cultured in conventional nutrient media modified as appropriate for inducing promoters, selecting transformants, or amplifying the genes encoding the desired sequences. The host cells used to produce the antibody constructs may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) may be suitable for culturing the host cells. In addition, any of the media described in Ham et al., Meth. Enz. 58:44, 1979; Barnes et al., Anal. Biochem. 102:255, 1980; U.S. Pat. Nos. 4,767,704; 4,657,866; 4,927,762; 4,560,655; or 5,122,469; WO 90/03430; or WO 87/00195 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression and will be apparent to the ordinary skilled artisan.

Upon culturing the host cells, the T-cell engaging molecule can be produced intracellularly, in the periplasmic space, or directly secreted into the medium. If the T-cell engaging molecule is produced intracellularly, as a first step, the host cells are lysed (e.g., by mechanical shear, osmotic shock, or enzymatic methods) and the particulate debris (e.g., host cells and lysed fragments), is removed, for example, by centrifugation, microfiltration, or ultrafiltration. If the T-cell engaging molecule is secreted into the culture medium, the T-cell engaging molecule can be separated from host cells through centrifugation or microfiltration, and optionally, subsequently concentrated through ultrafiltration. The bispecific T-cell engaging molecules can be further purified or partially purified using, for example, one or more chromatography steps, such as affinity chromatography (e.g., protein A, protein L, or protein G affinity chromatography), cation exchange chromatography, anion exchange chromatography, hydroxyapatite chromatography, hydrophobic interaction chromatography, or mixed mode chromatography.

In some embodiments, the bispecific T-cell engaging molecule is administered in a pharmaceutical composition further comprising a buffer, a surfactant, and a stabilizing agent. In some embodiments, the pharmaceutical composition comprises a bispecific T-cell engaging molecule, a glutamate buffer, polysorbate 20 or polysorbate 80, and sucrose, at a pH in the range of 4.0 to 4.4. In some embodiments, the pharmaceutical compositions may be lyophilized and reconstituted prior to administration to a patient.

Non-limiting example pharmaceutical compositions comprising bispecific T-cell engaging molecules are described in WO 2018/141910, which is hereby incorporated by reference in its entirety. In certain embodiments, a pharmaceutical composition useful for the treatment of cancer according to the methods described herein may comprise 0.5 mg/mL to 2 mg/mL of a bispecific T-cell engaging molecule, 5 mM to 20 mM L-glutamic acid, 0.005% to 0.015% weight/volume (w/v) polysorbate (e.g., polysorbate 20 or polysorbate 80), and 7% to 12% (w/v) sucrose. In other embodiments, the pharmaceutical composition comprises 0.5 mg/mL to 1.5 mg/mL of a bispecific T-cell engaging molecule, 8 mM to 12 mM L-glutamic acid, 0.008% to 0.012% (w/v) polysorbate (e.g., polysorbate 20 or polysorbate 80), and 8% to 10% (w/v) sucrose. In some embodiments, the pH of these compositions is in the range of 4.0 to 4.4 (e.g., pH of 4.0, 4.1, 4.2, 4.3, or 4.4).

Alternatively, the T-cell redirecting therapy used in a method described herein may be a CAR-expressing T-cell. CAR-expressing T-cells employed in the methods of the present disclosure typically comprise a first domain that binds to a target cancer cell antigen, a transmembrane domain, and an intracellular signaling domain. In some embodiments, the intracellular signaling domain comprises a costimulatory domain and/or a primary signaling domain.

In some embodiments, the first domain binds to a target cancer cell antigen selected from CEA, CD19, CD33, CD70, EGFRVIII, EpCAM, FLT3, GPRC5D, DLL3, BCMA, PSMA, STEAP1, STEAP2, MUC16, MUC17, and CLDN18.2.

In some embodiments, the CAR-expressing T-cells comprise a transmembrane domain that comprises a transmembrane domain of a protein, such as, e.g., a protein selected from the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, and CD154. In some embodiments, the first domain (i.e., the antigen binding domain) is connected to the transmembrane domain by a hinge region.

In some embodiments, the costimulatory domain is a functional signaling domain from a protein, such as, e.g., a protein selected from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11^{b}, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that binds with CD83.

In some embodiments, the primary signaling domain comprises a functional signaling domain of CD3 zeta.

In some embodiments, the intracellular signaling domain comprises a functional signaling domain of 4-1BB and/or a functional signaling domain of CD3 zeta. In some embodiments, the intracellular signaling domain comprises a functional signaling domain of CD27 and/or a functional signaling domain of CD3 zeta. In some embodiments, the intracellular signaling domain comprises a functional signaling domain of CD28 and/or a functional signaling domain of CD3 zeta. In some embodiments, the intracellular signaling domain comprises a functional signaling domain of ICOS and/or a functional signaling domain of CD3 zeta.

In some embodiments, the CAR further comprises a leader sequence.

CAR-expressing T-cells employed in the methods of the present disclosure may be allogenic or autologous and may be prepared according to standard techniques of the art.

In certain embodiments, one or more premedications can be administered to the subject prior to the administration of a first and/or subsequent dose of a T-cell redirecting therapy (e.g., a bispecific T-cell engaging molecule (e.g., tarlatamab), a CAR-expressing T-cell) or a first and/or subsequent dose of a heavy-chain antibody disclosed herein. Depending on the type of premedication used and the route by which it is administered, the premedication may, e.g., be administered 30-120 or 30-60 minutes prior to start of administration of the T-cell redirecting therapy (e.g., bispecific T-cell engaging molecule (e.g., tarlatamab), CAR-expressing T-cell) and/or the heavy-chain antibody. The premedication may be administered, for example, to prevent or reduce severity of infusion-related reactions and/or to prevent or reduce severity of cytokine release syndrome or its symptoms.

In some embodiments in which premedication is administered, the premedication is an antihistamine. The antihistamine can be administered orally or intravenously and can be administered at a dose equivalent to diphenhydramine 50 mg i.v. Example antihistamines that can be administered as a premedication include, but are not limited to, antihistamines suited to oral, parenteral, or rectal routes such as: azatadine (maximum dose, e.g., 4 mg/day), brompheniramine (maximum dose, e.g., 30 mg/day), cetirizine (maximum dose, e.g., 15 mg/day), chlorpheniramine (maximum dose, e.g., 30 mg/day), clemastine (maximum dose, e.g., 10 mg/day), cyproheptadine (maximum dose, e.g., 15 mg/day), desloratadine (maximum dose, e.g., 7 mg/day), dexchlorpheniramine (maximum dose, e.g., 15 mg/day), diphenhydramine (maximum dose, e.g., 350 mg/per day), doxylamine (maximum dose, e.g., 180 mg/day), fexofenadine (maximum dose, e.g., 200 mg/day), loratadine (maximum dose, e.g., 15 mg/day), and phenindamine (maximum dose, e.g., 180 mg/day).

In other embodiments in which premedication is administered, the premedication is a glucocorticoid. Glucocorticoids, also known as glucocorticosteroid, are a class of corticosteroids, which are a class of steroid hormones. Glucocorticoids are corticosteroids that bind to the glucocorticoid receptor. Cortisol (known as hydrocortisone when used as a medication) is an important human glucocorticoid. A variety of synthetic glucocorticoids, some far more potent than cortisol, have been created for therapeutic use. Cortisol is a common standard of comparison for glucocorticoid potency. One example for commonly prescribed replacement steroid equivalents may be prednisone (5 mg)=cortisone (25 mg)=dexamethasone (0.75 mg)=hydrocortisone (20 mg)=methylprednisolone (4 mg). These doses indicate the equivalent pharmacologic dose of systemic glucocorticoids. The glucocorticoid can be administered orally or intravenously and can be administered at a dose equivalent to 4-20 mg dexamethasone i.v. (the equivalence referring to the glucocorticoid potency). The dose of glucocorticoid can be the same at each administration (i.e., at each time the glucocorticoid premedication is administered). Alternatively, the dose of glucocorticoid can be reduced in subsequent administrations, e.g., by 50% of the previous dose, if there are no or minimal signs of infusion reactions and/or cytokine release syndrome (CRS) symptoms following the previous administration of the bispecific T-cell engaging molecule. In certain embodiments, glucocorticoids are only administered as premedications during the initiation cycle and are not administered in subsequent treatment cycles (e.g., maintenance cycles).

Examples of glucocorticoids that can be used as a premedication include, but are not limited to, cortisone, hydrocortisone, prednisone, prednisolone, methylprednisolone, dexamethasone, betamethasone, beclomethasone, budesonide, triamcinolone, cloprednol, deflazacort, fluocortolone, cortivazol, paramethasone, fluticasone, fluticasone propionate, and triamcinolone acetonide, as well as combinations and/or pharmaceutically acceptable derivatives thereof. The different glucocorticoids may be used alone or in combination. In certain embodiments, the glucocorticoid administered to the subject prior to administration of one or more (or all) doses of the T-cell redirecting therapy (e.g., bispecific T-cell engaging molecule, CAR-expressing T-cell) during the initiation cycle and/or maintenance cycle is dexamethasone. Dexamethasone can be administered at a dose of 4-20 mg, 6-18 mg, 8-16 mg, 16 mg, or 8 mg at each administration.

In certain embodiments in which a premedication is administered, the premedication can be an IL-6 receptor antagonist, such as tocilizumab. Tocilizumab has been reported to effectively reduce or reverse symptoms of CRS induced by T cell-engaging therapies. See, e.g., Maude et al., Cancer J., Vol. 20:119-122, 2014. Tocilizumab can be administered at a dose of 1 mg/kg to 20 mg/kg body weight, 8 mg/kg to 12 mg/kg body weight, or 4 mg/kg to 8 mg/kg body weight. Tocilizumab can be administered 1 hour to 2 hours prior to each dose of the T-cell redirecting therapy (e.g., bispecific T-cell engaging molecule, CAR-expressing T-cell) in the initiation cycle and/or one or more maintenance cycles. Additionally or alternatively, tocilizumab can be administered immediately after each dose of the T-cell directing therapy (e.g., bispecific T-cell engaging molecule, CAR-expressing T-cell) in the initiation cycle and/or one or more maintenance cycles. Other antagonists of IL-6/IL-6 receptor signaling, such as siltuximab, olokizumab, clazakizumab, sarilumab, and sirukumab, can also be used as a premedication to reduce the potential occurrence or severity of CRS.

In certain other embodiments in which a premedication is administered, the premedication is a tumor necrosis factor alpha (TNF-alpha) antagonist. CRS symptoms have been previously reported to be mediated in part by release of TNF-alpha (Lee et al., Blood, Vol. 124:188-195, 2014; Grupp et al., N Engl J Med., Vol. 368:1509-1518, 2013). Recent studies have suggested that treatment with TNF-alpha antagonists prior to administration of immunotherapy agents may mitigate CRS symptoms (Li et al., Sci Transl Med., Vol. 11 (508), 2019; Lee et al., 2014, supra; Grupp et al., 2013, supra). Accordingly, in certain embodiments, methods disclosed herein further comprise administering to the patient a TNF-alpha antagonist prior to administration of each dose of the T-cell redirecting therapy (e.g., bispecific T-cell engaging molecule, CAR-expressing T-cell) during the initiation cycle and/or one or more maintenance cycles. Examples of TNF-alpha antagonists that can be used as a premedication include, but are not limited to, etanercept, infliximab, adalimumab, certolizumab pegol, and golimumab.

Effective doses of the therapeutic agents described herein can vary depending upon many different factors, including means of administration, target site, physiological state of the patient, whether the patient is human or an animal, other medications administered, and whether treatment is prophylactic or therapeutic. Treatment dosages can be titrated to optimize safety and efficacy.

In some cases, the therapeutic doses of bispecific T-cell engaging molecules administered according to the methods of the present disclosure may range from 50 μg to 200 mg or from 200 μg to 80 mg depending on the specific bispecific T-cell engaging molecule employed and the type, grade, or stage of cancer to be treated in the patient. Non-limiting example ranges of therapeutic doses of a bispecific T-cell engaging molecule for the treatment of cancer may include, but are not limited to, doses of 50 μg to 200 mg, from 200 μg to 80 mg, from 90 μg to 30 mg, from 300 μg to 15 mg, from 150 μg to 2 mg, from 6 mg to 25 mg, from 1 mg to 20 mg, from 10 mg to 100 mg, or from 50 mg to 150 mg.

When the T-cell redirecting therapy is a CAR-expressing T-cell, a dose of CAR-expressing T-cells cells may comprise 10⁴ cells/kg to 10⁹ cells/kg, such as, e.g., 10⁴ cells/kg to 10⁵ cells/kg, 10⁵ cells/kg to 10⁶ cells/kg, 10⁶ cells/kg to 10⁷ cells/kg, 10⁷ cells/kg to 10⁸ cells/kg, or 10⁸ cells/kg to 10⁹ cells/kg; or at least one of: 1×10⁷ cells, 1.5×10⁷ cells, 2×10⁷ cells, 2.5×10⁷ cells, 3×10⁷ cells, 3.5×10⁷ cells, 4×10⁷ cells, 5×10⁷ cells, 1×10⁸ cells, 1.5×10⁸ cells, 2×10⁸ cells, 2.5×10⁸ cells, 3×10⁸ cells, 3.5×10⁸ cells, 4×10⁸ cells, 5×10⁸ cells, 1×10⁹ cells, 2×10⁹ cells, or 5×10⁹ cells. In some embodiments, a dose of CAR-expressing T-cells comprises at least 1-5×10⁷ cells to 1-5×10⁸ cells.

In some embodiments, the CAR-expressing T-cells are administered to the subject according to a dosing regimen comprising a total dose of cells administered to the subject by dose fractionation, e.g., one, two, three, or more separate administration of a partial dose. In some embodiments, a first percentage of the total dose is administered on a first treatment day, a second percentage of the total dose is administered on a subsequent (e.g., second, third, fourth, fifth, sixth, or seventh or later) treatment day, and optionally, a third percentage (e.g., the remaining percentage) of the total dose is administered on a yet subsequent (e.g., third, fourth, fifth, sixth, seventh, eighth, ninth, tenth, or later) treatment day. In some embodiments, the CAR-expressing T-cell is administered at a dose of 1-10×10⁸ cells per infusion, such as, e.g., 5×10⁸ cells per infusion.

Therapeutic agents of the present disclosure may be administered on multiple occasions, sequentially, or concurrently. Intervals between single dosages can be weekly, monthly, or yearly. Intervals can also be irregular as indicated by measuring blood levels of the therapeutic entity in the patient. Alternatively, therapeutic agents of the present disclosure can be administered as a sustained release formulation, in which case less frequent administration may be required. Dosage and frequency vary depending on the half-life of the therapeutic agent in the subject.

In some embodiments, the methods of the present disclosure comprise administering a T-cell redirecting therapy (e.g., a bispecific T-cell engaging molecule, a CAR-expressing T-cell) and/or a heavy-chain antibody disclosed herein to the patient in at least one initiation cycle. As used herein, an “initiation cycle” is a treatment cycle in which the therapeutic agent is administered at two or more different doses at a dosing frequency and mode of administration designed to minimize adverse events, for example, such as adverse events associated with CRS, while enabling exposure of the patient to a therapeutically effective dose of the therapeutic agent in the shortest time possible. An initiation cycle is preferably administered to a patient as the first treatment cycle when the patient begins a course of treatment with the therapeutic agent. An initiation cycle may also be administered to a patient when the patient re-starts a course of treatment with the therapeutic agent, for example, following a treatment-free period, dosing interruption (e.g., when a patient did not complete a previous treatment cycle), or a relapse or progression of a cancer in the patient. Although administration of one initiation cycle will typically be sufficient, in some embodiments of the methods of the present disclosure, administration of two or more initiation cycles is contemplated. In one particular embodiment, only one initiation cycle is administered to the patient.

Generally, the methods of the present disclosure comprise administering a T-cell redirecting therapy (e.g., a T-cell engaging molecule, a CAR-expressing T-cell) and a heavy-chain antibody disclosed herein to the subject in one or more treatment cycles. A “treatment cycle” or “cycle” refers to a period of administration of one or more therapeutic agent(s) at specific dosages and dosing intervals. According to the methods of the present disclosure, a subject can receive multiple treatment cycles (e.g., 2, 3, 4, 5, 6, 7, 8, 9, 10, 11, 12, 13, 14 or more cycles). The treatment cycles can be administered to the patient consecutively with no break or period without administration of one or more therapeutic agent(s) between the cycles. Alternatively, a period without administration of one or more therapeutic agent(s) (e.g., a “treatment-free period” or “break”) can be employed between the treatment cycles. The length of the treatment-free period can be adjusted based on the patient's characteristics and/or response to treatment. For example, the patient may receive treatment cycles of one or more therapeutic agent(s) until the patient achieves a desired level of response, such as a complete response or partial response.

Additionally, a patient may be treated according to the methods of the present disclosure for a set treatment period. A “treatment period” begins upon administration of a first dose of a therapeutic agent in an initiation cycle and ends upon administration of a final dose of a therapeutic agent in a maintenance cycle. The treatment period may be from 3 months to 36 months, from 12 months to 24 months, or from 6 months to 12 months. For instance, the treatment period may be 3 months, 4 months, 5 months, 6 months, 7 months, 8 months, 9 months, 10 months, 11 months, 12 months, 13 months, 14 months, 15 months, 18 months, 21 months, 24 months, 27 months, 30 months, 33 months, or 36 months. In some embodiments, the treatment period is 6 months. In other embodiments, the treatment period is 9 months. In yet other embodiments, the treatment period is 12 months. The treatment period can be adjusted for each patient depending on the patient's response to treatment. In one particular embodiment, the patient is treated according to the methods of the present disclosure until the patient achieves a complete response or until evidence of the particular cancer is otherwise undetectable in the patient.

Also provided herein is a heavy-chain antibody disclosed herein for use in the treatment of cancer in combination with a T-cell redirecting therapy. Further provided herein is a T-cell redirecting therapy for use in the treatment of cancer in combination with a heavy-chain antibody disclosed herein. In some embodiments, the T-cell redirecting therapy is a T-cell engaging molecule. In some embodiments, the T-cell redirecting therapy is a bispecific T-cell engaging molecule (e.g., tarlatamab).

For example, in some embodiments, provided herein is a heavy-chain antibody disclosed herein for use in the treatment of cancer (e.g., a DLL3-expressing cancer; small cell lung cancer or neuroendocrine prostate cancer) in combination with tarlatamab.

In some embodiments, the heavy-chain antibody comprises a first heavy chain that binds to IL2Rβ comprising the amino acid sequence of SEQ ID NO: 38 and a second heavy chain that binds to IL2RG comprising the amino acid sequence of SEQ ID NO: 39.

Also provided herein is a heavy-chain antibody disclosed herein for use in enhancing an anti-cancer effect associated with administration of a T-cell redirecting therapy in a subject diagnosed with cancer. Further provided herein is a T-cell redirecting therapy for use in enhancing an anti-cancer effect associated with administration of a heavy-chain antibody disclosed herein in a subject diagnosed with cancer. In some embodiments, the T-cell redirecting therapy is a T-cell engaging molecule. In some embodiments, the T-cell redirecting therapy is a bispecific T-cell engaging molecule (e.g., tarlatamab).

Illustratively, in some embodiments, provided herein is a heavy-chain antibody disclosed herein for use in enhancing an anti-cancer effect associated with administration of tarlatamab in a subject diagnosed with cancer (e.g., a DLL3-expressing cancer; small cell lung cancer or neuroendocrine prostate cancer).

In some embodiments, the heavy-chain antibody comprises a first heavy chain that binds to IL2Rβ comprising the amino acid sequence of SEQ ID NO: 38 and a second heavy chain that binds to IL2RG comprising the amino acid sequence of SEQ ID NO: 39.

Also provided herein is a use of a heavy-chain antibody disclosed herein in the manufacture of a medicament adapted for use in a method described herein. In some embodiments, the method possesses one or more of the features of a method described herein. In some embodiments, the heavy-chain antibody comprises a first heavy chain that binds to IL2Rβ comprising the amino acid sequence of SEQ ID NO: 38 and a second heavy chain that binds to IL2RG comprising the amino acid sequence of SEQ ID NO: 39.

Also provided herein is a use of a T-cell redirecting therapy in the manufacture of a medicament adapted for use in a method described herein. In some embodiments, the method possesses one or more of the features of a method described herein. In some embodiments, the T-cell redirecting therapy is a T-cell engaging molecule. In some embodiments, the T-cell redirecting therapy is a bispecific T-cell engaging molecule (e.g., tarlatamab).

Also provided herein is a use of a heavy-chain antibody disclosed herein and a T-cell redirecting therapy in the manufacture of a medicament adapted for use in a method described herein. In some embodiments, the method possesses one or more of the features of a method described herein. In some embodiments, the T-cell redirecting therapy is a T-cell engaging molecule. In some embodiments, the T-cell redirecting therapy is a bispecific T-cell engaging molecule (e.g., tarlatamab). In some embodiments, the heavy-chain antibody comprises a first heavy chain that binds to IL2Rβ comprising the amino acid sequence of SEQ ID NO: 38 and a second heavy chain that binds to IL2RG comprising the amino acid sequence of SEQ ID NO: 39.

Also provided herein is a pharmaceutical composition comprising a heavy-chain antibody disclosed herein for use in a method described herein. In some embodiments, the method possesses one or more of the features of a method described above. In some embodiments, the heavy-chain antibody comprises a first heavy chain that binds to IL2Rβ comprising the amino acid sequence of SEQ ID NO: 38 and a second heavy chain that binds to IL2RG comprising the amino acid sequence of SEQ ID NO: 39.

Also provided herein is a pharmaceutical composition comprising a T-cell redirecting therapy for use in a method described herein. In some embodiments, the method possesses one or more of the features of a method described above. In some embodiments, the T-cell redirecting therapy is a T-cell engaging molecule. In some embodiments, the T-cell redirecting therapy is a bispecific T-cell engaging molecule (e.g., tarlatamab).

Also provided herein is a pharmaceutical composition comprising a heavy-chain antibody disclosed herein and a T-cell redirecting therapy for use in a method disclosed herein. In some embodiments, the method possesses one or more of the features of a method described above. In some embodiments, the T-cell redirecting therapy is a T-cell engaging molecule. In some embodiments, the T-cell redirecting therapy is a bispecific T-cell engaging molecule (e.g., tarlatamab). In some embodiments, the heavy-chain antibody comprises a first heavy chain that binds to IL2Rβ comprising the amino acid sequence of SEQ ID NO: 38 and a second heavy chain that binds to IL2RG comprising the amino acid sequence of SEQ ID NO: 39.

Also provided herein is a combination product comprising a heavy-chain antibody disclosed herein and a T-cell redirecting therapy. In some embodiments, the combination product is adapted for use in a method described herein. In some embodiments, the method possesses one or more of the features of a method described above. In some embodiments, the T-cell redirecting therapy is a T-cell engaging molecule. In some embodiments, the T-cell redirecting therapy is a bispecific T-cell engaging molecule (e.g., tarlatamab).

For example, in some embodiments, provided herein is a combination product comprising a heavy-chain antibody disclosed herein and tarlatamab. In some embodiments, the combination product is adapted for use in a method described herein. In some embodiments, the method possesses one or more of the features of a method described above.

In some embodiments, the heavy-chain antibody comprises a first heavy chain that binds to IL2Rβ comprising the amino acid sequence of SEQ ID NO: 38 and a second heavy chain that binds to IL2RG comprising the amino acid sequence of SEQ ID NO: 39.

Also provided herein is a combination product for use in a method of treating cancer in a subject in need thereof, comprising a heavy-chain antibody disclosed herein and a T-cell redirecting therapy, wherein the heavy-chain antibody enhances an anti-cancer effect associated with administration of the T-cell redirecting therapy. In some embodiments, the T-cell redirecting therapy is a T-cell engaging molecule. In some embodiments, the T-cell redirecting therapy is a bispecific T-cell engaging molecule (e.g., tarlatamab).

Illustratively, in some embodiments, provided herein is a combination product for use in a method of treating cancer (e.g., a DLL3-expressing cancer; small cell lung cancer or neuroendocrine prostate cancer) in a subject in need thereof, comprising a heavy-chain antibody disclosed herein and tarlatamab, wherein the heavy-chain antibody enhances an anti-cancer effect associated with administration of tarlatamab.

In some embodiments, the heavy-chain antibody comprises a first heavy chain that binds to IL2Rβ comprising the amino acid sequence of SEQ ID NO: 38 and a second heavy chain that binds to IL2RG comprising the amino acid sequence of SEQ ID NO: 39.

In alternative aspects of the present disclosure, an NK-cell redirecting therapy (i.e., a therapeutic agent capable of recruiting T-cells to a target cell or tissue), such as an NK cell engaging molecule or a CAR-NK cell, may be used in place of a T-cell redirecting therapy in a method, use, pharmaceutical composition, or combination product described herein.

The present disclosure further provides methods for combination therapies in which an IL-2-based therapy is used in combination with a T-cell redirecting therapy that binds to DLL3 (i.e., an anti-DLL3 T-cell redirecting molecule) for the treatment of a DLL3-expressing cancer. In one aspect, such combination therapy provides a synergistic or additive therapeutic effect for the treatment of the DLL3-expressing cancer.

In some embodiments, the IL-2-based therapy is a recombinant IL-2 polypeptide (e.g., recombinant human IL-2, e.g., aldesleukin). In some embodiments, the IL-2-based therapy is a mutant IL-2 polypeptide. “IL-2 mutant” or “mutant IL-2 polypeptide,” as used herein, refers to a mutant form of an IL-2 molecule, including full-length IL-2, truncated forms of IL-2, and forms in which IL-2 is linked to another molecule, for example, by fusion or chemical conjugation. One non-limiting example of an IL-2 mutant is bempegaldesleukin, a pegylated recombinant form of IL-2.

In certain embodiments, the IL-2-based therapy preferentially binds to the heterodimeric receptor composed only of IL-2Rβ and IL-2Rγ over the trimeric IL-2Rαβγ form of the IL-2 receptor. Such an IL-2-based therapy may be referred to as a “non-α IL-2” or a “not-α IL-2” therapy herein. Non-α IL-2 therapies include, but are not limited to, pegylated forms of IL-2 and IL-2 mutants, such as bempegaldesleukin and THOR707 (SAR444245), as well as heavy-chain antibodies described herein. In addition, non-α IL-2 therapies may include one or more binding domains or masking motifs intended to target the IL-2-based therapy to a particular tumor or cell type.

The IL-2-based therapy can be administered simultaneously, separately, or sequentially with an effective amount of the anti-DLL3 T-cell redirecting therapy (e.g., an anti-DLL3 T-cell engaging molecule (e.g., tarlatamab)). In some cases, the IL-2-based therapy is administered in the form of a pharmaceutical composition. In some cases, the anti-DLL3 T-cell redirecting therapy (e.g., tarlatamab) is administered in the form of a pharmaceutical composition.

In some embodiments, an anti-DLL3 T-cell engaging molecule used in the methods described herein comprises:
a heavy chain variable (VH) region comprising a VH CDR1, a VH CDR2, and a VH CDR3, wherein the VH CDR1, VH CDR2, and VH CDR3 comprise the amino acid sequences of SEQ ID NOs: 62, 63, and 64, respectively; and
a light chain variable (VL) region comprising a VL CDR1, a VL CDR2, and a VL CDR3, wherein the VL CDR1, VL CDR2, and VL CDR3 comprise the amino acid sequences of SEQ ID NOs: 65, 66, and 67, respectively.

In some embodiments, an anti-DLL3 T-cell engaging molecule used in the methods described herein comprises a VH region comprising the amino acid sequence of SEQ ID NO: 68 and a VL region comprising the amino acid sequence of SEQ ID NO: 69.

In some embodiments, an anti-DLL3 T-cell engaging molecule used in the methods described herein comprises the amino acid sequence of SEQ ID NO: 70.

In some embodiments, an anti-DLL3 T-cell engaging molecule used in the methods described comprises a first domain that binds to DLL3 and a second domain that binds to CD3, wherein:
the first domain comprises a first heavy chain variable (VH) region comprising a VH CDR1, a VH CDR2, and a VH CDR3, wherein the VH CDR1, VH CDR2, and VH CDR3 comprise the amino acid sequences of SEQ ID NOs: 62, 63, and 64, respectively; and a first light chain variable (VL) region comprising a VL CDR1, a VL CDR2, and a VL CDR3, wherein the VL CDR1, VL CDR2, and VL CDR3 comprise the amino acid sequences of SEQ ID NOs: 65, 66, and 67, respectively; and
the second domain comprises a second VH region comprising a VH CDR1, a VH CDR2, and a VH CDR3, wherein the VH CDR1, VH CDR2, and VH CDR3 comprise the amino acid sequences of SEQ ID NOs: 71, 72, and 73, respectively; and a second VL region comprising a VL CDR1, a VL CDR2, and a VL CDR3, wherein the VL CDR1, VL CDR2, and VL CDR3 comprise the amino acid sequences of SEQ ID NOs: 74, 75, and 76, respectively.

In some embodiments, an anti-DLL3 T-cell engaging molecule used in the methods described comprises a first domain that binds to DLL3 and a second domain that binds to CD3, wherein:
the first domain comprises a first heavy chain variable (VH) region comprising the amino acid sequence of SEQ ID NO: 68 and a first light chain variable (VL) region comprising the amino acid sequence of SEQ ID NO: 69; and
the second domain comprises a second VH region comprising the amino acid sequence of SEQ ID NO: 77 and a second VL region comprising the amino acid sequence of SEQ ID NO: 78.

In some embodiments, an anti-DLL3 T-cell engaging molecule used in the methods described comprises a first domain that binds to DLL3 and a second domain that binds to CD3, wherein the first domain comprises the amino acid sequence of SEQ ID NO: 70 and the second domain comprises the amino acid sequence of SEQ ID NO: 79.

In some embodiments, an anti-DLL3 T-cell engaging molecule used in the methods described herein binds to DLL3 and CD3 and comprises the amino acid sequence of SEQ ID NO: 80 or SEQ ID NO: 81. In some embodiments, the anti-DLL3 T-cell engaging molecule comprises the amino acid sequence of SEQ ID NO: 80. In some embodiments, the anti-DLL3 T-cell engaging molecule comprises the amino acid sequence of SEQ ID NO: 81.

In some embodiments, the anti-DLL3 T-cell engaging molecule comprises a variant of the amino acid sequence of SEQ ID NO: 81 in which the C-terminal lysine of SEQ ID NO: 81 is clipped.

In some embodiments, an anti-DLL3 T-cell engaging molecule used in the methods described herein binds to DLL3 and CD3 and consists of the amino acid sequence of SEQ ID NO: 80 or SEQ ID NO: 81. In some embodiments, the anti-DLL3 T-cell engaging molecule consists of the amino acid sequence of SEQ ID NO: 80. In some embodiments, the anti-DLL3 T-cell engaging molecule consists of the amino acid sequence of SEQ ID NO: 81.

In some embodiments, an anti-DLL3 T-cell engaging molecule consists of a variant of the amino acid sequence of SEQ ID NO: 81 in which the C-terminal lysine of SEQ ID NO: 81 is clipped.

In some embodiments, an anti-DLL3 T-cell engaging molecule used in the methods described herein is tarlatamab.

Bispecific T-cell engaging molecules that specifically bind to DLL3 are disclosed, for example, in International Patent Publication Nos. WO2017/021349 and WO2023/215829, which are incorporated by reference herein in their entireties. For example, a bispecific T-cell engaging molecule used in the methods described herein can include one or more amino acid sequences set forth in TABLE 9, TABLE 10, or TABLE 11A. The specific CDRs identified in TABLE 9 and TABLE 10 are defined by Kabat.

In other embodiments, an anti-DLL3 T-cell engaging molecule used in the methods described herein is a single chain polypeptide comprising one or more scFvs fused to one or more single domain monoclonal antibodies (sdmAb). Optionally, the scFv-sdmAb format may comprise a domain that serves to extend the half-life of the protein. For example, the anti-DLL3 T-cell engaging molecule may comprise, from N to C terminus, an scFv that binds CD3, a linker peptide, a sdmAb that binds serum albumin, a peptide linker, and a sdmAb that binds DLL3, such as, for example, a polypeptide comprising the amino acid sequence of SEQ ID NO: 83. An alternate format for the anti-DLL3 T-cell engaging molecule comprises, from N- to C-terminus, a sdmAb that binds DLL3, a peptide linker, a sdmAb that binds serum albumin, a peptide linker, and an scFv that binds CD3. A non-limiting example of such an anti-DLL3 T-cell engaging molecule comprises the amino acid sequence of SEQ ID NO: 84. Anti-DLL3 T-cell engaging molecules comprising these formats are further described in, e.g., International Patent Publication No. WO 2020/069028, which is incorporated by reference with respect to its disclosure relating to anti-DLL3 T-cell engaging molecules and their associated amino acid sequences. For example, an anti-DLL3 T-cell engaging molecule used in the methods described herein can include one or more amino acid sequences set forth in TABLE 11B.

In other embodiments, an anti-DLL3 T-cell engaging molecule used in the methods described herein takes the form of a traditional bispecific IgG-like antibody, wherein one arm binds DLL3 and the second arm binds CD3. In some embodiments, such an antibody construct comprises two arms comprising, from N- to C-terminus, a VL domain, a CL domain, a peptide linker, a VH domain, a CHIdomain, a peptide linker, and an Fc domain (scFab-Fc). In some embodiments, the anti-DLL3 T-cell engaging molecule comprises a first domain binds to DLL3 (e.g., human DLL3) and comprises (a) a heavy chain variable region (VH) that comprises (i) a VH complementarity determining region one (CDR-H1) comprising the amino acid sequence of SEQ ID NO: 85, (ii) a CDR-H2 comprising the amino acid sequence of SEQ ID NO: 86, and (iii) a CDR-H3 comprising the amino acid sequence of SEQ ID NO: 87; and (b) a light chain variable region (VL) that comprises (i) a VL complementarity determining region one (CDR-L1) comprising the amino acid sequence of SEQ ID NO: 88, (ii) a CDR-L2 comprising the amino acid sequence of SEQ ID NO: 89, and (iii) a CDR-L3 comprising the amino acid sequence of SEQ ID NO: 90. Optionally, the first domain binds DLL3 and comprises a VH region comprising the amino acid sequence of SEQ ID NO: 91 and a VL region comprising the amino acid sequence of SEQ ID NO: 92. In some embodiments, the DLL3 binding region comprises SEQ ID NO: 93. The second domain binds to CD3 (e.g., human CD3) and comprises (a) a VH that comprises (i) a CDR-H1 comprising the amino acid sequence of SEQ ID NO: 95, (ii) a CDR-H2 comprising the amino acid sequence of SEQ ID NO: 96, and (iii) a CDR-H3 comprising the amino acid sequence of SEQ ID NO: 97; and (b) a VL that comprises (i) a CDR-L1 comprising the amino acid sequence of SEQ ID NO: 98, (ii) a CDR-L2 comprising the amino acid sequence of SEQ ID NO: 99, and (iii) a CDR-L3 comprising the amino acid sequence of SEQ ID NO: 100. Optionally, the second domain binds CD3 and comprises a VH region comprising the amino acid sequence of SEQ ID NO: 101 and a VL region comprising the amino acid sequence of SEQ ID NO: 102. In some embodiments, the CD3 binding region of the anti-DLL3 T-cell engaging molecule comprises SEQ ID NO: 103. In some embodiments, the anti-DLL3 T-cell engaging molecule optionally comprises the amino acid sequence of SEQ ID NO: 94 and the amino acid sequence of SEQ ID NO: 104. Anti-DLL3 T-cell engaging molecules are further described in, e.g., International Patent Publication No. WO 2019/234220, which is incorporated by reference with respect to its disclosure relating to anti-DLL3 T-cell engaging molecules and their associated amino acid sequences. Illustratively, an anti-DLL3 T-cell engaging molecule used in the methods described herein can include one or more amino acid sequences set forth in TABLE 11C.

In some embodiments, the DLL3-expressing cancer is a neuroendocrine cancer (i.e., neuroendocrine neoplasm). In this regard, the DLL3-expressing cancer may be a pulmonary neuroendocrine cancer or an extrapulmonary neuroendocrine cancer. In some embodiments, a subject to be treated with the combination therapy is suffering from a pulmonary neuroendocrine cancer, such as small cell lung cancer (SCLC), examples of which include relapsed/refractory SCLC (RR SCLC), extensive stage SCLC (ES SCLC) (which may be relapsed or refractory), and limited-stage SCLC (LS-SCLC) (which may be relapsed or refractory). LS-SCLC often refers to SCLC cancers found only on one side of the chest (e.g., only in one lung), enabling treatment with a single radiation field. In some embodiments, LS-SCLC has not progressed in the subject following concurrent chemoradiation therapy. ES SCLC includes cancers that have spread more widely throughout the lung and/or to the other lung, and often to other locations within the body. Relapsed SCLC include cancers which, e.g., reemerge within three months of a first line of treatment (e.g., chemotherapy and/or radiation).

In certain embodiments, the DLL3-expressing cancer is an extrapulmonary neuroendocrine cancer, such as glioma, glioblastoma, melanoma, prostate cancer (e.g., neuroendocrine prostate cancer), neuroendocrine pancreatic cancer, hepatoblastoma, large cell pulmonary neuroendocrine cancer, pancreatic neuroendocrine cancer, bladder neuroendocrine cancer, gastric neuroendocrine cancer, adrenal exocrine tumors, Merkel cell carcinoma, neuroblastoma, head and neck carcinoid or neuroendocrine cancer, head and neck paraganglioma, or cervical small cell neuroendocrine cancer.

In some embodiments, the extrapulmonary neuroendocrine cancer is neuroendocrine pancreatic cancer (NEPC).

In some embodiments, the subject to be treated with the combination therapy suffers from a DLL3-expressing cancer (e.g., SCLC) and has not received any prior line of cancer treatment. By “prior line of treatment” is meant a previous treatment with another anticancer therapeutic, e.g., a first- or second-line cancer therapy or standard of care therapy, before administration of the combination therapy. In other embodiments, the subject suffers from a DLL3-expressing cancer (e.g., SCLC) which has not progressed or relapsed after one or more prior lines of treatment. For example, in some embodiments, the subject has received a first-line cancer therapy, after which the subject's disease has not progressed (defined as ongoing response or stable disease per Response Evaluation Criteria in Solid Tumors Version 1.1 (RECIST 1.1)). In other embodiments, the subject suffers from a DLL3-expressing cancer (e.g., SCLC) which has recurred after one or more prior lines of treatment in the subject, such as a SCLC recurrence after two or more prior lines of treatment in the subject. Illustratively, in some embodiments, the subject has received at least one, two, three, four, five, or six prior lines of treatment for the DLL3-expressing cancer (e.g., SCLC). In some embodiments, at least one of the prior lines of treatment is a platinum-based chemotherapeutic. Alternatively or in addition, at least one of the prior lines of treatment is anti-PD1 antibody or anti-PD-L1 antibody therapy. For instance, the subject may have undergone a combination of therapies including platinum-based chemotherapy and anti-PD1 antibody or anti-PD-L1 antibody therapy. Where the subject has undergone prior lines of treatment, administration of the combination therapy may occur at any timepoint following completion of the prior lines of treatment. For instance, the combination therapy may be administered at least 28 days (e.g., at least 60 days or at least 90 days) after the completion of any prior lines of treatment for the DLL3-expressing cancer (e.g., SCLC).

Also provided herein is an IL-2-based therapy for use in the treatment of a DLL3-expressing cancer in combination with an anti-DLL3 T-cell redirecting therapy. Further provided herein is an anti-DLL3 T-cell redirecting therapy for use in the treatment of a DLL3-expressing cancer in combination with an IL-2-based therapy. In some embodiments, the anti-DLL3 T-cell redirecting therapy is an anti-DLL3 T-cell engaging molecule. In some embodiments, the anti-DLL3 T-cell redirecting therapy is a bispecific anti-DLL3 T-cell engaging molecule (e.g., tarlatamab).

Also provided herein is an IL-2-based therapy for use in enhancing an anti-cancer effect associated with administration of an anti-DLL3 T-cell redirecting therapy in a subject diagnosed with a DLL3-expressing cancer. Further provided herein is an anti-DLL3 T-cell redirecting therapy for use in enhancing an anti-cancer effect associated with administration of an IL-2-based therapy in a subject diagnosed with a DLL3-expressing cancer. In some embodiments, the anti-DLL3 T-cell redirecting therapy is an anti-DLL3 T-cell engaging molecule. In some embodiments, the T-cell redirecting therapy is a bispecific anti-DLL3 T-cell engaging molecule (e.g., tarlatamab).

Also provided herein is a use of an IL-2-based therapy in the manufacture of a medicament adapted for use in a method of treating a DLL3-expressing cancer in combination with an anti-DLL3 T-cell redirecting therapy. Also provided herein is a use of an anti-DLL3 T-cell redirecting therapy in the manufacture of a medicament adapted for use in a method of treating a DLL3-expressing cancer in combination with an IL-2-based therapy. In some embodiments, the anti-DLL3 T-cell redirecting therapy is an anti-DLL3 T-cell engaging molecule. In some embodiments, the T-cell redirecting therapy is a bispecific anti-DLL3 T-cell engaging molecule (e.g., tarlatamab).

Also provided herein is a use of an IL-2-based therapy and an anti-DLL3 T-cell redirecting therapy in the manufacture of a medicament adapted for use in a method of treating a DLL3-expressing cancer.

Also provided herein is a pharmaceutical composition comprising an IL-2-based therapy for use in a method of treating a DLL3-expressing cancer in combination with an anti-DLL3 T-cell redirecting therapy. Also provided herein is a pharmaceutical composition comprising an anti-DLL3 T-cell redirecting therapy for use in a method of treating a DLL3-expressing cancer in combination with an IL-2-based therapy.

### Kits

Also provided herein are kits for practicing the disclosed methods. Such kits can comprise a pharmaceutical composition, such as those described herein, which can be provided in a sterile container. Optionally, instructions on how to employ the provided pharmaceutical composition in therapy can also be included or be made available to a patient or a medical service provider.

In one aspect, a kit comprises (a) a pharmaceutical composition disclosed herein; and (b) one or more containers for the pharmaceutical composition. Such a kit can also comprise instructions for the use thereof; the instructions can be tailored to the patient population being treated. In some cases, the instructions can describe the use and nature of the materials provided in the kit. For example, in some embodiments, the kit comprises instructions for a patient to carry out administration.

The optional instructions can be printed on a substrate, such as paper or plastic, etc., and can be present in a kit as a package insert, in the labeling of the container of the kit or components thereof (e.g., associated with the packaging), etc. In some embodiments, the instructions are present as an electronic storage data file present on a suitable computer readable storage medium, such as, e.g., a CD-ROM, diskette, etc. In other embodiments, the actual instructions are not present in the kit, but means for obtaining the instructions from a remote source, such as over the internet, are provided. Illustratively, in some embodiments, the kit includes a web address where the instructions can be viewed and/or from which the instructions can be downloaded.

Often it will be desirable that some or all components of a kit are packaged in suitable packaging to maintain sterility. In some embodiments, the components of a kit can be packaged in a kit containment element to make a single, easily handled unit, where the kit containment element, e.g., box or analogous structure, may or may not be an airtight container, e.g., to further preserve the sterility of some or all of the components of the kit.

Illustratively, provided herein are kits comprising a pharmaceutical composition disclosed herein and instructions for using the pharmaceutical composition to prepare and deliver a T-cell redirecting therapy (e.g., a bispecific T-cell engaging molecule, a CAR-expressing T-cell) and/or a heavy-chain antibody disclosed herein for treating cancer in a subject in need thereof. In certain embodiments in which the pharmaceutical composition is provided in a lyophilized or dry powder form, the kit may comprise a diluent and instructions for reconstituting the pharmaceutical composition prior to administration. In some embodiments, the kits may further comprise one or more vials of intravenous solution stabilizer (IVSS) and instructions for using the IVSS for pre-treatment of IV bags prior to dilution of the pharmaceutical composition for delivery to the patient.

Also provided herein are kits comprising a combination product as described herein, wherein the combination product is present in a single container, or the components of the combination product are present individually, the kit optionally further comprising at least one of a group comprising instructions for use, a device for administration of the combination product or a component thereof, at least one separately packed medium for reconstitution, and a pharmaceutical carrier suitable for use with at least one of the combination partners.

The following examples are given for the purpose of illustrating various embodiments of the disclosure and are not meant to limit the present disclosure in any fashion. One skilled in the art will appreciate readily that the present disclosure is well-adapted to carry out the objects and obtain the ends and advantages mentioned, as well as those objects, ends, and advantages inherent herein. Changes therein and other uses which are encompassed within the spirit of the disclosure as defined by the scope of the claims will occur to those skilled in the art.

### EXAMPLES

This section provides specific examples of heavy-chain antibodies disclosed herein and methods of making and using the same.

List of Abbreviations

| | |
|---|---|
| ° | degree |
| % | percentage |
| 7-AAD | 7-aminoactinomycin D |
| ADA | anti-drug antibody |
| BSA | bovine serum albumin. |
| CD3 | cluster of differentiation 3 |
| CD4 | cluster of differentiation 4 |
| CD8 | cluster of differentiation 8 |
| CD25 | cluster of differentiation 25 |
| CD56 | cluster of differentiation 56 |
| CD127 | cluster of differentiation 127 |
| CD159a | cluster of differentiation 159a |
| CO₂ | carbon dioxide |
| DLL3 | delta-like ligand 3 |
| E:T | effector:target |
| EC₅₀ | half maximal effective concentration |
| Eₘₐₓ | maximum effect |
| ECL | electrochemiluminescent |
| FBS | fetal bovine serum |
| g | gram(s) |
| GM-CSF | granulocyte-macrophage colony-stimulating factor |
| gMFI | geometric mean fluorescent intensity |
| HCAb | heavy-chain antibody |
| HEPES | 4-(2-hydroxyethyl)-1-piperazineethanesulfonic acid |
| HPLC | high performance liquid chromatography |
| h or hr | hour(s) |
| IFNγ | interferon gamma |
| IL-1Ra | interleukin-1 receptor antagonist |
| IL-2 | interleukin-2 |
| IL-5 | interleukin-5 |
| IL-6 | interleukin-6 |
| IL-10 | interleukin-10 |
| L | liter(s) |
| LPDS | large protein depleted serum |
| Luc | luciferase |
| MCP-1 | monocyte chemoattractant protein-1 |
| min(s) | minute(s) |
| MIP-1β | macrophage inflammatory protein-1 beta |
| mg | milligram(s) |
| mL | milliliter(s) |
| NaN₃ | sodium azide |
| NEAA | non-essential amino acid(s) |
| ng | nanogram(s) |
| NK | natural killer |
| NSG | NOD-scid IL2Rgnull |
| PBMC | peripheral blood mononuclear cell |
| PBS | phosphate buffered |
| PS | polysorbate 80 |
| rh | recombinant human |
| RLU | relative luminescence units |
| RPMI | Roswell Park Memorial Institute |
| s | second(s) |
| SEC | size exclusion chromatography |
| SE-HPLC | size exclusion high performance liquid chromatography |
| SEFL | stable effector functionless variant |
| T-regs | regulatory T-cells |
| TDCC | T-cell dependent cellular cytotoxicity |
| TNFα | tumor necrosis factor alpha |
| μg | microgram(s) |
| μL | microliter(s) |
| UV | ultraviolet |

Two heavy-chain antibodies, HCAb1 and HCAb2, that function as agonists of the intermediate affinity IL-2 receptor are used in the following examples. HCAb1 is a heavy-chain antibody consisting of a first heavy chain polypeptide chain with the amino acid sequence of SEQ ID NO: 38 and a second heavy chain polypeptide chain with the amino acid sequence of SEQ ID NO: 39. HCAb2 is a heavy-chain antibody consisting of a first heavy chain polypeptide chain with the amino acid sequence of SEQ ID NO: 60 and a second heavy chain polypeptide chain with the amino acid sequence of SEQ ID NO: 61. HCAb2 was previously described and characterized in International Patent Application Publication No. WO 2022/212848 as “IL2Rβ_F09K hole ** IL2RG_F16B knob.” Other example IL2Rβ_F09, IL2Rβ_F18, ILRG_F16, and ILRG_F18 antibodies with IgG4-derived knob-in-hole Fc regions are also described and characterized in International Patent Application Publication No. WO 2022/212848.

HCAb1 includes the same VH regions as HCAb2. However, HCAb1 contains an IgG1-derived Fc region with SEFL2.2 mutations (R292C, N297G, V302C) (Liu et al., J Biol Chem. 2017 Feb. 3; 292 (5): 1876-1883) and charge-pair substitutions, while HCAb2 contains an IgG4-derived Fc region with knob-in-hole mutations. HCAb1 also includes a flexible GGGG linker (SEQ ID NO: 37) between each of its VH regions and its engineered Fc region. HCAb2 does not include a comparable flexible linker moiety.

EXAMPLE 1 provides in vitro characterization data for HCAb1. HCAb1 and HCAb2 perform similarly in in vitro human pSTAT5, cyno pSTAT5, and proliferation (% Ki67⁺) assays. However, HCAb1 exhibits reduced pre-existing reactivity in an anti-drug antibody bridging assay (EXAMPLE 2), reduced higher-order aggregation propensity (EXAMPLES 2, 4, 5), lower magnitude concentration-dependent viscosity changes (EXAMPLE 3), better stability over time at 40° C. (EXAMPLE 4), and better stability in large protein depleted serum (EXAMPLE 5) compared to HCAb2.

Moreover, HCAb1 improved the targeted cell killing activity associated with tarlatamab, a bispecific T-cell engaging molecule that binds to human CD3 and human DLL3, in an in vitro T-cell dependent cellular cytotoxicity (TDCC) assay (E: T=5:1, 72 hour assay time) (EXAMPLE 1). HCAb1 also limited T-cell engaging molecule-mediated T-cell dysfunction associated with the use of a T-cell engaging molecule that binds to human EpCAM and human CD3 in a serial T-cell engaging molecule exposure cytotoxicity assay (EXAMPLE 1). Additionally, in a SHP-77 Luc NSG mouse model under conditions resembling a T-cell low solid tumor microenvironment, the addition of HCAb1 to a sub-optimal dose of tarlatamab resulted in deep and durable anti-tumor efficacy, with an observed tumor regression of about 60% (EXAMPLE 6).

### Example 1. In Vitro Characterization of Hcab1

### Cell Lines and Peripheral Blood Mononuclear Cells (PBMCs)

Human PBMCs were isolated from fresh human peripheral blood leukopaks (StemCell Technologies) by Ficoll® Paque Premium (GE Healthcare Life Sciences) density gradient centrifugation, and subsequently cryopreserved in liquid nitrogen.

Cynomolgus monkey PBMCs were isolated from fresh whole blood that was collected in sodium heparin tubes from monkeys of Mauritian origin (AlphaGenesis Inc). The cell isolation was performed by Ficoll® Paque Premium (GE Healthcare Life Sciences) density gradient centrifugation, and the PBMCs were subsequently cryopreserved in liquid nitrogen.

The SHP-77 Luc cell line was grown at 37° C. in a 5% CO2 in a humidified incubator in RPMI 1640 (ATCC modification) medium (Gibco Life Technologies) further supplemented with 10% Fetal Bovine Serum (FBS), 1% Penicillin/Streptomycin. The SHP-77 Luc human tumor cell line exhibits endogenous DLL3 and EpCAM expression.

The NUGC4 CD58 knockout (KO) cell line (Shen et al., J Immunother Cancer. 2022; 10 (3): e004348) was grown at 37° C. in a 5% CO2 in a humidified incubator in RPMI 1640 medium supplemented with 10% FBS, 1% Penicillin/Streptomycin (50 IU/mL), 1% HEPES, 1% GlutaMAX™, 1% NEAA, and 1% sodium pyruvate (Gibco, Life Technologies).

### IL-2 Controls

IL-2v His (IL-2 variant) (Lake Pharma (Lot No. TP25895F)) was used as a control. This IL-2 variant contains mutations (F42A, Y45A, L72G) that have been shown to disrupt binding to IL-2Rα while retaining the ability to bind to and activate the intermediate affinity IL-2Rβγ receptor (Klein et al., Oncoimmunology 6:3 e1277306 (2017)). Recombinant human IL-2 (rhIL-2 (IL-2)) (R&D Biosystems Catalog No. 202-IL-050/CF) was also used as a control in certain assays. For the chronic TDCC assay, a wild-type human IL-2 molecule conjugated to the same Fc region used in HCAb1 (“wtIL2-Fc”) was also used as a control.

### Cell Binding by Flow Cytometry

All washes and dilutions of cells, antibodies, and reagents were performed using flow buffer (1×PBS, 1% BSA, 0.1% NaN3, pH 7.4). All incubations were performed at 4° C. or on ice. Freshly thawed PBMCs were seeded at 100,000 cells/well in a 96-well round bottom plate (Corning) and incubated for 30 minutes with serially diluted test articles in a total volume of 50 μL. Cells were washed twice with 200 μL flow buffer and subsequently stained with a 0.625 μg/mL Goat F (ab′) 2 Anti-Human IgG-PE (Southern Biotech) secondary detection antibody. Fluorescently conjugated anti-CD3 (OKT3), CD4 (OKT4), CD8 (RPA-T8), CD25 (M-A251), CD127 (A019D5), and CD56 (HCD56) antibodies (BioLegend) were also included at 1:20 dilution to enable the characterization of T-cell and NK cell populations. Following a 30-minute incubation, and 2 more washes, the cells were resuspended in a final volume of 120 μL of flow buffer containing 7-AAD viability dye. The cells were analyzed on a BD FACSCelesta™, maximum events were collected, and geometric mean fluorescence intensity was plotted as a fold-over-background (cells incubated with secondary detection antibody and cell lineage marker stains only).

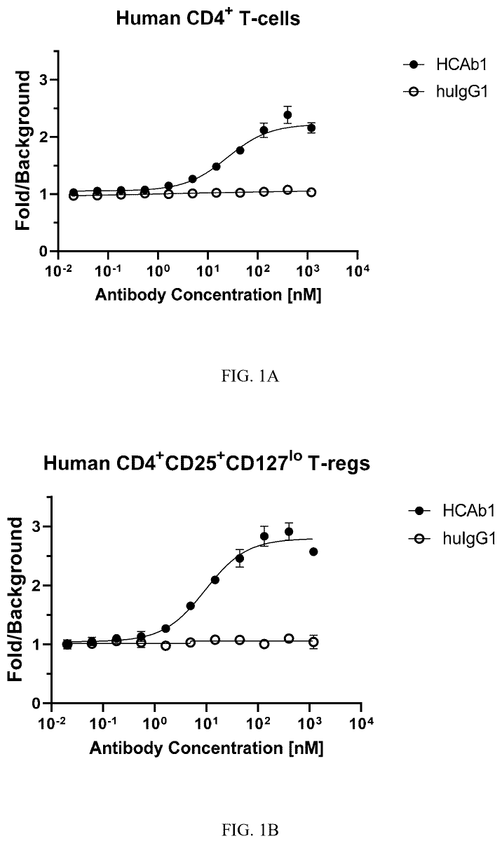

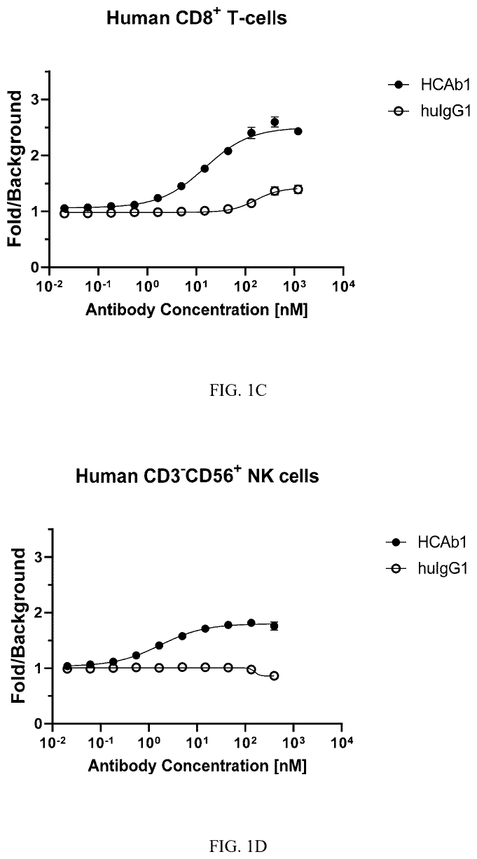

FIGS. 1A-1D show the binding of HCAb1 to human CD4⁺ T-cells (FIG. 1A), human CD4⁺CD25⁺CD1271^{b} T-regs (FIG. 1B), human CD8⁺ T-cells (FIG. 1C), and human CD3 CD56⁺ NK cells (FIG. 1D) relative to an IgG1 isotype control antibody as a function of concentration for each test article. Cell binding was determined by flow cytometry and reported as geometric mean fluorescent intensity (gMFI) over the gMFI of cells stained only with secondary detection antibody.

For cynomolgus monkey PBMCs, the procedure was conducted in a similar manner; however, an anti-CD3 (SP34) antibody was used in place of the OKT3 clone. CD56 antibody was also replaced with CD159a (Z199, Beckman Coulter).

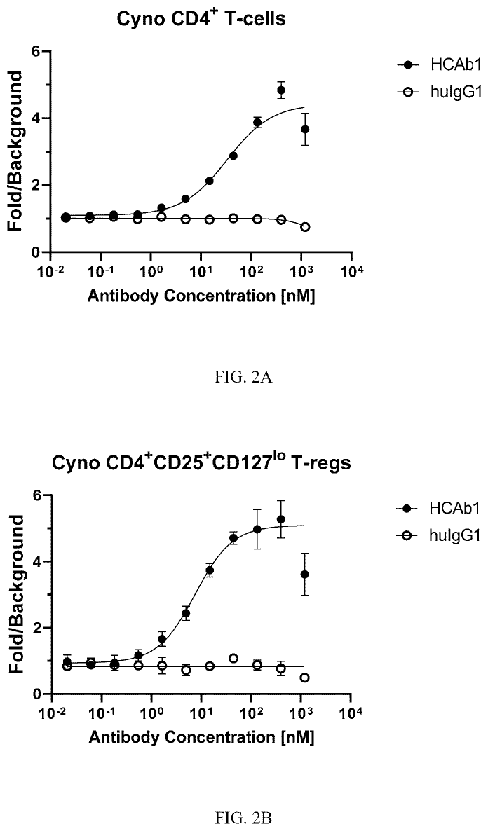

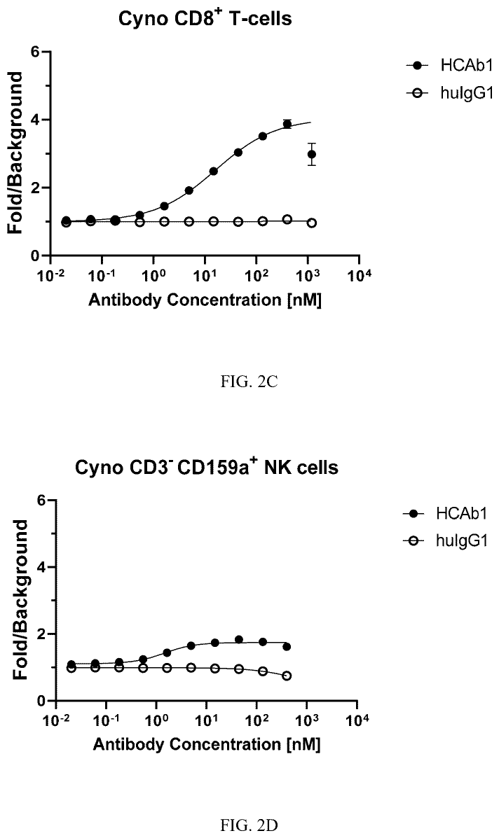

FIGS. 2A-2D show the binding of HCAb1 to cyno CD4⁺ T-cells (FIG. 2A), cyno CD4⁺CD25⁺CD1271^{b} T-regs (FIG. 2B), cyno CD8⁺ T-cells (FIG. 2C), and cyno CD3 CD159a⁺ NK cells (FIG. 2D) relative to an IgG1 isotype control antibody as a function of concentration for each test article. Cell binding was determined by flow cytometry and reported as geometric mean fluorescent intensity (gMFI) over the gMFI of cells that were not treated with the test articles.

pSTAT5

For detection of pSTAT5 by flow cytometry, cryopreserved human PBMCs were thawed, washed twice with complete RPMI medium (cRPMI), resuspended at 1×10⁶ cells/mL in cRPMI and rested overnight at 37° C. and 5% CO2 in a T-75 culture flask. The following morning, the cells were collected and resuspended at 5×10⁶ cells/mL in complete RPMI media. 100 μL of these cells was then transferred to each well of a sterile, round-bottom, 96-well plate (Corning), along with 100 μL of HCAb1 (or rhIL-2/IL-2 variant) diluted to 2× desired concentration. A final concentration of 10 nM rhIL-2 (R&D Systems) was used in control wells to ensure detectable pSTAT5 for setting flow cytometer gates. The plate was sealed with an AeraSeal™ (Excel Scientific) and incubated at 37° C. and 5% CO2 for 1 hour. After the incubation, the cells were centrifuged and washed twice with PBS pre-chilled to 4° C. The cells were then blocked with Human TruStain FcX™ (BioLegend) and subsequently stained for 30 minutes with Fixable Viability Dye (Invitrogen) and antibodies against CD3 (OKT3), CD4 (OKT4), CD8 (RPA-T8), CD25 (M-A251), and/or CD56 (HCD56). After staining, the cells were again centrifuged and washed twice with pre-chilled PBS. The cells were then incubated at room temperature for 30 minutes with 200 μL/well Foxp3/Transcription Factor Staining Buffer working solution (Invitrogen). After this fixation/permeabilization step, the cells were centrifuged, washed twice with 1× permeabilization buffer, stained for 30 minutes with anti-Foxp3 (206D, BioLegend), and washed twice again in permeabilization buffer.

Next, the cells were resuspended in 200 μL/well True-Phos™ buffer (BioLegend) pre-chilled to −20° C. and transferred to a −20° C. freezer overnight. The following morning, the cells were centrifuged, washed twice with flow buffer (1×PBS, 1% BSA, 0.1% NaN3, pH 7.4), and subsequently stained for 30 minutes with anti-pSTAT5 (47/(pY694), BD Biosciences). After two additional washes, the cells were resuspended in 125 μL/well flow buffer and the full sample volume was acquired on a BD FACSCelesta™

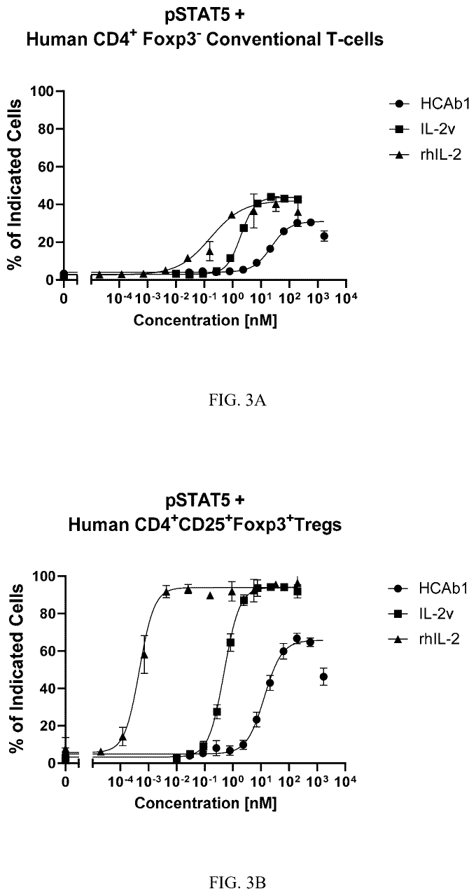

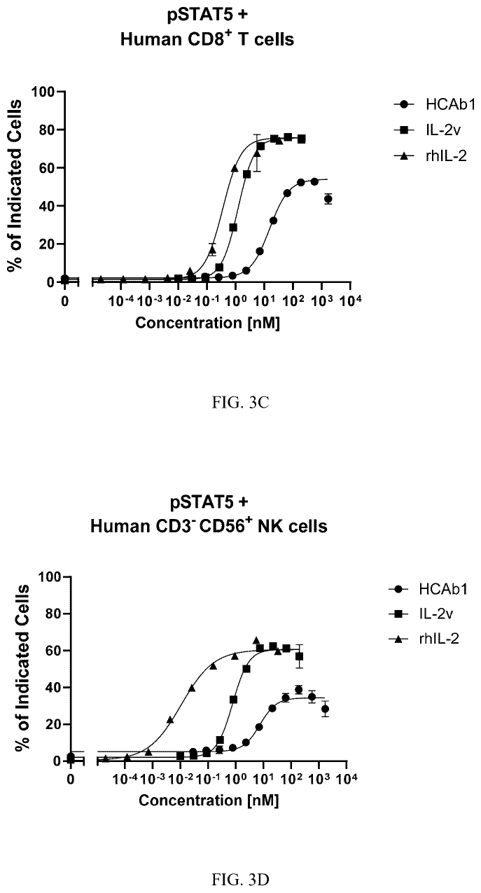

FIGS. 3A-3D depict STAT5 phosphorylation dose curves in human CD4⁺ Foxp3 T-cells (FIG. 3A), human CD4⁺CD25⁺ Foxp3⁺ regulatory T-cells (FIG. 3B), human CD8⁺ T-cells (FIG. 3C), and human CD3 CD56⁺ NK cells (FIG. 3D) as a function of concentration for HCAb1 and the control molecules (rhIL-2 and IL-2 variant). pSTAT5 levels were determined by flow cytometry and reported as a percentage of the indicated cell type. TABLE 12 summarizes EC₅₀ and Eₘₐₓ values for each test article across the assayed immune cell populations.

TABLE 12
EC₅₀ and Eₘₐₓ Values for HCAb1, rhIL-2, and IL-2v (pSTAT5)

| pSTAT5 | HCAb1 | | IL-2v | | rhIL-2 | |
|---|---|---|---|---|---|---|
| (Human) | EC₅₀ [nM] | Eₘₐₓ (%) | EC₅₀ [nM] | Eₘₐₓ (%) | EC₅₀ [nM] | Eₘₐₓ (%) |
| CD4⁺ T cells | 22.94 | 31 | 1.92 | 44 | 0.16 | 42 |
| T-regs | 13.14 | 66 | 0.49 | 94 | 0.0005 | 94 |
| CD8⁺ T cells | 15.47 | 54 | 1.21 | 76 | 0.36 | 76 |
| NK cells | 8.77 | 37 | 0.81 | 62 | 0.01 | 59 |

The ability of HCAb1 to stimulate IL-2R signaling in human CD4⁺ T-cells, CD8⁺ T-cells, regulatory T-cells, and NK cells was confirmed by a dose-dependent increase of STAT5 phosphorylation.

Like the non-α IL-2 variant, HCAb1 exhibits approximately equipotent STAT5 phosphorylation activity on regulatory T cells as the CD8⁺ effector T-cells (1.2-fold EC₅₀ differential) and NK cells (1.5-fold EC₅₀ differential). This is in stark contrast to rhIL-2, which has significantly higher potency and selectivity toward CD4⁺CD25⁺ Foxp3⁺ T-regulatory cells (720-fold vs CD8⁺ T cells). Thus, HCAb1 avoids the preferential activation of immunosuppressive T-regs. Proliferation (% Ki67⁺)

A cell proliferation assay was performed to further assess the downstream effect of IL-2 receptor stimulation. For detection of Ki67 by flow cytometry, frozen human PBMCs (previously isolated from a leukopak) were thawed and rested overnight in complete RPMI medium at 1×10⁶ cells/mL. The morning of the assay, the PBMCs were washed with complete RPMI and resuspended at 1×10⁶ cells/mL. Then, to each well of a sterile 96-well plate, 100 μL of PBMCs and 50 μL of 0.032× ImmunoCult™ (StemCell Technologies) were added. 50 μL of HCAb1 (or rhIL-2/IL-2 variant) were also added at 4× the desired concentration 0.5× ImmunoCult™ was used for staining controls to ensure detectable Ki67 and CD25 signal for fluorescence compensation. The plate was then covered and incubated at 37° C. and 5% CO2. After 3 days, the media was refreshed with 100 μL/well of the corresponding concentration antibody and ImmunoCult™ and then returned to the incubator. After 3 more days (6 days total), the cells were centrifuged and washed twice with 1×PBS pre-chilled to 4° C. The cells were then blocked with Human TruStain FcX™ (BioLegend) and subsequently stained for 30 minutes with Fixable Viability Dye (Invitrogen) and antibodies against CD3 (OKT3), CD4 (OKT4), CD8 (RPA-T8), CD25 (M-A251), and CD56 (HCD56) (BioLegend). After staining, the cells were again centrifuged and washed twice with pre-chilled PBS. The cells were then fixed and permeabilized for 1 hour with 200 μL/well Foxp3/Transcription Factor Staining Buffer working solution (Invitrogen). After permeabilization, the cells were centrifuged, washed twice with permeabilization buffer, and stained for 30 minutes with anti-Foxp3 (206D, BioLegend) and anti-Ki67 (Ki67, BioLegend). After two additional washes, the cells were resuspended in 125 μL/well flow buffer and the full sample volume was acquired on a BD FACSCelesta™.

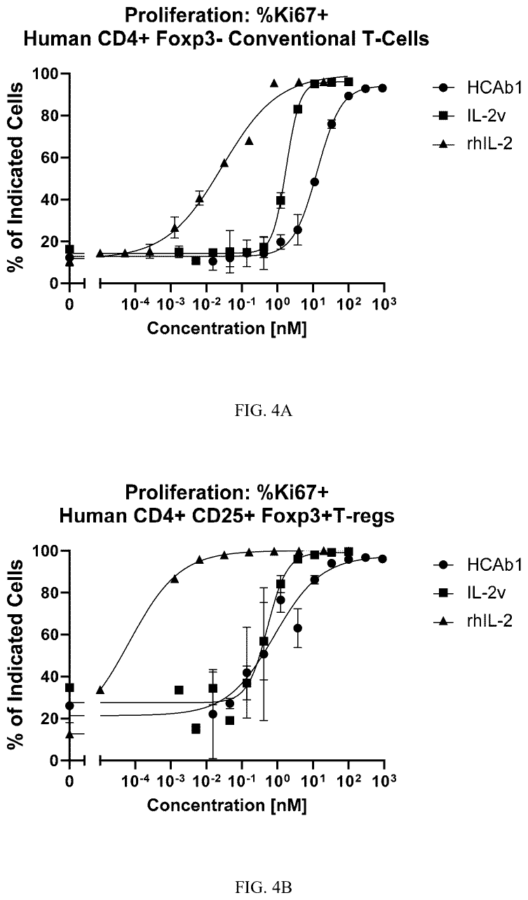

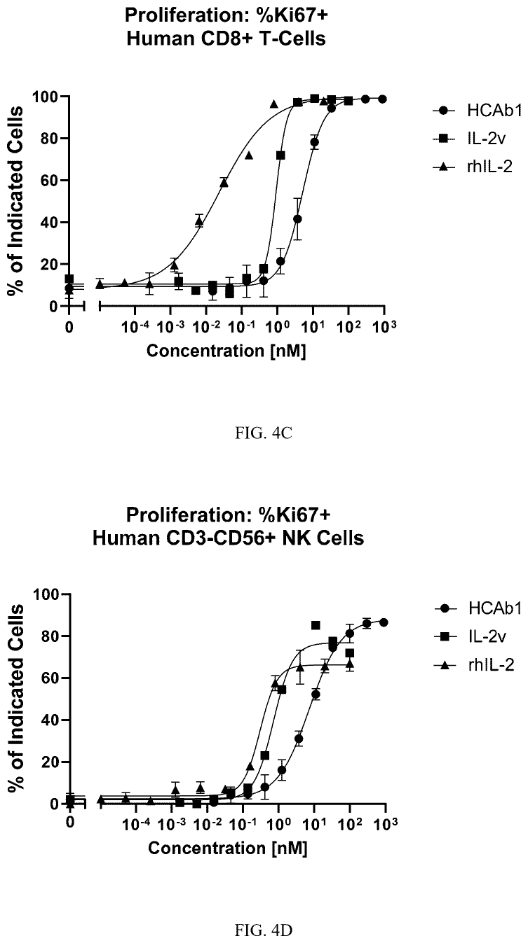

FIGS. 4A-4D show Ki67 dose curves for human CD4⁺ Foxp3-T-cells (FIG. 4A), human CD4⁺CD25⁺ Foxp3⁺ regulatory T-cells (FIG. 4B), human CD8⁺ T-cells (FIG. 4C), and human CD3 CD56⁺ NK cells (FIG. 4D) as a function of concentration for HCAb1 and the control molecules. Ki67 levels were determined by flow cytometry and were reported as a percentage of the indicated cell type. TABLE 13 summarizes EC₅₀ and Eₘₐₓ values for each test article across the assayed immune cell populations.

TABLE 13
EC₅₀ and Eₘₐₓ Values for HCAb1, rhIL-2, and IL-2v (Ki67 (Human))

| Ki67 | HCAb1 | | IL-2v | | rhIL-2 | |
|---|---|---|---|---|---|---|
| (Human) | EC₅₀ [nM] | Eₘₐₓ (%) | EC₅₀ [nM] | Eₘₐₓ (%) | EC₅₀ [nM] | Eₘₐₓ (%) |
| CD4⁺ T cells | 13.16 | 94 | 1.77 | 96 | 0.03 | 100 |
| T-regs | 0.81 | 98 | 0.5 | 99 | 0.0001 | 100 |
| CD8⁺ T cells | 5.17 | 99 | 0.93 | 99 | 0.02 | 100 |
| NK cells | 7.34 | 88 | 0.72 | 77 | 0.3 | 66 |

A similar protocol was used to assess the proliferation of PBMCs from cynomolgus monkeys. However, 0.4 ng/mL soluble anti-CD3 (SP34, BD Biosciences) was utilized instead of the ImmunoCult™ reagent. Additionally, anti-CD3 (OKT3) and CD56 (HCD56) were substituted with anti-CD3 (SP34, BD Biosciences) and anti-CD159a (Z199, Beckman Coulter) as cell surface markers for cyno PBMCs.

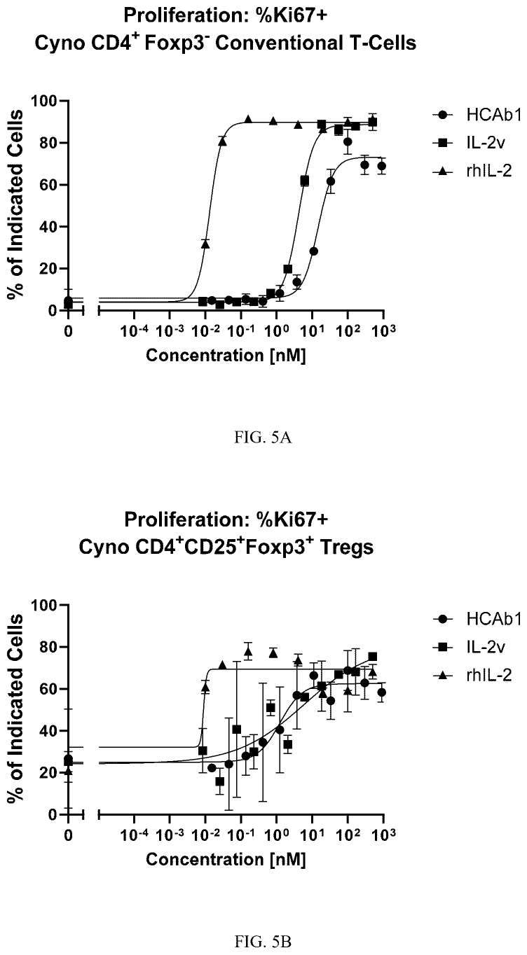

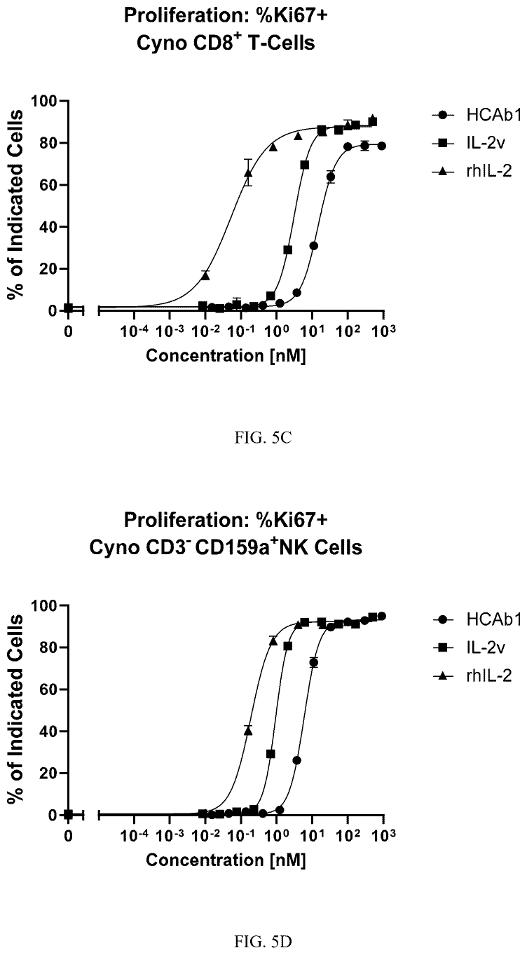

FIGS. 5A-5D show Ki67 dose curves for cyno CD4⁺ Foxp3 T-cells (FIG. 5A), cyno CD4⁺CD25⁺ Foxp3⁺ regulatory T-cells (FIG. 5B), cyno CD8⁺ T-cells (FIG. 5C), and cyno CD3 CD159a⁺ NK cells (FIG. 5D) as a function of concentration for HCAb1 and the control molecules. Ki67 levels were determined by flow cytometry and were reported as a percentage of the indicated cell type. TABLE 14 summarizes EC₅₀ and Eₘₐₓ values for each test article across the assayed immune cell populations.

TABLE 14
EC₅₀ and Eₘₐₓ Values for HCAb1, rhIL-2, and IL-2v (Ki67 (Cyno))

| Ki67 | HCAb1 | | IL-2v | | rhIL-2 | |
|---|---|---|---|---|---|---|
| (Cyno) | EC₅₀ [nM] | Emax (%) | EC₅₀ [nM] | Eₘₐₓ (%) | EC₅₀ [nM] | Eₘₐₓ (%) |
| CD4⁺ T cells | 14.74 | 73 | 4.19 | 89 | 0.01 | 90 |
| T-regs | 1.25 | 63 | 5.96 | 81 | 0.009 | 69 |
| CD8⁺ T cells | 14.83 | 79 | 3.11 | 88 | 0.05 | 88 |
| NK cells | 5.97 | 93 | 0.94 | 92 | 0.19 | 92 |

In response to treatment with HCAb1, or the IL-2/IL-2v cytokine controls, immune effector cells demonstrated dose-dependent proliferation.

HCAb1 showed similar or greater maximum induction relative to rhIL-2 and IL-2 variant in the proliferation of T-cells and NK cells, respectively. rhIL-2 was notably more active on human T-regs (200-fold higher potency on Tregs compared to CD8⁺ T-cells) than HCAb1 (6-fold) or the IL-2 variant control (2-fold).

HCAb1 was also confirmed to activate IL-2R signaling and induce proliferation of cynomolgus T-cells and NK cells with comparable functional efficacy as that observed on human T and NK cells, thereby establishing cynomolgus monkeys as a suitable non-human primate model for subsequent studies.

HCAb1 vs. HCAb2 Activity Separately, HCAb1 was evaluated alongside HCAb2 for functional equivalency in pSTAT5 and proliferation assays similar to those described above. The results of these side-by-side comparisons are detailed below in TABLES 15-17.

TABLE 15
EC₅₀ and Eₘₐₓ Values for HCAb1 and HCAb2 (pSTAT5 (Human)

| pSTAT5 | CD4⁺ T-cell | | CD8⁺ T-cell | | CD4⁺CD25⁺ T-regs | |
|---|---|---|---|---|---|---|
| (Human) | EC₅₀ [nM] | Eₘₐₓ (%) | EC₅₀ [nM] | Eₘₐₓ (%) | EC₅₀ [nM] | Eₘₐₓ (%) |
| HCAb1 | 34.88 | 26 | 21.49 | 60 | 10.71 | 34 |
| HCAb2 | 44.62 | 29 | 24.00 | 63 | 13.18 | 41 |

TABLE 16
EC₅₀ and Eₘₐₓ Values for HCAb1 and HCAb2 (pSTAT5 (Cyno))

| pSTAT5 | CD4⁺ T-cell | | CD8⁺ T-cell | | CD4⁺CD25⁺ T-regs | |
|---|---|---|---|---|---|---|
| (Cyno) | EC₅₀ [nM] | Eₘₐₓ (%) | EC₅₀ [nM] | Eₘₐₓ (%) | EC₅₀ [nM] | Eₘₐₓ (%) |
| HCAb1 | 7.72 | 39 | 3.306 | 75 | 5.374 | 84 |
| HCAb2 | 9.363 | 38 | 3.129 | 74 | 4.288 | 83 |

TABLE 17
EC₅₀ and Eₘₐₓ Values for HCAb1 and HCAb2 (Ki67 (Human)

| Ki67 | CD4⁺ T-cell | | CD8⁺ T-cell | | CD4⁺CD25⁺ T-regs | |
|---|---|---|---|---|---|---|
| (Human) | EC₅₀ [nM] | Eₘₐₓ (%) | EC₅₀ [nM] | Eₘₐₓ (%) | EC₅₀ [nM] | Eₘₐₓ (%) |
| HCAb1 | 23.7 | 51 | 25.06 | 66 | 9.26 | 66 |
| HCAb2 | 23.87 | 51 | 36.55 | 64 | 14.61 | 64 |

### In Vitro Combination TDCC

The ability of HCAb1 to improve the targeted cell killing activity associated with tarlatamab, a bispecific T-cell engaging molecule that binds to human CD3 and DLL3, was investigated using an in vitro T-cell dependent cellular cytotoxicity (TDCC) assay. In the TDCC assay, target cells (SHP-77 Luc) were co-cultured at 37° C. in a 96-well flat-bottom plate with human PBMCs for 72 hrs at a 5:1 (50,000:10,000) effector: target (E: T) ratio and increasing tarlatamab concentrations, with and without addition of HCAb1 (300 nM, 30 nM) or a human IgG1 isotype control (“hulgG1”), rhIL-2 (10 nM, 0.5 nM), or IL-2 variant (30 nM, 2 nM). Concentrations were selected based on Eₘₐₓ (“high”) and EC₅₀ (“medium”) values observed in functional assays. Target cell viability was determined using the Steady-Glo® Luciferase Assay System (Promega Catalog No. E2550). Relative Luminescence Units (RLU) were measured using a BioTek™ Neo2 microplate reader. Cytotoxicity was calculated using the formula:

$$[ 1 - ( RLU ( PBMCs + TARGET CELLS + ( HCAb 1 + TCE ) ) \text{} / ( RLU ( PBMCs + TARGET CELLS + HCAb 1 ) ) ] \times 100$$

Prior to the addition of the Steady-Glo® reagent, 100 μL of supernatant was collected from all wells for cytokine measurements.

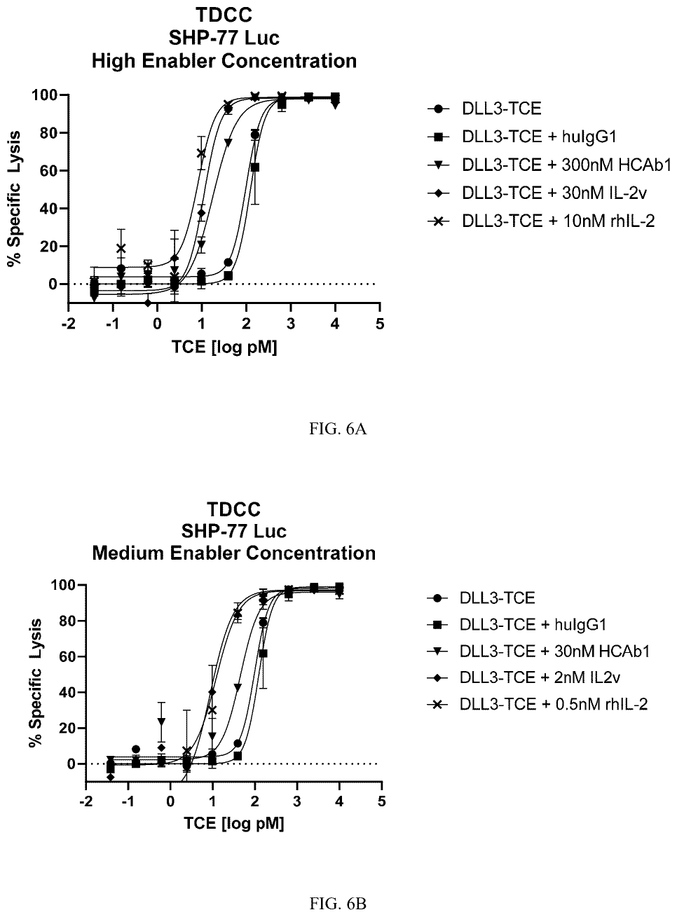

Combining tarlatamab (“DLL3-TCE”) with HCAb1, rhIL-2, or IL-2v was shown to enhance the DLL3-TCE-mediated killing of SHP-77 Luc cells as indicated by the potency shifts relative to DLL3-TCE monotherapy. FIGS. 6A and 6B depict the TDCC assay results (E: T=5:1, 72 hour assay time), and TABLE 18 summarizes EC₅₀ values and EC₅₀ fold change vs. tarlatamab (DLL3-TCE) alone values for different test conditions. A “high” enabler concentration refers to a 300 nM, 30 nM, or 10 nM concentration of HCAb1, IL-2v, or rhIL-2, respectively. A “medium” enabler concentration refers to a 30 nM, 2 nM, or 0.5 nM concentration of HCAb1, IL-2v, or rhIL-2, respectively.

TABLE 18
EC₅₀ and Fold Change Values

| | |
|---|---|
| High Enabler | Medium Enabler |
| Concentration | Concentration |

| | EC₅₀ | Fold | EC₅₀ | Fold |
|---|---|---|---|---|
| Test Article | [pM] | Change | [pM] | Change |
| DLL3-TCE | 97.9 | 1 | 97.9 | 1 |
| DLL3-TCE + huIgG1 | 122.9 | 0.8 | 122.9 | 0.8 |
| DLL3-TCE + HCAb1 | 17.7 | 5.5 | 45.12 | 2.2 |
| DLL3-TCE + IL-2v | 11.2 | 8.7 | 12.13 | 8.0 |
| DLL3-TCE + rhIL-2 | 8.3 | 11.8 | 8.79 | 11.1 |

Using supernatants that were collected from the TDCC assay, cytokine secretion was measured using a customized U-plex Meso Scale Discovery (MSD) panel per manufacturer protocol. Analytes measured include IFNγ, IL-2, IL-5, IL-6, IL-10, TNFα, MCP-1, MIP-1β, IL-1RA, GM-CSF, and Granzyme B. Data were analyzed using MSD Discovery Workbench software Version 4.0.

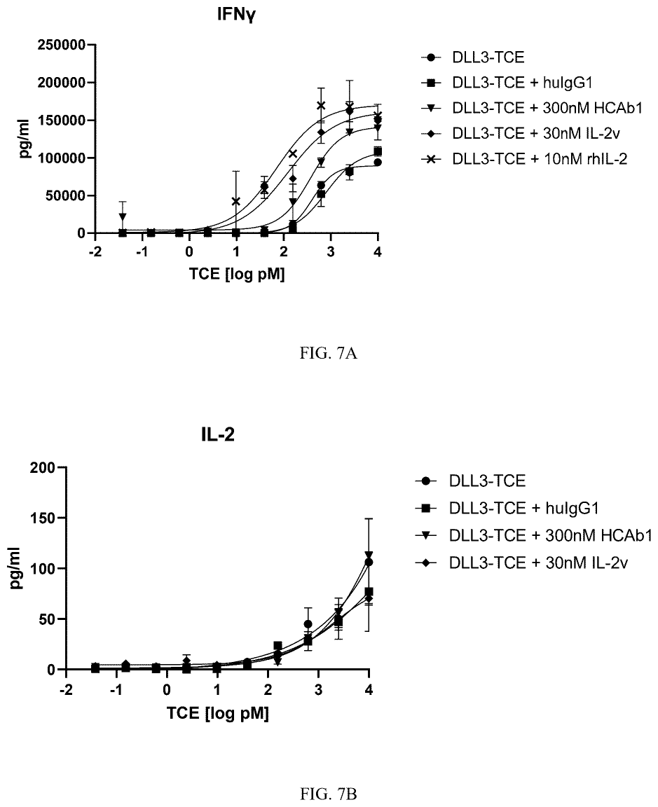

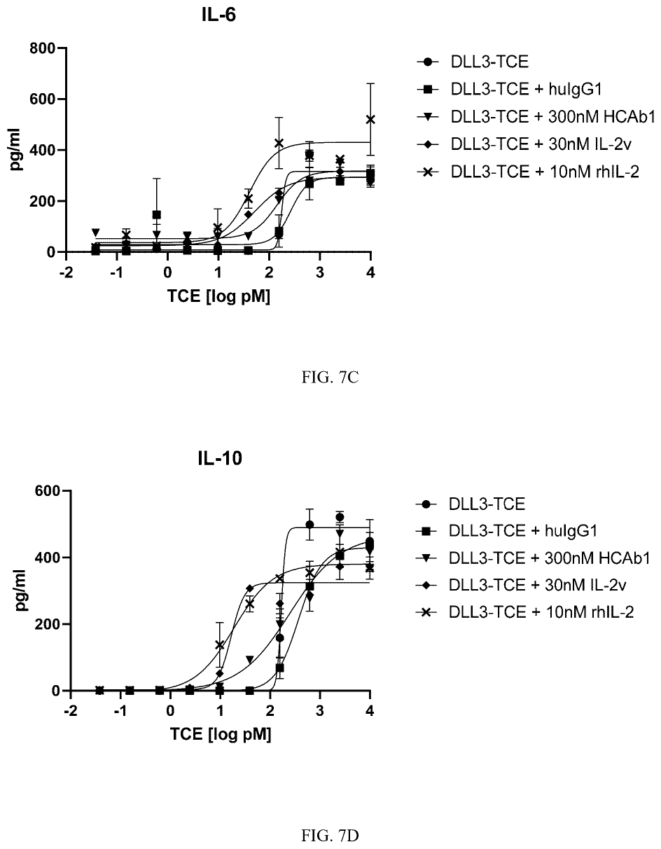

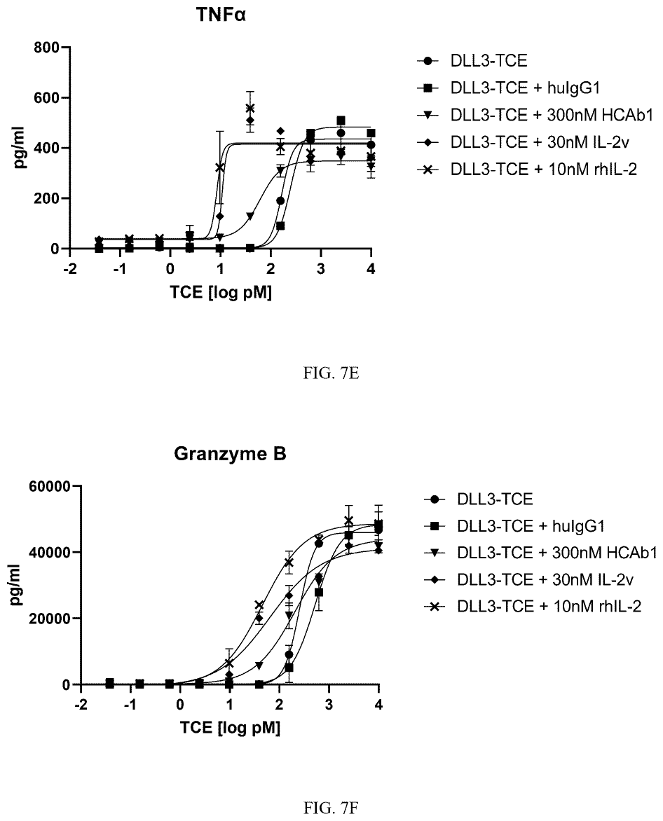

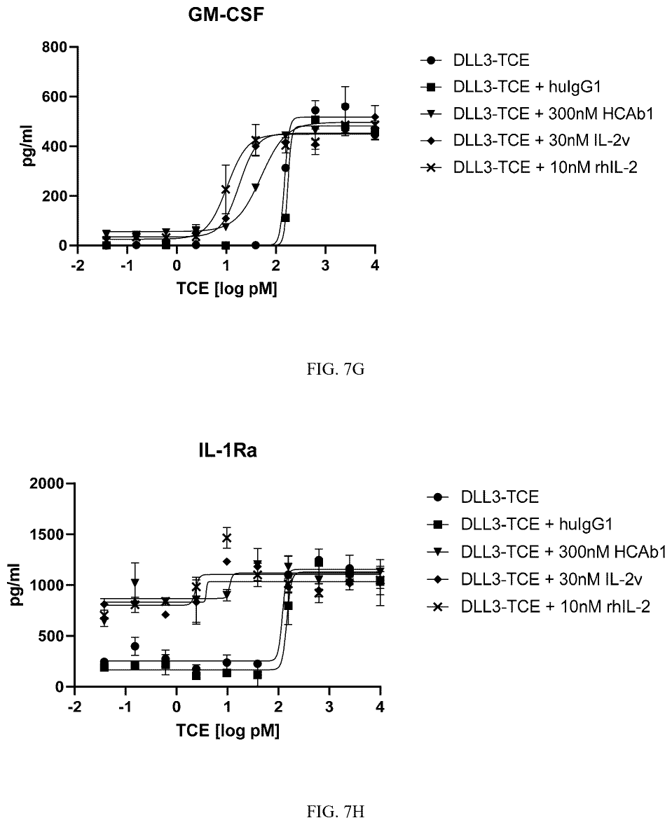

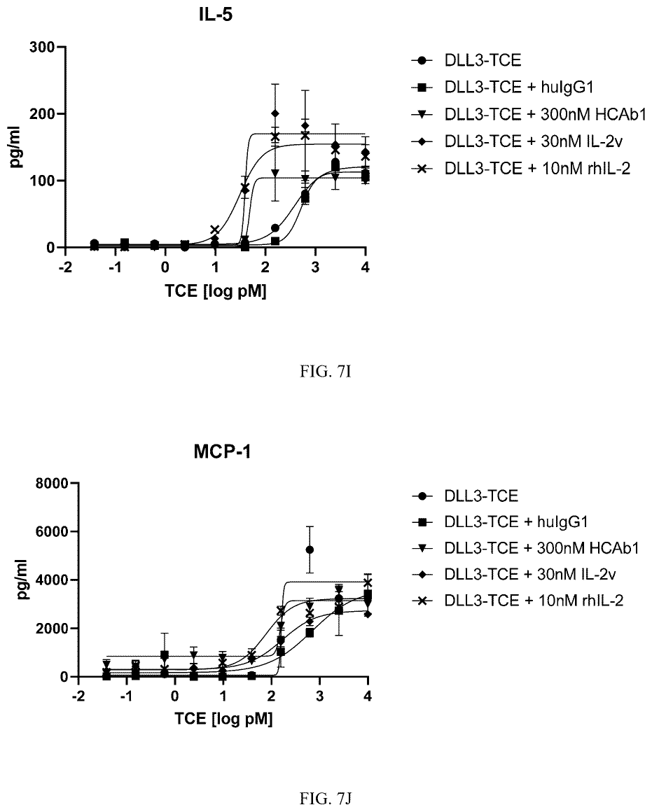

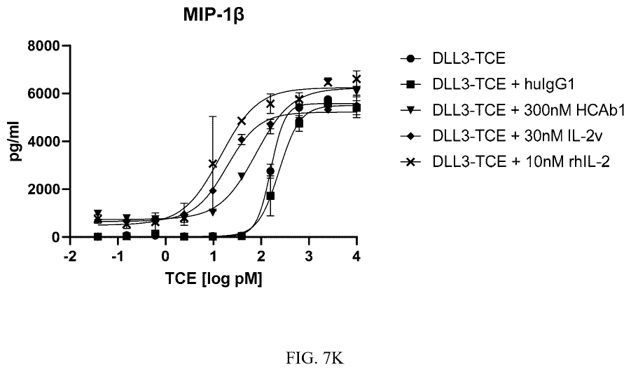

FIGS. 7A-7K show measured cytokine concentrations (IFNγ (FIG. 7A), IL-2 (FIG. 7B), IL-6 (FIG. 7C), IL-10 (FIG. 7D), TNFα (FIG. 7E), Granzyme B (FIG. 7F), GM-CSF (FIG. 7G), IL-1Ra (FIG. 7H), IL-5 (FIG. 7I), MCP-1 (FIG. 7J), and MIP-1β (FIG. 7K)) at various DLL3-TCE concentrations and high (300 nM, 30 nM, or 10 nM) concentrations of HCAb1, IL-2v, and rhIL-2, respectively.

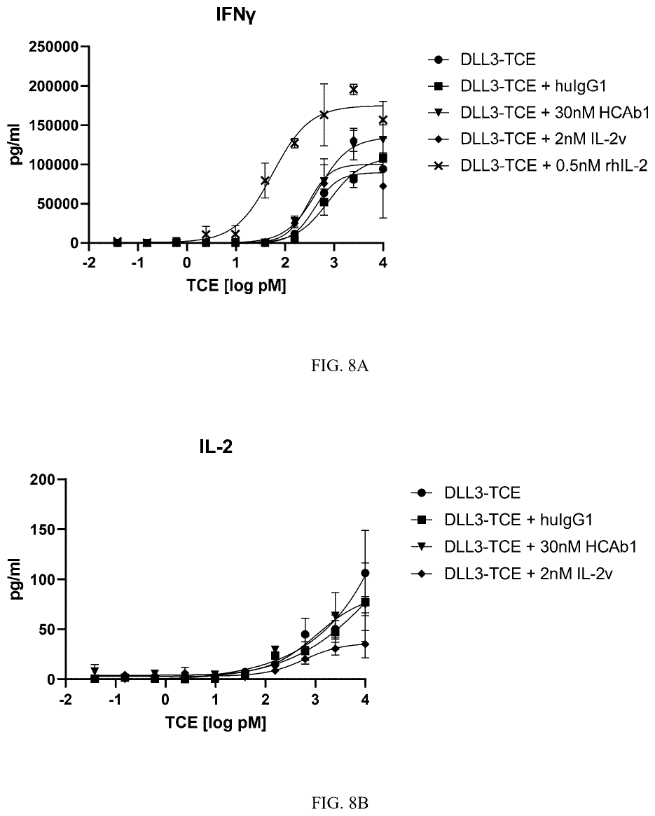

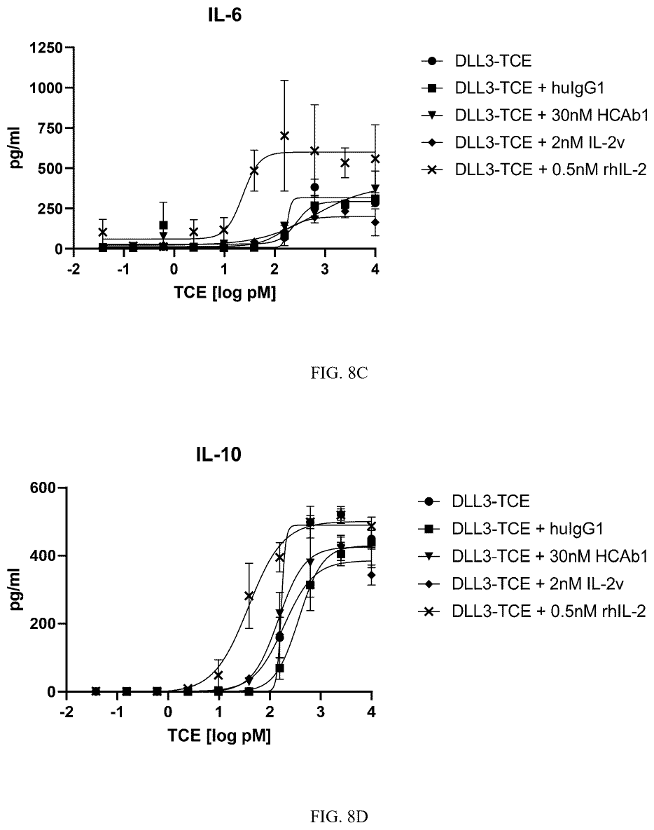

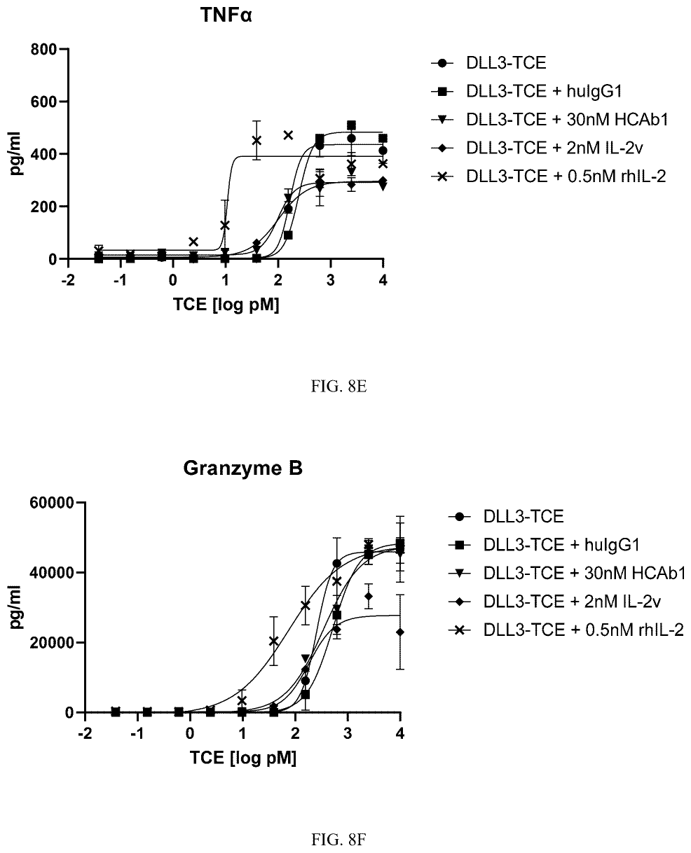

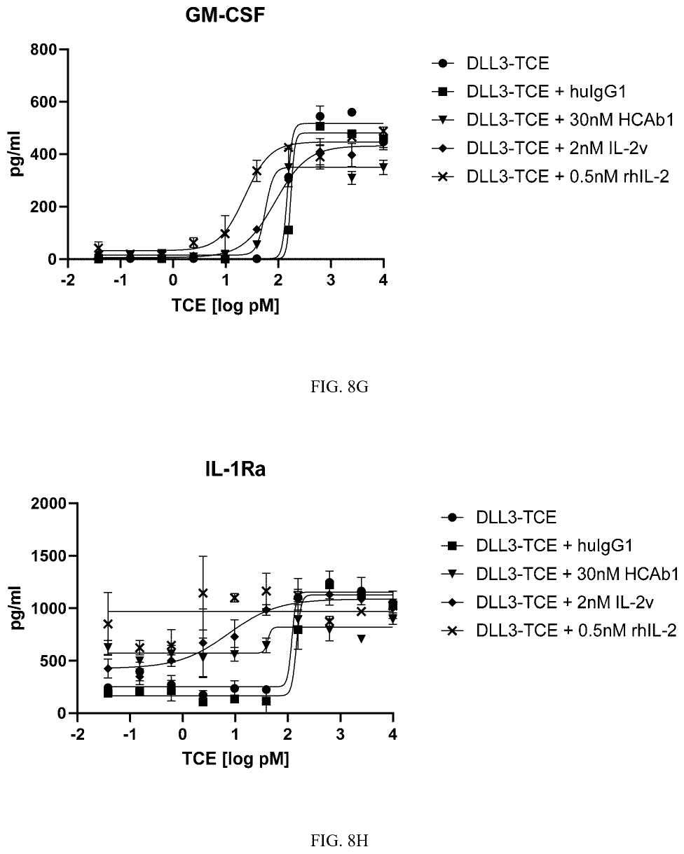

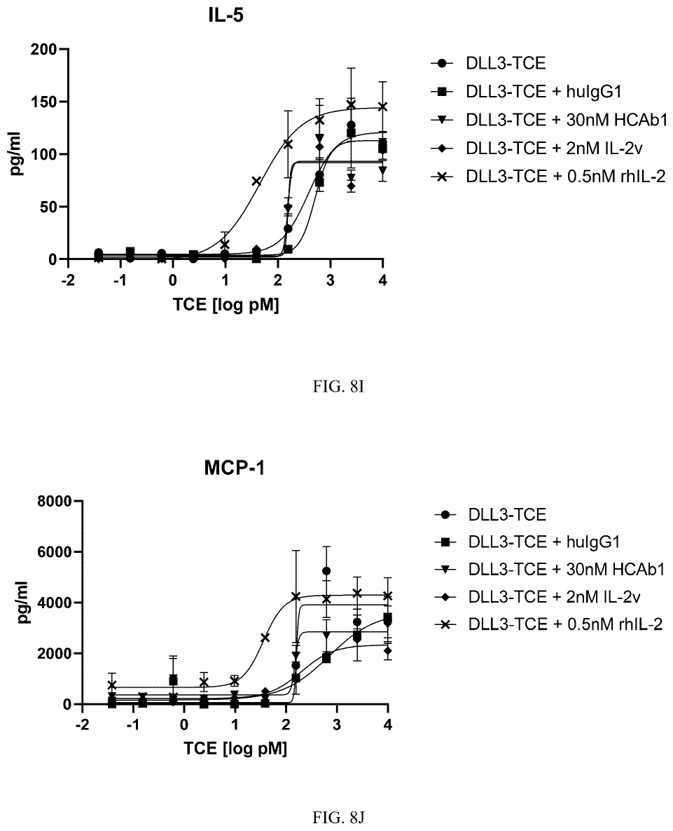

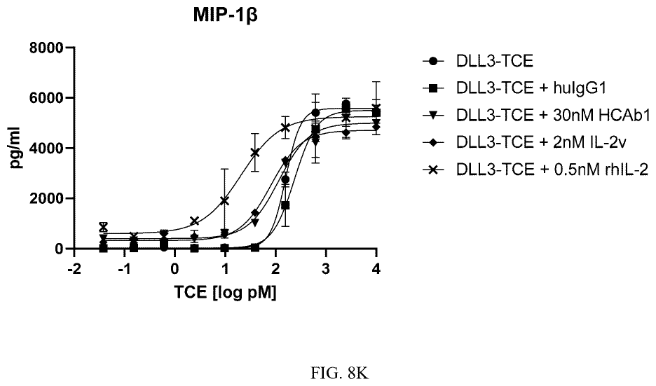

FIGS. 8A-8K show measured cytokine concentrations (IFNγ (FIG. 8A), IL-2 (FIG. 8B), IL-6 (FIG. 8C), IL-10 (FIG. 8D), TNFα (FIG. 8E), Granzyme B (FIG. 8F), GM-CSF (FIG. 8G), IL-1Ra (FIG. 8H), IL-5 (FIG. 8I), MCP-1 (FIG. 8J), and MIP-1β (FIG. 8K)) at various DLL3-TCE concentrations and medium (30 nM, 2 nM, or 0.5 nM) concentrations of HCAb1, IL-2v, and rhIL-2, respectively.

### Serial EpCAM-TCE Exposure Cytotoxicity Assay and In Vitro TDCC Assay

To further assess whether HCAb1 can mitigate T-cell engaging molecule-mediated T-cell dysfunction, a serial T-cell engaging molecule exposure cytotoxicity assay was performed as described previously described (Shen et al., J Immunother Cancer. 2022; 10 (3): e004348) using a T-cell engaging molecule that binds to human EpCAM and CD3 (“EpCAM-TCE”). In brief, on day 0, 6×10⁶ target cells (NUGC4-CD58 KO constitutively expressing firefly luciferase) were co-cultured with 12×10⁶ fresh thawed human T cells (E: T=2:1) in one T175 flask in the presence of 50 pM of EpCAM-TCE and 20 nM of HCAb1, IgG1 isotype control (“hulgG1”), IL-2v, or wtIL2-Fc. On days 4, and 7, T cells were collected from each T175 flask and washed once with PBS. Dead cells and tumor cells were removed by Ficoll-Paque™ PLUS (1.077 g/mL) (Cytiva) isolation followed by application of biotinylated anti-EpCAM (BioLegend)-anti-biotin-microbeads (Miltenyi) according to manufacturer's recommendations. Live T cells were counted using a Vi-CELL™ cell counter (Beckman Coulter) and resuspended in media containing new NUGC-CD58KO tumor cells at the same E: T ratio. The cells were then re-seeded in a new T175 flask containing fresh EpCAM-TCE (50 pM) in the presence of fresh HCAb1, IgG1 isotype control (“hulgG1”), IL-2v, or wtIL2-Fc (20 nM). On day 10, T cells were collected after 3 rounds of TCE exposure (“T3 cells”).

After removal of dead cells and tumor cells, the cytotoxic activity of T3 cells was assessed using an in vitro TDCC assay. In brief, target cells (NUGC4-CD58KO constitutively expressing firefly luciferase; 5000 cells/well in a 384-well plate) were co-cultured with T3 cells or with freshly thawed human CD3⁺ pan-T T0 cells (BioIVT) at an effector-to-target ratio (E: T) of 2:1 for 72 hours in the presence of 1:2 serial dilutions of EpCAM-TCE. Target cell viability was determined using the Bright-Glo™ Luciferase Assay System (Promega Catalog No. E-2610). Cytotoxicity was calculated using the formula:

$$[ 1 - ( RLU ( PBMCs + TARGET CELLS + ( HCAb 1 + TCE ) ) \text{} / ( RLU ( PBMCs + TARGET CELLS + HCAb 1 ) ) ] \times 100$$

Prior to Bright-Glo™ measurement, 30 μL/well of supernatant was removed for cytokine quantification. Human cytokine quantification was performed per manufacturer protocol using a customized U-plex Meso Scale Discovery (MSD) panel containing following analytes: IL-5, IL-6, IL-10, and TNFα.

TABLE 19 summarizes calculated EC₅₀ and Eₘₐₓ values for T0 cells and T3 cells treated with IgG1 isotype control (T3-hulgG1 cells), IL-2v (T3-IL-2v), HCAb1 (T3-HCAb1), and wtIL2-Fc (T3-wtIL2-Fc).

TABLE 19
EC₅₀ and Eₘₐₓ Values

| | T0 | T3-huIgG1 | T3-IL-2v | T3-HCAb1 | T3-wtIL2-Fc |
|---|---|---|---|---|---|
| | Cells | Cells | Cells | Cells | Cells |
| EC₅₀ [pM] | 7.2 | 376 | 8.0 | 6.5 | 5.6 |
| Eₘₐₓ (%) | 100 | 73 | 100 | 100 | 100 |

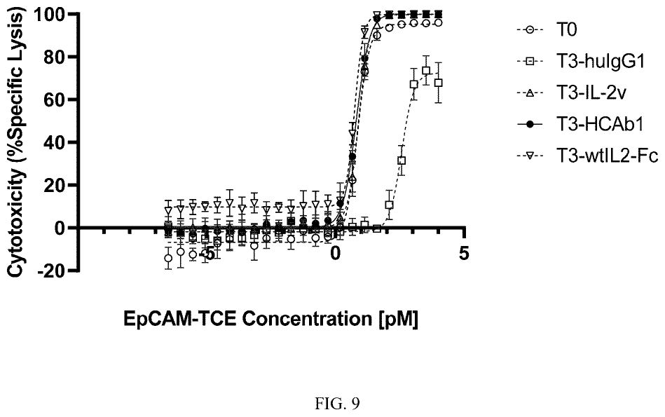

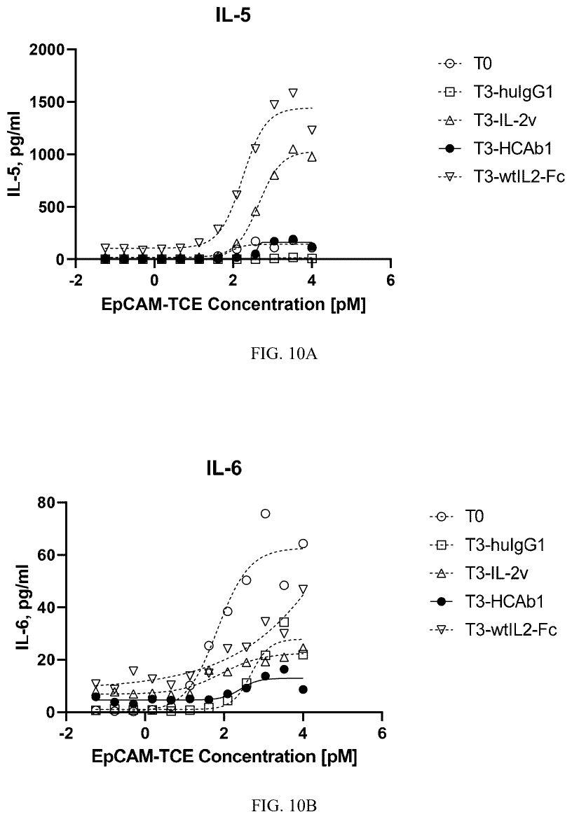

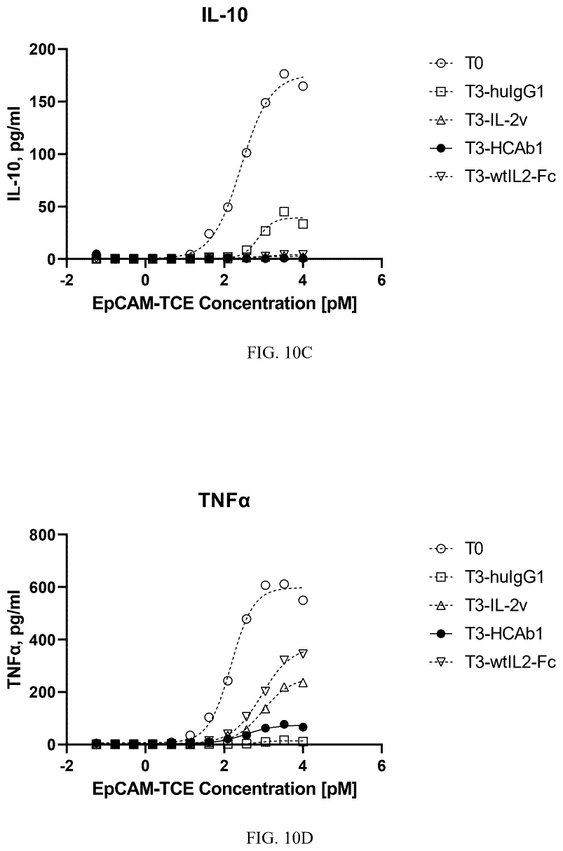

The killing potency of T3 cells alone was markedly decreased compared with freshly thawed T cells (TO), with 67-fold higher EC₅₀ values observed. By contrast, T3 cells incubated with HCAb1, IL-2v, or wtIL2-Fc retained similar killing potency to T0 cells (FIG. 9). Additionally, proinflammatory cytokine production from T3 cells treated with HCAb1 was significantly reduced across the tested panel (IL-5, IL-6, IL-10, and TNFα) (FIGS. 10A-10D).

### Example 2. Anti-Drug Antibody Assay

Immunogenicity concerns related to pre-existing anti-drug antibodies (ADAs) have increased in recent years as novel biotherapeutic modalities have entered the clinic in greater numbers. To assess the pre-existing reactivity of HCAb1 relative to HCAb2, the molecules were evaluated in a Meso Scale Discovery (MSD) bridging assay. In brief, the bridging assay procedure employed biotinylated and ruthenium-labeled drug molecules as bridge components. Upon sample incubation, anti-drug antibodies present in a donor human serum sample bind to both of the labeled drug molecules to form immunocomplexes, which are then captured on streptavidin-coated plates and detected by an electrochemiluminescent (ECL) signal measured on an MSD plate reader. In a confirmatory assay, unlabeled drug is spiked into the bridging assay, and the percent inhibition (%) can be calculated using assay signals from the same sample tested with and without spiked unlabeled drug.

### Materials

Human serum samples were purchased commercially from Bioreclamation IVT. These donor samples were from treatment-naïve, normal human individuals, equally distributed between genders. A generic positive control targeting the SEFL2 Fc region (“anti-SEFL2 mAb”) was used in all pre-existing reactivity screenings. All other materials are either commercially available from Mesoscale, Millipore Sigma, Thermo Scientific, or similar vendors, unless otherwise noted, or are known in the art and may be prepared by employing known procedures using ordinary skill.

### Labeling of Drug Molecules

HCAb1 and HCAb2 molecules were biotinylated using EZ-Link Sulfo-NHS Biotin, No-Weigh™ Format (Thermo Scientific, A39256) and ruthenylated using MSD SULFO-TAG NHS-Ester (Mesoscale, R91AO-1) in line with manufacturer protocol. The challenge ratios for the conjugation reactions were 10:1 for biotin and 5:1 for ruthenium. Labeling was confirmed by Size Exclusion High Performance Liquid Chromatography (SE-HPLC).

### Two-Tiered ECL-Bridging Assay

For the pre-existing reactivity evaluation, an ECL-based bridging assay approach was used. Briefly, human serum samples were diluted in 1% BSA in 1×PBS to 5%, 50 μL of diluted donor samples were then transferred to polypropylene 96-well plates containing 100 μL of 1.0 μg/mL each of biotinylated and ruthenium-labeled drug molecule and incubated overnight at room temperature. At the same time, MSD Gold 96-well streptavidin plates (Mesoscale L15SA-1) were blocked with 1% BSA in 1×PBS overnight at room temperature. The blocked MSD plates were then washed, and 50 μL of incubated samples were transferred from the polypropylene plate to the MSD plate and allowed to incubate for 1 hour. The plates were then washed three times with 1×PBS buffer, and 150 μL of MSD GOLD Read Buffer (Mesoscale, R92TC-2) was added to the wells. Plates were then read on an MSD SQ120 reader to provide the Tier 1 signal expressed as ECL units.

To generate the Tier 2 (also known as confirmatory) signal, unlabeled drug molecule was added to the mixture of 1.0 g/mL each of biotinylated and ruthenium-labeled drug at 25 μg/mL concentration. Tier 2 percent signal inhibition was then calculated using Tier 1 and Tier 2 signal according to the following formula:

$$[ ( Tier 1 signal ) - ( Tier 2 signal ) ] / ( Tier 1 signal ) \times 100$$

### Sample Pretreatment Using Protein G/L Agarose Beads

Agarose or protein G/L agarose beads (150 μL) were added to Millipore HV Filter Plate (Millipore MSHVS4510) and then washed with 200 μL 1×PBS and centrifuged at 1000-2000 rpm for 2-4 minutes twice. Samples were then diluted in 1% BSA in 1×PBS to 5% and then 100 μL of diluted samples were transferred to a filter plate containing washed beads to incubate 1-2 hours while shaking. After centrifuging at 1000-2000 rpm, 50 μL of the flow-throughs were then tested in a Tier 1 ECL-bridging assay to evaluate the pre-existing reactivity by the MSD bridging assay.

### Results

In the bridging assay, HCAb2 but not HCAb1 exhibited unusual pre-existing reactivity, which was defined as greater than 40% of donors depleting over 40%. HPLC analysis of unlabeled, biotinylated, and ruthenium-labeled HCAb2 indicated that low levels (about 0.05% to about 2.5%) of higher-order aggregates were present in the samples. In subsequent assays assessing pre-existing reactivity for multiple samples of HCAb2 with different aggregation levels, reactivity results were found to correlate with higher-order aggregate peak level (TABLE 20). To obtain a HCAb2 sample with a very low (0.01) aggregate peak level (Sample 4), HCAb2 was purified using multiple columns.

TABLE 20
HCAb2 Pre-Existing Reactivity and
Aggregate (>670 kDa) Peak Level

| | Donors Depleting >40% | Aggregate Peak Level |
|---|---|---|
| Sample 1 | 71% | 2.26 |
| Sample 2 | 47% | 0.49 |
| Sample 3 | 31% | 0.12 |
| Sample 4 | 0% | 0.01 |

By contrast, HCAb1, which includes the same VH domains as HCAb2 but a different Fc region and a flexible GGGG (SEQ ID NO: 37) linker between each of its VH domains and Fc region, showed significantly lower higher-order aggregate levels by HPLC analysis and did not exhibit unusual pre-existing reactivity in the MSD bridging assay (TABLE 21).

TABLE 21
HCAb1 Pre-Existing Reactivity and
Aggregate (>670 kDa) Peak Level

| | Donors Depleting >40% | Aggregate Peak Level |
|---|---|---|
| Sample 1 | 24% | 0 |
| Sample 2 | 20% | 0.05-0.11 |

### Example 3. Concentration—and Temperature-Dependent Viscosity

The viscosity of a formulation can affect both its syringeability and injectability. The viscosities of formulations comprising HCAb1 or HCAb2 at various concentrations and temperatures were compared as described below.

HCAb1 dialyzed in 10 mM sodium acetate, 9% sucrose, pH 5.2 and HCAb2 dialyzed in 10 mM sodium acetate, 9% sucrose, pH 5.2 were used as starting materials. To obtain a concentrated drug product at the desired maximum concentration, starting materials were loaded into a spin concentrator and centrifuged at 4000 rpm until the desired concentration was achieved, as assessed using a SoloVPE® system. Polysorbate 80 (PS80) was then spiked into the concentrated solution to 0.01%.

The concentrated solution was then diluted in formulation buffer (100 mM sodium acetate, 9% sucrose, 0.01% PS80, pH 5.2) to the target concentrations. For HCAb1, the target concentrations were 5 mg/mL, 25 mg/mL, 100 mg/mL, 120 mg/mL, 140 mg/mL, and 180 mg/mL. For HCAb2, the target concentrations were 5 mg/mL, 25 mg/mL, 75 mg/mL, 100 mg/mL, 120 mg/mL, 140 mg/mL, 160 mg/mL, and 180 mg/mL. TABLE 22 and TABLE 23 summarize the actual concentrations obtained for the HCAb1 and HCAb2 formulations, as assessed using a Solo VPE® system.

TABLE 22
Actual vs. Target HCAb1 Concentrations

| | Target (mg/mL) | | | | | |
|---|---|---|---|---|---|---|
| | 5 | 25 | 100 | 120 | 140 | 180 |
| Actual (mg/mL) | 4.8 | 24.7 | 99.3 | 119.2 | 142.0 | 182.9 |

TABLE 23
Actual vs. Target HCAb2 Concentrations

| | Target (mg/mL) | | | | | | | |
|---|---|---|---|---|---|---|---|---|
| | 5 | 25 | 75 | 100 | 120 | 140 | 160 | 180 |
| Actual | 4.6 | 23.4 | 72.5 | 101.2 | 119.9 | 137.8 | 155.5 | 181.4 |
| (mg/mL) | | | | | | | | |

Viscosity measurements were obtained using a cone-and-plate method on a TA Instruments DHR-2 rheometer at about 5° C., 10° C., 15° C., 20° C., 25° C., and 37° C. and a shear rate of 1,000 s⁻¹. TABLE 24 and TABLE 25 summarize the measured viscosity values for HCAb1 and HCAb2, respectively.

TABLE 24
Viscosity (cP) for HCAb1 Formulations

| Concentration | Temperature | | | | | |
|---|---|---|---|---|---|---|
| (mg/mL) | 5° C. | 10° C. | 15° C. | 20° C. | 25° C. | 37° C. |
| 4.8 | 2.15 | 1.83 | 1.59 | 1.40 | 1.24 | 0.96 |
| 24.7 | 2.51 | 2.13 | 1.84 | 1.61 | 1.43 | 1.09 |
| 99.3 | 5.86 | 4.82 | 4.05 | 3.48 | 3.05 | 2.27 |
| 119.2 | 8.29 | 6.74 | 5.60 | 4.78 | 4.18 | 3.11 |
| 142 | 12.60 | 10.05 | 8.21 | 6.94 | 6.04 | 4.45 |
| 182.9 | 37.51 | 28.57 | 22.44 | 18.40 | 15.72 | 11.20 |

TABLE 25
Viscosity (cP) for HCAb2 Formulations

| Concentration | Temperature | | | | | |
|---|---|---|---|---|---|---|
| (mg/mL) | 5° C. | 10° C. | 15° C. | 20° C. | 25° C. | 37° C. |
| 4.6 | 2.18 | 1.85 | 1.60 | 1.41 | 1.25 | 0.97 |
| 23.4 | 2.53 | 2.14 | 1.85 | 1.62 | 1.43 | 1.10 |
| 72.5 | 4.52 | 3.74 | 3.15 | 2.72 | 2.38 | 1.78 |
| 101.2 | 7.35 | 5.93 | 4.89 | 4.13 | 3.58 | 2.60 |
| 119.9 | 10.75 | 8.48 | 6.87 | 5.72 | 4.90 | 3.49 |
| 137.8 | 15.97 | 12.32 | 9.78 | 7.99 | 6.77 | 4.72 |
| 155.5 | 27.65 | 20.67 | 15.95 | 12.72 | 10.56 | 7.10 |
| 181.4 | 59.86 | 42.92 | 31.91 | 24.55 | 19.91 | 12.91 |

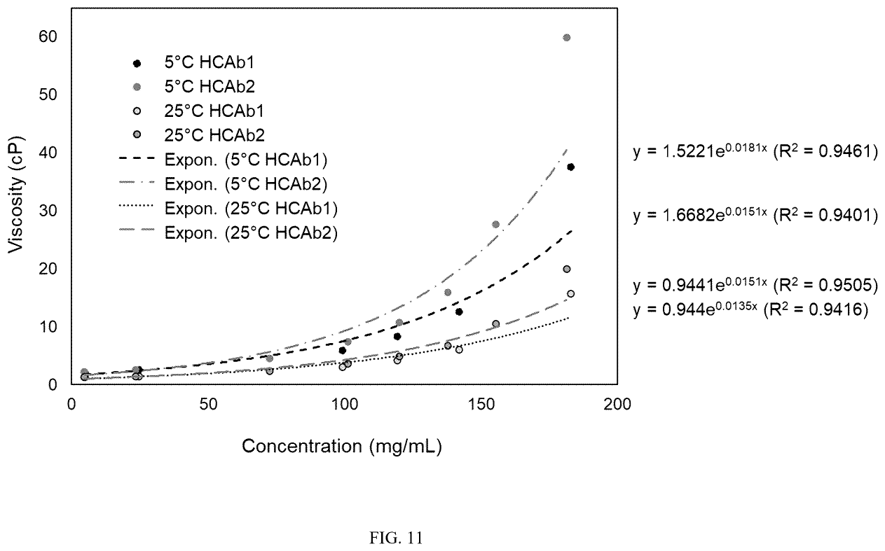

The tested HCAb1 and HCAb2 formulations were characterized by viscosities of less than 10 cP up to concentrations of about 140 mg/mL at 25° C. However, the measured viscosity was consistently higher for formulations comprising HCAb2 at all tested concentrations, especially at higher concentrations. Moreover, exponential fit curves showed a steeper fit for HCAb2 viscosity at both 5° C. and 25° C. (FIG. 11), indicating that viscosity increased more rapidly at increased concentration for HCAb2 relative to HCAb1.

### Example 4. Four Week Stability at 40° C.

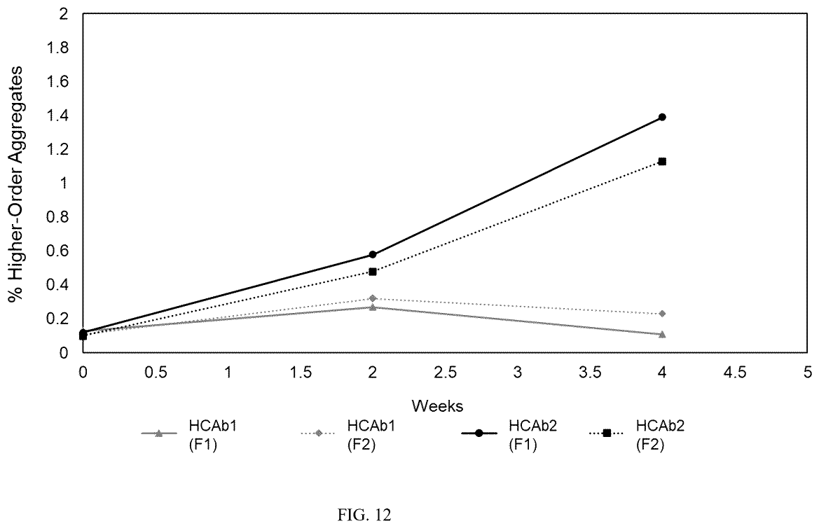

The stabilities of HCAb1 and HCAb2 at 40° C. over the course of 4 weeks were compared by SE-UHPLC analysis. In brief, higher-order aggregate percentages were compared over 4 weeks of incubation at 40° C. for HCAb1 and HCAb2 at a concentration of 5 mg/mL in two formulation buffers: (1) 10 mM histidine, 9% sucrose, 0.01% PS80, pH 6.2 (“F1”); and (2) 10 mM histidine, 9% sucrose, 0.01% PS80, pH 6.8 (“F2”). HCAb2 formed higher-order aggregates at a faster rate compared to HCAb1 at 40° C. in both formulation buffers (FIG. 12).

### Example 5. Stability in Large Protein Depleted Human Serum

The stability of HCAb1 and HCAb2 in large protein depleted serum (LPDS) was assessed in real-time by SEC-UV analysis. The LPDS stability assay was previously described in International Patent Application Publication No. WO 2020/180967. In brief, human serum samples were depleted of large proteins through processing with a 30K molecular weight cutoff filter such that the final depleted human serum retained inorganic and organic serum components with about 4-5% small serum proteins and peptides but did not interfere with SEC monitoring. HCAb1 or HCAb2 to a concentration of about 250 μg/mL was added to LPDS and incubated with gentle motion in closed vials in an oven without CO2 at 37° C. SEC-UV measurements were obtained at about 0 (initial neat), 0.05, 1, 6, 24, 48, 72, 96, and 120 hour time points.

TABLE 26 summarizes the observed aggregation (dimer and higher-order aggregate) behavior for both heavy-chain antibodies over the course of 120 hours. While both heavy-chain antibodies showed a time-dependent increase in aggregation, HCAb1 exhibited lower aggregation levels compared to HCAb2 in LPDS under the conditions tested.

TABLE 26
Stability in LPDS

| | HCAb1 (10 mM acetate, 9% | | HCAb2 (10 mM histidine, 9% | |
|---|---|---|---|---|
| | sucrose, 0.01% PS80, pH 5.2) | | sucrose, 0.01% PS80, pH 6.8) | |
| Time | % | % Higher-Order | % | % Higher-Order |
| (hr) | Dimer | Aggregate | Dimer | Aggregate |
| Initial | 0.32 | 0.14 | 0.34 | 0.13 |
| Neat | | | | |
| 0.05 | 0.44 | 0.78 | 0.35 | 0.59 |
| 1 | 0.10 | 0.19 | 0.31 | 2.15 |
| 6 | 0.09 | 0.24 | 0.22 | 0.25 |
| 24 | 0.07 | 0.10 | 0.2 | 0.27 |
| 48 | 0.05 | 0.12 | 0.18 | 0.4 |
| 72 | 0.07 | 0.37 | 0.18 | 0.44 |
| 96 | 0.06 | 0.10 | 0.18 | 0.51 |
| 120 | 0.06 | 0.08 | 0.19 | 0.27 |

### Example 6. Hcab1 and Tarlatamab In Vivo Combination Study

An in vivo combination study using HCAb1 and tarlatamab (“DLL3-TCE”) was performed in a SHP-77 Luc model in female NOD-scid IL2Rgnull (NSG) mice. The SHP-77 Luc human tumor cell line exhibits endogenous DLL3 expression. Under conditions resembling a T-cell low solid tumor microenvironment, the addition of HCAb1 to a sub-optimal dose of tarlatamab resulted in deep and durable anti-tumor efficacy, with tumor regression of about 60% observed by end of study.

Female NSG mice, age 6-8 weeks (Jackson Labs), were subcutaneously engrafted with SHP-77 human tumor cells (2×10⁶ cells per mouse in 1:1 RPMI media and Matrigel, Corning) in the right flank on study day 0. On study day 11, mice were randomized into treatment groups of n=10 based on tumor volume as measured by digital calipers (tumor volume=L×W×H, expressed in mm³). On study day 12, mice were engrafted with 1 million pan CD3⁺ human T cells to mimic a low T-cell tumor microenvironment; the T-cells were expanded in vitro for 11 days in the presence of rhIL-2 (10 ng/ml, StemCell Technologies) and ImmunoCult™ Human CD3/CD28/CD2 T Cell Activator (StemCell Technologies) in Immunocult™-XF media (StemCell Technologies). On study day 13, mice were treated with HCAb1 (100 μg/kg, given intravenously), tarlatamab (100 μg/kg, given intraperitoneally), or negative control antibodies, as indicated in the treatment groups. Mice were dosed once weekly for a total of 3 doses. Serum samples were collected via retro-orbital bleed at 24 and 120 hours post-first dose and analyzed for pharmacokinetics by ELISA and pharmacodynamics by serum cytokine levels (Meso Scale Discovery, MSD). Tumor volume and body weight were measured twice weekly, and mice were removed from study once tumor volume exceeded 10% of the mouse body weight (about 2000 mm³). Tumor growth inhibition was assessed once the first mouse was removed from the study. Statistical analysis was performed using IVEA mixed linear effects with Tukey's all groups comparison. P values of <0.05 were considered statistically significant.

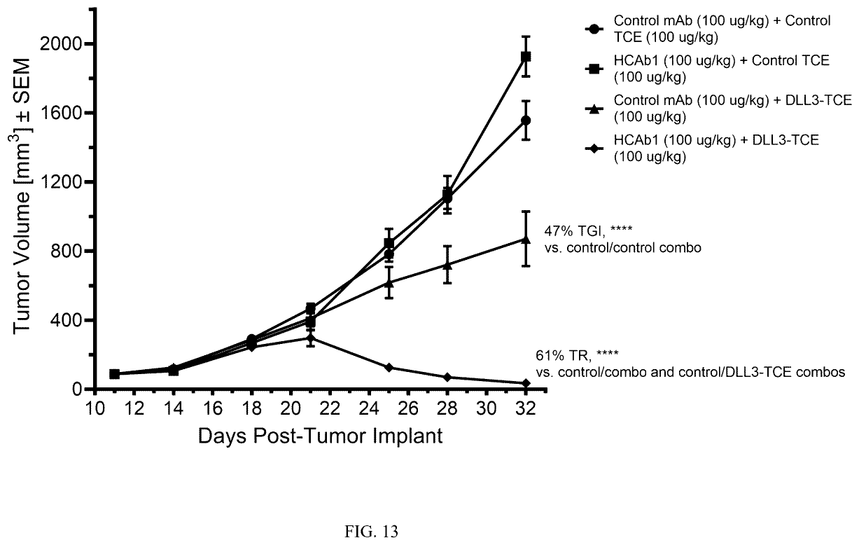

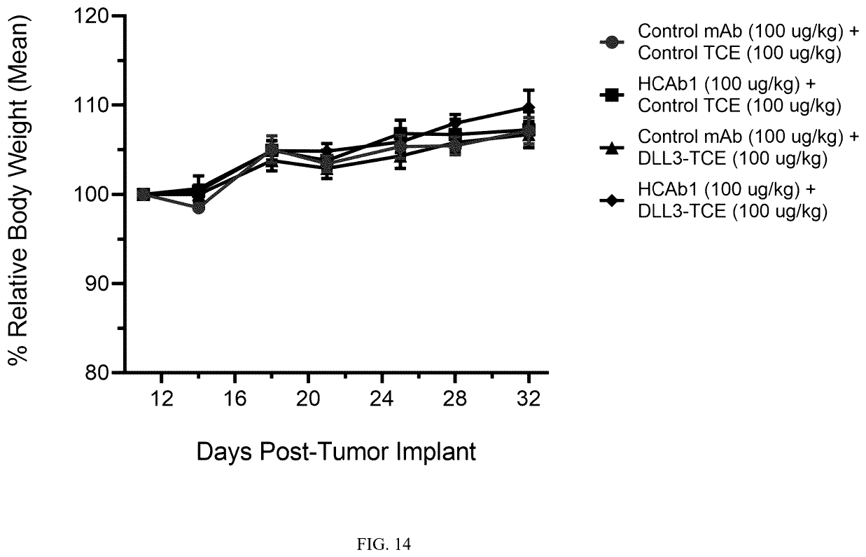

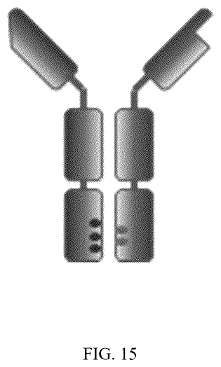

FIG. 13 depicts tumor volume between 11 and 32 days post-tumor implantation. The lowest tumor volumes were seen in mice treated with a combination of HCAb1 and tarlatamab (“DLL3-TCE”). Treatment with the combination therapy was well-tolerated, with mice showing similar changes in body weight across all treatment groups (FIG. 14).

### Example 7. Non-Human Primate (Nhp) Pharmacokinetic (Pk) Evaluation of HCAb1

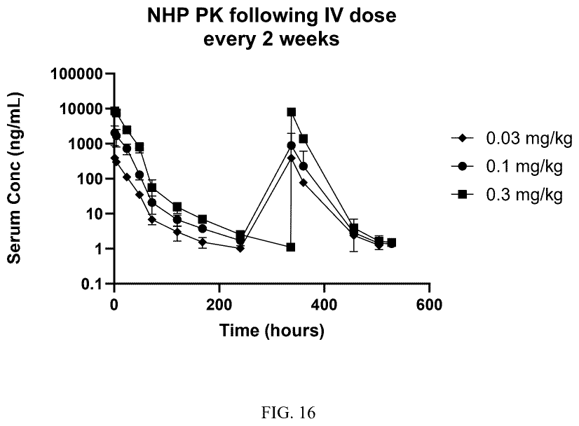

The pharmacokinetics (PK) of HcAb1 were evaluated in non-human primates (NHP) following two biweekly IV administrations at 0.03, 0.1, or 0.3 mg/kg (Charles River Laboratory, Reno, NV). Serum samples were collected at selected timepoints over the course of 28 days for PK analysis using an electrochemiluminescence method with anti-idiotypic monoclonal antibodies. The exposure increased in a dose dependent manner (FIG. 16).

### Example 8. Manufacturability and Stability of Additional Heavy Chain Antibodies Sharing VH Regions with HCAb1 And HCAb2

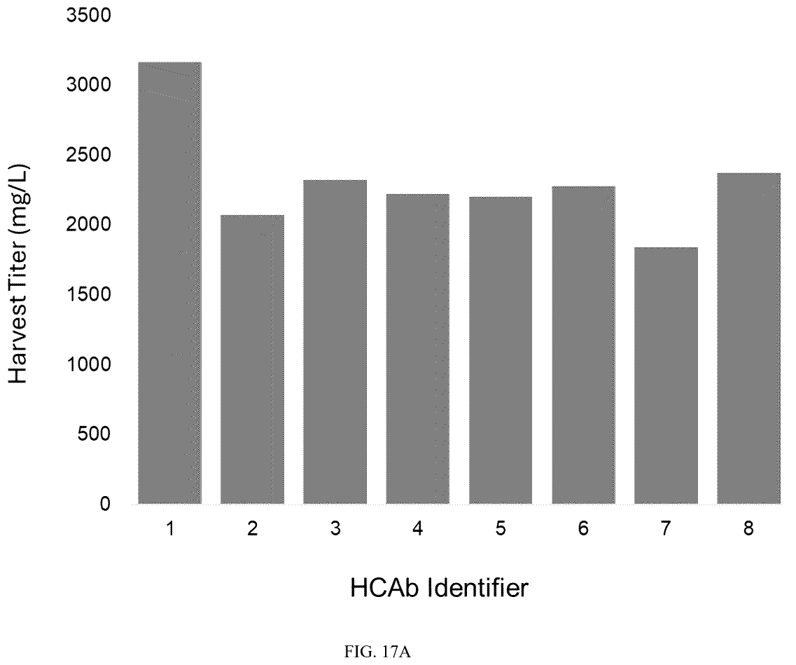

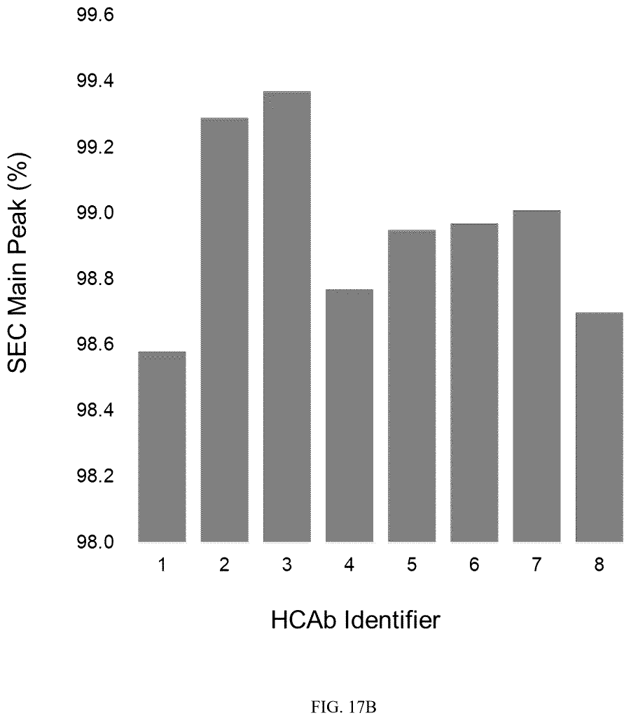

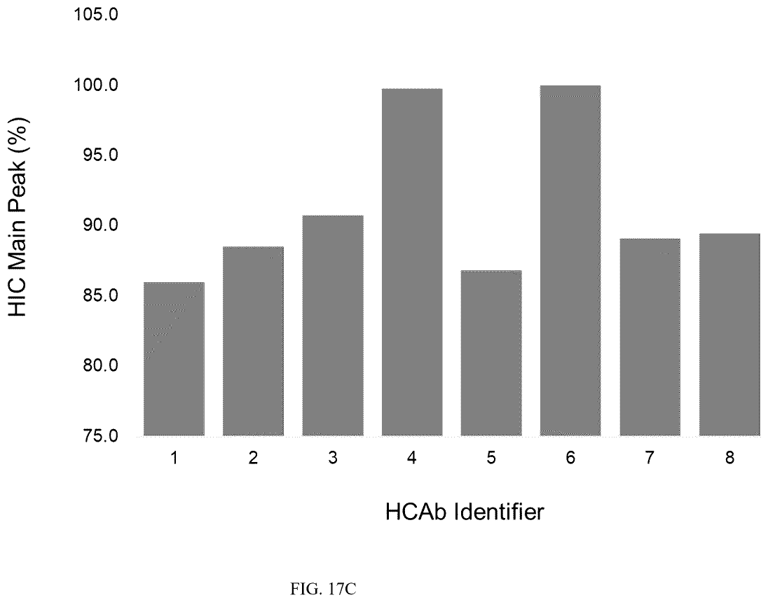

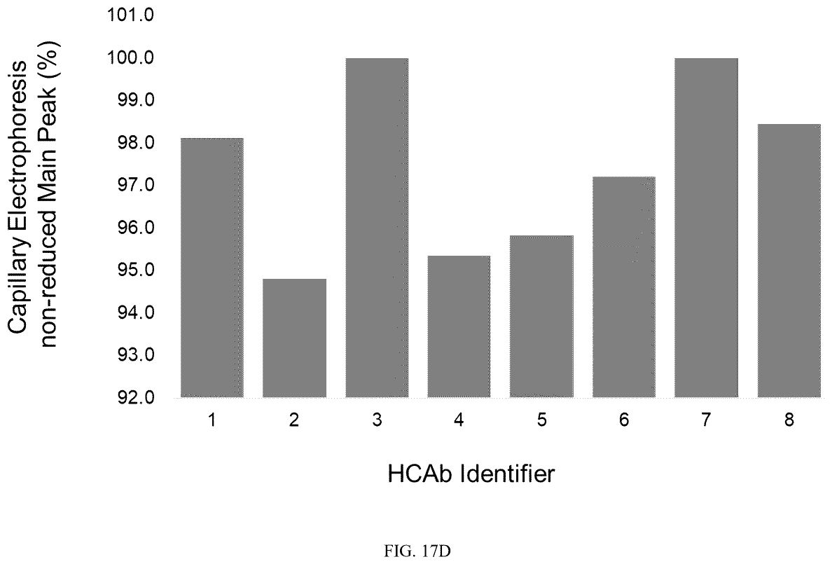

Six additional heavy chain antibodies (HCAb3, HCAb4, HCAb5, HCAb6, HCAb7, and HCAb8) with the same VH regions as HCAb1 and HCAb2 were designed by exchanging structural features related to the hinge/linker region and Fc domain (GGGG linker; effector function silencing mutations (SEFL, FALA); heterodimerizing mutations (knob-into-hole, charge pair)) (TABLE 27; TABLE 28). The manufacturability of each of the heavy chain antibodies (HCAb1, HCAb2, HCAb3, HCAb4, HCAb5, HCAb6, HCAb7, and HCAb8) was assessed by comparing Harvest Titer, % SEC Main Peak, % HIC Main Peak, and % Capillary Electrophoresis non-reduced Main Peak. Size exclusion chromatography (SEC) was performed using a Waters™ BEH200 SEC column (1.7 μm particle size, 4.6×300 mm) with a 100 mM Na Phosphate, 250 mM NaCl, pH 6.8 mobile phase, an injection volume of 1.23 μL, a flow rate of 0.4 mL/min, an injection amount of 6 μg, a run time of 12 minutes, and detection by UV at 220 nm. The assessed manufacturability attributes were found to be comparable for all eight molecules and indicative of manufacturability as therapeutics (FIGS. 17A-17D).

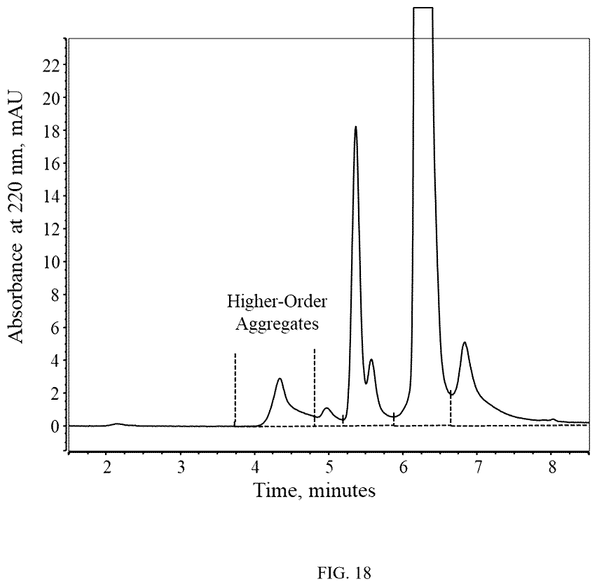

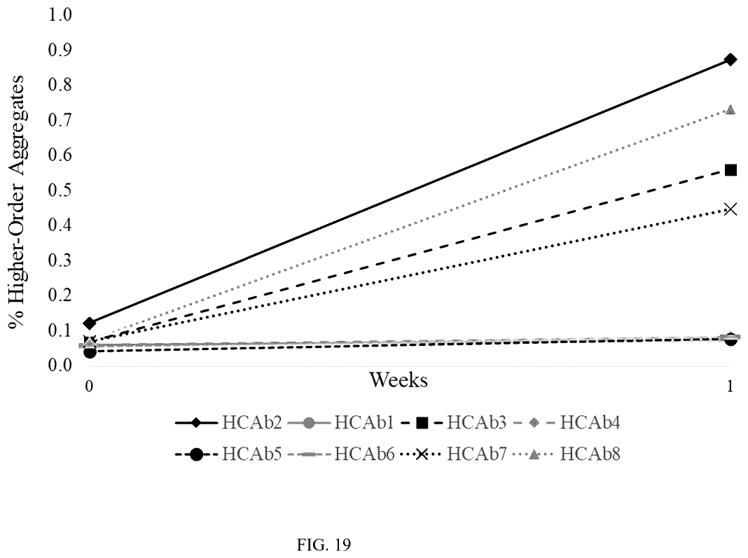

The stability of each of HCAb1, HCAb2, HCAb3, HCAb4, HCAb5, HCAb6, HCAb7, and HCAb8 at 40° C. over the course of one week was compared by SEC analysis. In brief, higher-order aggregate percentages were measured by SEC over the course of one week of incubation at 40° C. for each heavy chain antibody at a concentration of 10 mg/mL in a formulation containing 10 mM sodium acetate and 9% sucrose at pH 5.2 (FIG. 18). The heavy chain antibodies showed similar levels of higher-order aggregates, with small changes observed over the course of one week at 40° C. (FIG. 19). Levels of higher-order aggregates for all constructs were similar at time 0. After one week at 40° C., the levels remained approximately the same for HCAb1, HCAb4, HCAb5, and HCAb6, with increases observed for HCAb2, HCAb3, HCAb7, and HCAb8, all of which include an IgG4 hinge region and an IgG4 Fc region with FALA silencing mutations.

TABLE 27
Selected Structural Features of Evaluated Heavy Chain Antibodies

| | | | IgG1 | IgG4 | | |
|---|---|---|---|---|---|---|
| | | | hinge, | hinge, | Knob | |
| Construct | | | IgG1 Fc, | IgG4 Fc, | into | Charge |
| Name | Description | GGGG | and SEFL | and FALA | Hole | Pair |
| HCAb1 | HCAb1 | + | + | − | − | + |
| HCAb2 | HCAb2 | − | − | + | + | − |
| HCAb3 | HCAb2, but with charge | − | − | + | − | + |
| | pair Fc mutations instead | | | | | |
| | of knob-into-hole Fc | | | | | |
| | mutations | | | | | |
| HCAb4 | HCAb1, but with knob- | + | + | − | + | − |
| | into-hole Fc mutations | | | | | |
| | instead of charge pair Fc | | | | | |
| | mutations | | | | | |
| HCAb5 | HCAb1, but without | − | + | − | − | + |
| | GGGG | | | | | |
| HCAb6 | HCAb1, but without | − | + | − | + | − |
| | GGGG and with knob- | | | | | |
| | into-hole Fc mutations | | | | | |
| HCAb7 | HCAb2, but with GGGG | + | − | + | − | + |
| | and with charge pair Fc | | | | | |
| | mutations | | | | | |
| HCAb8 | HCAb2, but with GGGG | + | − | + | + | − |

TABLE 28
Amino Acid Sequences for HCAb3, HCAb4, HCAb5, HCAb6, HCAb7, and HCAb8

| Description | Amino Acid Sequences |
|---|---|
| HCAb3 | QVQLVESGGGLVKPGGSLRLSCAASGFTFSDYYMSWIRQAPGKGLEWV |
| | SYISSSGSTIYYADSVKGRFTISRDNAKNSLYLQMNSLRAEDTAVYYCA |
| | RGDAVSITGDYRGQGTLVTVSSESKYGPPCPPCPAPEAAGGPSVFLFPPK |
| | PKDTLMISRTPEVTCVVVDVSQEDPEVQFNWYVDGVEVHNAKTKPREE |
| | QFNSTYRVVSVLTVLHQDWLNGKEYKCKVSNKGLPSSIEKTISKAKGQ |
| | PREPQVYTLPPSQKEMTKNQVSLTCLVKGFYPSDIAVEWESNGQPENN |
| | YKTTPPVLKSDGSFFLYSRLTVDKSRWQEGNVFSCSVMHEALHNHYTQ |
| | KSLSLSLGK (SEQ ID NO: 105) |
| | QVQLQESSPGLVKPSETLSLTCTVSGGSISSSNWWSWVRQPPGK |
| | GLEWIGEISHSGSTNYNPSLKSRVTISVDKSKNQFSLRLSSVTAAD |
| | TAVYFCGRGSWELTDAFDIRGQGTLVTVSSESKYGPPCPPCPAPE |
| | AAGGPSVFLFPPKPKDTLMISRTPEVTCVVVDVSQEDPEVQFNW |
| | YVDGVEVHNAKTKPREEQFNSTYRVVSVLTVLHQDWLNGKEY |
| | KCKVSNKGLPSSIEKTISKAKGQPREPQVYTLPPSQEEMTKNQVS |
| | LTCLVKGFYPSDIAVEWESNGQPENNYDTTPPVLDSDGSFFLYSD |
| | LTVDKSRWQEGNVFSCSVMHEALHNHYTQDSLSLSLGK (SEQ |
| | ID NO: 106) |
| HCAb4 | QVQLVESGGGLVKPGGSLRLSCAASGFTFSDYYMSWIRQAPGKGLEWV |
| | SYISSSGSTIYYADSVKGRFTISRDNAKNSLYLQMNSLRAEDTAVYYCA |
| | RGDAVSITGDYRGQGTLVTVSSGGGGDKTHTCPPCPAPELLGGPSVFLF |
| | PPKPKDTLMISRTPEVTCVVVDVSHEDPEVKFNWYVDGVEVHNAKTKP |
| | CEEQYGSTYRCVSVLTVLHQDWLNGKEYKCKVSNKALPAPIEKTISKA |
| | KGQPREPQVYTLPPSREEMTKNQVSLWCLVKGFYPSDIAVEWESNGQP |
| | ENNYKTTPPVLDSDGSFFLYSKLTVDKSRWQQGNVFSCSVMHEALHNH |
| | YTQKSLSLSPGK (SEQ ID NO: 107) |
| | QVQLQESSPGLVKPSETLSLTCTVSGGSISSSNWWSWVRQPPGK |
| | GLEWIGEISHSGSTNYNPSLKSRVTISVDKSKNQFSLRLSSVTAAD |
| | TAVYFCGRGSWELTDAFDIRGQGTLVTVSSGGGGDKTHTCPPCP |
| | APELLGGPSVFLFPPKPKDTLMISRTPEVTCVVVDVSHEDPEVKF |
| | NWYVDGVEVHNAKTKPCEEQYGSTYRCVSVLTVLHQDWLNGK |
| | EYKCKVSNKALPAPIEKTISKAKGQPREPQVYTLPPSREEMTKNQ |
| | VSLSCAVKGFYPSDIAVEWESNGQPENNYKTTPPVLDSDGSFFLV |
| | SKLTVDKSRWQQGNVFSCSVMHEALHNHYTQKSLSLSPGK |
| | (SEQ ID NO: 108) |
| HCAb5 | QVQLVESGGGLVKPGGSLRLSCAASGFTFSDYYMSWIRQAPGKGLEWV |
| | SYISSSGSTIYYADSVKGRFTISRDNAKNSLYLQMNSLRAEDTAVYYCA |
| | RGDAVSITGDYRGQGTLVTVSSDKTHTCPPCPAPELLGGPSVFLFPPKPK |
| | DTLMISRTPEVTCVVVDVSHEDPEVKFNWYVDGVEVHNAKTKPCEEQ |
| | YGSTYRCVSVLTVLHQDWLNGKEYKCKVSNKALPAPIEKTISKAKGQP |
| | REPQVYTLPPSRKEMTKNQVSLTCLVKGFYPSDIAVEWESNGQPENNY |
| | KTTPPVLKSDGSFFLYSKLTVDKSRWQQGNVFSCSVMHEALHNHYTQK |
| | SLSLSPGK (SEQ ID NO: 109) |
| | QVQLQESSPGLVKPSETLSLTCTVSGGSISSSNWWSWVRQPPGKGLEWI |
| | GEISHSGSTNYNPSLKSRVTISVDKSKNQFSLRLSSVTAADTAVYFCGRG |
| | SWELTDAFDIRGQGTLVTVSSDKTHTCPPCPAPELLGGPSVFLFPPKPKD |
| | TLMISRTPEVTCVVVDVSHEDPEVKFNWYVDGVEVHNAKTKPCEEQY |
| | GSTYRCVSVLTVLHQDWLNGKEYKCKVSNKALPAPIEKTISKAKGQPR |
| | EPQVYTLPPSREEMTKNQVSLTCLVKGFYPSDIAVEWESNGQPENNYDT |
| | TPPVLDSDGSFFLYSDLTVDKSRWQQGNVFSCSVMHEALHNHYTQDSL |
| | SLSPGK (SEQ ID NO: 110) |
| HCAb6 | QVQLVESGGGLVKPGGSLRLSCAASGFTFSDYYMSWIRQAPGKGLEWV |
| | SYISSSGSTIYYADSVKGRFTISRDNAKNSLYLQMNSLRAEDTAVYYCA |
| | RGDAVSITGDYRGQGTLVTVSSDKTHTCPPCPAPELLGGPSVFLFPPKPK |
| | DTLMISRTPEVTCVVVDVSHEDPEVKFNWYVDGVEVHNAKTKPCEEQ |
| | YGSTYRCVSVLTVLHQDWLNGKEYKCKVSNKALPAPIEKTISKAKGQP |
| | REPQVYTLPPSREEMTKNQVSLWCLVKGFYPSDIAVEWESNGQPENNY |
| | KTTPPVLDSDGSFFLYSKLTVDKSRWQQGNVFSCSVMHEALHNHYTQK |
| | SLSLSPGK (SEQ ID NO: 111) |
| | QVQLQESSPGLVKPSETLSLTCTVSGGSISSSNWWSWVRQPPGKGLEWI |
| | GEISHSGSTNYNPSLKSRVTISVDKSKNQFSLRLSSVTAADTAVYFCGRG |
| | SWELTDAFDIRGQGTLVTVSSDKTHTCPPCPAPELLGGPSVFLFPPKPKD |
| | TLMISRTPEVTCVVVDVSHEDPEVKFNWYVDGVEVHNAKTKPCEEQY |
| | GSTYRCVSVLTVLHQDWLNGKEYKCKVSNKALPAPIEKTISKAKGQPR |
| | EPQVYTLPPSREEMTKNQVSLSCAVKGFYPSDIAVEWESNGQPENNYK |
| | TTPPVLDSDGSFFLVSKLTVDKSRWQQGNVFSCSVMHEALHNHYTQKS |
| | LSLSPGK (SEQ ID NO: 112) |
| HCAb7 | QVQLVESGGGLVKPGGSLRLSCAASGFTFSDYYMSWIRQAPGKG |
| | LEWVSYISSSGSTIYYADSVKGRFTISRDNAKNSLYLQMNSLRAE |
| | DTAVYYCARGDAVSITGDYRGQGTLVTVSSGGGGESKYGPPCPP |
| | CPAPEAAGGPSVFLFPPKPKDTLMISRTPEVTCVVVDVSQEDPEV |
| | QFNWYVDGVEVHNAKTKPREEQFNSTYRVVSVLTVLHQDWLN |
| | GKEYKCKVSNKGLPSSIEKTISKAKGQPREPQVYTLPPSQKEMTK |
| | NQVSLTCLVKGFYPSDIAVEWESNGQPENNYKTTPPVLKSDGSFF |
| | LYSRLTVDKSRWQEGNVFSCSVMHEALHNHYTQKSLSLSLGK |
| | (SEQ ID NO: 113) |
| | QVQLQESSPGLVKPSETLSLTCTVSGGSISSSNWWSWVRQPPGK |
| | GLEWIGEISHSGSTNYNPSLKSRVTISVDKSKNQFSLRLSSVTAAD |
| | TAVYFCGRGSWELTDAFDIRGQGTLVTVSSGGGGESKYGPPCPP |
| | CPAPEAAGGPSVFLFPPKPKDTLMISRTPEVTCVVVDVSQEDPEV |
| | QFNWYVDGVEVHNAKTKPREEQFNSTYRVVSVLTVLHQDWLN |
| | GKEYKCKVSNKGLPSSIEKTISKAKGQPREPQVYTLPPSQEEMTK |
| | NQVSLTCLVKGFYPSDIAVEWESNGQPENNYDTTPPVLDSDGSFF |
| | LYSDLTVDKSRWQEGNVFSCSVMHEALHNHYTQDSLSLSLGK |
| | (SEQ ID NO: 114) |
| HCAb8 | QVQLVESGGGLVKPGGSLRLSCAASGFTFSDYYMSWIRQAPGKG |
| | LEWVSYISSSGSTIYYADSVKGRFTISRDNAKNSLYLQMNSLRAE |
| | DTAVYYCARGDAVSITGDYRGQGTLVTVSSGGGGESKYGPPCPP |
| | CPAPEAAGGPSVFLFPPKPKDTLMISRTPEVTCVVVDVSQEDPEV |
| | QFNWYVDGVEVHNAKTKPREEQFNSTYRVVSVLTVLHQDWLN |
| | GKEYKCKVSNKGLPSSIEKTISKAKGQPREPQVYTLPPSQEEMTK |
| | NQVSLWCLVKGFYPSDIAVEWESNGQPENNYKTTPPVLDSDGSF |
| | FLYSRLTVDKSRWQEGNVFSCSVMHEALHNHYTQKSLSLSLGK |
| | (SEQ ID NO: 115) |
| | QVQLQESSPGLVKPSETLSLTCTVSGGSISSSNWWSWVRQPPGK |
| | GLEWIGEISHSGSTNYNPSLKSRVTISVDKSKNQFSLRLSSVTAAD |
| | TAVYFCGRGSWELTDAFDIRGQGTLVTVSSGGGGESKYGPPCPP |
| | CPAPEAAGGPSVFLFPPKPKDTLMISRTPEVTCVVVDVSQEDPEV |
| | QFNWYVDGVEVHNAKTKPREEQFNSTYRVVSVLTVLHQDWLN |
| | GKEYKCKVSNKGLPSSIEKTISKAKGQPREPQVYTLPPSQEEMTK |
| | NQVSLSCAVKGFYPSDIAVEWESNGQPENNYKTTPPVLDSDGSF |
| | FLVSRLTVDKSRWQEGNVFSCSVMHEALHNHYTQKSLSLSLGK |
| | (SEQ ID NO: 116) |

What is claimed is:

1. A heavy-chain antibody comprising:
a first heavy chain variable (VH) region that binds to IL2Rβ comprising:
(a) a VH complementarity determining region one (CDR1) comprising the amino acid sequence:
(SEQ ID NO: 26)
G G S I S S S X1 W,
wherein X1 is D or N;
(b) a VH CDR2 comprising the amino acid sequence:
(SEQ ID NO: 27)
I X2 H S G S T,
wherein X2 is D or S; and
(c) a VH CDR3 comprising the amino acid sequence:
(SEQ ID NO: 28)
X3 R G X4 W E L X5 D A F D I,
wherein X3 is G or A; X4 is S or Q; and X5 is S or T;
a second heavy chain variable region that binds to IL2RG comprising:
(a) a VH CDR1 comprising the amino acid sequence:
(SEQ ID NO: 32)
G F X1 X2 X3 X4 Y Y,
wherein X1 is T or I; X2 is For V; X3 is S, N, or G; and X4 is D or N;
(b) a VH CDR2 comprising the amino acid sequence:
(SEQ ID NO: 33)
I S X5 S G X6 X7 I,
wherein X5 is S or N; X6 is D, S, G, or N; and X7 is T or I; and
(c) a VH CDR3 comprising the amino acid sequence ARGDAVSITGDY (SEQ ID NO: 20); and
an Fc region comprising an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering.

2. The heavy-chain antibody of claim 1, wherein the Fc region comprises an amino acid sequence that is at least 95% identical to the amino acid sequence of SEQ ID NO: 34.

3. The heavy-chain antibody of claim 1, wherein the Fc region comprises:
a first polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering; and
a second polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

4. The heavy-chain antibody of claim 3, wherein the first polypeptide chain comprises an amino acid sequence that is at least 95% identical to the amino acid sequence of SEQ ID NO: 34, and the second polypeptide chain comprises an amino acid sequence that is at least 95% identical to the amino acid sequence of SEQ ID NO: 34.

5. The heavy-chain antibody of claim 3 or claim 4, wherein the first polypeptide chain comprises the amino acid sequence of SEQ ID NO: 35, and the second polypeptide chain comprises the amino acid sequence of SEQ ID NO: 36.

6. The heavy-chain antibody of any one of claims 1-5, wherein the first heavy chain variable region is connected to the Fc region by a first peptide linker, and the second heavy chain variable region is connected to the Fc region by a second peptide linker.

7. The heavy-chain antibody of claim 6, wherein the first peptide linker and the second peptide linker are independently poly-Gly linkers.

8. The heavy-chain antibody of claim 6 or claim 7, wherein the first peptide linker and the second peptide linker both comprise the amino acid sequence of GGGG (SEQ ID NO: 37).

9. A heavy-chain antibody comprising:
a first heavy chain variable (VH) region that binds to IL2Rβ comprising:
(a) a VH complementarity determining region one (CDR1) comprising the amino acid sequence:
(SEQ ID NO: 26)
G G S I S S S X1 W,
wherein X1 is D or N;
(b) a VH CDR2 comprising the amino acid sequence:
(SEQ ID NO: 27)
I X2 H S G S T,
wherein X2 is D or S; and
(c) a VH CDR3 comprising the amino acid sequence:
(SEQ ID NO: 28)
X3 R G X4 W E L X5 D A F D I,
wherein X3 is G or A; X4 is S or Q; and X5 is S or T;
a second heavy chain variable region that binds to IL2RG comprising:
(a) a VH CDR1 comprising the amino acid sequence:
(SEQ ID NO: 32)
G F X1 X2 X3 X4 Y Y,
wherein X1 is T or I; X2 is For V; X3 is S, N, or G; and X4 is D or N;
(b) a VH CDR2 comprising the amino acid sequence:
(SEQ ID NO: 33)
I S X5 S G X6 X7 I,
wherein X5 is S or N; X6 is D, S, G, or N; and X7 is T or I; and
(c) a VH CDR3 comprising the amino acid sequence ARGDAVSITGDY (SEQ ID NO: 20); and
an Fc region, wherein the first heavy chain variable region is connected to the Fc region by a first peptide linker comprising at least four amino acids, and the second heavy chain variable region is connected to the Fc region by a second peptide linker comprising at least four amino acids.

10. The heavy-chain antibody of claim 9, wherein the first peptide linker and the second peptide linker are both flexible linkers.

11. The heavy-chain antibody of claim 9 or claim 10, wherein the first peptide linker and the second peptide linker are independently poly-Gly linkers.

12. The heavy-chain antibody of any one of claims 9-11, wherein the first peptide linker and the second peptide linker both comprise the amino acid sequence of GGGG (SEQ ID NO: 37).

13. The heavy-chain antibody of any one of claims 9-12, wherein the Fc region comprises an amino acid sequence that is at least 90% identical to the amino acid sequence of SEQ ID NO: 34, wherein the amino acid sequence comprises a glycine at position 297, a cysteine at position 292, and a cysteine at position 302, all according to EU numbering.

14. The heavy-chain antibody of any one of claims 9-13, wherein the Fc region comprises an amino acid sequence that is at least 95% identical to the amino acid sequence of SEQ ID NO: 34.

15. The heavy-chain antibody of any one of claims 9-12, wherein the Fc region comprises:
a first polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, a lysine at position 356, and a lysine at position 399, all according to EU numbering; and
a second polypeptide chain comprising an amino acid sequence that is at least 90% identical to the amino acid sequence of SEQ ID NO: 34, wherein the amino acid sequence comprises a glycine at position 297, a cysteine at position 292, a cysteine at position 302, an aspartic acid at position 392, an aspartic acid at position 409, and an aspartic acid at position 439, all according to EU numbering.

16. The heavy-chain antibody of claim 15, wherein the first polypeptide chain comprises an amino acid sequence that is at least 95% identical to the amino acid sequence of SEQ ID NO: 34, and the second polypeptide chain comprises an amino acid sequence that is at least 95% identical to the amino acid sequence of SEQ ID NO: 34.

17. The heavy-chain antibody of claim 15 or claim 16, wherein the first polypeptide chain comprises the amino acid sequence of SEQ ID NO: 35, and the second polypeptide chain comprises the amino acid sequence of SEQ ID NO: 36.

18. The heavy-chain antibody of claim 9, wherein:
the Fc region comprises a first polypeptide chain comprising the amino acid sequence of SEQ ID NO: 35 and a second polypeptide chain comprising the amino acid sequence of SEQ ID NO: 36; and
the first peptide linker and the second peptide linker are independently poly-Gly linkers.

19. The heavy-chain antibody of claim 9 or claim 18, wherein:
the Fc region comprises a first polypeptide chain comprising the amino acid sequence of SEQ ID NO: 35 and a second polypeptide chain comprising the amino acid sequence of SEQ ID NO: 36; and
the first peptide linker and the second peptide linker both comprise the amino acid sequence of GGGG (SEQ ID NO: 37).

20. The heavy-chain antibody of any one of claims 1-19, wherein the VH CDR1, the VH CDR2, and the VH CDR3 of the first VH region comprise the amino acid sequences of SEQ ID NO: 1 or SEQ ID NO: 2; SEQ ID NO: 4 or SEQ ID NO: 5; and SEQ ID NO: 7, SEQ ID NO: 8, or SEQ ID NO: 9, respectively.

21. The heavy-chain antibody of any one of claims 1-20, wherein the VH CDR1, the VH CDR2, and the VH CDR3 of the first VH region comprise the amino acid sequences of:
(a) SEQ ID NO: 1, SEQ ID NO: 4, and SEQ ID NO: 7, respectively; or
(b) SEQ ID NO: 1, SEQ ID NO: 4, and SEQ ID NO: 8, respectively; or
(c) SEQ ID NO: 2, SEQ ID NO: 5, and SEQ ID NO: 9, respectively.

22. The heavy-chain antibody of any one of claims 1-21, wherein the first VH region comprises an amino acid sequence that is at least 95% identical to any one of SEQ ID NOs: 11-13.

23. The heavy-chain antibody of any one of claims 1-22, wherein the first VH region comprises an amino acid sequence selected from SEQ ID NOs: 11-13.

24. The heavy-chain antibody of any one of claims 1-23, wherein the VH CDR1, the VH CDR2, and the VH CDR3 of the second VH region comprise the amino acid sequences of SEQ ID NO: 15 or SEQ ID NO: 16; SEQ ID NO: 17 or SEQ ID NO: 18; and SEQ ID NO: 20, respectively.

25. The heavy-chain antibody of any one of claims 1-24, wherein the VH CDR1, the VH CDR2, and the VH CDR3 of the second VH region comprise the amino acid sequences of:
(a) SEQ ID NO: 15, SEQ ID NO: 17, and SEQ ID NO: 20, respectively; or
(b) SEQ ID NO: 15, SEQ ID NO: 18, and SEQ ID NO: 20, respectively; or
(c) SEQ ID NO: 16, SEQ ID NO: 18, and SEQ ID NO: 20, respectively.

26. The heavy-chain antibody of any one of claims 1-25, wherein the second VH region comprises an amino acid sequence that is at least 95% identical to any one of SEQ ID NOs: 22-24.

27. The heavy-chain antibody of any one of claims 1-26, wherein the second VH region comprises an amino acid sequence selected from SEQ ID NOs: 22-24.

28. The heavy-chain antibody of any one of claims 1-27, wherein the first VH region comprises the amino acid sequence of SEQ ID NO: 13, and the second VH region comprises the amino acid sequence of SEQ ID NO: 23.

29. A heavy-chain antibody comprising:
a first heavy chain that binds to IL2Rβ comprising the amino acid sequence of SEQ ID NO: 38; and
a second heavy chain that binds to IL2RG comprising the amino acid sequence of SEQ ID NO: 39.

30. A pharmaceutical composition comprising:
a heavy-chain antibody of any one of claims 1-29; and
a pharmaceutically acceptable excipient.

31. The pharmaceutical composition of claim 30, wherein the pharmaceutical composition is adapted for intravenous or subcutaneous administration.

32. A method of treating cancer in a subject in need thereof, comprising administering to the subject a heavy-chain antibody of any one of claims 1-29 or a pharmaceutical composition of claim 30 or claim 31.

33. A method of treating cancer in a subject in need thereof, comprising administering to the subject a heavy-chain antibody of any one of claims 1-29 or a pharmaceutical composition of claim 30 or claim 31 in combination with a T-cell redirecting therapy.

34. A method of enhancing an anti-cancer effect associated with administration of a T-cell redirecting therapy in a subject diagnosed with cancer, comprising administering to the subject a heavy-chain only antibody of any one of claims 1-29 or a pharmaceutical composition of claim 30 or claim 31 in combination with the T-cell redirecting therapy.

35. The method of claim 33 or claim 34, wherein the T-cell redirecting therapy is a bispecific T-cell engaging molecule.

36. The method of claim 35, wherein the bispecific T-cell engaging molecule is tarlatamab.

37. A method of treating a DLL3-expressing cancer in a subject in need thereof, comprising administering to the subject an IL-2-based therapy in combination with a T-cell redirecting therapy that binds to DLL3.

38. The method of claim 37, wherein the DLL3-expressing cancer is a neuroendocrine cancer.

39. The method of claim 37 or claim 38, wherein the T-cell redirecting therapy that binds to DLL3 is tarlatamab.

40. The method of any one of claims 37-39, wherein the IL-2-based therapy preferentially binds to the heterodimeric receptor composed only of IL 2Rβ and IL 2Rγ over the trimeric IL-2Raβγ form of the IL-2 receptor.


SEQUENCE LISTING (116 sequences)

SEQ ID NO: 1 - protein, 9 aa, synthetic construct
GGSISSSDW

SEQ ID NO: 2 - protein, 9 aa, synthetic construct
GGSISSSNW

SEQ ID NO: 3 - protein, 8 aa, synthetic construct
GFTFSSYG

SEQ ID NO: 4 - protein, 7 aa, synthetic construct
IDHSGST

SEQ ID NO: 5 - protein, 7 aa, synthetic construct
ISHSGST

SEQ ID NO: 6 - protein, 8 aa, synthetic construct
ISYDGSNK

SEQ ID NO: 7 - protein, 13 aa, synthetic construct
GRGSWELSDAFDI

SEQ ID NO: 8 - protein, 13 aa, synthetic construct
ARGSWELTDAFDI

SEQ ID NO: 9 - protein, 13 aa, synthetic construct
GRGSWELTDAFDI

SEQ ID NO: 10 - protein, 19 aa, synthetic construct
ARDLDYDVLTGDPVGGFDI

SEQ ID NO: 11 - protein, 120 aa, synthetic construct
QVQLQESGPGLVKPSGTLSLTCAVSGGSISSSDWWSWVRQPPGKGLEWIGEIDHSGSTNYNPSLMSRVTISVDKSKNQFSLKLSSVTAADTAVYFCGRGSWELSDAFDIRGQGTLVTVSS

SEQ ID NO: 12 - protein, 120 aa, synthetic construct
QVQLQESGPGLVKSSETLSLTCTVSGGSISSSDWWSWVRQPPGKGLEWIGEIDHSGSTNYNPSLMSRVTISVDKSKNQFSLKLSSVTAADTAVYFCARGSWELTDAFDIRGQGTLVTVSS

SEQ ID NO: 13 - protein, 120 aa, synthetic construct
QVQLQESSPGLVKPSETLSLTCTVSGGSISSSNWWSWVRQPPGKGLEWIGEISHSGSTNYNPSLKSRVTISVDKSKNQFSLRLSSVTAADTAVYFCGRGSWELTDAFDIRGQGTLVTVSS

SEQ ID NO: 14 - protein, 126 aa, synthetic construct
QVQLVESGGGVVQPGRSLRLSCAASGFTFSSYGMHWVRQAPGKEREWVAVISYDGSNKYYTDSVKGRFTISRDNSKNTLYLEMNSLRAEDTAVYYCARDLDYDVLTGDPVGGFDIWGQGTLVTVSS

SEQ ID NO: 15 - protein, 8 aa, synthetic construct
GFTFSDYY

SEQ ID NO: 16 - protein, 8 aa, synthetic construct
GFTFNDYY

SEQ ID NO: 17 - protein, 8 aa, synthetic construct
ISSSGDTI

SEQ ID NO: 18 - protein, 8 aa, synthetic construct
ISSSGSTI

SEQ ID NO: 19 - protein, 8 aa, synthetic construct
ISSSGTTT

SEQ ID NO: 20 - protein, 12 aa, synthetic construct
ARGDAVSITGDY

SEQ ID NO: 21 - protein, 12 aa, synthetic construct
ARGAAVAPGFDS

SEQ ID NO: 22 - protein, 119 aa, synthetic construct
QVQLVESGGGLVKPGGSLRLSCAASGFTFSDYYMSWIRQAPGKGLEWVSSISSSGDTIYYADSVQGRFTLSRDNAENSLFLQMNSLRAEDTAVYYCARGDAVSITGDYRGQGTLVTVSS

SEQ ID NO: 23 - protein, 119 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSS

SEQ ID NO: 24 - protein, 119 aa, synthetic construct
QVQLVESGGGLVKPGGSLRLSCAASGFTFNDYYMSWIRQAPGKGLEWVSHISSSGSTIYYADSVKGRFTVSRDNANNSLYLQMHSLRAEDTAVYYCARGDAVSITGDYRGQGTLVTVSS

SEQ ID NO: 25 - protein, 119 aa, synthetic construct
QVQLVESGGDLVKPGGSLRLSCAASGFTFSDYYMSWLRQAPGKELEWVSHISSSGTTTYYADSVEGRFTITRDNAKNSLYLQMNSLRAEDTAVYYCARGAAVAPGFDSRGQGTLVTVSS

SEQ ID NO: 26 - protein, 9 aa, synthetic construct
GGSISSSBW

SEQ ID NO: 27 - protein, 7 aa, synthetic construct
  SITE 2: note = D or S
IXHSGST

SEQ ID NO: 28 - protein, 13 aa, synthetic construct
  SITE 1: note = G or A
  SITE 4: note = S or Q
  SITE 8: note = S or T
XRGXWELXDAFDI

SEQ ID NO: 29 - protein, 8 aa, synthetic construct
  SITE 6: note = S or T
GFTFSXYG

SEQ ID NO: 30 - protein, 8 aa, synthetic construct
  SITE 8: note = K or R
ISYDGSNX

SEQ ID NO: 31 - protein, 19 aa, synthetic construct
  SITE 8: note = V or I
ARDLDYDXLTGDPVGGFDI

SEQ ID NO: 32 - protein, 8 aa, synthetic construct
  SITE 3: note = T or I
  SITE 4: note = F or V
  SITE 5: note = S, N, or G
GFXXXBYY

SEQ ID NO: 33 - protein, 8 aa, synthetic construct
  SITE 3: note = S or N
  SITE 6: note = D, S, G, or N
  SITE 7: note = T or I
ISXSGXXI

SEQ ID NO: 34 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 227 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 36 - protein, 227 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQDSLSLSPGK

SEQ ID NO: 37 - protein, 4 aa, synthetic construct
GGGG

SEQ ID NO: 38 - protein, 351 aa, synthetic construct
QVQLQESSPGLVKPSETLSLTCTVSGGSISSSNWWSWVRQPPGKGLEWIGEISHSGSTNYNPSLKSRVTISVDKSKNQFSLRLSSVTAADTAVYFCGRGSWELTDAFDIRGQGTLVTVSSGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQDSLSLSPGK

SEQ ID NO: 39 - protein, 350 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSSGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 40 - protein, 5 aa, synthetic construct
  SITE 1..5: note = This sequence may encompass 1-5 'Gly Gly Gly Gly Ser' repeating units
GGGGS

SEQ ID NO: 41-42 - skipped (2 entries)

SEQ ID NO: 43 - protein, 5 aa, synthetic construct
GGGGS

SEQ ID NO: 44 - protein, 10 aa, synthetic construct
GGGGSGGGGS

SEQ ID NO: 45 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 46 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 47 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 48 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 49 - protein, 330 aa, Homo sapiens [sequence omitted]

SEQ ID NO: 50 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 13 aa, Homo sapiens
ESKYGPPCPSCPA

SEQ ID NO: 54 - protein, 13 aa, synthetic construct
ESKYGPPCPPCPA

SEQ ID NO: 55 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 56 - protein, 110 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 57 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 107 aa, synthetic construct
GQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 107 aa, synthetic construct
GQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 60 - protein, 349 aa, synthetic construct
QVQLQESSPGLVKPSETLSLTCTVSGGSISSSNWWSWVRQPPGKGLEWIGEISHSGSTNYNPSLKSRVTISVDKSKNQFSLRLSSVTAADTAVYFCGRGSWELTDAFDIRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 61 - protein, 348 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 5 aa, synthetic construct
SYYWS

SEQ ID NO: 63 - protein, 16 aa, synthetic construct
YVYYSGTTNYNPSLKS

SEQ ID NO: 64 - protein, 10 aa, synthetic construct
IAVTGFYFDY

SEQ ID NO: 65 - protein, 12 aa, synthetic construct
RASQRVNNNYLA

SEQ ID NO: 66 - protein, 7 aa, synthetic construct
GASSRAT

SEQ ID NO: 67 - protein, 9 aa, synthetic construct
QQYDRSPLT

SEQ ID NO: 68 - protein, 118 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 69 - protein, 108 aa, synthetic construct
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 70 - protein, 241 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 71 - protein, 5 aa, synthetic construct
KYAMN

SEQ ID NO: 72 - protein, 19 aa, synthetic construct
RIRSKYNNYATYYADSVKD

SEQ ID NO: 73 - protein, 14 aa, synthetic construct
HGNFGNSYISYWAY

SEQ ID NO: 74 - protein, 14 aa, synthetic construct
GSSTGAVTSGNYPN

SEQ ID NO: 75 - protein, 7 aa, synthetic construct
GTKFLAP

SEQ ID NO: 76 - protein, 9 aa, synthetic construct
VLWYSNRWV

SEQ ID NO: 77 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 78 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 79 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 80 - protein, 496 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 81 - protein, 982 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 5 aa, synthetic construct
  SITE 1..5: note = This sequence may encompass 1-5 'GGGGQ' repeating units
GGGGQ

SEQ ID NO: 83 - protein, 505 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLTLSCAASSSSVSLLSLAWYRQAPGKKRELVAGISDDGSIVYMDSVKGRFTISRDNAKNSVYLQMNSLRAEDTAVYYCYAYSWITRSPYWGQGTLVTVSSHHHHHH

SEQ ID NO: 84 - protein, 505 aa, synthetic construct
EVQLVESGGGLVQPGGSLTLSCAASSSSVSLLSLAWYRQAPGKKRELVAGISDDGSIVYMDSVKGRFTISRDNAKNSVYLQMNSLRAEDTAVYYCYAYSWITRSPYWGQGTLVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 85 - protein, 10 aa, synthetic construct
GYTFTSYYVH

SEQ ID NO: 86 - protein, 17 aa, synthetic construct
IINPGGGTTSYAQKFLG

SEQ ID NO: 87 - protein, 15 aa, synthetic construct
GEAVTGNYFYYGMDV

SEQ ID NO: 88 - protein, 11 aa, synthetic construct
RASQGISNYLV

SEQ ID NO: 89 - protein, 7 aa, synthetic construct
AVSSLYS

SEQ ID NO: 90 - protein, 9 aa, synthetic construct
LQHDSYPYT

SEQ ID NO: 91 - protein, 124 aa, synthetic construct
QVQLVQSGAEVKKPGASVKVSCKASGYTFTSYYVHWVRQAPGQGLEWMVIINPGGGTTSYAQKFLGRVTMTRDTSTNTVYMELKSLRSEDTAVYYCARGEAVTGNYFYYGMDVWGQGTTVTVSS

SEQ ID NO: 92 - protein, 107 aa, synthetic construct
DIQMTQSPSAMSASVGDRVTITCRASQGISNYLVWFQQKPGKAPKRLIYAVSSLYSGVPSRFSGSGSGTEFTLTISSLQPEDFATYYCLQHDSYPYTFGQGTKLEIK

SEQ ID NO: 93 - protein, 479 aa, synthetic construct
DIQMTQSPSAMSASVGDRVTITCRASQGISNYLVWFQQKPGKAPKRLIYAVSSLYSGVPSRFSGSGSGTEFTLTISSLQPEDFATYYCLQHDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTSYYVHWVRQAPGQGLEWMVIINPGGGTTSYAQKFLGRVTMTRDTSTNTVYMELKSLRSEDTAVYYCARGEAVTGNYFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 94 - protein, 705 aa, synthetic construct
DIQMTQSPSAMSASVGDRVTITCRASQGISNYLVWFQQKPGKAPKRLIYAVSSLYSGVPSRFSGSGSGTEFTLTISSLQPEDFATYYCLQHDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTSYYVHWVRQAPGQGLEWMVIINPGGGTTSYAQKFLGRVTMTRDTSTNTVYMELKSLRSEDTAVYYCARGEAVTGNYFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 10 aa, synthetic construct
GFTFNTYAMN

SEQ ID NO: 96 - protein, 19 aa, synthetic construct
RIRSKYNNYATYYADSVKD

SEQ ID NO: 97 - protein, 14 aa, synthetic construct
HGNFGNSYVSWFAY

SEQ ID NO: 98 - protein, 14 aa, synthetic construct
RSSTGAVTTSNYAN

SEQ ID NO: 99 - protein, 7 aa, synthetic construct
GTNKRAP

SEQ ID NO: 100 - protein, 9 aa, synthetic construct
ALWYSNLWV

SEQ ID NO: 101 - protein, 125 aa, synthetic construct
EVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSA

SEQ ID NO: 102 - protein, 109 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVL

SEQ ID NO: 103 - protein, 480 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVLGQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECSGGGGSEGKSSGSGSESKSTEGKSSGSGSESKSTGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKS

SEQ ID NO: 104 - protein, 707 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVLGQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECSGGGGSEGKSSGSGSESKSTEGKSSGSGSESKSTGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 105 - protein, 348 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLKSDGSFFLYSRLTVDKSRWQEGNVFSCSVMHEALHNHYTQKSLSLSLGK

SEQ ID NO: 106 - protein, 349 aa, synthetic construct
QVQLQESSPGLVKPSETLSLTCTVSGGSISSSNWWSWVRQPPGKGLEWIGEISHSGSTNYNPSLKSRVTISVDKSKNQFSLRLSSVTAADTAVYFCGRGSWELTDAFDIRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYDTTPPVLDSDGSFFLYSDLTVDKSRWQEGNVFSCSVMHEALHNHYTQDSLSLSLGK

SEQ ID NO: 107 - protein, 350 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSSGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 351 aa, synthetic construct
QVQLQESSPGLVKPSETLSLTCTVSGGSISSSNWWSWVRQPPGKGLEWIGEISHSGSTNYNPSLKSRVTISVDKSKNQFSLRLSSVTAADTAVYFCGRGSWELTDAFDIRGQGTLVTVSSGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 109 - protein, 346 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 110 - protein, 347 aa, synthetic construct
QVQLQESSPGLVKPSETLSLTCTVSGGSISSSNWWSWVRQPPGKGLEWIGEISHSGSTNYNPSLKSRVTISVDKSKNQFSLRLSSVTAADTAVYFCGRGSWELTDAFDIRGQGTLVTVSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQDSLSLSPGK

SEQ ID NO: 111 - protein, 346 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 347 aa, synthetic construct
QVQLQESSPGLVKPSETLSLTCTVSGGSISSSNWWSWVRQPPGKGLEWIGEISHSGSTNYNPSLKSRVTISVDKSKNQFSLRLSSVTAADTAVYFCGRGSWELTDAFDIRGQGTLVTVSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 113 - protein, 352 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSSGGGGESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLKSDGSFFLYSRLTVDKSRWQEGNVFSCSVMHEALHNHYTQKSLSLSLGK

SEQ ID NO: 114 - protein, 353 aa, synthetic construct
QVQLQESSPGLVKPSETLSLTCTVSGGSISSSNWWSWVRQPPGKGLEWIGEISHSGSTNYNPSLKSRVTISVDKSKNQFSLRLSSVTAADTAVYFCGRGSWELTDAFDIRGQGTLVTVSSGGGGESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYDTTPPVLDSDGSFFLYSDLTVDKSRWQEGNVFSCSVMHEALHNHYTQDSLSLSLGK

SEQ ID NO: 115 - protein, 352 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSSGGGGESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 353 aa, synthetic construct
QVQLQESSPGLVKPSETLSLTCTVSGGSISSSNWWSWVRQPPGKGLEWIGEISHSGSTNYNPSLKSRVTISVDKSKNQFSLRLSSVTAADTAVYFCGRGSWELTDAFDIRGQGTLVTVSSGGGGESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK